(19) United States
(12) Patent Application Publication
(10) Pub. No.: US 2006/0223131 A1
(43) Pub. Date: Oct. 5, 2006

(54) Protein arrays and methods of use thereof

(76) Inventors: Barry Schweitzer, Cheshire, CT (US); James A. Ball, Ledyard, CT (US); Paul F. Predki, Carlsbad, CA (US); Fang X. Zhou, New Haven, CT (US); Gregory A. Michaud, Clinton, CT (US)

Correspondence Address: JONES DAY, 222 EAST 41ST ST, NEW YORK, NY, 10017, US

(21) Appl. No.: 11/229,258

(22) Filed: Sep. 15, 2005

Related U.S. Application Data
(60) Provisional application No. 60/610,444, filed on Sep. 15, 2004.
(60) Provisional application No. 60/610,446, filed on Sep. 15, 2004.
(60) Provisional application No. 60/620,193, filed on Oct. 18, 2004.
(60) Provisional application No. 60/620,233, filed on Oct. 18, 2004.
(60) Provisional application No. 60/653,585, filed on Feb. 15, 2005.
(60) Provisional application No. 60/665,486, filed on Mar. 25, 2005.

(51) Int. Cl. G01N 33/53; G06Q 10/00; C12M 1/34

(52) U.S. Cl. 435/7.92; 435/287.2; 705/2

(57) ABSTRACT

The present invention provides human protein arrays that include at least 1000 human proteins. In another embodiment, the present invention provides a method for identifying a substrate of an enzyme, comprising contacting the enzyme with a positionally addressable array comprising at least 100 proteins immobilized on functionalized glass surface, and identifying a protein on the positionally addressable array that is bound and/or modified by the enzyme, wherein a binding or modifying of the protein by the enzyme indicates that the protein is a substrate for the enzyme. In additional embodiments, provided herein are methods for making an array of at least 1000 human proteins under non-denaturing conditions, including human proteins that are difficult to express and/or difficult to isolate in a non-denatured state.

The present application claims priority benefit under 35 U.S.C. § 119(e) of U.S. Provisional Application No. 60/610,444 filed Sep. 15, 2004, U.S. Provisional Application No. 60/610,446 filed Sep. 15, 2004, U.S. Provisional Application No. 60/620,193 filed Oct. 18, 2004, U.S. Provisional Application No. 60/620,233 filed Oct. 18, 2005, U.S. Provisional Application No. 60/653,585 filed Feb. 15, 2005 and U.S. Provisional Application No. 60/665,486 filed Mar. 25, 2005, the disclosure of each of which is incorporated by reference herein in its entirety.

Incorporated by reference herein in their entireties are Table 1, which is contained in the file named “Table 1,” (size 3,427 KB, created Sep. 15, 2005); Table 2, which is contained in the file named “Table 2” (size 7,350 KB, created Sep. 15, 2005); Table 3, which is contained in the file named “Table 3” (size 4,037 KB, created Sep. 15, 2005); Table 9, which is contained in the file named “Table 9” (size 849 KB, created Sep. 15, 2005); Table 10, which is contained in the file named “Table 10” (size 2,046 KB, created Sep. 15, 2005); Table 11, which is contained in the file named “Table 11” (size 1,316 KB, created Sep. 15, 2005), Table 13, which is contained in the file named “Table 13” (size 2,278 KB, created Sep. 15, 2005), and Table 18, which is contained in the file named “Table 18” (size 945 KB, created Sep. 15, 2005) which are all included on the Compact Disc that is filed herewith in duplicate labeled as “Copy 1” and “Copy 2.”

### 1. FIELD OF THE INVENTION

The present invention relates to the study of large numbers of proteins. More particularly, the present invention relates to protein microarrays and enzyme assays performed using positionally addressable arrays of proteins.

### 2. BACKGROUND OF THE INVENTION

A daunting task in the post-genome sequencing era is to understand the functions, modifications, and regulation of proteins (Fields et al., 1999, Proc Natl Acad. Sci. 96:8825; Goffeau et al., 1996, Science 274:563). This understanding will lead to the development of new and more effective diagnostic assays and medical treatments for human diseases. Although the human genome has been sequenced, making large numbers of molecules from the functional manifestation of the genome, the human proteome, available in a convenient format for analysis is likely to lead to tremendous increases in the speed at which new medical discoveries are made. However, it has not been demonstrated that high throughput recombinant methods, especially those using eurkaryotic expression systems, can be successfully employed to express, isolate, and array 1000s of human proteins. This is especially true for microarrays that include difficult to express proteins and proteins that are difficult to isolate in a properly folded form, such as membrane proteins.

One subset of proteins, called protein kinases, are enzyme that modify and thereby regulate the function of other proteins, which are especially important targets for future medical therapies and diagnostics. The importance of protein kinases in virtually all processes regulating cell transduction illustrates the potential for kinases and their cellular substrates as targets for therapeutics. Considerable efforts have been made to elucidate kinase biology by identifying the substrate specificity of kinases and using this information for the prediction of new substrates. Some of the approaches used to date include creation of a database from annotated phosphorylation sites, prediction of substrate sequence patterns from available structures of kinase/peptide substrate complexes, and screening of peptide libraries and peptide arrays (MacBeath G, and Schreiber S L, Science, 2000, 289:1760-1763; Zhu H, et al., Science, 2001, 293:2101-2105.). More recent efforts include attempts to map the phosphoproteome using mass spectroscopy-based techniques. While these studies have provided some information about kinase biology, they have been severely limited by their complexity, expense, lack of sensitivity, the use of non-structured peptides and by poor representation of potential substrates in the screens. There is a need for methods and compositions that provide large numbers of kinases and/or kinase substrates in a form that retains their 3-dimensional structure, and in a configuration that can be used to identify these substrates and compounds that affect phosphorylation of the substrates.

Citation or identification of any reference in this section and in any other section of this application, shall not be considered an admission that such reference is available as prior art to the present invention. Furthermore, section headers used herein are for the reader's convenience only.

### 3. SUMMARY OF THE INVENTION

The present invention is based, in part, on the successful expression, isolation, and microarray spotting of greater than 5000 human proteins, including numerous proteins of categories that are believed to be difficult-to-express proteins and that are also difficult to isolate in a non-denatured state, such as membrane proteins, especially transmembrane proteins. At least some of the proteins that have been successfully expressed, isolated, and microarray spotted retain their 3 dimensional structure and are functional. Certain embodiments of the present invention are also based, in part, on the discovery that functionalized glass substrates, especially those functionalized with a polymer that includes an acrylate functional group, are particularly effective for enzymatic assays performed using protein microarrays, especially kinase substrate identification assays.

The present invention is directed to a positionally addressable array comprising 100 human proteins from the proteins listed in Table 9, Table 11, and Table 13, immobilized on a substrate. In particular embodiments, the array comprises 500, 1000, 2500, or 5000 human proteins from the proteins listed in Table 9, Table 11, and Table 13. In another embodiment, the positionally addressable array comprises 100 of the membrane proteins of Table 15 or comprises 250 of the membrane proteins of Table 15. In yet another embodiment, the positionally addressable array comprises 50 of the transmembrane proteins of Table 16 or all of the transmembrane proteins of Table 16. In yet another embodiment, the positionally addressable array comprises at least 25 of the G protein coupled receptors (GPCRs) of Table 17 or all of the GPCRs of Table 17. The proteins on the positionally addressable array can be present on the array at a density of between 500 proteins/cm² and 10,000 proteins/cm². In particular embodiments, the proteins are non-denatured proteins, full-length proteins, non-denatured, full-length, recombinant fusion proteins comprising a tag.

The substrate on which the proteins are immobilized can be a functionalized glass slide. In a particular embodiment, the functionalized glass slide comprises a polymer comprising an acrylate group, wherein the polymer overlays a glass surface. In yet another embodiment, the substrate is a Protein slides II functionalized glass protein microarray substrate available from Full Moon Biosystems, Inc. (Sunnyvale, Calif.).

In another embodiment, the present invention is directed to a method for detecting a binding protein, comprising (a) contacting a probe with a positionally addressable array comprising at least 1000 human proteins of the proteins listed in Table 9, Table 11, and Table 13; and (b) detecting a protein-protein interaction between the probe and a protein of the array. In one embodiment, the proteins are produced in a eukaryotic cell and isolated under non-denaturing conditions. In another embodiment, the proteins are full-length proteins. In yet another embodiment, the proteins are non-denatured, full-length, recombinant fusion proteins comprising a GST or 6×HIS tag.

The present invention is also directed to a method for identifying a substrate of an enzyme, comprising contacting the enzyme with a positionally addressable array comprising at least 100 proteins immobilized on a functionalized glass slide, and identifying a protein on the positionally addressable array that is modified by the enzyme, wherein a modifying of the protein by the enzyme indicates that the protein is a substrate for the enzyme. The modifying of the protein by the enzyme can be identified by detecting on the array, signals generated from the protein that are at least 2-fold greater than signals obtained using the protein in a negative control assay; or detecting signals generated from the protein that are greater than 3 standard deviations greater than the median signal value for all negative control spots on the array. The enzyme activity that modifies the protein can be a chemical group transferring enzymatic activity. In another embodiment, the enzyme activity can be kinase activity, protease activity, phosphatase activity, glycosidase, or acetylase activity.

In another embodiment, the method for identifying a substrate of an enzyme further comprising contacting the probe with the functionalized glass slide in the presence and absence of a small molecule and determining whether the small molecule affects enzymatic modification of the substrate by the enzyme.

In particular embodiments, the functionalized glass slide comprises a three-dimensional porous surface comprising a polymer overlaying a glass surface. In another embodiment, the polymer overlying the glass surface comprises acrylate. The functionalized glass substrate can comprise multiple functional protein-specific binding sites. In a particular embodiment, the substrate is a Protein slides II protein microarray substrate available from Full Moon Biosystems, Inc. (Sunnyvale, Calif.).

In another embodiment, the array on the functionalized glass slide comprises at least 1000 human proteins of the proteins listed in Table 9, Table 11, and Table 13; at least 10,000 proteins expressed from the human genome; or at least 2500 human proteins of the proteins encoded by the sequences listed in Table 2. The proteins on the array can be produced under non-denaturing conditions. The proteins on the array can be full length human proteins produced in eukaryotic cells as non-denatured recombinant fusion proteins comprising a tag. The proteins on the array can comprise at least 50 transmembrane proteins of Table 16.

The present invention is also directed to a method for generating revenue, comprising (a) proving a service to a customer for identifying one or more enzyme substrates by performing a method for identifying a substrate of an enzyme, comprising contacting the enzyme with a positionally addressable array comprising at least 100 proteins immobilized on a functionalized glass slide, and identifying a protein on the positionally addressable array that is modified by the enzyme, wherein a modifying of the protein by the enzyme indicates that the protein is a substrate for the enzyme.

The present invention is also directed to a method for identifying a first kinase substrate for a customer, comprising, (a) providing access to the customer, to a service for identifying a substrate of a kinase, comprising (i) receiving an identity of a first kinase from a customer; (ii) contacting the first kinase under reaction conditions with a positionally addressable array comprising at least 100 proteins immobilized on a functionalized glass substrate; and (iii) identifying a protein on the positionally addressable array that is modified by the first kinase, wherein a modifying of the protein by the first kinase indicates that the protein is a substrate for the first kinase; and (b) providing an identity of the substrate to the customer. The method can further comprise repeating the service with a second kinase. In one embodiment, at least 100 immobilized proteins are from a first mammalian species. In another embodiment, the service is repeated using a positionally addressable array comprising at least 100 proteins from a second species, immobilized on a functionalized glass substrate. The method can also further comprise providing the substrate in an isolated form to the client. The method can also further comprise providing access to the customer to a purchasing function for purchasing any cell of a population of cells that express the substrate.

The present invention is also directed to a method for making an array of proteins, which method comprises cloning each open reading frame from a population of open reading frames into a baculovirus vector to generate a recombinant baculovirus vector, said vector comprising a promoter that directs expression of a fusion protein, which fusion protein comprising the open reading frame linked to a tag; expressing the fusion proteins generated for each of the population of open reading frames using insect cells; isolating the fusion proteins using affinity chromatography directed to the tag; and spotting the isolated proteins on a substrate. In one embodiment, the cells are sf9 cells. In another embodiment, the tag is a GST tag. The array of proteins can comprise 1000 full length mammalian proteins. Optionally, the proteins are human proteins. Further, the array can comprise at least 250 membrane proteins of Table 15, at least 50 transmembrane proteins of Table 16, or at least 25 G-protein coupled receptor proteins of Table 17. In another embodiment, the proteins are expressed, isolated, and spotted in a high-thoughput manner, under non-denaturing conditions.

The present invention is also directed to a positionally addressable array comprising at least 100 human proteins from the proteins encoded by the sequences whose accession numbers are listed in Table 1, Table 3, Table 5, Table 6, Table 9, Table 11, or Table 13 immobilized on a substrate. The present invention is also directed to a positionally addressable array comprising at least 50% of the proteins of a grouping listed in Table 10 immobilized on a substrate.

The present invention is also directed to a positionally addressable array comprising at least 50 human proteins that are difficult to express and/or difficult to isolate in a non-denatured state immobilized on a substrate. In one embodiment, the array comprises 50 human transmembrane proteins. The transmembrane proteins can comprise 50 of the transmembane proteins listed in Table 16 or can comprise 25 of the G-protein coupled receptors listed in Table 17. In another embodiment, the array comprises 100 human transmembrane proteins. In yet another embodiment, the transmembrane proteins are non-denatured transmembrane proteins. In yet another embodiment, at least one of the transmembrane proteins comprises a post-translational modification.

### 4. BRIEF DESCRIPTION OF THE FIGURES

FIG. 1. Kinase Substrate Profiling Service Workflow

FIG. 2. A. Negative Control (Autophosphorylation) Experiment with the Yeast ProtoArray™ KSP Proteome Positionally addressable array. B. Positive Control (PKA) Experiment with the Yeast ProtoArray™ KSP Proteome Positionally addressable array.

FIG. 3. Phosphorylation of unique substrates by on-test kinase. Selected subarrays from Yeast ProtoArray KSP Proteome Positionally addressable arrays incubated with ³³P-ATP only (left), ³³P-ATP and PKA (middle), and ³³P-ATP plus on-test kinase are shown.

FIG. 4. Top 200 proteins phosphorylated by an on-test kinase. The dark gray line indicates 3 standard deviations over the background. The light gray line indicates 5 standard deviations over the background.

### 5. DETAILED DESCRIPTION OF THE INVENTION

Protein Arrays

The present invention is based, in part, on Applicants' construction of a positionally addressable array of proteins containing over 5000 human proteins. The positionally addressable arrays of human proteins (also referred to as “protein chips” herein) provided herein can be used for global analyses of protein interactions and activities, such as enzymatic activities, as well as for the analysis of the affect of small molecules and other on-test molecules on these protein interactions and activities. The inventors have for the first time, successfully expressed in eukaryotic cells at a level of at least 19 nM, thousands of human proteins under non-denaturing conditions, including numerous human proteins of a class of proteins that are considered difficult to express proteins and difficult to isolate in a non-denatured state, including over 50 transmembrane proteins. The inventors subsequently isolated the proteins using a GST fusion tag and microarrayed the proteins. The inventors have confirmed that at least some of the expressed and arrayed human proteins appear to retain their 3-dimensional structure using epitope specific antibodies that require proper 3-dimensional folding, and by confirming protein-protein interactions identified on the array, using other methods that are also performed under non-denaturing conditions.

Table 1, filed herewith on CD in the file named “Table 1,” lists the coding sequences encoding human proteins that the inventors attempted to express and isolate using the protein production and isolation methods disclosed in Example 1 herein. Table 2, filed herewith on CD, includes the identities of coding sequences encoding human proteins that include the proteins encoded by the coding sequences of Table 1 and additional coding sequences to which the inventors have obtained clones whose human open reading frame inserts can be removed and inserted into a pDEST20 vector, in a manner similar to that which was successfully performed for the majority of coding sequences encoding the proteins of Tables 9, 11, and 13. Table 3 provides a list, including coding sequences, of proteins that the inventors expressed at a concentration of at least 19.2 nM, isolated, and microarrayed according to the method provided in Example 1 in production lot 4.1. Tables 5 and 7 provide a list including concentration information (Table 7 last column (nM)) of proteins that were successfully expressed, isolated, and microarrayed according to the methods provided in Example 1 in production lot 4.1. Table 6 provides a list of the 176 human kinases that were expressed, isolated, and microarrayed using the methods provided in Example 1. Table 8 provides a list of human kinases that were expressed, isolated, and microarrayed using the methods provided in Example 1. Tables 9 and 11 provide the sequences of proteins that were successfully expressed, isolated and microarrayed using the methods provided in Example 1 in different production lots (4.1 and 5.1 respectively). Table 10 lists the proteins and associated Gene Ontology (GO) information for proteins that were successfully expressed, isolated, and microarrayed using the methods of Example 1 in production lot 5.1.

Table 13, filed herewith on CD in the file named “Table 13,” provides the amino acid sequences, accession numbers, ORF identifier, and FASTA header for 5034 human proteins that the inventors have expressed at a concentration of at least 19.2 nM, isolated, and microarrayed using the protein production, isolation, and microarray system provided in Example 1 herein as production lot 5.2. Table 15, provided herewith provides the 429 proteins classified in the GO categories as “membrane proteins,” that were expressed, isolated, and microarrayed as part of production lot 5.2, using the methods provided in Example 1. Table 16, provided herewith, provides the 88 proteins classified in the GO categories as “transmembrane proteins,” that were expressed, isolated, and microarrayed as part of production lot 5.2, using the methods provided in Example 1. Table 17, provided herewith, provides a list of 42 G-protein coupled receptors that have been expressed, isolated, and microarrayed using the methods provided in Example 1 as part of production lot 5.2. Table 18, filed herewith on CD in the file named “Table 18,” provides the names, identifiers and concentrations at the time of microarray spotting (number in “name” column after “-”) for proteins expressed in production lot 5.2, as well as microarray positional information.

The present invention is directed to a positionally addressable array comprising 100 human proteins from the proteins listed in Table 9, Table 11, and Table 13, immobilized on a substrate. In particular embodiments, the array comprises 500, 1000, 2500, or 5000 human proteins from the proteins listed in Table 9, Table 11, and Table 13. In another embodiment, the positionally addressable array comprises 100 of the membrane proteins of Table 15 or comprises 250 of the membrane proteins of Table 15. In yet another embodiment, the positionally addressable array comprises 50 of the transmembrane proteins of Table 16 or all of the transmembrane proteins of Table 16. In yet another embodiment, the positionally addressable array comprises at least 25 of the G protein coupled receptors (GPCRs) of Table 17 or all of the GPCRs of Table 17. The proteins on the positionally addressable array can be present on the array at a density of between 500 proteins/cm² and 10,000 proteins/cm². In particular embodiments, the proteins are non-denatured proteins, full-length proteins, non-denatured, full-length, recombinant fusion proteins comprising a tag.

The substrate on which the proteins are immobilized can be a functionalized glass slide. In a particular embodiment, the functionalized glass slide comprises a polymer comprising an acrylate group, wherein the polymer overlays a glass surface. In yet another embodiment, the substrate is a Protein slides II functionalized glass protein microarray substrate available from Full Moon Biosystems, Inc. (Sunnyvale, Calif.).

In another embodiment, the present invention is directed to a method for detecting a binding protein, comprising (a) contacting a probe with a positionally addressable array comprising at least 1000 human proteins of the proteins listed in Table 9, Table 11, and Table 13; and (b) detecting a protein-protein interaction between the probe and a protein of the array. In one embodiment, the proteins are produced in a eukaryotic cell and isolated under non-denaturing conditions. In another embodiment, the proteins are full-length proteins. In yet another embodiment, the proteins are non-denatured, full-length, recombinant fusion proteins comprising a GST or 6×HIS tag.

The present invention is also directed to a method for identifying a substrate of an enzyme, comprising contacting the enzyme with a positionally addressable array comprising at least 100 proteins immobilized on a functionalized glass slide, and identifying a protein on the positionally addressable array that is modified by the enzyme, wherein a modifying of the protein by the enzyme indicates that the protein is a substrate for the enzyme. The modifying of the protein by the enzyme can be identified by detecting on the array, signals generated from the protein that are at least 2-fold greater than signals obtained using the protein in a negative control assay; or detecting signals generated from the protein that are greater than 3 standard deviations greater than the median signal value for all negative control spots on the array. The enzyme activity that modifies the protein can be a chemical group transferring enzymatic activity. In another embodiment, the enzyme activity can be kinase activity, protease activity, phosphatase activity, glycosidase, or acetylase activity.

In another embodiment, the method for identifying a substrate of an enzyme further comprising contacting the probe with the functionalized glass slide in the presence and absence of a small molecule and determining whether the small molecule affects enzymatic modification of the substrate by the enzyme.

In particular embodiments, the functionalized glass slide comprises a three-dimensional porous surface comprising a polymer overlaying a glass surface. In another embodiment, the polymer overlying the glass surface comprises acrylate. The functionalized glass substrate can comprise multiple functional protein-specific binding sites. In a particular embodiment, the substrate is a Protein slides II protein microarray substrate available from Full Moon Biosystems, Inc. (Sunnyvale, Calif.).

In another embodiment, the array on the functionalized glass slide comprises at least 1000 human proteins of the proteins listed in Table 9, Table 11, and Table 13; at least 10,000 proteins expressed from the human genome; or at least 2500 human proteins of the proteins encoded by the sequences listed in Table 2. The proteins on the array can be produced under non-denaturing conditions. The proteins on the array can be full length human proteins produced in eukaryotic cells as non-denatured recombinant fusion proteins comprising a tag. The proteins on the array can comprise at least 50 transmembrane proteins of Table 16.

The present invention is also directed to a method for generating revenue, comprising (a) proving a service to a customer for identifying one or more enzyme substrates by performing a method for identifying a substrate of an enzyme, comprising contacting the enzyme with a positionally addressable array comprising at least 100 proteins immobilized on a functionalized glass slide, and identifying a protein on the positionally addressable array that is modified by the enzyme, wherein a modifying of the protein by the enzyme indicates that the protein is a substrate for the enzyme.

The present invention is also directed to a method for identifying a first kinase substrate for a customer, comprising, (a) providing access to the customer, to a service for identifying a substrate of a kinase, comprising (i) receiving an identity of a first kinase from a customer; (ii) contacting the first kinase under reaction conditions with a positionally addressable array comprising at least 100 proteins immobilized on a functionalized glass substrate; and (iii) identifying a protein on the positionally addressable array that is modified by the first kinase, wherein a modifying of the protein by the first kinase indicates that the protein is a substrate for the first kinase; and (b) providing an identity of the substrate to the customer. The method can further comprise repeating the service with a second kinase. In one embodiment, at least 100 immobilized proteins are from a first mammalian species. In another embodiment, the service is repeated using a positionally addressable array comprising at least 100 proteins from a second species, immobilized on a functionalized glass substrate. The method can also further comprise providing the substrate in an isolated form to the client. The method can also further comprise providing access to the customer to a purchasing function for purchasing any cell of a population of cells that express the substrate.

The present invention is also directed to a method for making an array of proteins, which method comprises cloning each open reading frame from a population of open reading frames into a baculovirus vector to generate a recombinant baculovirus vector, said vector comprising a promoter that directs expression of a fusion protein, which fusion protein comprising the open reading frame linked to a tag; expressing the fusion proteins generated for each of the population of open reading frames using insect cells; isolating the fusion proteins using affinity chromatography directed to the tag; and spotting the isolated proteins on a substrate. In one embodiment, the cells are sf9 cells. In another embodiment, the tag is a GST tag. The array of proteins can comprise 1000 full length mammalian proteins. Optionally, the proteins are human proteins. Further, the array can comprise at least 250 membrane proteins of Table 15, at least 50 transmembrane proteins of Table 16, or at least 25 G-protein coupled receptor proteins of Table 17. In another embodiment, the proteins are expressed, isolated, and spotted in a high-thoughput manner, under non-denaturing conditions.

The present invention is also directed to a positionally addressable array comprising at least 100 human proteins from the proteins encoded by the sequences whose accession numbers are listed in Table 1, Table 3, Table 5, Table 6, Table 9, Table 11, or Table 13 immobilized on a substrate. The present invention is also directed to a positionally addressable array comprising at least 50% of the proteins of a grouping listed in Table 10 immobilized on a substrate.

The present invention is also directed to a positionally addressable array comprising at least 50 human proteins that are difficult to express and/or difficult to isolate in a non-denatured state immobilized on a substrate. In one embodiment, the array comprises 50 human transmembrane proteins. The transmembrane proteins can comprise 50 of the transmembane proteins listed in Table 16 or can comprise 25 of the G-protein coupled receptors listed in Table 17. In another embodiment, the array comprises 100 human transmembrane proteins. In yet another embodiment, the transmembrane proteins are non-denatured transmembrane proteins. In yet another embodiment, at least one of the transmembrane proteins comprises a post-translational modification.

Proteins that are difficult-to-express proteins and that are also difficult to isolate in a non-denatured state, include proteins that were previously believed to require special conditions in order to be successfully expressed and isolated in a native form. For example, proteins such as those associated with membranes, especially transmembrane proteins were previously believed to require special conditions to be successfully expressed and isolated in a native form.

In another embodiment, the present invention provides a positionally addressable array comprising at least 10, 20, 25, 50, 75, 100, 150, 200, 250, 500, 750, 1000, 1500, 2000, 2500, 3000, or all human proteins from the proteins encoded by the sequences listed in Table 1, immobilized on a substrate. Table 1 is provided in computer readable form on the CD filed herewith, as the file named “Table 1.”

In yet another embodiment, the present invention provides a positionally addressable array comprising at least 10, 20, 25, 50, 75, 100, 150, 200, 250, 500, 750, 1000, 1500, 2000, 2500, 3000, 4000, 5000, 6000, 7000, 7500, or all human proteins encoded by the sequences listed in Table 2, immobilized on a solid support. Table 2 is provided in computer readable form on the CD filed herewith, as the file named “Table 2.”

In certain embodiments, the present invention provides a positionally addressable array comprising at least 10, 20, 25, 50, 75, 100, 150, 200, 250, 500, 750, 1000, 1500, 2000, 2500, 3000, or all human proteins from the proteins encoded by the sequences listed in Table 1;

at most 10, 20, 25, 50, 75, 100, 150, 200, 250, 500, 750, 1000, 1500, 2000, 2500, 3000, or all human proteins from the proteins encoded by the sequences listed in Table 1;

at least 3500, 4000, 4500, 5000, 7500, 10,000, substantially all, or all human proteins expressed from the human genome;

at most 3500, 4000, 4500, 5000, 7500, 10,000, substantially all, or all human proteins expressed from the human genome;

at least 10, 20, 25, 50, 75, 100, 150, 200, 250, 500, 750, 1000, 1500, 2000, 2500, 3000, 4000, or 5000, 6000, 7000, 7500, or all proteins encoded by the sequences listed in Table 2;

at most 10, 20, 25, 50, 75, 100, 150, 200, 250, 500, 750, 1000, 1500, 2000, 2500, 3000, 4000, or 5000, 6000, 7000, 7500, or all proteins encoded by the sequences listed in Table 2;

at least 10, 20, 25, 50, 75, 100, 150, 200, 250, 500, 750, 1000, 1500, or all human proteins from the proteins encoded by the sequences listed in Table 3;

at most 10, 20, 25, 50, 75, 100, 150, 200, 250, 500, 750, 1000, 1500, or all human proteins from the proteins encoded by the sequences listed in Table 3;

at least 10, 20, 25, 50, 75, 100, 150, 200, 250, 500, 750, 1000, 1500 or all human proteins from the proteins encoded by the sequences whose accession numbers are listed in Table 5 or Table 7 or Table 9;

at most 10, 20, 25, 50, 75, 100, 150, 200, 250, 500, 750, 1000, 1500 or all human proteins from the proteins encoded by the sequences whose accession numbers are listed in Table 5 or Table 7;

at least 10, 20, 25, 50, 75, 100, 150, or all human proteins from the proteins encoded by the sequences whose accession numbers are listed in Table 6 or Table 8;

at most 10, 20, 25, 50, 75, 100, 150, or all human proteins from the proteins encoded by the sequences whose accession numbers are listed in Table 6 or Table 8;

at least 10, 20, 25, 50, 75, 100, 150, 200, 250, 500, 750, 1000, 1500, 2000, 2500, 3000, 4000, 5000, 6000, 7000, 8000, 9000, 10000, 11000, 12000, 13000, 14000, 15000, 16000, 17000, 17500, or all proteins listed in Table 10;

at most 10, 20, 25, 50, 75, 100, 150, 200, 250, 500, 750, 1000, 1500, 2000, 2500, 3000, 4000, or 5000, 6000, 7000, 8000, 9000, 10000, 11000, 12000, 13000, 14000, 15000, 16000, 17000, 17500, or all proteins listed in Table 10;

at least 10, 20, 25, 50, 75, 100, 150, 200, 250, 500, 750, 1000, 1500, 2000, 2500, 3000, or all proteins listed in Table 9 and/or Table 11; or at most 10, 20, 25, 50, 75, 100, 150, 200, 250, 500, 750, 1000, 1500, 2000, 2500, 3000, or all proteins listed in Table 9 and/or Table 11;

at least 10, 20, 25, 50, 75, 100, 150, 200, 250, 500, 750, 1000, 1500, 2000, 2500, 3000, 4000, 5000, or all proteins listed in Table 13; or at most 10, 20, 25, 50, 75, 100, 150, 200, 250, 500, 750, 1000, 1500, 2000, 2500, 3000, 4000, 5000 or all proteins listed in Table 13.

In certain aspects, arrays of the present invention include at least 1, and typically at least 25, 50, 100, 200, 300, or 400 difficult-to-express proteins that are also difficult to isolate in a non-denatured state. Preferably, these proteins are arrayed in a non-denatured state. For example, in illustrative aspects, the arrays comprise at least 400 or all proteins of the membrane proteins of Table 15, at least 50 or all of the transmembrane proteins of Table 16, and/or at least 25 or all of the GPCRs of Table 17.

In certain embodiments, the present invention provides a positionally addressable array comprising at least 5%, 10%, 15%, 20%, 25%, 30%, 40%, 50%, 60%, 70%, 80%, 85%, 90%, 95%, 96%, 97%, 98%, 99%, or all human proteins of a grouping of proteins listed in Table 10. In certain embodiments, the present invention provides a positionally addressable array comprising at most 5%, 10%, 15%, 20%, 25%, 30%, 40%, 50%, 60%, 70%, 80%, 85%, 90%, 95%, 96%, 97%, 98%, 99%, or all human proteins of a grouping of proteins listed in Table 10. Each grouping provides proteins with a particular functional aspect. The groupings listed in Table 10 are gene ontology, biological process, behavior, biological process unknown, cell communication, cell-cell signaling, signal transduction, development, cell differentiation, embryonic development, growth, cell growth, morphogenesis, regulation of gene expression, reproduction, physiological process, cell death, cell growth and/or maintenance, cell homeostasis, cell organization and biogenesis, cytoplasm organization and biogenesis, organelle organization and biogenesis, cytoskeleton organization and biogenesis, cell proliferation, cell cycle, transport, ion transport, protein transport, death, metabolism, amino acid and derivative metabolism, biosynthesis, protein biosynthesis, carbohydrate metabolism, catabolism, coenzyme and prosthetic group metabolism, electron transport, energy pathways, lipid metabolism, nucleobase, nucleoside, nucleotide and nucleic acid metabolism, DNA metabolism, transcription, protein metabolism, protein biosynthesis, protein modification, secondary metabolism, response to biotic stimulus, response to endogenous stimulus, response to external stimulus, response to abiotic stimulus, cellular component, cell, external encapsulating structure, cell envelope, cell wall, intracellular, chromosome, nuclear chromosome, cytoplasm, cytoplasmic vesicle, cytoskeleton, cytosol, endoplasmic reticulum, endosome, golgi apparatus, microtubule organizing center, mitochondrion, peroxisome, ribosome, vacuole, lysosome, nucleus, nuclear chromosome, nuclear membrane, nucleolus, nucleoplasm, ribosome, nuclear membrane, plasma membrane, cellular_component unknown, extracellular, extracellular matrix, extracellular space, unlocalized, molecular_function, antioxidant activity, binding, calcium ion binding, carbohydrate binding, lipid binding, nucleic acid binding, DNA binding, chromatin binding, transcription factor activity, RNA binding, translation factor activity, nucleic acid binding, nucleotide binding, protein binding, ytoskeletal protein binding, actin binding, receptor binding, catalytic activity, hydrolase activity, nuclease activity, peptidase activity, phosphoprotein phosphatase activity, kinase activity, protein kinase activity, transferase activity, enzyme regulator activity, molecular_function unknown, motor activity, signal transducer activity, receptor activity, receptor binding, structural molecule activity, transcription regulator activity, translation regulator activity, translation factor activity nucleic acid binding, transporter activity, electron transporter activity, ion channel activity, neurotransmitter transporter activity.

In certain embodiments, the invention provides a protein microarray with proteins of at least 1, 2, 3, 4, 5, 6, 7, 8, 9, 10, 15, 20, 25, 50, 75, or at least 100 or all groupings of the proteins in Table 10. In certain embodiments, the invention provides a protein microarray with proteins of at most 1, 2, 3, 4, 5, 6, 7, 8, 9, 10, 15, 20, 25, 50, 75, or at least 100 or all groupings of the proteins in Table 10.

Furthermore, the invention provides a positionally addressable protein microarray comprising at least 1, 2, 3, 4, 5, 6, 7, 8, 9, 10, 15, 20, 25, 30, 35, 40, 50, 75, 100, 200, 250, 500, 750, 1000, 1500, or all human proteins of a grouping of proteins listed in Table 10. Furthermore, the invention provides a positionally addressable protein microarray comprising at most 1, 2, 3, 4, 5, 6, 7, 8, 9, 10, 15, 20, 25, 30, 35, 40, 50, 75, 100, 200, 250, 500, 750, 1000, 1500, or all human proteins of a grouping of proteins listed in Table 10.

Furthermore, the invention provides a positionally addressable protein microarray comprising at least 1, 2, 3, 4, 5, 6, 7, 8, 9, 10, 15, 20, 25, 30, 35, 40, 50, 75, 100, 200, 250, 500, 750, 1000, 1500, 2000, 2500, 3000, or all human proteins of a grouping of proteins listed in Table 9, Table 11, and/or Table 13. Furthermore, the invention provides a positionally addressable protein microarray comprising at most 1, 2, 3, 4, 5, 6, 7, 8, 9, 10, 15, 20, 25, 30, 35, 40, 50, 75, 100, 200, 250, 500, 750, 1000, 1500, 2000, 2500, 3000, or all human proteins of a grouping of proteins listed in Table 9, Table 11, and/or Table 13. The proteins in illustrative embodiments are non-denatured, full-length, and/or recombinant fusion proteins, that preferably include a tag, especially a GST tag, and optionally at least one of which, and more preferably at least 100 of which, include at least one post-translational modification. In illustrative aspects, the proteins include a non-native TAG stop codon. In certain illustrative embodiments, the arrays include at least 10 human autoantigens, preferably non-denatured autoantigens.

In certain aspects, the array comprises no more than 3000, 3500, 4000, 5000, 6000, 7000, 8000, 9000, or 10000 proteins. In another embodiment, the present invention provides a positionally addressable array of at least 3500, 4000, 4500, 5000, 7500, 10,000, substantially all, or all human proteins expressed from the human genome, immobilized on a solid support. In another related embodiment, the present invention provides a positionally addressable array of at least 10%, 20%, 25%, 30%, 35%, 40%, 50%, 60%, 70%, 75%, 80%, 90%, 95%, 96%, 97%, 98%, or 99% of human proteins expressed from the human genome, immobilized on a solid support. Isoforms and variants of a protein are considered 1 protein for this percentage determination. In certain aspects of this embodiment, the human proteins comprise at least 1000 proteins from the proteins encoded by the sequences listed in Table 1 and/or Table 2, immobilized on a solid support. In certain illustrative examples, the array is a functional protein array.

Positionally addressable arrays provided herein are typically a high-density positionally addressable array of proteins, comprising a density of at least 500 proteins/cm², at least 1000 proteins/cm², at least 2000 proteins/cm², at least 3000 proteins/cm², at least 5000 proteins/cm², or at least 10,000 proteins/cm². In certain aspects, the density is between 500 proteins/cm² and 5000 proteins/cm². In certain aspects, the positionally addressable arrays comprise at least 1, 2, 3, 4, 5, 6, 7, 8, 9, 10, 11, 12, 13, 14, 15, 20, 25, 50, 75, 100, or all members of a class or a plurality of classes of human proteins. The plurality of classes includes 2, 3, 4, 5, 6, 7, 8, 9, 10, 11, 12, 13, 14, 15, 20, or 25 classes, for example. Typically, for arrays comprising less than 5 members of any class, there are at least 5 classes of functional proteins represented on the array. A class can be a group of gene products that are related according to molecular function, biological process, or cellular component. Such a relationship can be established, for example, using the gene ontology-based system available on the worldwide web at geneontology.org, incorporated herein by reference in its entirety. For example, the positionally addressable array can include at least 1 member of at least 10 different molecular function ontology-based classifications of proteins. In certain aspects, the positionally addressable arrays include at least 1 member of human proteins for each known ontology-based molecular function, biological process, and/or cellular component classification for human proteins.

The proteins on the positionally addressable arrays provided herein are typically produced under non-denaturing conditions. Furthermore, the proteins in illustrative examples, are full-length proteins, and can include additional tag sequences. Accordingly, the proteins in certain aspects, are full-length recombinant fusion proteins. Therefore, the invention encompasses a method for detecting a binding protein comprising the steps of contacting a probe with a positionally addressable array comprising a plurality of fusion proteins, with each protein being at a different position on a solid support, wherein the fusion protein comprises a first tag and a protein sequence encoded by genomic nucleic acid of an organism, and detecting any protein-probe interaction. As described above, in certain embodiments, the two tags are His or GST.

Also provided are methods for using positionally addressable arrays of proteins provided herein. The positionally addressable array of proteins of the invention can be used, for example, to identify protein-protein interactions, to identify a binding protein, or to identify enzymatic activity. Thus, the invention encompasses a method for detecting a binding protein comprising contacting a probe with a positionally addressable array comprising a plurality of proteins, with each protein being at a different position on a solid support, and detecting the binding of the probe to a protein on the array, wherein the plurality of proteins comprises one of the following:

at least 10, 20, 25, 50, 75, 100, 150, 200, 250, 500, 750, 1000, 1500, 2000, 2500, 3000, or all human proteins from the proteins encoded by the sequences listed in Table 1;

at least 3500, 4000, 4500, 5000, 7500, 10,000, substantially all, or all human proteins expressed from the human genome;

at least 10, 20, 25, 50, 75, 100, 150, 200, 250, 500, 750, 1000, 1500, 2000, 2500, 3000, 4000, or 5000, 6000, 7000, 7500, or all proteins encoded by the sequences listed in Table 2; or

at least 10%, 20%, 25%, 30%, 35%, 40%, 50%, 60%, 70%, 75%, 80%, 90%, 95%, 96%, 97%, 98%, or 99% of human proteins expressed from the human genome.

The present invention also provides a method for detecting a binding protein comprising the steps of contacting a sample of biotinylated proteins with a positionally addressable array comprising a plurality of proteins, with each protein being at a different position on a solid support, contacting the array with streptavidin conjugated to a detectable label, such as a fluorescent label, and detecting positions on the array at which fluorescence occurs, wherein the fluorescence is indicative of an interaction between a biotinylated protein and a protein on the array. The positionally addressable array is a protein microarray provided herein.

The present invention also provides a method for detecting a binding protein comprising the steps of contacting a biotinylated protein or a sample of biotinylated proteins with a positionally addressable array comprising a plurality of proteins, with each protein being at a different position on a solid support, contacting the array with streptavidin conjugated to a detectable label, such as a fluorescent label, and detecting positions on the array at which fluorescence occurs, wherein the fluorescence is indicative of an interaction between a biotinylated protein and a protein on the array. The positionally addressable array is a protein microarray provided herein. The biotinylated protein or the sample of biotinylated proteins can be biotinylated in vitro or in vivo. For example the biotinylated protein can be biotinylated using commercially available products. In one example, the biotinylated protein is biotinylated in vivo using a Bioease tag (Invitrogen, Carlsbad, Calif.).

The present invention encompasses a positionally addressable array comprising a plurality of proteins, with each protein being at a different position on a solid support, wherein the plurality of proteins comprises at least one protein encoded by at least 10%, 20%, 30%, 40%, 50%, 60%, 70%, 80%, 90%, 95%, or 99% of the known human genes, i.e., all protein isoforms and splice variants derived from a gene are considered one protein.

A positionally addressable array provides a configuration such that each probe or protein of interest is at a known position on the solid support thereby allowing the identity of each probe or protein to be determined from its position on the array. Accordingly, each protein on an array is preferably located at a known, predetermined position on the solid support such that the identity of each protein can be determined from its position on the solid support.

Proteins of the positionally addressable arrays of proteins of the invention include full-length proteins, portions of full-length proteins, and peptides, which can be prepared by recombinant overexpression, fragmentation of larger proteins, or chemical synthesis. In certain illustrative examples, the proteins are full-length proteins, such as full-length recombinant fusion proteins. Proteins can be overexpressed in cells derived from, for example, yeast, bacteria, insects, humans, or non-human mammals such as mice, rats, cats, dogs, pigs, cows and horses. The proteins can be native or denatured, but are preferably native or at least isolated under non-denaturing conditions. Furthermore, the proteins can be devoid of post-translational modifications, for example by expression in a bacteria or by enzymatic treatment, or can include post-translational modifications, for example by expression in eukaryotic cells. Further, fusion proteins comprising a defined domain attached to a natural or synthetic protein can be used. Proteins of the protein arrays can be purified prior to being attached to the solid support of the chip. Also the proteins of the proteome purified can be purified, or further purified, during attachment to the positionally addressable array of proteins.

The solid support used for the positionally addressable arrays of proteins of the present invention can be constructed from materials such as, but not limited to, silicon, glass, quartz, polyimide, acrylic, polymethylmethacrylate (LUCITE®, Lucite International, Southhampton, UK), ceramic, nitrocellulose, amorphous silicon carbide, polystyrene, and/or any other material suitable for microfabrication, microlithography, or casting. For example, the solid support can be a hydrophilic microtiter plate (e.g., MILLIPORE™, Millipore Corp., Billerica, Mass.) or a nitrocellulose-coated glass slide. Nitrocellulose-coated glass slides for making protein (and DNA) positionally addressable arrays are commercially available (e.g., from Schleicher & Schuell (Keene, N.H.), which sells glass slides coated with a nitrocellulose based polymer (Cat. no. 10 484 182)).

In illustrative aspects, proteins of the array are immobilized on a functionalized glass substrate. This aspect is particularly useful for embodiments that include methods for determining enzyme activity, especially kinase activity, or for methods for identifying enzyme substrates, such as kinase substrate identification methods. In certain embodiments, a glass slide can be functionalized with an epoxy silane (Available from, for example, Schott-Nexterion and Erie Scientific).

In preferred embodiments, the functionalized glass slides can be functionalized with a polymer that contains an acrylate functional group, optionally including cellulose. Furthermore, in these preferred embodiments, the functionalized glass substrate can be a substrate with a three-dimensional porous surface comprising a polymer overlaying a glass surface. The three-dimensional porous surface comprising a polymer overlaying a glass surface, in certain aspects, typically allows proteins to be nested therein. The surface typically includes multiple functional protein-specific binding sites. The surface in illustrative examples, is hydrophobic. In especially preferred aspects of these preferred embodiments, the substrate is Protein slides I or Protein slides II (catalog numbers 25, 25B, 50, or 50B) available from Full Moon Biosystems, Sunnyvale, Calif. In certain aspects, the substrate is Protein slides II (cat. No. 25, 25B, 50, or 50B) from Full Moon Biosystems. In other aspects, the positionally addressable array of proteins utilize substrates such as a Corning UltraGAPS (Corning, Cat. No. 40015), GAPS II (Corning, Cat. No. 40003), Super Epoxy slides (TeleChem), Nickel Chelate-coated slides (available for example from Greiner Bio-One Inc., Longwood, Fla. or from Xenopore, Hawthorne, N.J.), or Low Background Aldehyde slides (available from Microsurfaces Inc., Minneapolis, Minn.).

Accordingly, in one embodiment, the positionally addressable array of proteins comprises a plurality of proteins that are applied to the surface of a solid support, wherein the density of the sites at which protein are applied is at least 100 sites/cm², 1000 sites/cm², 10,000 sites/cm², 100,000 sites/cm², or 1,000,000 sites/cm². Each individual isolated protein sample is preferably applied to a separate site on the array, typically a microarray. The identity of the protein(s) at each site on the chip is/are known. Typically duplicates of individual isolated proteins are applied to spots on the array.

In order to produce arrays of hundreds or thousands of proteins, it was necessary to convert genetic information into hundreds or thousands of pure proteins. As illustrated in the Examples provided herein, although the basic technologies necessary for producing this content for a few proteins at a time have been in place for a number of years, the high-throughput method disclosed herein for cloning, expression, purification, and microarraying of thousands of functional proteins is unique. Using this method, open reading frames encoding over 3400 recombinant human fusion proteins were cloned, expressed, purified and arrayed. The human cDNAs were cloned into a Gateway entry vector, completely sequence-verified, expressed as GST and/or 6×His-fusions in a high-throughput baculovirus-based system, and purified using affinity chromatography. Purified proteins along with appropriate controls were arrayed on functionalized glass slides.

Accordingly, the present invention provides a method for making an array of proteins, comprising:

cloning each open reading from of a population of open reading frames into a baculovirus vector to generate a recombinant baculovirus vector comprising a promoter that directs expression of a fusion protein comprising the open reading frame linked to a tag;

expressing the fusion proteins generated for each of the population of open reading frames using insect cells;

isolating the fusion proteins using affinity chromatography directed to the tag; and

spotting the isolated protein on a substrate.

In certain aspects, the proteins are mammalian proteins, for example, human proteins, preferably at least 100, 200, 250, 500, 1000, 2000, 2500, 3000, 4000, 5000, or all of the proteins in Table 9, Table 11, and/or Table 13, preferably recombinantly expressed in a eukaryotic system, and most preferably isolated under non-denaturing conditions as a fusion protein with a tag. In preferred aspects, the arrays include at least 50 difficult to express proteins that are also difficult to isolate in a non-denatured state, such as membrane proteins, especially transmembrane proteins, at least some of which can be GPCRs. In illustrative embodiments, the proteins are expressed at a concentration of at least 1, 5, 10, 15, 16, 17, 18, 19, or 19.2 nM. Furthermore, at least 40 ul of the protein can be expressed, and preferably at least 100 ul or 200 ul of protein is expressed. Any expression construct having an inducible promoter to drive protein synthesis can be used in accordance with the methods of the invention. Preferably, the expression construct is tailored to the cell type to be used for transformation. Compatibility between expression constructs and host cells are known in the art, and use of variants thereof are also encompassed by the invention. In certain illustrative embodiments, the expression construct is a baculovirus construct.

Methods are known to clone open reading frames into a baculovirus vector such that a promoter on the baculovirus vector directs expression of a fusion protein comprising the open reading frame linked to a tag. The open reading frame can be cloned from virtually any source including genomic DNA and cDNA. In certain aspects, the open reading frame is cloned into a vector such that it is in frame with the tag. In certain aspects, the multiple open reading frames are cloned into a vector such that a complex comprising more than one subunit open reading frame products is formed in the insect cells and purified using a tag on at least one of the proteins of the multi-protein complex (See e.g., Berger et al., Nature Biotechnology 22, 1583-1587 (2004)).

A variety of tags (i.e. heterologous domains, typically with affinity for a compound) are known in the art and can be used. Accordingly, in an illustrative embodiment, proteins of the positionally addressable array of proteins are expressed as fusion proteins having at least one heterologous domain with an affinity for a compound that is attached to the surface of the solid support or that is used to purify the protein using, for example, affinity chromatoagraphy. Suitable compounds useful for binding fusion proteins onto the solid support (i.e., acting as binding partners) include, but are not limited to, trypsin/anhydrotrypsin, glutathione, immunoglobulin domains, maltose, nickel, or biotin and its derivatives, which bind to bovine pancreatic trypsin inhibitor, glutathione-S-transferase, Protein A or antigen, maltose binding protein, poly-histidine (e.g., HisX6 tag), and avidin/streptavidin, respectively. For example, Protein A, Protein G and Protein A/G are proteins capable of binding to the Fc portion of mammalian immunoglobulin molecules, especially IgG. These proteins can be covalently coupled to, for example, a Sepharose® support to provide an efficient method of purifying fusion proteins having a tag comprising an Fc domain.

In certain aspects of the invention, at least 2 tags are present on the protein, one of which can be used to aid in purification and the other can be used to aid in immobilization. In certain illustrative aspects, the tag is a His tag, a GST tag, or a biotin tag. Where the tag is a biotin tag, the tag can be associated with a protein in vitro or in vivo using commercially available reagents (Invitrogen, Carlsbad, Calif.). In aspects where the tag is associated with the protein in vitro, a Bioease tag can be used (Invitrogen, Carlsbad, Calif.).

In certain examples, a eukaryotic cell (e.g., yeast, human cells) is preferably used to synthesize eukaryotic proteins. Further, a eukaryotic cell amenable to stable transformation, and having selectable markers for identification and isolation of cells containing transformants of interest, is preferred. Alternatively, a eukaryotic host cell deficient in a gene product is transformed with an expression construct complementing the deficiency. Cells useful for expression of engineered viral, prokaryotic or eukaryotic proteins are known in the art, and variants of such cells can be appreciated by one of ordinary skill in the art. The cells can include yeast, insect, and mammalian cells. In certain aspects, corn cells are used to produce the recombinant human proteins.

For example, the InsectSelect system from Invitrogen (Carlsbad, Calif., catalog no. K800-01), a non-lytic, single-vector insect expression system that simplifies expression of high-quality proteins and eliminates the need to generate and amplify virus stocks, can be used. An illustrative vector in this system is pIB/V5-His TOPO TA vector (catalog no. K890-20). Polymerase chain reaction (“PCR”) products can be cloned directly into this vector, using the protocols described by the manufacturer, and the proteins can be expressed with N-terminal histidine tags useful for purifying the expressed protein.

Another eukaryotic expression system in insect cells, the BAC-TO-BAC™ system (Invitrogen™, Carlsbad, Calif.), can also be used. Rather than using homologous recombination, the BAC-TO-BAC™ system generates recombinant baculovirus by relying on site-specific transposition in E. coli. Gene expression is driven by the highly active polyhedrin promoter, and therefore can represent up to 25% of the cellular protein in infected insect cells. In another aspect, a BaculoDirect™ Baculovirus Expression System (Invitrogen™) is used.

In certain aspects, each open reading frame is initially cloned into a recombinational cloning vector such as a Gateway™ entry vector, and then shuttled into a into a baculovirus vector. Methods are known in the art for performing these cloning and shuttling experiments. The open reading frame can be partially or completely sequenced to assure that sequence integrity has been maintained, by comparing the sequence to sequences available from public or private databases of human genes.

In certain examples, the open reading frame can be cloned into a Gateway entry vector (Invitrogen) or cloned directly into pDEST20 (Invitrogen). In other aspects, the entry vector and/or the pDEST20 vector are linearized, for example using BssII, before or during a recombination reaction. In certain aspects, an open reading frame cloned into a pDEST20 vector can be transfected directly into DH10Bac cells. Alternatively, a vector can be constructed with the important functional elements of pDEST20 and used to transfect DH10Bac cells directly. An open reading frame of interest can be cloned directly into the vector using, for example, restriction enzyme cleavages and ligations.

Systems are available for expressing open reading frames in baculovirus. For example, insect cells are typically used for this expression. Any host cell that can be grown in culture can be used to synthesize the proteins of interest. Preferably, host cells are used that can overproduce a protein of interest, resulting in proper synthesis, folding, and posttranslational modification of the protein. Preferably, such protein processing forms epitopes, active sites, binding sites, etc. useful for assays to characterize molecular interactions in vitro that are representative of those in vivo.

In certain illustrative embodiments, the host cell is an insect host cell. A variety of insect cells are commercially available (see, e.g., Invitrogen). The cells can be, for example, Hi-5 cells (available from the University of Virginia, Tissue Culture Facility), sf9 cells (Invitrogen), or SF21 cells (Invitrogen). In certain illustrative embodiments, the insect cells are sf9 cells. In a particular embodiment, yeast cultures are used to synthesize eukaryotic fusion proteins. In one aspect, the yeast Pichia pastoris is used. Fresh cultures are preferably used for efficient induction of protein synthesis, especially when conducted in small volumes of media. Also, care is preferably taken to prevent overgrowth of the yeast cultures. In addition, yeast cultures of about 3 ml or less are preferable to yield sufficient protein for purification. To improve aeration of the cultures, the total volume can be divided into several smaller volumes (e.g., four 0.75 ml cultures can be prepared to produce a total volume of 3 ml).

Cells are then contacted with an inducer (e.g., galactose), and harvested. Induced cells are washed with cold (i.e., 4° C. to about 15° C.) water to stop further growth of the cells, and then washed with cold (i.e., 4° C. to about 15° C.) lysis buffer to remove the culture medium and to precondition the induced cells for protein purification, respectively. Before protein purification, the induced cells can be stored frozen to protect the proteins from degradation. In a specific embodiment, the induced cells are stored in a semi-dried state at −80° C. to prevent or inhibit protein degradation.

Cells can be transferred from one array to another using any suitable mechanical device. For example, arrays containing growth media can be inoculated with the cells of interest using an automatic handling system (e.g., automatic pipette). In a particular embodiment, 96-well arrays containing a growth medium comprising agar can be inoculated with yeast cells using a 96-pronger. Similarly, transfer of liquids (e.g., reagents) from one array to another can be accomplished using an automated liquid-handling device (e.g., Q-FILL™, Genetix, UK).

Although proteins can be harvested from cells at any point in the cell cycle, cells are preferably isolated during logarithmic phase when protein synthesis is enhanced. For example, yeast cells can be harvested between OD₆₀₀=0.3 and OD₆₀₀=1.5, preferably between OD₆₀₀=0.5 and OD₆₀₀=1.5. In a particular embodiment, proteins are harvested from the cells at a point after mid-log phase. Harvested cells can be stored frozen for future manipulation.

The harvested cells can be lysed by a variety of methods known in the art, including mechanical force, enzymatic digestion, and chemical treatment. The method of lysis should be suited to the type of host cell. For example, a lysis buffer containing fresh protease inhibitors is added to yeast cells, along with an agent that disrupts the cell wall (e.g., sand, glass beads, zirconia beads), after which the mixture is shaken violently using a shaker (e.g., vortexer, paint shaker).

In a specific embodiment, zirconia beads are contacted with the yeast cells, and the cells lysed by mechanical disruption by vortexing. In a further embodiment, lysing of the yeast cells in a high-density array format is accomplished using a paint shaker. The paint shaker has a platform that can firmly hold at least eighteen 96-well boxes in three layers, thereby allowing for high-throughput processing of the cultures. Further the paint shaker violently agitates the cultures, even before they are completely thawed, resulting in efficient disruption of the cells while minimizing protein degradation. In fact, as determined by microscopic observation, greater than 90% of the yeast cells can be lysed in under two minutes of shaking.

The resulting cellular debris can be separated from the protein and/or other molecules of interest by centrifugation. Additionally, to increase purity of the protein sample in a high-throughput fashion, the protein-enriched supernatant can be filtered, preferably using a filter on a non-protein-binding solid support. To separate the soluble fraction, which contains the proteins of interest, from the insoluble fraction, use of a filter plate is highly preferred to reduce or avoid protein degradation. Further, these steps preferably are repeated on the fraction containing the cellular debris to increase the yield of protein.

Proteins can then be purified from a protein-enriched cell supernatant using a variety of affinity purification methods known in the art. Affinity tags useful for affinity purification of fusion proteins by contacting the fusion protein preparation with the binding partner to the affinity tag, include, but are not limited to, calmodulin, trypsin/anhydrotrypsin, glutathione, immunoglobulin domains, maltose, nickel, or biotin and its derivatives, which bind to calmodulin-binding protein, bovine pancreatic trypsin inhibitor, glutathione-S-transferase (“GST tag”), antigen or Protein A, maltose binding protein, poly-histidine (“His tag”), and avidin/streptavidin, respectively. Other affinity tags can be, for example, myc or FLAG. Fusion proteins can be affinity purified using an appropriate binding compound (i.e., binding partner such as a glutathione bead), and isolated by, for example, capturing the complex containing bound proteins on a non-protein-binding filter. Placing one affinity tag on one end of the protein (e.g., the carboxy-terminal end), and a second affinity tag on the other end of the protein (e.g., the amino-terminal end) can aid in purifying full-length proteins.

In a particular embodiment, the fusion proteins have GST tags and are affinity purified by contacting the proteins with glutathione beads. In further embodiment, the glutathione beads, with fusion proteins attached, can be washed in a 96-well box without using a filter plate to ease handling of the samples and prevent cross contamination of the samples.

In addition, fusion proteins can be eluted from the binding compound (e.g., glutathione bead) with elution buffer to provide a desired protein concentration. In a specific embodiment, fusion proteins are eluted from the glutathione beads with 30 ml of elution buffer to provide a desired protein concentration.

For purified proteins that will eventually be spotted onto microscope slides, the glutathione beads are separated from the purified proteins. Preferably, all of the glutathione beads are removed to avoid blocking of the positionally addressable arrays pins used to spot the purified proteins onto a solid support. In a preferred embodiment, the glutathione beads are separated from the purified proteins using a filter plate, preferably comprising a non-protein-binding solid support. Filtration of the eluate containing the purified proteins should result in greater than 90% recovery of the proteins.

The elution buffer preferably comprises a liquid of high viscosity such as, for example, 15% to 50% glycerol, preferably about 25% glycerol. The glycerol solution stabilizes the proteins in solution, and prevents dehydration of the protein solution during the printing step using a positionally addressable arrayer.

The elution buffer preferably comprises a liquic containing a non-ionic detergent such as, for example, 0.02-2% Triton-100, preferably about 0.1% Triton-100. The detergent promotes the elution of the protein during purification and stabilizes the protein in solution.

Purified proteins are preferably stored in a medium that stabilizes the proteins and prevents dessication of the sample. For example, purified proteins can be stored in a liquid of high viscosity such as, for example, 15% to 50% glycerol, preferably in about 40% glycerol. It is preferred to aliquot samples containing the purified proteins, so as to avoid loss of protein activity caused by freeze/thaw cycles.

The skilled artisan can appreciate that the purification protocol can be adjusted to control the level of protein purity desired. In some instances, isolation of molecules that associate with the protein of interest is desired. For example, dimers, trimers, or higher order homotypic or heterotypic complexes comprising an overproduced protein of interest can be isolated using the purification methods provided herein, or modifications thereof. Furthermore, associated molecules can be individually isolated and identified using methods known in the art (e.g., mass spectroscopy).

Typically a quality control step is performed to confirm that a protein expressed from the open reading frame is isolated and purified. For example, an immunoblot can be performed using an antibody against the tag to detect the expressed protein. Furthermore, an algorithm can be used to compare the size of the expressed protein with that expected based on the open reading frame, and proteins whose size is not within a certain percentage of the expected size, for example, not within 10%, 20%, 25%, 30%, 40%, or 50% of the expected size of the protein can be rejected.

Isolated proteins can be placed on an array using a variety of methods known in the art. In one embodiment, the proteins are printed onto the solid support. Both contact and non-contact printing can be used to spot the isolated protein. In a specific embodiment, each protein is spotted onto the substrate using an OMNIGRID™ (GeneMachines, San Carlos, Calif.) and quil-type pins, for example available from Telechem (Sunnyvale, Calif.). In a further embodiment, the proteins are attached to the solid support using an affinity tag. Use of an affinity tag different from that used to purify the proteins is preferred, since further purification is achieved when building the protein array.

Accordingly, in a further embodiment, the proteins are bound directly to the solid support. In another further embodiment, the proteins are bound to the solid support via a linker. In a particular embodiment, the proteins are attached to the solid support via a His tag. In another particular embodiment, the proteins are attached to the solid support via a 3-glycidooxypropyltrimethoxysilane (“GPTS”) linker. In a specific embodiment, the proteins are bound to the solid support via His tags, wherein the solid support comprises a flat surface. In a preferred embodiment, the proteins are bound to the solid support via His tags, wherein the solid support comprises a nickel-coated glass slide. In a further embodiment, the proteins are bound to the solid support via biotin tags, wherein the solid support comprises a streptavidin-coated glass slide. In a specific embodiment, the proteins are biotinylated at a specific site in vivo. In a certain illustrative embodiment, the specific site on the protein that is biotinylated in vivo is a BioEase tag (Invitrogen).

The positionally addressable arrays of proteins of the present invention are not limited in their physical dimensions and can have any dimensions that are useful. Preferably, the positionally addressable array of proteins has an array format compatible with automation technologies, thereby allowing for rapid data analysis. Thus, in one embodiment, the positionally addressable array of proteins format is compatible with laboratory equipment and/or analytical software. In an illustrative embodiment, the positionally addressable array is a microarray of proteins and is the size of a standard microscope slide. In another preferred embodiment, the positionally addressable array is a microarray of proteins designed to fit into a sample chamber of a mass spectrometer.

The present invention also relates to methods for making a positionally addressable array comprising the step of attaching to a surface of a solid support, at least 100 proteins of Table 1 or Table 2, with each protein being at a different position on the solid support, wherein the protein comprises a first tag. In certain aspects, the protein comprises a second tag. The advantages of using double-tagged proteins include the ability to obtain highly purified proteins, as well as providing a streamlined manner of purifying proteins from cellular debris and attaching the proteins to a solid support. In a particular aspect, the first tag is a glutathione-S-transferase tag (“GST tag”) and the second tag is a poly-histidine tag (“His tag”).

Protein microarrays used in methods provided herein can be produced by attaching a plurality of proteins to a surface of a solid support, with each protein being at a different position on the solid support, wherein the protein comprises at least one tag. The advantages of using double-tagged proteins include the ability to obtain highly purified proteins, as well as providing a streamlined manner of purifying proteins from cellular debris and attaching the proteins to a solid support. The tag can be for example, a glutathione-S-transferase tag (“GST tag”), a poly-histidine tag (His tag”), or a biotin tag. The biotin tag can be associated with a protein in vivo or in vitro. Where in vivo biotinylation is used, a peptide for directing in vivo biotinylation can be fused to a protein. For example, a Bioease™ tag can be used. In certain aspects, a biotin tag is used for protein immobilization on a protein microarray substrate and/or to isolate a recombinant fusion protein before it is immobilized on a substrate at a positionally addressable location. In a particular embodiment, the first tag is a glutathione-S-transferase tag (“GST tag”) and the second tag is a poly-histidine tag (“His tag”). In a further embodiment, the GST tag and the His tag are attached to the amino-terminal end of the protein. Alternatively, the GST tag and the His tag are attached to the carboxy-terminal end of the protein.

Methods for Identifying Enzyme Substrates.

The protein arrays and methods of making protein arrays provided herein, are exemplified for human proteins. However, it will be understood that the methods can be used for any mammalian species to make mammalian protein arrays from one species or from several species on a single array. Accordingly, provided herein are protein arrays, and methods of making the same, that include at least 100, 200, 250, 500, 1000, 2000, 2500, 3000, 4000, 5000, or all proteins from one or more mammalian species, such as mouse, rat, rabbit, monkey, etc. The proteins can be orthologs of the proteins of Table 9, Table 11, and/or Table 13, for example. In illustrative embodiments the arrays and methods of making arrays include 25, 50, 100, 200, 250, 300, 400, or more proteins that are difficult to express and difficult to isolate in a non-denatured state, such as the human proteins and mammalian orthologs of the human proteins provided in Table 15, Table 16, and/or Table 17. It will be understood that the conserved structure of many difficult to express proteins combined with the present invention establishes by illustrating for the proteins of Table 15, 16, and 17 and other difficult to express proteins that are also difficult to isolate in a native form that are present among the proteins listed in Table 9, Table 11, and/or Table 13, that high throughput methods can be used to express, isolate, and microarry these proteins from any mammalian species. In illustrative aspects, the high throughput methods provided herein for expressing, isolating, and microarraying large numbers of proteins can be used to array both difficult to express proteins that are difficult to isolate in a native form and proteins that do not fall within this category together in the same production batch. For example, at least 25. 50, 100, 200, 300, or 400 difficult to express proteins that are also difficult to isolate in a non-denatured state can be processed with at least 100, 200, 250, 500, 1000, 2000, 3000, 4000, 5000, 6000, 7000, 8000, 90000, or 10,000 proteins that do not fall in this categories, under the same expression, isolation, and microarraying conditions.

In another embodiment, the present invention provides a method for identifying a substrate of an enzyme, comprising contacting the enzyme with a positionally addressable array comprising at least 100 proteins immobilized on functionalized glass surface, and identifying a protein on the positionally addressable array that is bound and/or modified by the enzyme, wherein a binding or modifying of the protein by the enzyme indicates that the protein is a substrate for the enzyme. The contacting is typically performed under effective reaction conditions for the on-test enzyme. In contrast to the limitations of the substrate identification approaches discussed in the Background section above, advantages of positionally addressable arrays of proteins include low reagent consumption, rapid interpretation of results, and the ability to easily control experimental conditions. Another major advantage of a positionally addressable array of protein approach, is the ability to rapidly and simultaneously screen large numbers of proteins for enzyme-substrate relationships. Using positionally addressable arrays of proteins that include at least 100, 200, 250, 500, and more particularly at least 1000, 2000, 2500, 3000, 4000, 5000, substantially all, or all of the proteins of a species, especially, for example, human proteins, one can, in principle, determine all of the substrates for a protein-modifying enzyme in a single experiment. Furthermore, methods are provided herein that include superior slide chemistries for performing enzyme substrate determinations.

In certain aspects, the enzyme activity is, for example, kinase activity, protease activity, phosphatase activity, glycosidase, acetylase activity, and other chemical group transferring enzymatic activity. The proteins on the positionally addressable array in certain illustrative embodiments are from the same species, with the possible exception of control proteins included on the positionally addressable array to confirm that the method was carried out properly and/or to facilitate data analysis. In another embodiment, the present invention provides a method for identifying a small molecule, such as a drug or drug candidate, that affects enzymatic modification of a substrate by an enzyme, comprising contacting the drug or drug candidate and the enzyme, with a positionally addressable array comprising a plurality of proteins, for example at least 100 proteins, and identifying a protein on the positionally addressable array that is bound and/or modified by the enzyme, wherein a binding or modifying of the protein by the enzyme indicates that the protein is a substrate for the enzyme. In certain aspects, the positionally addressable arrays of proteins used in the method are the positionally addressable arrays of proteins of the present invention.

In certain aspect, wherein a binding or modifying of the protein by the enzyme is identified by detecting on the array, signals that are (1) at least 2-fold greater than the equivalent proteins in a negative control assay, and/or (2) greater than 3 standard deviations over the median signal/background value for all negative control spots on the array.

In embodiments provided herein for identifying substrates of an enzyme, the present invention provides a positionally addressable array of proteins comprising a solid support that is a flat surface such as, but not limited to, a glass slide. Dense protein arrays can be produced on, for example, glass slides, such that assays for the presence, amount, and/or functionality of proteins can be conducted in a high-throughput manner.

In certain aspects, the proteins immobilized on the positionally addressable array are spaced apart such that the distance between protein spots is between 250 microns and 1 mm, in a preferred embodiment, a distance of between 275 microns and 1 mm is found between each protein spot, and in an illustrative example the distance is 275 microns.

Preferred glass substrates for enzyme substrate determination, include those that are functionalized with a polymer that contains an acrylate functional group, optionally including cellulose. In further embodiments, a glass slide can be functionalized with an epoxy silane (Available from, for example, Schott-Nexperion and Erie Scientific). The functionalized glass substrate can be a substrate with a three-dimensional porous surface comprising a polymer overlaying a glass surface, such as a polymer that contains an acrylate functional group, and optionally including cellulose. The three-dimensional porous surface comprising a polymer overlaying a glass surface, in certain aspects, typically allows proteins to be nested therein. The surface typically includes multiple functional protein-specific binding sites. The surface in illustrative examples, is hydrophobic. In certain illustrative embodiments, the substrate is a positionally addressable array of proteins substrate, such as Protein slides I or Protein slides II (catalog numbers 25, 25B, 50, or 50B) available from Full Moon Biosystems, Sunnyvale, Calif. In certain aspects, the substrate is Protein slides II (cat. No. 25, 25B, 50, or 50B) from Full Moon Biosystems. In other aspects, the positionally addressable array of proteins utilize substrates such as a Corning UltraGAPS (Corning, Cat. No. 40015), GAPS II (Corning, Cat. No. 40003), Super Epoxy slides (TeleChem), Nickel Chelate-coated slides (available for example from Greiner Bio-One Inc., Longwood, Fla. or from Xenopore, Hawthorne, N.J.), or Low Background Aldehyde slides (available from Microsurfaces Inc., Minneapolis, Minn.).

Not to be limited by theory, a glass slide in certain illustrative examples, is used that includes a functionalized surface comprised of a polymer where monomer ratios to make the polymer are adjusted such that the polymer is sufficiently hydrophobic to allow adequate binding, but not too hydrophobic to cause protein denaturation. In one aspect, a substrate profiling method provided herein is repeated with different functionalized glass substrates to help to assure that all substrates for a kinase are identified. Furthermore, a functionalized glass substrate can be tested with a particular kinase to assure that the kinase phosphorylates substrates on the particular functionalized glass substrate before proceeding with an experiment analyzing unknown proteins spotted on the glass substrate. If a kinase autophorphorylates, it can be spotted directly onto the particular functionalized glass substrate to assure that it is compatible with the substrate.

In certain aspects, a kinase known to autophosphorylate is spotted on the array as a control to assure that the reaction was successful and/or to identify a location on the array.

The plurality of proteins can be from one or more species of organism, such as yeast, mammalian, canine, equine, or human. Furthermore, the plurality of proteins can comprise one of the following:

at least 10, 20, 25, 50, 75, 100, 150, 200, 250, 500, 750, 1000, 1500, 2000, 2500, 3000, or all human proteins from the proteins encoded by the sequences listed in Table 1;

at most 10, 20, 25, 50, 75, 100, 150, 200, 250, 500, 750, 1000, 1500, 2000, 2500, 3000, or all human proteins from the proteins encoded by the sequences listed in Table 1;

at least 3500, 4000, 4500, 5000, 7500, 10,000, substantially all, or all human proteins expressed from the human genome;

at least 10, 20, 25, 50, 75, 100, 150, 200, 250, 500, 750, 1000, 1500, 2000, 2500, 3000, 4000, or 5000, 6000, 7000, 7500, or all proteins encoded by the sequences listed in Table 2;

at most 10, 20, 25, 50, 75, 100, 150, 200, 250, 500, 750, 1000, 1500, 2000, 2500, 3000, 4000, or 5000, 6000, 7000, 7500, or all proteins encoded by the sequences listed in Table 2;

at least 10, 20, 25, 50, 75, 100, 150, 200, 250, 500, 750, 1000, 1500, or all human proteins from the proteins encoded by the sequences listed in Table 3;

at most 10, 20, 25, 50, 75, 100, 150, 200, 250, 500, 750, 1000, 1500, or all human proteins from the proteins encoded by the sequences listed in Table 3;

at least 10, 20, 25, 50, 75, 100, 150, 200, 250, 500, 750, 1000, 1500 or all human proteins from the proteins encoded by the sequences whose accession numbers are listed in Table 5 or Table 7;

at most 10, 20, 25, 50, 75, 100, 150, 200, 250, 500, 750, 1000, 1500 or all human proteins from the proteins encoded by the sequences whose accession numbers are listed in Table 5 or Table 7;

at least 10, 20, 25, 50, 75, 100, 150, or all human proteins from the proteins encoded by the sequences whose accession numbers are listed in Table 6 or Table 8;

at most 10, 20, 25, 50, 75, 100, 150, or all human proteins from the proteins encoded by the sequences whose accession numbers are listed in Table 6 or Table 8;

at least 10%, 20%, 25%, 30%, 35%, 40%, 50%, 60%, 70%, 75%, 80%, 90%, 95%, 96%, 97%, 98%, or 99% of human proteins expressed from the human genome;

at least 10, 20, 25, 50, 75, 100, 150, 200, 250, 500, 750, 1000, 1500, 2000, 2500, 3000, 4000, 5000, 6000, 7000, 8000, 9000, 10000, 11000, 12000, 13000, 14000, 15000, 16000, 17000, 17500, or all proteins listed in Table 10;

at most 10, 20, 25, 50, 75, 100, 150, 200, 250, 500, 750, 1000, 1500, 2000, 2500, 3000, 4000, or 5000, 6000, 7000, 8000, 9000, 10000, 11000, 12000, 13000, 14000, 15000, 16000, 17000, 17500, or all proteins listed in Table 10;

at least 10, 20, 25, 50, 75, 100, 150, 200, 250, 500, 750, 1000, 1500, 2000, 2500, 3000, or all proteins listed in Table 9 and/or Table 11; or at most 10, 20, 25, 50, 75, 100, 150, 200, 250, 500, 750, 1000, 1500, 2000, 2500, 3000, or all proteins listed in Table 9 and/or Table 11;

at least 10, 20, 25, 50, 75, 100, 150, 200, 250, 500, 750, 1000, 1500, 2000, 2500, 3000, 4000, 5000 or all proteins listed in Table 13; or at most 10, 20, 25, 50, 75, 100, 150, 200, 250, 500, 750, 1000, 1500, 2000, 2500, 3000, 4000, 5000, or all proteins listed in Table 13.

In certain embodiments, the plurality of proteins can comprise one of the following: at least 5%, 10%, 15%, 20%, 25%, 30%, 40%, 50%, 60%, 70%, 80%, 85%, 90%, 95%, 96%, 97%, 98%, 99%, or all human proteins of a grouping of proteins listed in Table 10. In certain embodiments, the plurality of proteins can comprise one of the following: at most 5%, 10%, 15%, 20%, 25%, 30%, 40%, 50%, 60%, 70%, 80%, 85%, 90%, 95%, 96%, 97%, 98%, 99%, or all human proteins of a grouping of proteins listed in Table 10. Each grouping provides proteins with a particular functional aspect. The groupings listed in Table 10 are gene ontology, biological process, behavior, biological process unknown, cell communication, cell-cell signaling, signal transduction, development, cell differentiation, embryonic development, growth, cell growth, morphogenesis, regulation of gene expression, reproduction, physiological process, cell death, cell growth and/or maintenance, cell homeostasis, cell organization and biogenesis, cytoplasm organization and biogenesis, organelle organization and biogenesis, cytoskeleton organization and biogenesis, cell proliferation, cell cycle, transport, ion transport, protein transport, death, metabolism, amino acid and derivative metabolism, biosynthesis, protein biosynthesis, carbohydrate metabolism, catabolism, coenzyme and prosthetic group metabolism, electron transport, energy pathways, lipid metabolism, nucleobase, nucleoside, nucleotide and nucleic acid metabolism, DNA metabolism, transcription, protein metabolism, protein biosynthesis, protein modification, secondary metabolism, response to biotic stimulus, response to endogenous stimulus, response to external stimulus, response to abiotic stimulus, cellular component, cell, external encapsulating structure, cell envelope, cell wall, intracellular, chromosome, nuclear chromosome, cytoplasm, cytoplasmic vesicle, cytoskeleton, cytosol, endoplasmic reticulum, endosome, golgi apparatus, microtubule organizing center, mitochondrion, peroxisome, ribosome, vacuole, lysosome, nucleus, nuclear chromosome, nuclear membrane, nucleolus, nucleoplasm, ribosome, nuclear membrane, plasma membrane, cellular_component unknown, extracellular, extracellular matrix, extracellular space, unlocalized, molecular_function, antioxidant activity, binding, calcium ion binding, carbohydrate binding, lipid binding, nucleic acid binding, DNA binding, chromatin binding, transcription factor activity, RNA binding, translation factor activity, nucleic acid binding, nucleotide binding, protein binding, ytoskeletal protein binding, actin binding, receptor binding, catalytic activity, hydrolase activity, nuclease activity, peptidase activity, phosphoprotein phosphatase activity, kinase activity, protein kinase activity, transferase activity, enzyme regulator activity, molecular_function unknown, motor activity, signal transducer activity, receptor activity, receptor binding, structural molecule activity, transcription regulator activity, translation regulator activity, translation factor activity nucleic acid binding, transporter activity, electron transporter activity, ion channel activity, neurotransmitter transporter activity.

In certain embodiments, the plurality of proteins can comprise one of the following: at least 1, 2, 3, 4, 5, 6, 7, 8, 9, 10, 15, 20, 25, 50, 75, or at least 100 or all groupings of the proteins in Table 10. at most 1, 2, 3, 4, 5, 6, 7, 8, 9, 10, 15, 20, 25, 50, 75, or at least 100 or all groupings of the proteins in Table 10;

at least 1, 2, 3, 4, 5, 6, 7, 8, 9, 10, 15, 20, 25, 30, 35, 40, 50, 75, 100, 200, 250, 500, 750, 1000, 1500, or all human proteins of a grouping of proteins listed in Table 10; at most 2, 3, 4, 5, 6, 7, 8, 9, 10, 15, 20, 25, 30, 35, 40, 50, 75, 100, 200, 250, 500, 750, 1000, 1500, or all human proteins of a grouping of proteins listed in Table 10;

at least 1, 2, 3, 4, 5, 6, 7, 8, 9, 10, 15, 20, 25, 30, 35, 40, 50, 75, 100, 200, 250, 500, 750, 1000, 1500, 2000, 2500, 3000, or all human proteins of a grouping of proteins listed in Table 11; at most 2, 3, 4, 5, 6, 7, 8, 9, 10, 15, 20, 25, 30, 35, 40, 50, 75, 100, 200, 250, 500, 750, 1000, 1500, 2000, 2500, 3000, or all human proteins of a grouping of proteins listed in Table 11; or

at least 1, 2, 3, 4, 5, 6, 7, 8, 9, 10, 15, 20, 25, 30, 35, 40, 50, 75, 100, 200, 250, 500, 750, 1000, 1500, 2000, 2500, 3000, 4000, 5000 or all human proteins of a grouping of proteins listed in Table 13; at most 2, 3, 4, 5, 6, 7, 8, 9, 10, 15, 20, 25, 30, 35, 40, 50, 75, 100, 200, 250, 500, 750, 1000, 1500, 2000, 2500, 3000, 4000, 5000, or all human proteins of a grouping of proteins listed in Table 13.

It is understood that the actual numbers of proteins on the microarrays provided herein can be different from the number of the upper and lower limits of proteins on the microarrays. For example, a microarray with 24 proteins encoded by the sequences listed in Table 1 would be encompassed by the invention because the microarray encompasses more than 20 and less than 25 proteins encoded by the sequences listed in Table 1.

The proteins on the positionally addressable arrays provided herein are typically produced under non-denaturing conditions. In an even more specific aspect of the invention, the proteins on the positionally addressable arrays provided herein are non-denatured. Furthermore, the proteins in illustrative examples, are full-length proteins, and can include additional tag sequences. Accordingly, the proteins in certain aspects, are full-length recombinant fusion proteins.

In a specific aspect of the invention, each protein is printed on a microarray at the respective concentration listed in Table 7 or Table 8.

In certain embodiments, a microarray of the invention comprises one or more control proteins. In one aspect, the microarray comprises at least 1, 2, 3, 4, 5, 6, 7, 8, 9, 10, 11, 12, or 13 of the control proteins listed in Table 12. In another aspect, a microarray comprises at most 1, 2, 3, 4, 5, 6, 7, 8, 9, 10, 11, 12, or 13 of the control proteins listed in Table 9. or Table 18.

TABLE 12

| Protein | Source | Catalog # | Purposes |
|---|---|---|---|
| Alexa-488 Antibody | Invitrogen | A11059 | Fiduciary marker |
| Alexa-555 Antibody | Invitrogen | A21427 | Fiduciary marker |
| Alexa-647 Antibody | Invitrogen | A21239 | Fiduciary marker |
| Anti-biotin Antibody | Sigma | A0185 | Detection of |
| (mouse) | | | biotinylated probe |
| BSA | Sigma | A8577 | Negative control |
| GST | Sigma | G5663 | GST concentration |
| | | | calculation |
| Biotin-Antibody (goat | Invitrogen | B2763 | Detection of |
| anti-mouse) | | | streptavidin; |
| | | | anti-mouse antibody |
| | | | detection |
| Yeast Calmodulin | Invitrogen | Protometrix- | Protein-protein |
| | | made | interaction control |
| BioEaseCMK(V5) | Invitrogen | Carlsbad- | Protein-protein |
| | | made | interaction control; |
| | | | V5-detection control |
| Anti-GST Antibody | Santa Cruz | SC-459 | Anti-rabbit antibody |
| (rabbit) | | | control |
| Yes Kinase | Invitrogen | P3078 | Fiduciary marker |
| PKC eta | Invitrogen | P2634 | Fiduciary marker |
| YIL033C | Invitrogen | Protometrix- | Control Kinase |
| | | made | substrate |

In another embodiment, kinase substrates, for example all substrates in a species if the protein array comprises all of the proteins of the species, can be identified by, for example, contacting a kinase with a positionally addressable array of proteins, and in the presence of labeled phosphate, detecting phosphorylated interactors using methods known in the art. Alternatively, essentially all kinases in a species can be identified by contacting a substrate that can be phosphorylated with a positionally addressable array of proteins of the invention, and assaying the presence and/or level of phosphorylated substrate by, for example, using an antibody specific to a phosphorylated amino acid. In another embodiment, essentially all kinase inhibitors in a species can be identified by contacting a kinase and its substrate with a positionally addressable array of proteins of the invention, and determining whether phosphorylation of the substrate is reduced as compared with the level of phosphorylation in the absence of the protein on the chip.

Detection methods for kinase activity are known in the art, and include, but are not limited to, the use of radioactive labels (e.g., ³³P-ATP and ³⁵S-g-ATP), fluorescent antibody probes that bind to phosphoamino acids, or fluorescent dyes that bind phosphates (e.g. ProQ Diamond (Invitrogen)).

Similarly, assays can be conducted to identify all phosphatases, and inhibitors of a phosphatase, in a species. For example, whereas incorporation into a protein of radioactively labeled phosphorus indicates kinase activity in one assay, another assay can be used to measure the release of radioactively labeled phosphorus into the media, indicating phosphatase activity.

Enzymatic reactions can be performed and enzymatic activity measured using the positionally addressable arrays of proteins of the present invention. In a specific embodiment, test compounds that modulate the enzymatic activity of a protein or proteins on a positionally addressable array of proteins can be identified. For example, changes in the level of enzymatic activity can be detected and quantified by incubating a compound or mixture of compounds with an enzymatic reaction mixture, thereby producing a signal (e.g., from substrate that becomes fluorescent upon enzymatic activity). Differences between the presence and absence of a test compound can be characterized. Furthermore, the differences in a compound's effect on enzymatic activities can be detected by comparing their relative effect on samples within the positionally addressable array of proteins and between chips.

In an aspect of methods for identifying enzyme substrates provided herein, the methods further include inferring the concentration of the immobilized proteins by immobilizing the proteins on a second positionally addressable array by contacting a substrate with a portion of isolated protein samples that are used to immobilize the proteins on the positionally addressable protein array that is contacted with an enzyme, and determining the concentration of the immobilized proteins on the second positionally addressable array. This aspect assures that negative results from a substrate identification method are not unknowingly caused by a lack of a protein on the positionally addressable array contacted with the enzyme. This is especially important in a parallel processing method in which at least 100, 200, 250, 500, 750, 1000, 1500, 2000, 2500, 3000, 4000, 5000, 6000, 7000, 7500, 8000, 9000, or 10,000 different proteins are expressed in parallel using cell culture methods, and immobilized at high density on a positionally addressable protein array.

The substrate of the second positionally addressable array is typically different than the substrate of the positionally addressable array that is contacted with the enzyme. In one illustrative example, the proteins in the second positionally addressable array are immobilized on a nitrocellulose substrate. Furthermore, in this aspect of the invention, the first positionally addressable protein array is typically a functionalized glass substrate with a three-dimensional porous surface comprising a polymer overlaying a glass surface, including, for example, Protein slides I or Protein slides II available from Full Moon Biosystems (Sunnyvale, Calif.).

The proteins of the isolated protein samples are typically bound to a tag, for example as a fusion protein. The concentration of the immobilized proteins can be determined by immobilizing on the substrate of the second positionally addressable protein microarray, a series of different known concentrations of the tag and/or a control protein bound to the tag, wherein the tag and/or the control protein are derived from solutions comprising different known concentrations of the tag or the control protein. Immobilized proteins on the second positionally addressable array are then contacted with a first specific binding pair member that binds the tag and the level of binding of the first specific binding pair member to the tag on the proteins and the series of tags or control proteins on the second positionally addressable array is used to construct a standard curve to determine the concentration of the proteins on the second positionally addressable array. That is the concentration of the proteins is determined using the level of binding of the first specific binding pair member to the tag on a target protein and the level of binding of the first specific binding pair member to the different known concentrations of the immobilized tag or control protein comprising the tag. The concentration in illustrative embodiments, is determined using a cubic curve fitting method.

The number of tags on the control protein and the target protein are typically known. For example the control protein and the target protein can include one tag molecule per protein molecule. Therefore, the method typically involves immobilizing a series of tagged control proteins of different known concentrations at a series of locations on a microarray to provide a series of spots of the tagged control proteins. Signals obtained for the series of tagged control protein spots after probing, for example with a fluorescently labeled antibody against the tag, are used to generate a standard curve that is used to determine a concentration of one or more target polypeptides. In an illustrative embodiment, the tag is glutathione S-transferase.

For example, the tagged control protein on the series of spots can be present in a concentration of between about 0.001 ng/ul and about 10 ug/ul, between 0.01 ng/ul and 1 ug/ul, between 0.025 ng/ul and 100 ng/ul, between 0.050 ng/ul and 75 ng/ul, between 0.075 ng/ul and 50 ng/ul, or, for example, between 0.1 ng/ul and 25 ng/ul. In one specific embodiment, the tagged control protein can be present at a series of spots at a concentration of tagged control protein of between 0.1 ng/ul and 12.8 ng/ul.

Each protein of the proteins that are immobilized on the first positionally addressable array and the second positionally addressable array and the control protein are usually spotted in more than one spot to provide further statistical confidence in values obtained. In certain example, concentration is determined for a plurality of target proteins, for example at least 100, 200, 250, 500, 750, 1000, 2000, 2500, 5000, 10,000, 20,000, 25,000, 50,000 or 100,1000 target proteins.

In methods provided herein, the concentration is typically determined using a cubic curve fitting method having the following formula:
Y=a*X³+b*X²+c*X

Where X is the spot relative intensity and the Y is the spot protein concentration. The fitting formula is used to calculate all other proteome spots in the slides. Open source software Polyfit is applied for this curve fitting purpose. In order to get a designed polynomial like Y=a*X³+b*X²+c*X+d with d=0, instead of using Polyfit the usual way, we create a new function Y′=Y/X=a*X²+b*X+c, using Polyfit for 2nd order, we get coefficients a, b, c, then use this a, c, b for the 3-rd order polynomial.

Because the protein concentration of the control spots is known and the intensity can be obtained from the uploaded result file, a fitting curve can be created and the correspondent fitting formula based on the control spots' intensity and concentration. The cubic curve fitting method is applied.

The tag on the tagged control can be an affinity purification tag as discussed in further detail herein. The affinity purification tag can be, for example, glutathione S-transferase. A concentration series is a series of protein spots of different known concentrations used to construct a standard curve and associated formula for determining a concentration of an unknown protein. For example, a microarray can include 1, 2, 3, 4, 5, 6, 7, 8, 9, 10, 15, 20, or 25 separate concentration series, and although each tagged protein of a series typically includes the same tag, tagged control proteins of different series can include different tags. Therefore, a microarray with multiple concentration series can be used in determining protein concentrations for proteins that are tagged with any tag represented in a series that is attached to a target protein. In other words, a microarray with multiple concentration series with different tags provides a robust tool that can be used to determine concentration of a target protein for many different tags.

In certain embodiments of the present invention, the concentration of a protein on an array refers to the concentration of the protein in solution when the protein was initially deposited on the array. Therefore, although the contacting and detecting are performed when the target protein is immobilized, the concentration of the target protein in solution is determined using the standard curve. Thus, the method provides a concentration determination not only for the proteins on the positionally addressable array that is contacted with the substrate, but also for the second positionally addressable array.

The method for determining the concentration of a target protein can be used to determine the concentration of 10, 15, 20, 25, 50, 75, 100, 200, 250, 500, 750, 1000, 2000, 2500, 5000, 10,000, 20,000, 25,000, 50,000, 100,000, 200,000, 250,000, 500,000, 750,000, 1,000,000 proteins or more target proteins. The target proteins can be spotted onto 1, 2, 3, 4, 5, 6, 7, 8, 9 or 10 microarrays.

In one aspect of the method provided herein, protein concentrations are determined by using an equivalent solution protein concentration calculation. Each lot of microarray slides is spotted with a known concentration gradient of purified GST protein. Representative arrays are probed with an anti-GST antibody and the resulting signal is used to calculate a standard curve. This standard curve is then used to calculate the equivalent solution protein concentration of the proteins spotted on the arrays. The intensity of signals for the GST protein gradient present in every subarray is used to calculate a standard curve from which the equivalent solution concentrations of all the proteins are extrapolated. This measure is not an absolute amount of protein on the array but reflects the expected solution concentration for each protein. For a protein reported as having an “equivalent solution concentration” of 10 ng/μl, one can use the quantity spotted to determine the quantity of protein on the microarray. For example, 10 pg of protein can be spotted in a single spot.

Methods for Using a Proteome Array

The invention is also directed to methods for using positionally addressable arrays of proteins to assay the presence, amount, and/or functionality of proteins present in at least one sample. Using the positionally addressable arrays of proteins of the invention, chemical reactions and assays in a large-scale parallel analysis can be performed to characterize biological states or biological responses, and determine the presence, amount, and/or biological activity of proteins.

Biological activity that can be determined using a positionally addressable array of proteins of the invention includes, but is not limited to, enzymatic activity (e.g., kinase activity, protease activity, phosphatase activity, glycosidase, acetylase activity, and other chemical group transferring enzymatic activity), nucleic acid binding, hormone binding, etc. High density and small volume chemical reactions can be advantageous for the methods relating to using the positionally addressable arrays of proteins of the invention.

Upon contacting the proteins of a positionally addressable array of proteins of the invention with one or more probes, protein-probe interactions can be assayed using a variety of techniques known in the art. For example, the positionally addressable array of proteins can be assayed using standard enzymatic assays that produce chemiluminescence or fluorescence. Various protein modifications can be detected by, for example, photoluminescence, chemiluminescence, or fluorescence using non-protein substrates, enzymatic color development, mass spectroscopic signature markers, or amplification of oligonucleotide tags.

The probe is labeled or tagged with a marker so that its binding can be detected, directly or indirectly, by methods commonly known in the art. Any art-known marker may be used, including but not limited to tags such as epitope tags, haptens, and affinity tags, antibodies, labels, etc., providing that it is not the same as the affinity tag or reagent used to attach the protein(s) of the positionally addressable array of proteins to the solid substrate of the chip. For example, if biotin is used as a linker to attach proteins to a positionally addressable array of proteins array, then another tag not present in the protein(s) of the positionally addressable array of proteins, e.g., His or GST, is used to label the probe and to detect a protein-probe interaction. In certain embodiments, a photoluminescent, chemiluminescent, fluorescent, or enzymatic tag is used. In other embodiments, a mass spectroscopic signature marker is used. In yet other embodiments, an amplifiable oligonucleotide, peptide or molecular mass label is used.

Any method known to the skilled artisan can be used to label a probe. The probe can be, but is not limited to, a peptide, polypeptide, protein, nucleic acid, or organic molecule. The label can be, but is not limited to, biotin, avidin, a peptide tag, or a small organic molecule. The label can be attached to the probe in vivo or in vitro. Where the label is biotin, the label can be bound to the probe in vitro or vivo using commercially available reagents (Invitrogen, Carlsbad, Calif.). For example, the probe can be a protein probe labeled in vivo with a biotin label, using a fusion protein that includes a peptide to which biotin is covalently attached in vivo. For example, a Bioease™ tag (Invitrogen, Carlsbad, Calif.) can be used. The BioEase™ tag is a 72 amino acid peptide derived from the C-terminus (amino acids 524-595) of the Klebsiella pneumoniae oxalacetate decarboxylase α subunit (Schwarz et al., 1988). Biotin is covalently attached to the oxalacetate decarboxylase α subunit and peptide sequencing has identified a single biotin binding site at lysine 561 of the protein (Schwarz et al., 1988, The Sodium Ion Translocating Oxalacetate Decarboxylase of Klebsiella pneumoniae, J. Biol. Chem. 263, 9640-9645, incorporated herein in its entirety by reference). When fused to a heterologous protein, the BioEase™ tag is both necessary and sufficient to facilitate in vivo biotinylation of the recombinant protein of interest. The entire 72 amino acid domain is required for recognition by the cellular biotinylation enzymes. For more information about the cellular biotinylation enzymes and the mechanism of biotinylation, refer to the review by Chapman-Smith and Cronan, 1999 (Chapman-Smith, A., and J. E. Cronan, J. (1999). Molecular Biology of Biotin Attachment to Proteins, J. Nutr. 129, 477S-484S. incorporated herein in its entirety). In certain specific embodiments, the label is attached to the probe via a covalent bond. The methods of the invention allow verification of the labeling of the probe. In certain, more specific embodiments, the methods of the invention also allow quantification of the labeling of the probe, i.e., what proportion of the probe in a sample of the probe is labeled.

In a specific embodiment, the invention provides a method for detecting a protein-probe interaction comprising the steps of contacting a sample of labeled probe (e.g., labeled protein) with a positionally addressable array comprising at least 100 human proteins from the proteins encoded by the sequences listed in Table 1 or Table 2, with each protein being at a different position on a solid support; and detecting any positions on the array wherein interaction between the labeled probe and a protein on the array occurs.

Accordingly, protein-probe interactions can be detected by, for example, 1) using radioactively labeled ligand followed by autoradiography and/or phosphoimager analysis; 2) binding of hapten, which is then detected by a fluorescently labeled or enzymatically labeled antibody or high-affinity hapten ligand such as biotin or streptavidin; 3) mass spectrometry; 4) atomic force microscopy; 5) fluorescent polarization methods; 6) infrared red labeled compounds or proteins; 7) amplifiable oligonucleotides, peptides or molecular mass labels; 8) stimulation or inhibition of the protein's enzymatic activity; 9) rolling circle amplification-detection methods (Hatch et al., 1999, “Rolling circle amplification of DNA immobilized on solid surfaces and its application to multiplex mutation detection”, Genet. Anal. 15:35-40); 10) competitive PCR (Fini et al., 1999, “Development of a chemiluminescence competitive PCR for the detection and quantification of parvovirus B19 DNA using a microplate luminometer”, Clin Chem. 45:1391-6; Kruse et al., 1999, “Detection and quantitative measurement of transforming growth factor-beta1 (TGF-beta1) gene expression using a semi-nested competitive PCR assay”, Cytokine 11:179-85; Guenthner and Hart, 1998, “Quantitative, competitive PCR assay for HIV-1 using a microplate-based detection system”, Biotechniques 24:810-6); 11) colorimetric procedures; and 12) biological assays (e.g., for virus titers).

In a particular embodiment, protein-probe interactions are detected by direct mass spectrometry. In a further embodiment, the identity of the protein and/or probe is determined using mass spectrometry. For example, one of more probes that have bound to a protein on the positionally addressable array of proteins can be dissociated from the array, and identified by mass spectrometry (see, e.g., WO 98/59361). In another example, enzymatic cleavage of a protein on the positionally addressable array of proteins can be detected, and the cleaved protein fragments or other released compounds can be identified by mass spectrometry.

In one embodiment, each protein on the positionally addressable array of proteins is contacted with a probe, and the protein-probe interactions are detected and quantified. In another embodiment, each protein on the positionally addressable array of proteins is contacted with multiple probes, and the protein-probe interaction is detected and quantified. For example, the positionally addressable array of proteins can be simultaneously screened with multiple probes including, but not limited to, complex mixtures (e.g., cell extracts), intact cellular components (e.g., organelles), whole cells, and probes pooled from several sources. The protein-probe interactions are then detected and quantified. Useful information can be obtained from assays using mixtures of probes due, in part, to the positionally addressable nature of the arrays of the present invention, i.e., via the placement of proteins at known positions on the protein chip, the protein to which the probe binds (“interactor”) can be characterized.

In accordance with the methods of the invention, a probe can be a cell, cell membrane, subcellular organelles, protein-containing cellular material, protein, oligonucleotide, polynucleotide, DNA, RNA, small molecule (i.e., a compound with a molecular weight of less than 500), substrate, drug or drug candidate, receptor, antigen, steroid, phospholipid, antibody, immunoglobulin domain, glutathione, maltose, nickel, dihydrotrypsin, lectin, or biotin.

Probes can be biotinylated for use in contacting a protein array so as to detect protein-probe interactions. Weakly biotinylated proteins are more likely to maintain the biological activity of interest. Thus, a gentler biotinylation procedure is preferred so as to preserve the protein's binding activity or other biological activity of interest. Accordingly, in a particular embodiment, probe proteins are biotinylated to differing degrees using a biotin-transferring compound (e.g., Sulfo-NHS-LC-LC-Biotin; PIERCE™ Cat. No. 21338, USA).

Interactions of small molecules (i.e., compounds smaller than MW=500) with the proteins on a positionally addressable array of proteins also can be assayed in a cell-free system by probing with small molecules such as, but not limited to, ATP, GTP, cAMP, phosphotyrosine, phosphoserine, and phosphothreonine. Such assays can identify all proteins in a species that interact with a small molecule of interest. Small molecules of interest can include, but are not limited to, pharmaceuticals, drug candidates, fungicides, herbicides, pesticides, carcinogens, and pollutants. Small molecules used as probes in accordance with the methods of the invention preferably are non-protein, organic compounds.

Protein Kinase Substrate Profiling Service Business Method.

In another embodiment provided herein, is a method for generating revenue by proving access to a customer, to a product or service for identifying one or more enzyme substrates using a positionally addressable array of proteins. Access can be provided, for example over a telephone line, a direct salesperson contact, or an Internet or other wide area network. The positionally addressable array of proteins used in the product or service can include, in certain illustrative examples, at least 1000, 2000, 2500, 3000, 4000, 5000, 6000, 7000, 7500, 8000, 9000, 10000, or all proteins in a single species, such as a yeast, animal, mammalian, or human species.

The method according to illustrative examples of this embodiment, comprises, providing access to a customer, to a service for identifying a substrate for an enzyme, wherein the service comprises receiving an identity of a target enzyme from a customer; contacting the target enzyme under reaction conditions with a positionally addressable array comprising at least 100 proteins immobilized on a substrate; and identifying a protein on the positionally addressable array that is bound and/or modified by the enzyme, wherein a binding or modifying of the protein by the enzyme indicates that the protein is a substrate for the enzyme; and providing an identity of the substrate to the customer.

In an illustrative aspect, the method identifies kinase substrates. In certain aspects, such as certain illustrative examples for identifying kinase substrates, the positionally addressable array substrate comprises a three-dimensional porous surface comprising a polymer overlaying a glass support.

In one aspect of the service of this embodiment, at least 1000, 2000, 2500, 3000, 4000, 5000, 6000, or 6280 proteins from the yeast Saccharomyces cerevisae are immobilized on the positionally addressable array of proteins. The majority of the proteins from the yeast Saccharomyces cerevisae genome were previously cloned, over expressed, purified and arrayed in an addressable format on chemically modified glass slides (Zhu H, et al., Science, 2001). In another aspect, at least 1000, 2000, 2500, 3000, 4000, 5000, 6000, 7000, 7500, 8000, 9000, 10000, 11000, 125000, or all human proteins are immobilized on the positionally addressable array of proteins.

The Kinase Substrate Profiling method provided herein, can be repeated using a different enzyme of the same family or class of enzymes, to confirm the specificity of the substrates that were identified in a first performance of the method. Furthermore, the substrate profiling method can be repeated using a protein array of at least 1000, 2000, 2500, 3000, 4000, 5000, 6000, 7000, 7500, 8000, 9000, 10000, 11000, 125000, or all proteins from another species. For example, a first array used in the method can be a yeast protein array and a second protein array can be a human protein array. Furthermore, an inhibitor for an enzyme, such as a kinase, can be analyzed using the array to confirm the specificity of the substrate. Alternatively, test compounds can be screened to identify a test compound that affects the ability of the enzyme to catalyze a reaction involving the substrate. Finally, purified proteins identified as substrates in the substrate profiling method can be sold to customers for use in kinase assay development.

In another embodiment, presented herein is a method of purchasing a population of cells comprising, providing a positionally addressable array comprising at least 100 proteins from the proteins encoded by the sequences listed in Table 1 and/or Table 2, providing a link to purchase a population of clones each expressing one of the at least 100 proteins. In another embodiment, provided herein is a population of fusion proteins comprising at least 10, 20, 25, 50, 75, 100, 150, 200, 250, 500, 750, 1000, 1500, 2000, 2500, 3000 isolated proteins from the proteins encoded by the sequences listed in Table 1 or Table 2, each linked to a tag. In certain aspects, the tag linked to the at least 100 proteins is the same for each of the at least 100 proteins, for example a His tag or a glutathione S-transferase (GST) tag. The tag is in certain illustrative embodiments, is linked to the protein by a covalent bond.

In one example, a kinase and a compound are received from a customer on date 1. Three concentrations of the kinase (0.1, 1.0, and 10 nM) are assayed on a Kinase Substrate Profiling (KSP) positionally addressable array of proteins, for example a positionally addressable array of proteins with over 3000 yeast proteins, in the presence of ³³P-ATP. A positive control utilizing a protein kinase, such as PKA, and a negative control consisting of ³³P-ATP alone are run in parallel. Both control experiments are performed according to established parameters, and the optimal concentration of the customer's kinase is determined. Analysis of the data that is obtained from determining the optimal concentration of kinase, reveals the number of proteins that are phosphorylated sufficiently to give signals that are greater than 3 standard deviations over background. Furthermore, analysis of the data provide the number of proteins that are determined to be specific to the customer's kinase (i.e. not observed in the PKA assay).

A method according to another illustrative example of this embodiment, comprises providing access to a customer, to a product for identifying one or more substrates for an enzyme, wherein the product is a high density addressable protein array comprising at least 100, 200, 250, 500, 750, 1000, 1500, 2000, 2500, 3000, 4000, 5000, 6000, 7000, 7500, 8000, 9000, 10000, or all human proteins. In certain embodiments, the product is a high density addressable protein array comprising at least 100, 200, 250, 500, 750, 1000, 1500, or all of the human proteins listed in Table 1 or 2. In an illustrative aspect, the product is marketed as a product for identifying kinase substrates. In certain examples, the human proteins in on the high density addressable protein array are immobilized on a functionalized glass slide.

Methods for Identifying Molecules that Affect Phosphorylation of a Substrate

In certain embodiments, provided herein are methods for identifying a molecule that affects phosphorylation of a substrate, comprising contacting a kinase with an identified substrate selected from one or more substrates in the presence of the molecule, and determining whether the molecule affects phosphorylation of the identified substrate by the kinase. The molecule can be a small organic molecule or a biomolecule such as a peptide, oligonucleotide, polypeptide, polynucleotide, lipid, or a carbohydrate, for example. In certain aspects, the biomolecule is a hormone, a growth factor, or an apoptotic factor.

The kinase, the identified substrate, and the molecule are contacted under effective reaction conditions (i.e., reaction conditions under which the kinase phosphorylates the identified substrate(s) in the absence of the molecule). It will be understood that many methods are known for testing phosphorylation of a substrate by a kinase. Illustrative examples include array-based methods, such as those provided in the illustrative embodiment entitled “ProtoArray™ Kinase Substrate Identification,” as well as solution-based assays, as provided in the section entitled “VALIDATION OF ARRAY IDENTIFIED PROTEIN SUBSTRATES” in the illustrative embodiment entitled “ProtoArray™ Kinase Substrate Identification.” For a solution-based assay for kinase-substrate phosphorylation, a kinase and one or more of its substrates are incubated in the presence of an on-test molecule and labeled ATP, such as radioactively-labeled ATP. After an appropriate incubation, it is determined whether the substrate is phosphorylated by the kinase in the presence of the on-test molecule. Furthermore, the level of phosphorylation can be determined and compared to the level of phosphorylation in the absence of the on-test molecule.

The molecule can affect phosphorylation by partially or completely inhibiting or enhancing phosphorylation of the substrate. Since phosphorylation is known to play an important role in many physiologically relevant processes, the method is useful for identifying candidate molecules as therapeutic agents. In certain aspects, an inhibitory or stimulatory effect on phosphorylation can be determined using statistical methods such that an affect is identified with greater than or equal to 85% confidence. In certain illustrative examples, an affect is identified with greater than or equal to 95% confidence.

Kinases and identified substrates are disclosed”in the illustrative embodiment entitled “ProtoArray™ Kinase Substrate Identification.” These include substrates that were identified in immobilized array-based format or a solution-based assay. Particularly relevant are substrates that were identified in both an array-based format and validated in a solution-based study, as summarized in the illustrative embodiment entitled “ProtoArray™ Kinase Substrate Identification.” For example, if the kinase is CK2 kinase, the substrate is BC001600, BC014658, BC004440, NM-015938, BC016979, and/or NM-001819, and in illustrative examples the substrate is BC001600, BC014658, BC004440, and/or NM_{—}015938. If the kinase is Protein Kinase A, the substrates is NM-004331, NM_{—}023940, BC000463 BC032852, NM_{—}014326, BC002520, BC033005, NM_{—}006521, BC034318, BC047393, NM_{—}003576, NM_{—}138808, NM_{—}014310, BC020221, NM_{—}014012, BC002493, BC011526, NM_{—}032214, and/or NM_{—}138333. In certain illustrative examples where the kinase is Protein Kinase A, the substrate is NM_{—}023940, BC000463 BC032852, BC002520, BC033005, NM_{—}006521, BC034318, BC047393, BC020221, NM_{—}014012, BC002493, BC011526, NM_{—}032214, and/or NM_{—}138333. In examples where the kinase is LCK, the substrate is BC003065, NM_{—}005207, BC020746, NM_{—}004442, NM_{—}004935, and/or NM_{—}003242. In an illustrative example where the kinase is LCK, the substrate is BC003065.

In one aspect, the method for identifying a molecule that affects phosphorylation of a substrate is a microtiter assay. For example, in the microtiter assay the identified substrate, the relevant kinase and one or more test molecules can be combined in the well of a microtiter plate and the level of phosphorylation can be measured and compared to a control reaction not containing the test molecules. If there is a higher level of phosphorylation, the test molecules stimulate phosphorylation of the identified substrate, if there is a lower level of phosphorylation, the test molecules inhibit phosphorylation of the identified substrate.

Cell-based methods also can be used to identify compounds capable of modulating identified substrate phosphorylation levels. Such assays can also identify compounds which affect substrate expression levels or gene activity directly. Compounds identified via such methods can, for example, be utilized in methods for treating disease or disorders in which the substrate is involved.

In one embodiment, an assay is a cell based assay in which a cell which expresses a membrane bound form of the identified substrate, or a biologically active portion thereof, on the cell surface is contacted with a test molecule and the ability of the test molecule to bind to the substrate determined. In another embodiment the substrate is cytosolic. The cell, for example, can be a yeast cell or a cell of mammalian origin. Determining the ability of the test compound to bind to the substrate can be accomplished, for example, by coupling the test compound with a radioisotope or enzymatic label such that binding of the test compound to the identified substrate or biologically active portion thereof can be determined by detecting the labeled compound in a complex. For example, test compounds can be labeled with 125I, 35S, 14C, or 3H, either directly or indirectly, and the radioisotope detected by direct counting of radio-emission or by scintillation counting. Alternatively, test molecules can be enzymatically labeled with, for example, horseradish peroxidase, alkaline phosphatase, or luciferase, and the enzymatic label detected by determination of conversion of an appropriate substrate to product. In a preferred embodiment, the assay comprises contacting a cell which expresses a membrane bound form of the identified kinase substrate, or a biologically active portion thereof, on the cell surface with a known molecule which binds the substrate to form an assay mixture, contacting the assay mixture with a test molecule, and determining the ability of the test molecule to interact with the substrate, wherein determining the ability of the test molecule to interact with the substrate comprises determining the ability of the test molecule to preferentially bind to the substrate or a biologically active portion thereof as compared to the known molecule.

In another embodiment, an assay is a cell based assay in which a cell which expresses a membrane bound form of the identified substrate, or a biologically active portion thereof, on the cell surface is contacted with the appropriate kinase and one or more test molecules and the ability of the test molecules to affect the level of phosphorylation of the identified substrate is determined. In another embodiment the identified substrate is cytosolic. The cell, for example, can be a yeast cell or a cell of mammalian origin. In a preferred embodiment, the assay comprises contacting a cell which expresses the identified kinase substrate, or a biologically active portion thereof, and expresses the appropriate kinase to form an assay mixture, contacting the assay mixture with one or more test molecules, and determining the ability of the test compounds to modulate the level of phosphorylation of the substrate.

In another aspect, a Km is determined for phosphorylation of an identified substrate by a kinase identified herein as phosphorylating the substrate in the presence of an on-test molecule. The Km is compared to the Km known for the phosphorylation of the identified substrate in the absence of the on-test molecule. A change in the Km indicates that the test molecule affects phosphorylation of the identified substrate by the kinase.

In certain aspects, a determination of whether the test molecule affects phosphorylation of an identified substrate by a kinase identified herein to phosphorylate the identified substrate, is performed using an indirect method. For example, affect on various cellular components and processes can be identified, for example affects on cell proliferation can be determined.

In certain aspects, the test molecule is an antibody or fragment thereof. Where the test molecule is a small molecule, it can be an organic molecule or an inorganic molecule. (e.g., steroid, pharmaceutical drug). A small molecule is considered a non-peptide compound with a molecular weight of less than 500 daltons.

This embodiment of the invention is well suited to screen chemical libraries for molecules that modulate the level of phosphorylation of the substrates identified by the methods of the present invention. The chemical libraries can be peptide libraries, peptidomimetic libraries, chemically synthesized libraries, recombinant, e.g., phage display libraries, and in vitro translation-based libraries, other non-peptide synthetic organic libraries, etc.

Exemplary libraries are commercially available from several sources (ArQule, Tripos/PanLabs, ChemDesign, Pharmacopoeia). In some cases, these chemical libraries are generated using combinatorial strategies that encode the identity of each member of the library on a substrate to which the member compound is attached, thus allowing direct and immediate identification of a molecule that is an effective modulator. Thus, in many combinatorial approaches, the position on a plate of a compound specifies that compound's composition. Also, in one example, a single plate position may have from 1-20 chemicals that can be screened by administration to a well containing the interactions of interest. Thus, if modulation is detected, smaller and smaller pools of interacting pairs can be assayed for the modulation activity. By such methods, many candidate molecules can be screened.

Many diversity libraries suitable for use are known in the art and can be used to provide compounds to be tested according to the present invention. Alternatively, libraries can be constructed using standard methods. Chemical (synthetic) libraries, recombinant expression libraries, or polysome-based libraries are exemplary types of libraries that can be used.

The libraries can be constrained or semirigid (having some degree of structural rigidity), or linear or nonconstrained. The library can be a cDNA or genomic expression library, random peptide expression library or a chemically synthesized random peptide library, or non-peptide library. Expression libraries are introduced into the cells in which the assay occurs, where the nucleic acids of the library are expressed to produce their encoded proteins.

In one embodiment, peptide libraries that can be used in the present invention may be libraries that are chemically synthesized in vitro. Examples of such libraries are given in Houghten et al., 1991, Nature 354:84-86, which describes mixtures of free hexapeptides in which the first and second residues in each peptide were individually and specifically defined; Lam et al., 1991, Nature 354:82-84, which describes a “one bead, one peptide” approach in which a solid phase split synthesis scheme produced a library of peptides in which each bead in the collection had immobilized thereon a single, random sequence of amino acid residues; Medynski, 1994, Bio/Technology 12:709-710, which describes split synthesis and T-bag synthesis methods; and Gallop et al., 1994, J. Medicinal Chemistry 37(9):1233-1251. Simply by way of other examples, a combinatorial library may be prepared for use, according to the methods of Ohlmeyer et al., 1993, Proc. Natl. Acad. Sci. USA 90:10922 10926; Erb et al., 1994, Proc. Natl. Acad. Sci. USA 91:11422 11426; Houghten et al., 1992, Biotechniques 13:412; Jayawickreme et al., 1994, Proc. Natl. Acad. Sci. USA 91:1614 1618; or Salmon et al., 1993, Proc. Natl. Acad. Sci. USA 90:11708 11712. PCT Publication No. WO 93/20242 and Brenner and Lerner, 1992, Proc. Natl. Acad. Sci. USA 89:5381 5383 describe “encoded combinatorial chemical libraries,” that contain oligonucleotide identifiers for each chemical polymer library member.

In a preferred embodiment, the library screened is a biological expression library that is a random peptide phage display library, where the random peptides are constrained (e.g., by virtue of having disulfide bonding).

Further, more general, structurally constrained, organic diversity (e.g., nonpeptide) libraries, can also be used. By way of example, a benzodiazepine library (see e.g., Bunin et al., 1994, Proc. Natl. Acad. Sci. USA 91:4708 4712) may be used.

Conformationally constrained libraries that can be used include but are not limited to those containing invariant cysteine residues which, in an oxidizing environment, cross-link by disulfide bonds to form cystines, modified peptides (e.g., incorporating fluorine, metals, isotopic labels, are phosphorylated, etc.), peptides containing one or more non naturally occurring amino acids, non-peptide structures, and peptides containing a significant fraction of γ carboxyglutamic acid.

Libraries of non-peptides, e.g., peptide derivatives (for example, that contain one or more non-naturally occurring amino acids) can also be used. One example of these are peptoid libraries (Simon et al., 1992, Proc. Natl. Acad. Sci. USA 89:9367 9371). Peptoids are polymers of non-natural amino acids that have naturally occurring side chains attached not to the alpha carbon but to the backbone amino nitrogen. Since peptoids are not easily degraded by human digestive enzymes, they are advantageously more easily adaptable to drug use. Another example of a library that can be used, in which the amide functionalities in peptides have been permethylated to generate a chemically transformed combinatorial library, is described by Ostresh et al., 1994, Proc. Natl. Acad. Sci. USA 91:11138 11142). Another illustrative example of a non-peptide library is a benzodiazepine library. See, e.g., Bunin et al., 1994, Proc. Natl. Acad. Sci. USA 91:4708-4712.

The members of the peptide libraries that can be screened according to the invention are not limited to containing the 20 naturally occurring amino acids. In particular, chemically synthesized libraries and polysome based libraries allow the use of amino acids in addition to the 20 naturally occurring amino acids (by their inclusion in the precursor pool of amino acids used in library production). In specific embodiments, the library members contain one or more non-natural or non classical amino acids or cyclic peptides. Non-classical amino acids include but are not limited to the D-isomers of the common amino acids, α-amino isobutyric acid, 4-aminobutyric acid, Abu, 2-amino butyric acid; γ-Abu, ε-Ahx, 6-amino hexanoic acid; Aib, 2-amino isobutyric acid; 3-amino propionic acid; ornithine; norleucine; norvaline, hydroxyproline, sarcosine, citrulline, cysteic acid, t-butylglycine, t butylalanine, phenylglycine, cyclohexylalanine, β-alanine, designer amino acids such as 13-methyl amino acids, Cα-methyl amino acids, Nα-methyl amino acids, fluoro-amino acids and amino acid analogs in general. Furthermore, the amino acid can be D (dextrorotary) or L (levorotary).

In another embodiment of the present invention, combinatorial chemistry can be used to identify agents that modulate the level of phosphorylation of the substrate. Combinatorial chemistry is capable of creating libraries containing hundreds of thousands of compounds, many of which may be structurally similar. While high throughput screening programs are capable of screening these vast libraries for affinity for known targets, new approaches have been developed that achieve libraries of smaller dimension but which provide maximum chemical diversity. (See e.g., Matter, 1997, Journal of Medicinal Chemistry 40:1219-1229).

Kay et al., 1993, Gene 128:59-65 (Kay) discloses a method of constructing peptide libraries that encode peptides of totally random sequence that are longer than those of any prior conventional libraries. The libraries disclosed in Kay encode totally synthetic random peptides of greater than about 20 amino acids in length. Such libraries can be advantageously screened to identify the phosphorylation modulators. (See also U.S. Pat. No. 5,498,538 dated Mar. 12, 1996; and PCT Publication No. WO 94/18318 dated Aug. 18, 1994).

A comprehensive review of various types of peptide libraries can be found in Gallop et al., 1994, J. Med. Chem. 37:1233-1251.

In related embodiments, the present invention further provides screening methods for the identification of compounds that increase or decrease the level of phosphorylation of kinase substrates identified by the methods of the present invention by screening a series of molecules, such as a library of molecules. Methods for screening that can be used to carry out the foregoing are commonly known in the art. See, e.g., the following references, which disclose screening of peptide libraries: Parmley and Smith, 1989, Adv. Exp. Med. Biol. 251:215-218; Scott and Smith, 1990, Science 249:386-390; Fowlkes et al., 1992, BioTechniques 13:422-427; Oldenburg et al., 1992, Proc. Natl. Acad. Sci. USA 89:5393-5397; Yu et al., 1994, Cell 76:933-945; Staudt et al., 1988, Science 241:577-580; Bock et al., 1992, Nature 355:564-566; Tuerk et al., 1992, Proc. Natl. Acad. Sci. USA 89:6988-6992; Ellington et al., 1992, Nature 355:850-852; U.S. Pat. No. 5,096,815; U.S. Pat. No. 5,223,409; U.S. Pat. No. 5,198,346; Rebar and Pabo, 1993, Science 263:671-673; and International Patent Publication No. WO 94/18318.

In another embodiment, a method is provided for identifying molecules that interact with the identified substrate. This embodiment identified molecules that have a greater chance of affecting phosphorylation of the identified substrate by a kinase identified herein as phosphorylating the identified substrate. The principle of the assays used to identify compounds that interact with the identified substrate involves preparing a reaction mixture of the identified substrate and the test compound under conditions and for a time sufficient to allow the two components to interact with, e.g., bind to, thus forming a complex, which can represent a transient complex, which can be removed and/or detected in the reaction mixture. These assays can be conducted in a variety of ways. For example, one method to conduct such an assay involves anchoring the identified substrate or the test substance onto a solid phase and detecting substrate gene product/test compound complexes anchored on the solid phase at the end of the reaction. In one embodiment of such a method, the identified substrate is anchored onto a solid surface, and the test compound, which is not anchored, may be labeled, either directly or indirectly. Those test compounds that bind to the identified substrate can then be further tested on their ability to effect the level of phosphorylation of the substrate using methods know in the art, including those described, infra.

In practice, microtiter plates may conveniently be utilized as the solid phase. The anchored component may be immobilized by non-covalent or covalent attachments. Non-covalent attachment may be accomplished by simply coating the solid surface with a solution of the protein and drying. Alternatively, an immobilized antibody, preferably a monoclonal antibody, specific for the substrate protein to be immobilized may be used to anchor the protein to the solid surface. The surfaces may be prepared in advance and stored.

In order to conduct the assay, the nonimmobilized component is added to the coated surface containing the anchored component. After the reaction is complete, unreacted components are removed (e.g., by washing) under conditions such that any complexes formed will remain immobilized on the solid surface. The detection of complexes anchored on the solid surface can be accomplished in a number of ways. Where the previously nonimmobilized component is pre-labeled, the detection of label immobilized on the surface indicates that complexes were formed. Where the previously nonimmobilized component is not pre-labeled, an indirect label can be used to detect complexes anchored on the surface; e.g. using a labeled antibody specific for the previously nonimmobilized component (the antibody, in turn, may be directly labeled or indirectly labeled with a labeled anti-Ig antibody).

Alternatively, a reaction can be conducted in a liquid phase, the reaction products separated from unreacted components, and complexes detected; e.g., using an immobilized antibody specific for the identified substrate gene product or the test compound to anchor any complexes formed in solution, and a labeled antibody specific for the other component of the possible complex to detect anchored complexes.

Any method suitable for detecting protein-protein interactions may be employed for identifying identified substrate-protein interactions, including kinase-substrate interactions. Proteins that interact with the substrate and inhibit or enhance the level of substrate phosphorylation will be potential therapeutics for the treatment of diseases and disorders, including cancer, which involve the identified substrate. Proteins that interact with the identified substrate can also be used in the diagnosis of such diseases and disorders.

Among the traditional methods which may be employed are co immunoprecipitation, crosslinking and co-purification through gradients or chromatographic columns (e.g. size exclusion chromatography). Utilizing procedures such as these allows for the isolation of intracellular proteins which interact with the identified substrate, sometimes referred to herein as the substrate gene products. Once isolated, such an intracellular protein can be identified and can, in turn, be used, in conjunction with standard techniques, to identify additional proteins with which it interacts. For example, at least a portion of the amino acid sequence of the intracellular protein which interacts with the identified substrate can be ascertained using techniques well known to those of skill in the art, such as via the Edman degradation technique (see, e.g., Creighton, 1983, Proteins: Structures and Molecular Principles, W.H. Freeman & Co., N.Y., pp. 34-49). The amino acid sequence obtained may be used as a guide for the generation of oligonucleotide mixtures that can be used to screen for gene sequences encoding such intracellular proteins. Screening may be accomplished, for example, by standard hybridization or PCR techniques. Techniques for the generation of oligonucleotide mixtures and the screening are well-known. (See, e.g., Ausubel, supra., and PCR Protocols: A Guide to Methods and Applications, 1990, Innis, M. et al., eds. Academic Press, Inc., New York).

Additionally, methods may be employed which result in the simultaneous identification of genes which encode a protein interacting with the substrate protein. These methods include, for example, probing expression libraries with labeled substrate protein, using substrate protein in a manner similar to the well known technique of antibody probing of λgt11 libraries.

One method which detects protein interactions in vivo, the two-hybrid system, can be used. One version of this system has been described (Chien et al., 1991, supra.) and is commercially available from Clontech (Palo Alto, Calif.).

Kits

The invention also provides kits that include human positionally addressable arrays of proteins of the present invention and/or that are used for carrying out the methods of the present invention. Such kits may further comprise, in one or more containers, reagents useful for assaying biological activity of a protein or molecule, reagents useful for assaying protein-probe interaction, and/or one or more probes, proteins or other molecules. The reagents useful for assaying biological activity of a protein or other molecule, or assaying interactions between a probe and a protein or other molecule, can be applied with the probe, attached to a positionally addressable array of proteins, or contained in one or more wells on a positionally addressable array of proteins. Such reagents can be in solution or in solid form. The reagents may include either or both the proteins or other molecules and the probes required to perform the assay of interest.

In another embodiment, the kit can include the reagent(s) or reaction mixture useful for assaying biological activity, such as enzymatic activity, of a protein or other molecule. The kit typically includes a positionally addressable array of proteins and one or more containers holding a solution reaction mixture for assaying biological activity of a protein or molecule.

The present invention may be better understood by reference to the following non-limiting Examples, which are provided as exemplary of the invention. The following examples are presented in order to more fully illustrate the preferred embodiments of the invention. They should in no way be construed, however, as limiting the broad scope of the invention.

### EXAMPLE 1

### Method for Making a Protein Microarray with Greater Than 3000 Human Proteins

This Example illustrates a method that can be employed to make protein microarrays of large numbers of human proteins.

Cloning, Expression, Purification and Arraying of Human Proteins

A. Cloning

Experimental design, procedures, and protocols. The entire cloning, expression, purification, and arraying performed in this Example were linked to a database and workflow management system that both organizes and tracks the progress from gene sequences to validation of printed protein arrays. Primer pairs were automatically designed using known design parameters to amplify coding sequences and produce fragments with termini that were appropriate for cloning into the Gateway entry vector pENTR221.

PCR amplification from cDNA was carried out in 96-well plates, using a high fidelity polymerase to minimize introduction of spurious mutations. The resulting amplified products were tested for the correct or expected size using a Caliper AMS-90 analyzer. These data were uploaded to the database for an automatic comparison to the gene size expected for each sample clone. A data management system used the results of the Caliper analysis to automatically direct a robotic re-array which consolidated PCR products that have passed QC into a single plate for recombinational cloning into pENTR221. All cloning steps were carried out in bar-coded 96-well plates using robotic liquid handling equipment. These steps included solid-phase DNA purification, BP recombinational cloning reactions, and transformation into competent E. coli. Four colonies were picked from each transformation using a colony-picking robot. PCR reactions and QC of each reaction were carried out on each colony in an automated fashion as described above. Two colonies with the correct sized PCR fragment were robotically consolidated into bar-coded 96-well plates, and the product Templiphi™ (Amersham Biosciences) was used to create templates for automated DNA sequencing.

Analysis, interpretation, and validation. Clones were sequence-verified through the entire length of their inserts. A set of highly efficient algorithms were employed to automatically determine whether the sequence of a clone matched the intended gene, whether there were any deleterious mutations, and whether the ORF was correctly inserted into the vector; only clones that meet these criteria were made available for protein expression.

Benchmarking of this automated system against manual sequence analysis by trained technicians revealed that analysis of 200 clones required 75 hours by manual analysis versus 3 minutes by automation. Further inspection of the results indicated that 9 of the clones passed by manual analysis actually contained sequence errors, and 1 of the clones that failed manual sequence analysis actually had a correct sequence. In contrast, none of the sequences were inappropriately passed or failed by the automated system.

Potential difficulties & solutions. It is inevitable that some sequences will not amplify. One possible cause is errors in the oligonucleotide primers used for PCR. The simplest solution to this problem is to resynthesize primers that fail to amplify. Another possible cause of non-amplification is non-specificity of the oligonucleotides. Although specificity is optimized in the PCR primer design software, it is not possible to always achieve complete specificity. Therefore, we employed a ‘nested primer’ strategy to deal with this; template was amplified by flanking primers prior to specific PCR of the protein or kinase domain. This effectively increased the relative amount of target template, and minimized the effects of non-specificity.

B. Expression and Purification of Human Proteins

Experimental design, procedures and protocols. The goal of this portion of the project was to produce sufficient amounts of recombinant human proteins for production of protein microarrays. We use an insect cell based system for protein production. Recombinant proteins expressed in insect cells have a high frequency of proper folding, high yield, and post-translational modifications (e.g. phosphorylation and glycosylation) that are similar to mammalian cells (Zhu H, et al., Science 2001, 293:2101-2105; and Schweitzer B, and Kingsmore S. F., Curr Opin Biotechnol 2002, 13:14-19; Snyder M, et al., Science 2003, 300:258-260). These desirable features are in contrast to proteins expressed in E. coli, which are often not folded properly and lack post-translational modifications. We have adapted a baculovirus-based system for highly efficient expression of mammalian proteins in a 96-well format. Optimization of this process has allowed us to routinely achieve an 80% or higher success rate in obtaining soluble recombinant proteins from 96-well insect cell cultures; this rate of success represents a significant improvement over the 42% success rate that had been previously reported in this format.

Protein Expression. The baculovirus-based expression system involves the use of a bacmid shuttle vector in an E. coli host containing a transposase. Thus, the vectors used have sequences needed for direct incorporation into the bacmid, as well as the additional elements required for baculovirus driven over-expression: an antibiotic resistance marker, a polyhedrin promoter, an epitope tag (either GST or 6Xhis, or both), and a polyadenylation signal. Just as in the cloning process described previously, sets of cDNAs queued for expression were created and processed as single units of bar-coded 96-well plates. Selected cDNAs (and controls) were robotically re-arrayed for transformation into the bacmid-containing E. coli strain. Following transformation, colonies were picked robotically, and correct integration of the cloned cDNA into the bacmid was automatically checked by an in house data analysis system after PCR. Isolated bacmid DNA was transfected into insect cells where it is believed to form competent virus particles that are propagated by successive insect cell infections and are amplified to a high titer. Amplified viral stocks are stable over many months and allow for multiple separate inoculations and protein expression cycles from each amplification round. Aliquots of amplified viral stocks were used to infect insect cell cultures in bar-coded 96 deep-well plates. Following a 3-day growth, the insect cells containing expressed proteins were collected and lysed in preparation for purification.

Purification. The method for making a protein provided herein optimizes and automates a high-throughput protein purification process so that more than 5000 different proteins can be purified in a single day in a 96-well format. All steps of the process including cell lysis, binding to affinity resins, washing, and elution, were integrated into a fully automated robotic process which was carried out at 4° C. Insect cells were lysed under non-denaturing conditions and lysates were loaded directly into 96-well plates containing glutathione or Ni-NTA resin. After washing, purified proteins were eluted under conditions designed to obtain native proteins.

Analysis, interpretation, and validation. After purification, samples of the purified material were directly compared with crude protein samples obtained from aliquots of cells that have been vigorously lysed and denatured. The two sample sets were run out on SDS-PAGE gels and immuno-detected by Western blot. The gel images were electronically captured and processed to generate a table of all the protein molecular weights detected for each sample that was uploaded into the database. The protein sizing data for both crude and purified protein fractions were automatically scored for the presence or absence of a dominant band at the correct expected molecular weight.

Potential difficulties & solutions. Using this method, in one validation run, 632 out of the 657 (96%) clones submitted for expression passed a crude lysate Western QC. 550 (87%) of these 632 proteins passed Western QC after purification. This validation run clearly demonstrates a high success rate in expressing recombinant proteins using the baculoviral system. In the rare cases when expression is not observed, the protein can be expressed with the fusion tag on the 3′ instead of the 5′ terminus, as this may aid expression or purification. Additional steps that can be taken to increase yield of total protein is to use alternate insect cells, optimize the multiplicity of infection, and examine the effect of culture time on protein yields.

C. Generation of a Positionally Addressable Array of Large Numbers of Human Proteins

Experimental design, procedures and protocols. Microarrays printed with hundreds to thousands of different purified functional proteins were routinely generated. These arrays can be used for a wide variety of applications, including mapping protein-protein, protein-lipid, protein-DNA, and protein-small molecule interactions, enzyme substrate determination, measuring post-translational modifications, and carrying out biochemical assays. The production of these microarrays requires only a small amount of each protein, 1 ug of each protein is sufficient to print hundreds of arrays. Aliquots of each purified protein were robotically dispensed in buffer optimized for microarray printing into microarrayer-compatible bar-coded 384-well plates. The contents of these plates along with plates of proteins used as positive (e.g. fluorescently-labeled proteins, biotinylated proteins, etc.) and negative (e.g. BSA) controls were spotted onto 1″×3″ microscope slides using a microarrayer robot equipped with 48 quill-type pins (Telechem). Each protein was spotted in duplicate with a spot-to-spot spacing of 250 um. Pins were extensively washed and dried after each dispensing cycle to prevent sample carry-over. Up to 10,000 different spots were placed on each slide.

Analysis, interpretation, and validation. A typical lot of microarrays generated from one printing run included 100 slides. Since each of the proteins was tagged with an epitope (e.g. GST or 6×His), representative slides from each printing lot were QC′d using a labeled antibody that is directed against this epitope. Every slide was printed with a dilution series of known quantities of a protein containing the epitope tag. QC images were uploaded into ProtoMine™, a computer system that runs software that calculates a standard curve and converts the signal intensities for each spot into the amount of protein deposited. The intra-slide and intra-lot variability in spot intensity and morphology was measured using automated equipment to determine the number of missing spots, and the presence of control spots. Slides which pass a defined set of QC criteria were stored at −20° C. until use.

Potential difficulties & solutions. One potential difficulty with protein microarrays is denaturation of proteins on the microarray surface. To avoid this problem, we have optimized printing conditions and buffer composition for arraying thousands of different proteins, and have demonstrated stability and functionality of these arrays for at least one year when stored at −20° C. Since proteins sometimes behave differently on different surfaces, when printing an array several different slide types should be analyzed including but not limited to membrane-coated (e.g. nitrocellulose), hydrophobic (e.g. gamma-aminopropylsilane), and covalent (e.g. aldehyde) chemistries. Another issue that arises from time to time is insufficient protein adhering to the surface of the array. A QC process is designed to alert us to this problem, so that proteins that fail to print will be identified. Although a success rate for printing purified proteins is typically 95% or higher, if necessary proteins that fail to print can be further concentrated to increase the likelihood of some protein adhering to the slide.

Table 13, filed herewith on CD in the file named “Table 13,” provides the amino acid sequences, accession numbers, ORF identifier, and FASTA header for 5034 human proteins that the inventors have expressed at a concentration of at least 19.2 nM, isolated, and microarrayed as production lot 5.2, using the protein production, isolation, and microarray methods provided in this Example, and a GST tag. Surprisingly, as indicated in Tables 15-17, the inventors have been able to successfully express numerous difficult-to-express proteins, that are also difficult to isolate in a non-denatured state, such as membrane proteins, including transmembrane proteins and GPCRs, using the same high-throughput methods that were used to expressed other human proteins, including cytoplasmic proteins. Table 15, provided herewith, provides the 429 proteins classified in the Gene Ontology (GO) categories (provided on the Worldwide web at geneontology.org, incorporated herein in its entirety by reference) as “membrane proteins,” that were expressed, isolated, and microarrayed as part of production lot 5.2, using the methods provided in Example 1. Table 16, provided herewith, provides the 88 proteins classified in the GO categories as “transmembrane proteins,” that were expressed, isolated, and microarrayed as part of production lot 5.2, using the methods provided in Example 1. Table 17, provided herewith, provides a list of 42 G-protein coupled receptors that have been expressed, isolated, and microarrayed using the methods provided in Example 1 as part of production lot 5.2. Table 18, filed herewith on CD in the file named “Table 18,” provides the names, identifiers and concentrations at the time of microarray spotting (number in “name” column after “-”) for proteins expressed in production lot 5.2, as well as microarray positional information.

Tables 5 and 7 provide a list including concentration information (Table 7 last column (nM)) of the over 1500 proteins that were successfully expressed, isolated, and microarrayed according to the methods provided in this Example in production lot 4.1. Table 3 provides a list, including coding sequences, of proteins that the inventors expressed at a concentration of at least 19.2 nM, isolated, and microarrayed according to the method provided in Example 1 in production lot 4.1. Table 6 provides a list of the 176 human kinases that were expressed, isolated, and microarrayed using the methods provided in this Example. Table 8 provides a list of human kinases that were expressed, isolated, and microarrayed using the methods provided in this Example. Tables 9 and 11 provide the sequences of proteins that were successfully expressed, isolated and microarrayed using the methods provided in this Example, in different production lots (4.1 and 5.1 respectively). Table 10 lists the human proteins according to Gene Ontology (GO) categories, that were successfully expressed, isolated, and microarrayed using the methods of Example 1 in production lot 5.1. Table 1, filed herewith on CD in the file named “Table 1,” lists the coding sequences encoding human proteins that the inventors attempted to express and isolate using the protein production and isolation methods disclosed in Example 1 herein. Table 2, filed herewith, includes the identities of coding sequences encoding human proteins that include the proteins encoded by the which can be cut out of the clones and ligated into expression vectors. Table 4 provides a list of protein interactions that were identified using the human protein arrays of the present invention. The identification of these interactions further establishes that proteins that were expressed, isolated, and spotted using the methods provided herein are non-denatured proteins retaining their 3-dimensional structure.

To test if human protein arrrays of the present invention could be used to identify novel protein-protein interactions, we expressed and purified 12 his6-V5-bioEase-EK-Human fusions. Among these proteins there were transcricption factors, protein kinases, and cell cycle regulators. To reveal novel protein interactions, the proteins were probed against a human protein array containing approximately 3300 human proteins that were expressed, isolated, and spotted on nitrocellulose slides essentially according to the methods provided in this Example. Interactions were revealed using anti-V5 antibody conjugated to AlexaFluor 647 (anti-V5-AF647) for detection. These interactions were visualized by acquiring images with a fluorescent microarray scanner and displaying with microarray analysis software. For all of the proteins tested, we observed protein interactions with proteins on the array. These interactions are defined as “significant signals” not observed on the negative control slides. The number of interactions ranged from 6 to 30.

From the interactions observed, we identified 19 protein-protein (Table 4) interactions to further examine. The selection was based on interactions that either had very high signals or are consistent with the literature. Some examples of interactions that are consistent with the literature are the interaction of 1) the tyrosine 3-monooxygenase/tryptophan 5-monooxygenase activation protein (YWHAB, 10H3955) with the deathassociated protein kinase 2 (DAPK2, NM014326), 2) the calcium/calmodulin-dependent protein kinase I (CAMK1, IOH21059) with calmodulin-like 5 (CALML5, BC039172) and 3) the CDC37 homolog (CDC37, IOH6219) with the cyclin-dependent kinase 2 (CDK2, NM_{—}001798).

To address if these interactions could be demonstrated by another means, the his6-V5-bioEase-EKhuman fusions were spotted on nitrocellulose coated slides. We then expressed and purified the corresponding GST-fusion interactors using glutathione affinity chromatography. These GST-fusions were then used to probe arrays containing the immobilized his6-V5-bioEase-EK-human fusions. Because the immobilized proteins do not contain a GST tag, we employed an anti-GST based detection strategy.

Of 18 interactions that we expected to observe, 13 were indeed observed. Some of the interactions that were not observed were likely due to the fact that the concentration of the probe was extremely low (0.03 ng/μL). Overall, we observed that the correlation between interactions detected using anti-V5-AlexaFluor647 based detection and interactions detected in a reciprocal interaction assay using anti-GST based detection was approximately 80% (Table 5).

Next, it was confirmed that another lot of human protein arrays of the present invention made according to the present Example at a production scale with respect to the amount of protein expressed and number of slides that were printed, and designated production lot 4.1 (Human Protoarray 4.1 (See Table 9)), could be successfully used to observe protein-protein interactions. To do so, Human Protoarray 4.1 was probed with four his6-V5-bioEase-EK-Human fusions (CALM2, ATF2, CKN1B, and CDC37). Expected interactions for all the probes were observed. CALM2 interacted with CAMKIV (NM_{—}001744). ATF2 interacted with BC029046/PAIP2. CDKN1B interacted with BC005298/CDK7. CDC37 interacted with BC033035, NM-006658 and NM_{—}022720/DGCR8.

TABLE 4
Protein interactions observed using human protein arrays according
to the present invention. The probe (Invitrogen Clone ID) and the
protein immobilized on the slide (Array protein, annotated with MGC
or RefSeq accession) number are listed.

| Interactions Observed | Probe | Array Protein |
|---|---|---|
| IOH3955_BC001709 | IOH3955 | BC001709 |
| IOH12735_BC001716 | IOH12735 | BC001716 |
| IOH3138_BC005298 | IOH3138 | BC005298 |
| IOH6416_BC017348 | IOH6416 | BC017348 |
| IOH1805_BC025700 | IOH1805 | BC025700 |
| IOH12735_BC029046 | IOH12735 | BC029046 |
| IOH3955_BC030253 | IOH3955 | BC030253 |
| IOH6219_BC033035 | IOH6219 | BC033035 |
| IOH21059_BC039172 | IOH21059 | BC039172 |
| IOH5984_NM_001744 | IOH5984 | NM_001744 |
| IOH6219_NM_001798 | IOH6219 | NM_001798 |
| IOH3277_NM_002095 | IOH3277 | NM_002095 |
| IOH26401_NM_002830 | IOH26401 | NM_002830 |
| IOH3277_NM_006307 | IOH3277 | NM_006307 |
| IOH6219_NM_006658 | IOH6219 | NM_006658 |
| IOH3955_NM_014326 | IOH3955 | NM_014326 |
| IOH5984_NM_014326 | IOH5984 | NM_014326 |
| IOH6219_NM_022720 | IOH6219 | NM_022720 |
| IOH3955_NM_138333 | IOH3955 | NM_138333 |

The proteins were spotted on nitrocellulose slides for protein interaction experiments, and Full Moon glass slides (Protein slides II, available from Full Moon Biosystems, Inc., Sunnyvale, Calif.), for kinase substrate profiling experiments.

### EXAMPLE 2

### Kinase Substrate Assay on Protein Arrays

This Example illustrates that kinase substrate assays performed using the protein arrays of the present invention identify specific substrate phosphorylation. One goal of this study was to demonstrate that kinases exhibit specific substrate phosphorylation on protein arrays.

Materials and Methods:

Analysis of Known Kinase Substrates:

pE/Y, myelin basic protein (MBP) and crosstide were handspotted on aldehyde (Telekem) slides and probed with 40 nM Blk with ′γ³³P-ATP B) Crosstide, histone, bio-PKA, bio-PKC printed on aldehyde slides with a SpotBot (Telekem) noncontact arrayer and probed with 40 nM Akt3 with ′γ³³P-ATP. Blk and Akt3 enzymes were purchased from Upstate Signaling Solutions. (product literature for Blk and Akt3 states that the enzymes phosphorylate pE/Y and Crosstide in solution assays respectively).

Analysis of Human Protein Arrays:

1500 human proteins were spotted on aldehyde slides and probed with ′γ³³P-ATP, ′γ³³P-ATP and 40 nM Akt3 or 40 nM Blk and ′γ³³P-ATP. Signals on ′γ³³P-ATP only slide are due to mainly immobilized kinases autophosphorylating on the slide. No substrates were observed for Akt3 but at least four substrates (boxed in red) could be distinquished for Blk.

Results:

To test specific substrated phosphorylation using protein microarrays, we spotted some general substrates on functionalized glass slides. These slides were then probed with two kinases, a tyrosine kinase (Blk) and a serine/threonine kinase (Akt3). Blk is known to phosphorylate the general substrate polyE/Y and Akt3 phosphorylates crosstide in standard solution assays. We observed on protein arrays that Blk preferentially phosphoryaltes pE/Y and Akt3 phosphorylates Crosstide. Akt3 does not phosphorylate pE/Y. Of interest was that Akt3 preferred the general substrates histone, bio-PKA, and bio-PKC over crosstide. The utility of the assay is very apparent because kinases demonstrate specific substrate phosphorylation using the protein microarray assay, and secondly several potential substrates can be screened and identified in one experiment. Lastly, quantitative analyses of the signals can be applied to rank substrates.

Given the ability to show that two commercial enzymes were active against proteins immobilized on glass slides, we decided to test if H. sapiens proteins cloned, expressed in insect cells as GST-fusions and purified by glutathione-affinity chromatography and subsequently immobilized on glass slides with an Omnigrid (Genemachines) noncontact arrayer are suitable substrate arrays for exogenously added kinases. 40 nM Akt3 and 40 nM Blk were added to human protein arrays having approximately 1500 unique proteins.

When we add only a solution of radioactive ′γ³³P-ATP to the human protein array, we observe a number of immobilized proteins that have signal. We believe the signals are the result of kinases autophosphorylating on the array. We also can not exclude the possibility signals result from just ATP binding. It is interesting to note that several proteins not annotated as kinases are ATP reactive. This data argues strongly that proteins are indeed functional on the array. We did not observe any substrate phosphorylation for Akt3 but do observe a number of substrates for Blk. Therefore, we have demonstrated that our process of protein expression, purification and immobilization on arrays produces functional protein arrays that act as ideal substrates for high throughput assessment of protein kinase activity.

Having developed an effective protocol for the printing and probing of substrate arrays with kinases, we reasoned that signals that are only observed in the presence of kinase could be due to two possibilities, either phosphorylation of substrate or autophosphorylation of kinase with subsequent interaction with immobilized protein. To enrich for phosphorylation of immobilized substrate, we reasoned that denaturing washes of kinase-probed arrays would significantly decrease the occurrence of autosphorylated kinase interacting with immobilized protein. We tested 1M NaCl, 1% Triton X-100, 0.5% SDS, 100 mM HCL and 10 mM NaOH on the immobilization of proteins to Ultra GAPS. Most of these treatments had no significant effect on the immobilization of GST fusions. 10 nM NaOH was the only treatment that significantly effected protein immobilization. In certain illustrative embodiments, we used 0.5% SDS washes for the kinase assays.

Initially, we used aldehyde coated slides sold by TeleChem for kinase-substrate assays. Many commercial vendors produce coated (i.e. functionalized) glass slides and we assessed these various slides to determine which chemistry provided the best signal relative to background. Therefore, we purchased 11 different slides from 7 different companies (Table 14). We then printed over a thousand human proteins on these chemistries, probed the slides with a kinase with ′γ³³P-ATP and qualitatively ranked the slides based on signal and background values. We observed that many slides performed similarly with small differences in signal and/or background. The most effective slides were given a score of 2. Less optimal chemistries were given a score of 1 mainly because these slides exhibited higher background. One slide that exhibited extremely high background is the Micromax SuperChip 1 sold by Perkin Elmer. Ultra GAPS slides made by Corning was one particularly effective slide because the proteins exhibited good signal to background ratios and the slides are suitable for other assays types as well.

After the analysis performed as discussed above and summarized in Table 1, reformulated Full Moon glass slides (Protein slides II, available from Full Moon Biosystems, cat. No. 25, 25B, 50, or 50B) were obtained. The reformulated Full Moon functionalized glass slides were found to be particularly effective for use in the kinase assay with contact-printed proteins.

TABLE 14

| Slide Type | Manufacturer | Score |
|---|---|---|
| Super Epoxy | TeleChem | 2 |
| Power Matrix | Full Moon | 2 |
| (Protein slides I) | | |
| Nickel Chelate | Greiner | 2 |
| Nickel Chelate | Xenopore | 2 |
| Low Background Aldehyde | Microsurface | 2 |
| Ultra GAPS | Corning | 2 |
| Super Clean | TeleChem | 1 |
| Super Amine | TeleChem | 1 |
| Super Aldehyde | TeleChem | 1 |
| Aminoslides | Greiner | 1 |
| MicroMax Super Chip I | Perkin Elmer | 0 |

### EXAMPLE 3

### Substrate Profiling Service

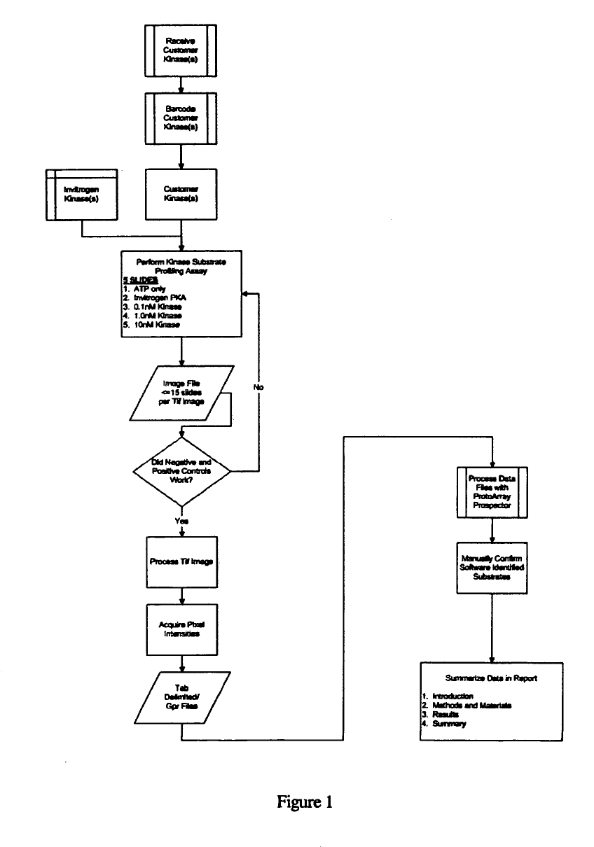

Kinase Substrate Profiling Service. The kinase service method of the present invention was carried out as shown in FIG. 1. This first step was to determine the optimal conditions for kinase substrate discovery. This is accomplished by incubating the kinase at three different concentrations with the Yeast ProtoArray KSP Proteome Positionally addressable array in the presence of ³³P-ATP. A positive control utilizing the protein kinase PKA and a negative control consisting of ³³P-ATP alone was also run in parallel to provide quality assurance for the assay. This data was used to determine which concentration of kinase provides the best signal to background levels while maintaining the presence of fiduciary spots that are necessary for data processing.

Materials and Methods:

Expression of Yeast Proteins. The yeast proteome collection was derived from the yeast clone collection of 5800 yeast ORFs generated by the Snyder lab as described in Zhu et al. (2001). The identity of each clone was verified at Protometrix using 5′ end sequencing. In addition, expression of GST-tagged protein by each clone was tested using Western blotting and detection with an anti-GST antibody. 4088 clones that passed both QC measures were rearrayed into 96-well boxes for long-term storage. One well in each box was also left empty as a negative/contamination control. Frozen yeast 96-well stocks were pronged on to SC/URA growth plates and incubated at 30° C. for 2-3 days. Yeast cells were transferred to 96 well boxes (six replicates per box) containing 1 mL of SC/URA/Raffinose, induced with 4% galactose for 16 hours, the cells pelleted, glass/zirconia beads were added and frozen at −80° C.

Protein Purification. Boxes were thawed at 4° C., lysed four times using a Harbil paint shaker (1 minute shaking periods) in 50 μL lysis buffer with protease inhibitors. To the lysate, 600 μL of buffer with protease inhibitors was added, lysed with the paint shaker and the lysates clarified by centrifugation. 75 μL of glutathione-Sepharose 4B (Amersham Pharmacia) was added, incubated at 6° C. for 1 hr with shaking, the slurries transferred to 96 well PVDF filter plates (Whatman) and washed three times with 200 μL of HEPES wash buffer. Proteins were eluted with 75 μL of Elution Buffer and consolidated into 384 well plates.

Manufacture of Yeast ProtoArray™ KSP Proteome Positionally Addressable Arrays

Proteins. Proteins were purified and distributed in 384-well plates as described above. Four 384-well plates of control proteins were prepared in the elution buffer to ensure consistency of the spots on the arrays. Plates were barcoded, sealed and stored at −80° C. until use.

Array substrate. The array substrate was a 1″×3″ glass microscope slide that was derivatized with chemicals to promote protein binding (Full Moon Biosystems, Sunnyvale, Calif.).

Array Design. The arrays are designed to accommodate 12288 spots. Samples were printed in 48 subarrays (4000-μm² each) and were equally spaced in both vertical and horizontal directions. For the Yeast ProtoArray™ KSP positionally addressable arrays, spots were printed with a 275 μm spot-to-spot spacing. An extra 500-μm gap exists between adjacent subarrays to allow quick identification of subarrays.

Arrayer. The production arrayer was a GeneMachines OmniGrid 100 (Genomic Solutions) equipped with 48 quill-type pins (Telechem International, Sunnyvale, Calif.).

Kinase Substrate Profiling. Positionally addressable array slides were blocked in 30 mL PBS/1% BSA in plastic trays for 2-3 hrs at 4° C. with gentle shaking. After blocking, arrays were removed from the blocking solution and tapped gently on a Kimwipe to remove excess liquid from the slide surface. Arrays were placed in a 50 mL conical tube, and then 120 μL of 0.1, 1, or 10 nM kinase in kinase buffer containing ³³P-ATP or kinase buffer with ³³P-ATP alone (Negative Control) was added. Arrays were covered with a Hybrislip, and the conical tube was capped and placed in an incubator at 30° C. for 1 hr. The tubes were then removed from the incubator and 40 mls of 0.5% SDS in water was added to the tube. The Hybrislip was removed from the tube with tweezers and discarded. The tube was then recapped and gently inverted several times. After a 15 minute incubation at room temperature, the wash buffer was discarded, and another 40 mls of 0.5% SDS in water was added to the tube for a 15 minute incubation. Following this incubation, the wash buffer was discarded and 40 ml of water was added to the tube for a 15 minute incubation at room temperature. After discarding this wash buffer, arrays were placed in a slide holder which was spun in a table top microfuge equipped with microplate rotor at 2000 RPM for 1 minute. Arrays were then placed in an X-ray film cassette, covered with clear plastic wrap and then with a phosphoimaging screen. Exposure of the arrays to the phosphoroimaging screen was carried out for 18 hrs prior to scanning on the phosphorimager.

Data Analysis. The TIFF file produced from the scanning was processed using Adobe Photoshop as follows:

1. 1″×3″ fixed rectangular areas corresponding to each array were cropped from each file.

2. The data was inverted.

3. The image file was changed to 2550×7650 pixels (constrained proportions).

4. The cropped image was saved to a new file.

Pixel intensities for each spot on the array were obtained using GenePix 6.0 software and the array list file supplied with each lot of arrays. Average background for the entire array was used for background subtraction. Local background subtraction was not applied.

Results:

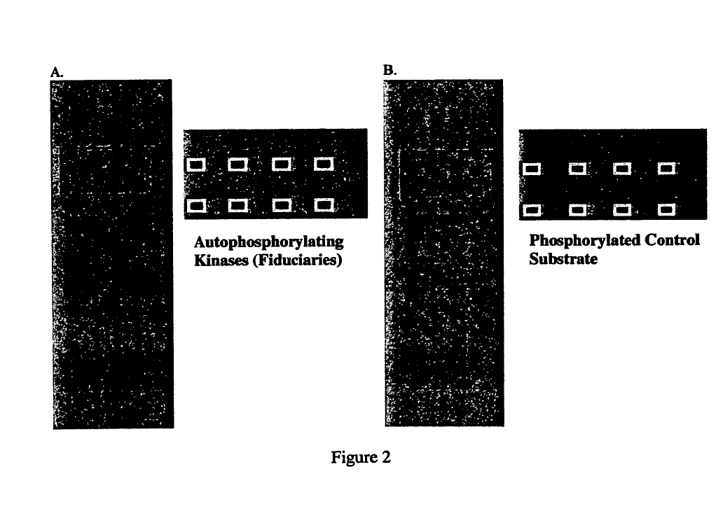

Assay Optimization. In the preliminary phase of this work, three different concentrations of the customer's kinase were incubated with the Yeast ProtoArray™ KSP Proteome Positionally addressable array in the presence of ³³P-ATP. Two types of control assays were also performed in parallel. In the negative control assay, a Yeast ProtoArray™ KSP Proteome Positionally addressable array was incubated with ³³P-ATP alone. FIG. 2A shows the regular pattern of fiduciary spots in each subarray originating from control protein kinases which autophosphorylate. Other pairs of spots are also observed which are derived from autophosphorylating yeast kinases that are part of the yeast proteome collection. In the positive control assay, a Yeast ProtoArray™ KSP Proteome Positionally addressable array was incubated with the protein kinase PKA (FIG. 2B). The image from this experiment shows the same pattern of fiduciary spots as seen in FIG. 2A; however, a significant number of additional proteins show signals as a result of phosphorylation by the added PKA. Of particular note is the control protein shown in the inset; phosphorylation of this protein by PKA indicates that the assay functioned properly.

The customer's kinase was assayed at concentrations of 0.1, 1.0, and 10 nM. A working concentration was selected by identifying the concentration that produces images wherein spots that were specific for the on-test kinase were observable that were not also observed in the negative control experiment from autophosphorylation. At too high of a concentration high background resulted that made data interpretation difficult.

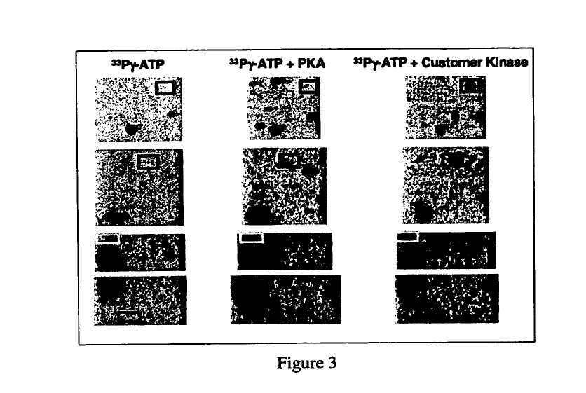

The image obtained from the 1.0 nM concentration of kinase was found to be suitable for data analysis. All spots on all subarrays could be located using the GenePix 6.0 software (data not shown), allowing extraction of signal intensities from the spots. Examples of specific substrates that were identified for the on-test kinase are seen in the subarrays shown in FIG. 3.

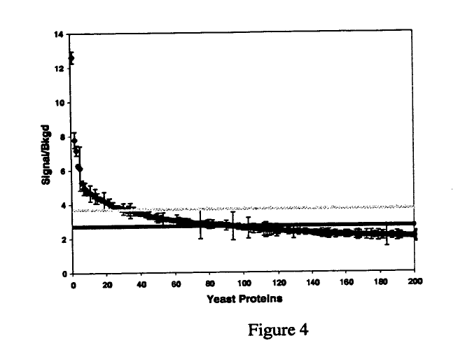

The data file of these intensities, along with similar files for the negative and positive control assays, are made available for downloading on Invitrogen's customer-secure FTP site. ProtoArray™ Prospector (available on the world-wide web at invitrogen.com) was used to analyze the data in these files. Signals for each spot were calculated by dividing the spot feature median pixel intensity by the median pixel intensity for all of the negative control spots on the array. Substrates are defined as proteins on the array having signals that are (1) at least 2-fold greater than the equivalent proteins in the negative control (ATP only) assay, and (2) greater than 3 standard deviations over the median signal/background value for all negative control spots on the array. Using these definitions, ProtoArray™ Prospector identified proteins that were substrates for the customer's kinase. Many of these proteins were not observed to be phosphorylated by PKA, suggesting that these substrates are specific to the customer's kinase. A graphical analysis of the 200 proteins on the array with the highest signals is shown in FIG. 4.

Discussion:

The Kinase Substrate Profiling Service provided herein, identified a significant number of substrates for the on-test kinase. One possible next step includes repeating the assay with the same kinase and a different kinase to confirm the specificity of the substrates that were identified. The Kinase Substrate Profiling Service also offers assays on arrays of greater than 2000 Human proteins. Furthermore, an inhibitor for the kinase can be analyzed on either the Yeast or Human ProtoArrays™. Finally, purified proteins identified as substrates in the substrate profiling method can be sold to clients for use in kinase assay development.

TABLE 5

| COLONY_NAME | COLONY_ID | ACCNO | truncAcc |
|---|---|---|---|
| IOH10670 | 216928 | NM_001637.1 | NM_001637 |
| IOH13082 | 216944 | BC013393.2 | BC013393 |
| IOH10699 | 216927 | BC024187.2 | BC024187 |
| IOH13295 | 216946 | BC012330.1 | BC012330 |
| IOH12655 | 216947 | BC012072.1 | BC012072 |
| IOH12800 | 216948 | BC014194.1 | BC014194 |
| IOH10808 | 216949 | NM_152613.1 | NM_152613 |
| IOH11247 | 216950 | NM_024411.1 | NM_024411 |
| IOH13403 | 216952 | BC011878.2 | BC011878 |
| IOH13383 | 216954 | NM_145042.1 | NM_145042 |
| IOH13411 | 216955 | BC009253.1 | BC009253 |
| IOH12828 | 216956 | NM_145061.1 | NM_145061 |
| IOH12732 | 216957 | NM_052838.2 | NM_052838 |
| IOH13260 | 216943 | NM_145043.1 | NM_145043 |
| IOH13348 | 216903 | NM_144676.1 | NM_144676 |
| IOH12335 | 216890 | BC022319.1 | BC022319 |
| IOH12946 | 216891 | BC022300.1 | BC022300 |
| IOH10305 | 221173 | BC020555.1 | BC020555 |
| IOH12236 | 216895 | BC013902.1 | BC013902 |
| IOH27257 | 220804 | NM_000286.1 | NM_000286 |
| IOH5639 | 219024 | BC004505.1 | BC004505 |
| IOH4675 | 219025 | BC000742.1 | BC000742 |
| IOH4986 | 219026 | BC004965.1 | BC004965 |
| IOH4978 | 219028 | BC003604.1 | BC003604 |
| IOH9638 | 219029 | BC010464.1 | BC010464 |
| IOH10382 | 219032 | BC017085.1 | BC017085 |
| IOH26854 | 220773 | BC030578.1 | BC030578 |
| IOH10365 | 219020 | NM_152269.1 | NM_152269 |
| IOH21921 | 220806 | NM_000566.1 | NM_000566 |
| IOH5155 | 218987 | BC004219.1 | BC004219 |
| IOH10191 | 219007 | BC009108.1 | BC009108 |
| IOH4935 | 218990 | NM_006272.1 | NM_006272 |
| IOH4375 | 218991 | NM_058199.1 | NM_058199 |
| IOH10070 | 218993 | BC016280.1 | BC016280 |
| IOH10110 | 218994 | BC015904.1 | BC015904 |
| IOH10190 | 218995 | NM_152471.1 | NM_152471 |
| IOH5559 | 219000 | NM_032676.1 | NM_032676 |
| IOH5231 | 219023 | BC004233.1 | BC004233 |
| IOH4958 | 219002 | NM_004781.2 | NM_004781 |
| IOH5629 | 219012 | NM_032691.1 | NM_032691 |
| IOH5397 | 219015 | NM_024319.1 | NM_024319 |
| IOH4971 | 219016 | NM_021974.2 | NM_021974 |
| IOH10125 | 219018 | NM_020422.2 | NM_020422 |
| IOH10205 | 219019 | NM_138470.1 | NM_138470 |
| IOH5544 | 219001 | NM_031448.2 | NM_031448 |
| IOH13364 | 216994 | BC012176.1 | BC012176 |
| IOH12495 | 216977 | NM_018959.1 | NM_018959 |
| IOH12981 | 216978 | NM_001084.2 | NM_001084 |
| IOH13450 | 216979 | NM_178858.3 | NM_178858 |
| IOH12049 | 216980 | BC009510.1 | BC009510 |
| IOH13360 | 216981 | NM_020375.1 | NM_020375 |
| IOH12590 | 216983 | NM_144492.1 | NM_144492 |
| IOH12410 | 216989 | NM_004838.2 | NM_004838 |
| IOH13398 | 216995 | NM_005710.1 | NM_005710 |
| IOH3084 | 219820 | NM_005000.2 | NM_005000 |
| IOH13361 | 217005 | BC014658.1 | BC014658 |
| IOH12774 | 217006 | BC014146.2 | BC014146 |
| IOH11070 | 216986 | BC025990.1 | BC025990 |
| IOH5547 | 219013 | NM_030572.1 | NM_030572 |
| IOH12531 | 218983 | BC011906.1 | BC011906 |
| IOH10550 | 219021 | BC012373.1 | BC012373 |
| IOH11753 | 217714 | BC028351.1 | BC028351 |
| IOH12886 | 216852 | BC022272.1 | BC022272 |
| IOH13125 | 216851 | BC020749.1 | BC020749 |
| IOH1900 | 216848 | NM_000067.1 | NM_000067 |
| IOH13346 | 216859 | NM_005702.1 | NM_005702 |
| IOH13409 | 216846 | BC022043.1 | BC022043 |
| IOH13256 | 216850 | BC017347.1 | BC017347 |
| IOH12757 | 216867 | NM_032601.2 | NM_032601 |
| IOH13382 | 216880 | NM_173825.1 | NM_173825 |
| IOH12113 | 216877 | BC020630.1 | BC020630 |
| IOH12966 | 216876 | NM_152396.1 | NM_152396 |
| IOH12079 | 216875 | BC022258.1 | BC022258 |
| IOH12061 | 216856 | BC022257.1 | BC022257 |
| IOH12653 | 216871 | BC017249.1 | BC017249 |
| IOH12055 | 216853 | BC020843.1 | BC020843 |
| IOH12078 | 216864 | NM_005797.2 | NM_005797 |
| IOH12327 | 216863 | NM_138957.1 | NM_138957 |
| IOH1903 | 216860 | NM_004929.2 | NM_004929 |
| IOH13380 | 216838 | NM_138818.1 | NM_138818 |
| IOH13388 | 216857 | BC020835.1 | BC020835 |
| IOH1913 | 216872 | NM_005138.1 | NM_005138 |
| IOH13476 | 216827 | BC026236.1 | BC026236 |
| IOH22638 | 221174 | NM_003006.2 | NM_003006 |
| IOH3506 | 221175 | BC000450.1 | BC000450 |
| IOH23036 | 221176 | BC022429.1 | BC022429 |
| IOH14340 | 221178 | NM_021158.1 | NM_021158 |
| IOH13630 | 221179 | NM_021104.1 | NM_021104 |
| IOH5674 | 221180 | NM_015510.2 | NM_015510 |
| IOH5508 | 221181 | BC004242.1 | BC004242 |
| IOH5450 | 221182 | NM_020531.2 | NM_020531 |
| IOH9642 | 221183 | BC013609.1 | BC013609 |
| IOH3753 | 221186 | BC001064.1 | BC001064 |
| IOH1875 | 216824 | NM_015971.2 | NM_015971 |
| IOH12140 | 216840 | BC017780.1 | BC017780 |
| IOH12138 | 216842 | NM_130782.1 | NM_130782 |
| IOH12143 | 216828 | BC017781.1 | BC017781 |
| IOH13022 | 216830 | BC020898.1 | BC020898 |
| IOH12831 | 216832 | BC020658.1 | BC020658 |
| IOH13254 | 216835 | NM_173474.2 | NM_173474 |
| IOH1877 | 216836 | NM_005086.3 | NM_005086 |
| IOH14765 | 217704 | BC015634.1 | BC015634 |
| IOH10856 | 217700 | NM_145021.1 | NM_145021 |
| IOH2052 | 216837 | NM_006755.1 | NM_006755 |
| IOH1960 | 216896 | NM_018438.2 | NM_018438 |
| IOH12921 | 216839 | NM_000536.1 | NM_000536 |
| IOH12434 | 216887 | BC017873.1 | BC017873 |
| IOH12104 | 216841 | NM_080816.1 | NM_080816 |
| IOH2022 | 216825 | NM_002198.1 | NM_002198 |
| IOH12569 | 216945 | BC012124.1 | BC012124 |
| IOH13432 | 216894 | BC019080.2 | BC019080 |
| IOH12840 | 216930 | NM_022720.2 | NM_022720 |
| IOH13462 | 216932 | NM_138453.1 | NM_138453 |
| IOH13484 | 216934 | NM_138408.1 | NM_138408 |
| IOH12045 | 216935 | NM_005220.1 | NM_005220 |
| IOH12802 | 216936 | BC014218.2 | BC014218 |
| IOH10695 | 216938 | NM_000442.2 | NM_000442 |
| IOH10975 | 216940 | NM_138722.1 | NM_138722 |
| IOH12682 | 216941 | BC011924.1 | BC011924 |
| IOH12796 | 216942 | NM_030815.1 | NM_030815 |
| IOH12116 | 221169 | BC018928.1 | BC018928 |
| IOH2323 | 216897 | NM_000526.3 | NM_000526 |
| IOH13489 | 216898 | BC022377.1 | BC022377 |
| IOH12322 | 216899 | BC017864.1 | BC017864 |
| IOH13453 | 216929 | BC011923.1 | BC011923 |
| IOH5756 | 216902 | BC008069.2 | BC008069 |
| IOH12194 | 216888 | BC017786.1 | BC017786 |
| IOH12152 | 216910 | BC020688.1 | BC020688 |
| IOH12442 | 216911 | NM_138701.1 | NM_138701 |
| IOH13027 | 216912 | BC022407.1 | BC022407 |
| IOH13026 | 216913 | NM_014485.1 | NM_014485 |
| IOH12740 | 216914 | BC020596.1 | BC020596 |
| IOH12057 | 216915 | BC020620.1 | BC020620 |
| IOH12704 | 216920 | NM_052978.1 | NM_052978 |
| IOH13276 | 216922 | NM_022780.2 | NM_022780 |
| IOH13355 | 216923 | BC014409.1 | BC014409 |
| IOH12778 | 216924 | BC014148.2 | BC014148 |
| IOH13019 | 216901 | BC022405.1 | BC022405 |
| IOH4364 | 221066 | BC000116.1 | BC000116 |
| IOH9626 | 221172 | BC011353.1 | BC011353 |
| IOH5552 | 221051 | NM_032303.1 | NM_032303 |
| IOH5433 | 221052 | BC002834.1 | BC002834 |
| IOH3146 | 221053 | BC006769.1 | BC006769 |
| IOH4355 | 221054 | BC004349.1 | BC004349 |
| IOH3554 | 221055 | NM_003908.1 | NM_003908 |
| IOH3644 | 221056 | NM_002861.1 | NM_002861 |
| IOH6092 | 221060 | NM_001324.1 | NM_001324 |
| IOH4946 | 221061 | NM_058179.1 | NM_058179 |
| IOH5673 | 221062 | BC004889.1 | BC004889 |
| IOH5205 | 221063 | NM_032314.1 | NM_032314 |
| IOH4905 | 221049 | BC001600.1 | BC001600 |
| IOH3221 | 221065 | BC001250.1 | BC001250 |
| IOH5918 | 221048 | NM_015926.2 | NM_015926 |
| IOH3569 | 221067 | NM_004632.2 | NM_004632 |
| IOH3655 | 221068 | NM_004990.2 | NM_004990 |
| IOH6219 | 221072 | NM_007065.2 | NM_007065 |
| IOH3126 | 221073 | NM_018091.2 | NM_018091 |
| IOH5713 | 221074 | NM_024322.1 | NM_024322 |
| IOH3438 | 221077 | NM_006623.1 | NM_006623 |
| IOH4383 | 221078 | NM_004698.1 | NM_004698 |
| IOH3592 | 221079 | BC000463.1 | BC000463 |
| IOH3468 | 221084 | BC000440.1 | BC000440 |
| IOH4508 | 221087 | BC000277.1 | BC000277 |
| IOH4388 | 221089 | NM_000026.1 | NM_000026 |
| IOH5448 | 221064 | BC004258.1 | BC004258 |
| IOH6052 | 221033 | BC004359.1 | BC004359 |
| IOH3720 | 221018 | BC001946.1 | BC001946 |
| IOH4312 | 221019 | NM_017727.2 | NM_017727 |
| IOH3627 | 221020 | BC000525.1 | BC000525 |
| IOH6947 | 221023 | BC008337.1 | BC008337 |
| IOH5867 | 221024 | BC005889.2 | BC005889 |
| IOH4822 | 221025 | NM_006194.1 | NM_006194 |
| IOH5666 | 221026 | BC005134.1 | BC005134 |
| IOH5475 | 221027 | BC004248.1 | BC004248 |
| IOH5395 | 221028 | NM_006303.2 | NM_006303 |
| IOH4609 | 221029 | BC000788.1 | BC000788 |
| IOH3758 | 221030 | BC003595.1 | BC003595 |
| IOH5671 | 221050 | NM_013319.1 | NM_013319 |
| IOH3630 | 221032 | BC002361.1 | BC002361 |
| IOH22295 | 221095 | NM_014364.1 | NM_014364 |
| IOH3490 | 221034 | NM_003756.1 | NM_003756 |
| IOH5905 | 221036 | NM_002298.2 | NM_002298 |
| IOH4855 | 221037 | BC001889.1 | BC001889 |
| IOH5668 | 221038 | BC004888.2 | BC004888 |
| IOH5513 | 221039 | NM_032704.1 | NM_032704 |
| IOH5136 | 221041 | NM_000358.1 | NM_000358 |
| IOH4045 | 221042 | BC001449.1 | BC001449 |
| IOH3508 | 221043 | NM_002805.1 | NM_002805 |
| IOH3633 | 221044 | NM_000284.1 | NM_000284 |
| IOH6276 | 221045 | BC006191.1 | BC006191 |
| IOH6997 | 221047 | BC008023.1 | BC008023 |
| IOH4328 | 221031 | BC000698.1 | BC000698 |
| IOH3022 | 221154 | BC000953.2 | BC000953 |
| IOH9675 | 221137 | BC011460.1 | BC011460 |
| IOH10459 | 221139 | BC013119.1 | BC013119 |
| IOH21691 | 221140 | BC030525.1 | BC030525 |
| IOH23012 | 221141 | NM_080423.1 | NM_080423 |
| IOH22682 | 221142 | NM_005060.2 | NM_005060 |
| IOH22374 | 221143 | BC029660.1 | BC029660 |
| IOH21440 | 221144 | BC022237.1 | BC022237 |
| IOH12694 | 221146 | NM_032775.1 | NM_032775 |
| IOH3606 | 221147 | BC002360.1 | BC002360 |
| IOH4968 | 221148 | NM_018070.2 | NM_018070 |
| IOH10105 | 221149 | BC015814.1 | BC015814 |
| IOH22892 | 221093 | BC012824.1 | BC012824 |
| IOH23015 | 221153 | BC021701.1 | BC021701 |
| IOH14075 | 221132 | NM_013446.2 | NM_013446 |
| IOH22379 | 221155 | BC028983.1 | BC028983 |
| IOH21478 | 221156 | BC013796.1 | BC013796 |
| IOH12752 | 221157 | NM_015938.2 | NM_015938 |
| IOH9977 | 221160 | BC015805.1 | BC015805 |
| IOH22604 | 221162 | NM_021969.1 | NM_021969 |
| IOH23025 | 221163 | NM_139062.1 | NM_139062 |
| IOH21412 | 221164 | NM_014702.1 | NM_014702 |
| IOH10956 | 221166 | NM_006147.1 | NM_006147 |
| IOH14558 | 221168 | BC022329.1 | BC022329 |
| IOH12628 | 216967 | NM_018696.1 | NM_018696 |
| IOH4593 | 221170 | BC000001.1 | BC000001 |
| IOH5520 | 221150 | BC004925.1 | BC004925 |
| IOH21571 | 221114 | BC030290.1 | BC030290 |
| IOH12584 | 216958 | NM_020384.1 | NM_020384 |
| IOH13621 | 221096 | BC016276.1 | BC016276 |
| IOH12547 | 221097 | BC021101.1 | BC021101 |
| IOH12702 | 221098 | BC012079.1 | BC012079 |
| IOH4842 | 221099 | NM_130788.1 | NM_130788 |
| IOH3832 | 221100 | BC000769.1 | BC000769 |
| IOH9647 | 221101 | BC011454.1 | BC011454 |
| IOH2968 | 221103 | NM_000282.1 | NM_000282 |
| IOH22910 | 221105 | BC004122.1 | BC004122 |
| IOH22301 | 221107 | BC030773.2 | BC030773 |
| IOH13631 | 221108 | BC013005.2 | BC013005 |
| IOH4671 | 221136 | NM_004401.1 | NM_004401 |
| IOH9673 | 221113 | BC018426.1 | BC018426 |
| IOH12481 | 221134 | BC009249.1 | BC009249 |
| IOH22973 | 221117 | BC011713.2 | BC011713 |
| IOH22341 | 221119 | BC030592.2 | BC030592 |
| IOH14429 | 221120 | BC010047.1 | BC010047 |
| IOH12488 | 221121 | BC024272.1 | BC024272 |
| IOH13023 | 221122 | NM_015193.1 | NM_015193 |
| IOH9674 | 221125 | BC011519.1 | BC011519 |
| IOH21874 | 221126 | NM_015696.2 | NM_015696 |
| IOH6993 | 221128 | BC008359.1 | BC008359 |
| IOH22994 | 221129 | BC014237.1 | BC014237 |
| IOH22345 | 221131 | NM_006948.1 | NM_006948 |
| IOH22631 | 221094 | BC029054.1 | BC029054 |
| IOH4976 | 221111 | NM_002708.1 | NM_002708 |
| IOH14131 | 217555 | BC021561.1 | BC021561 |
| IOH12494 | 216965 | NM_004105.2 | NM_004105 |
| IOH14207 | 217538 | NM_033317.1 | NM_033317 |
| IOH14124 | 217539 | NM_017952.2 | NM_017952 |
| IOH13986 | 217541 | BC017262.1 | BC017262 |
| IOH14004 | 217543 | BC021559.1 | BC021559 |
| IOH14178 | 217544 | NM_144608.1 | NM_144608 |
| IOH14458 | 217548 | BC017237.1 | BC017237 |
| IOH14168 | 217549 | BC010176.1 | BC010176 |
| IOH14717 | 217550 | NM_138443.1 | NM_138443 |
| IOH14361 | 217552 | NM_152373.2 | NM_152373 |
| IOH14488 | 217536 | BC010137.1 | BC010137 |
| IOH14682 | 217554 | BC021551.1 | BC021551 |
| IOH14151 | 217531 | NM_033161.2 | NM_033161 |
| IOH13887 | 217556 | BC028840.1 | BC028840 |
| IOH14194 | 217557 | BC025345.1 | BC025345 |
| IOH14694 | 217558 | NM_002539.1 | NM_002539 |
| IOH13839 | 217559 | NM_145063.1 | NM_145063 |
| IOH13752 | 217560 | NM_007111.2 | NM_007111 |
| IOH13703 | 217565 | BC021930.1 | BC021930 |
| IOH14146 | 217566 | NM_006567.1 | NM_006567 |
| IOH14071 | 217567 | BC025281.1 | BC025281 |
| IOH14021 | 217569 | NM_016641.2 | NM_016641 |
| IOH14539 | 217570 | BC011779.2 | BC011779 |
| IOH13727 | 217571 | BC010081.2 | BC010081 |
| IOH14674 | 217553 | NM_016093.2 | NM_016093 |
| IOH14513 | 217514 | BC011888.1 | BC011888 |
| IOH14554 | 217500 | NM_017660.2 | NM_017660 |
| IOH14463 | 217501 | BC011739.2 | BC011739 |
| IOH14811 | 217502 | NM_058163.1 | NM_058163 |
| IOH14566 | 217503 | NM_003315.1 | NM_003315 |
| IOH14819 | 217504 | BC018667.1 | BC018667 |
| IOH14669 | 217505 | NM_138355.1 | NM_138355 |
| IOH14855 | 217506 | NM_138387.2 | NM_138387 |
| IOH14059 | 217507 | NM_016207.2 | NM_016207 |
| IOH14693 | 217508 | BC026032.1 | BC026032 |
| IOH13934 | 217509 | BC024269.1 | BC024269 |
| IOH14625 | 217537 | NM_002622.3 | NM_002622 |
| IOH14650 | 217513 | BC011812.1 | BC011812 |
| IOH4058 | 218328 | BC002526.1 | BC002526 |
| IOH14526 | 217515 | NM_005435.2 | NM_005435 |
| IOH14106 | 217518 | BC018736.1 | BC018736 |
| IOH14632 | 217519 | NM_004722.2 | NM_004722 |
| IOH14623 | 217521 | NM_032855.1 | NM_032855 |
| IOH14622 | 217524 | BC010064.2 | BC010064 |
| IOH13517 | 217525 | NM_052844.1 | NM_052844 |
| IOH14206 | 217526 | BC011885.1 | BC011885 |
| IOH13544 | 217527 | NM_052845.1 | NM_052845 |
| IOH13653 | 217528 | BC016381.1 | BC016381 |
| IOH14642 | 217529 | BC021263.1 | BC021263 |
| IOH14571 | 217512 | NM_145169.1 | NM_145169 |
| IOH5665 | 216458 | NM_033003.1 | NM_033003 |
| IOH3593 | 218467 | BC002373.1 | BC002373 |
| IOH23043 | 218476 | NM_0144055.1 | NM_014055 |
| IOH9811 | 218487 | BC009696.1 | BC009696 |
| IOH9857 | 218499 | NM_138730.1 | NM_138730 |
| IOH5745 | 218504 | BC006199.1 | BC006199 |
| IOH3515 | 218513 | BC000503.1 | BC000503 |
| IOH4929 | 216447 | NM_003405.2 | NM_003405 |
| IOH6324 | 216448 | NM_031464.1 | NM_031464 |
| IOH6735 | 216449 | NM_006374.2 | NM_006374 |
| IOH10972 | 216451 | NM_007202.2 | NM_007202 |
| IOH14689 | 217572 | BC011811.1 | BC011811 |
| IOH14401 | 216454 | BC017236.1 | BC017236 |
| IOH23069 | 218442 | NM_018439.1 | NM_018439 |
| IOH5842 | 216459 | NM_016283.2 | NM_016283 |
| IOH6368 | 216460 | NM_003821.2 | NM_003821 |
| IOH5022 | 216461 | NM_020990.2 | NM_020990 |
| IOH10843 | 216463 | BC014794.1 | BC014794 |
| IOH13323 | 216464 | BC020225.1 | BC020225 |
| IOH5678 | 216470 | BC004518.1 | BC004518 |
| IOH6779 | 216472 | BC007872.1 | BC007872 |
| IOH7258 | 216473 | NM_001239.2 | NM_001239 |
| IOH9871 | 216474 | NM_002658.1 | NM_002658 |
| IOH11046 | 216475 | NM_016282.2 | NM_016282 |
| IOH13291 | 216476 | BC020221.1 | BC020221 |
| IOH13877 | 216453 | NM_001744.2 | NM_001744 |
| IOH4360 | 218352 | NM_016497.2 | NM_016497 |
| IOH14020 | 217497 | NM_006521.3 | NM_006521 |
| IOH4285 | 218330 | BC002484.1 | BC002484 |
| IOH4338 | 218331 | NM_058217.1 | NM_058217 |
| IOH3166 | 218332 | BC006838.1 | BC006838 |
| IOH3230 | 218333 | BC000884.1 | BC000884 |
| IOH3518 | 218334 | BC000452.1 | BC000452 |
| IOH4354 | 218340 | NM_024043.1 | NM_024043 |
| IOH4341 | 218343 | BC000691.1 | BC000691 |
| IOH3171 | 218344 | BC006839.1 | BC006839 |
| IOH3523 | 218346 | NM_024348.2 | NM_024348 |
| IOH4232 | 218347 | NM_003609.2 | NM_003609 |
| IOH9793 | 218463 | BC016582.1 | BC016582 |
| IOH4083 | 218350 | BC001426.1 | BC001426 |
| IOH6290 | 218447 | NM_032933.1 | NM_032933 |
| IOH4381 | 218353 | NM_004832.1 | NM_004832 |
| IOH4301 | 218354 | NM_017706.2 | NM_017706 |
| IOH4343 | 218355 | NM_006651.2 | NM_006651 |
| IOH3421 | 218357 | NM_004493.1 | NM_004493 |
| IOH4362 | 218364 | BC000226.1 | BC000226 |
| IOH3196 | 218380 | NM_003254.1 | NM_003254 |
| IOH3469 | 218381 | NM_006110.1 | NM_006110 |
| IOH7008 | 218436 | BC008031.1 | BC008031 |
| IOH7570 | 218437 | BC008461.1 | BC008461 |
| IOH9772 | 218439 | BC013158.1 | BC013158 |
| IOH13543 | 217573 | BC014001.1 | BC014001 |
| IOH3352 | 218348 | NM_080658.1 | NM_080658 |
| IOH7547 | 217298 | BC007110.1 | BC007110 |
| IOHI1281 | 216999 | BC025700.1 | BC025700 |
| IOH12571 | 217000 | NM_016310.2 | NM_016310 |
| IOH12379 | 217001 | BC026126.1 | BC026126 |
| IOH12355 | 217002 | NM_016484.1 | NM_016484 |
| IOHI2380 | 217004 | BC012109.1 | BC012109 |
| IOH10848 | 217008 | NM_024685.1 | NM_024685 |
| IOH10731 | 217009 | BC021172.2 | BC021172 |
| IOH10645 | 217010 | NM_000023.1 | NM_000023 |
| IOH12850 | 217011 | BC011916.1 | BC011916 |
| IOH9833 | 217294 | NM_145244.1 | NM_145244 |
| IOH14129 | 217316 | BC018625.1 | BC018625 |
| IOH9972 | 217297 | BC013571.1 | BC013571 |
| IOH13199 | 216992 | NM_145041.1 | NM_145041 |
| IOH5749 | 217300 | NM_001168.1 | NM_001168 |
| IOH5792 | 217301 | NM_004051.1 | NM_004051 |
| IOH6546 | 217303 | NM_014571.2 | NM_014571 |
| IOH9908 | 217307 | BC013437.1 | BC013437 |
| IOH9978 | 217309 | NM_006333.1 | NM_006333 |
| IOH7548 | 217310 | BC005911.1 | BC005911 |
| IOH7567 | 217311 | NM_080650.1 | NM_080650 |
| IOH5751 | 217312 | NM_001673.2 | NM_001673 |
| IOH5797 | 217313 | NM_004309.2 | NM_004309 |
| IOH5956 | 217314 | BC007658.1 | BC007658 |
| IOH9906 | 217295 | NM_145306.1 | NM_145306 |
| IOH10642 | 217688 | NM_138812.1 | NM_138812 |
| IOH10722 | 216961 | BC018063.1 | BC018063 |
| IOH10800 | 216963 | NM_152314.1 | NM_152314 |
| IOH12777 | 216964 | BC011936.1 | BC011936 |
| IOH12909 | 216966 | NM_016836.1 | NM_016836 |
| IOH4597 | 221014 | NM_003801.2 | NM_003801 |
| IOH12068 | 216968 | BC009506.1 | BC009506 |
| IOH13265 | 216969 | NM_053050.2 | NM_053050 |
| IOH13248 | 216971 | BC011576.1 | BC011576 |
| IOH11158 | 216972 | BC026325.1 | BC026325 |
| IOH10837 | 216973 | NM_145047.1 | NM_145047 |
| IOH10911 | 216974 | NM_024695.1 | NM_024695 |
| IOH10910 | 216998 | BC014607.2 | BC014607 |
| IOH13320 | 216976 | NM_024610.2 | NM_024610 |
| IOH11253 | 216997 | NM_015417.2 | NM_015417 |
| IOH13855 | 217679 | NM_138392.1 | NM_138392 |
| IOH10664 | 217677 | NM_144647.1 | NM_144647 |
| IOH10958 | 217676 | NM_016230.2 | NM_016230 |
| IOH10809 | 216984 | NM_145314.1 | NM_145314 |
| IOH11034 | 216985 | BC022462.1 | BC022462 |
| IOH10931 | 216987 | BC025729.1 | BC025729 |
| IOH13153 | 216988 | NM_032122.2 | NM_032122 |
| IOH12635 | 216990 | BC024208.1 | BC024208 |
| IOH13079 | 216991 | NM_021809.2 | NM_021809 |
| IOH13483 | 216993 | NM_138415.1 | NM_138415 |
| IOH9858 | 217318 | NM_019103.1 | NM_019103 |
| IOH11059 | 216975 | NM_021245.2 | NM_021245 |
| IOH14073 | 217485 | BC024281.1 | BC024281 |
| IOH14750 | 217365 | NM_002028.2 | NM_002028 |
| IOH9894 | 217366 | BC009674.1 | BC009674 |
| IOH9968 | 217368 | BC013569.1 | BC013569 |
| IOH7532 | 217369 | BC007104.1 | BC007104 |
| IOH7438 | 217371 | BC008407.1 | BC008407 |
| IOH5772 | 217372 | BC005823.1 | BC005823 |
| IOH5829 | 217373 | NM_017966.1 | NM_017966 |
| IOH6528 | 217374 | BC005055.1 | BC005055 |
| IOH9947 | 217378 | NM_138787.1 | NM_138787 |
| IOH14704 | 217387 | NM_002648.1 | NM_002648 |
| IOH6566 | 217315 | NM_024493.1 | NM_024493 |
| IOH14846 | 217484 | BC021120.1 | BC021120 |
| IOH5828 | 217361 | NM_007255.1 | NM_007255 |
| IOH13935 | 217486 | NM_022369.2 | NM_022369 |
| IOH14671 | 217487 | NM_003104.2 | NM_003104 |
| IOH13726 | 217488 | BC011710.2 | BC011710 |
| IOH13845 | 217489 | NM_032476.1 | NM_032476 |
| IOH14544 | 217490 | BC014057.1 | BC014057 |
| IOH13943 | 217491 | NM_001679.1 | NM_001679 |
| IOH14624 | 217493 | BC021253.2 | BC021253 |
| IOH14788 | 217494 | BC018749.1 | BC018749 |
| IOH14790 | 217495 | BC022098.1 | BC022098 |
| IOH14762 | 217496 | NM_005347.2 | NM_005347 |
| IOH12587 | 216959 | NM_022154.2 | NM_022154 |
| IOH13954 | 217483 | NM_025108.1 | NM_025108 |
| IOH9864 | 217342 | NM_145252.1 | NM_145252 |
| IOH9933 | 217319 | NM_138793.1 | NM_138793 |
| IOH9993 | 217321 | NM_015987.2 | NM_015987 |
| IOH7549 | 217322 | BC005930.1 | BC005930 |
| IOH7571 | 217323 | NM_006366.1 | NM_006366 |
| IOH5753 | 217324 | NM_001561.3 | NM_001561 |
| IOH5964 | 217326 | NM_006460.1 | NM_006460 |
| IOH9861 | 217330 | BC009738.1 | BC009738 |
| IOH9936 | 217331 | BC015169.1 | BC015169 |
| IOH7553 | 217334 | BC005902.1 | BC005902 |
| IOH5054 | 217335 | NM_004649.1 | NM_004649 |
| IOH5754 | 217336 | NM_001983.1 | NM_001983 |
| IOH14081 | 217364 | BC021105.1 | BC021105 |
| IOH14058 | 217341 | BC018732.1 | BC018732 |
| IOH14069 | 217363 | BC019102.1 | BC019102 |
| IOH9940 | 217343 | NNLOO4853 | NM_004853 |
| IOH7554 | 217346 | NM_014267.2 | NM_014267 |
| IOH5824 | 217349 | BC007414.2 | BC007414 |
| IOH6582 | 217351 | NM_032712.1 | NM_032712 |
| IOH14878 | 217353 | NM_003794.1 | NM_003794 |
| IOH9941 | 217355 | NM_022152.2 | NM_022152 |
| IOH9965 | 217356 | NM_000317.1 | NM_000317 |
| IOH7556 | 217358 | BC008435.1 | BC008435 |
| IOH7416 | 217359 | BC008440.1 | BC008440 |
| IOH5762 | 217360 | NM_032359.1 | NM_032359 |
| IOH13894 | 217498 | NM_021822.1 | NM_021822 |
| IOH13547 | 217340 | BC018766.1 | BC018766 |
| IOH21605 | 220775 | BC031265.1 | BC031265 |
| IOH4717 | 219063 | NM_014358.1 | NM_014358 |
| IOH10010 | 219064 | BC017117.1 | BC017117 |
| IOH9694 | 219065 | NM_001986.1 | NM_001986 |
| IOH10184 | 219066 | BC010518.1 | BC010518 |
| IOH10251 | 219067 | BC013069.1 | BC013069 |
| IOH127248 | 220866 | NM_003358.1 | NM_003358 |
| IOH27133 | 220772 | BC035028.1 | BC035028 |
| IOH28287 | 220867 | AB065662.1 | AB065662 |
| IOH5012 | 217929 | NM_024668.1 | NM_024668 |
| IOH7202 | 217927 | BC005259.1 | BC005259 |
| IOH5335 | 221016 | BC002751.1 | BC002751 |
| IOH23248 | 220774 | BC033196.1 | BC033196 |
| IOH5409 | 219059 | NM_024314.1 | NM_024314 |
| IOH28296 | 220870 | AB065621.1 | AB065621 |
| IOH25778 | 220776 | NM_003878.1 | NM_003878 |
| IOH22820 | 220777 | NM_022141.1 | NM_022141 |
| IOH27453 | 220778 | NM_080745.1 | NM_080745 |
| IOH3090 | 220872 | BC001284.1 | BC001284 |
| IOH22254 | 220779 | NM_139169.2 | NM_139169 |
| IOH21330 | 220873 | NM_002739.1 | NM_002739 |
| IOH27325 | 220874 | NM_000486.2 | NM_000486 |
| IOH27700 | 220780 | BC037333.1 | BC037333 |
| IOH27414 | 220875 | NM_016511.1 | NM_016511 |
| IOH28297 | 220868 | AB065619.1 | AB065619 |
| IOH10418 | 219044 | BC020960.1 | BC020960 |
| IOH10216 | 219031 | BC016464.1 | BC016464 |
| IOH10556 | 219033 | NM_006681.1 | NM_006681 |
| IOH4589 | 219034 | NM_000262.1 | NM_000262 |
| IOH5233 | 219035 | NM_024114.1 | NM_024114 |
| IOH5499 | 219036 | BC004277.1 | BC004277 |
| IOH4704 | 219037 | BC000772.1 | BC000772 |
| IOH5492 | 219038 | NM_004887.2 | NM_004887 |
| IOH3851 | 219039 | BC001129.1 | BC001129 |
| IOH4814 | 219040 | BC005004.1 | BC005004 |
| IOH9639 | 219041 | BC008624.1 | BC008624 |
| IOH4772 | 219061 | NM_004965.3 | NM_004965 |
| IOH10240 | 219043 | NM_033414.1 | NM_033414 |
| IOH5507 | 219060 | NM_032301.1 | NM_032301 |
| IOH5121 | 219046 | NM_080702.1 | NM_080702 |
| IOH5351 | 219047 | BC002752.1 | BC002752 |
| IOH9768 | 219049 | NM_080664.1 | NM_080664 |
| IOH3853 | 219051 | BC001132.1 | BC001132 |
| IOH9964 | 219052 | NM_004545.1 | NM_004545 |
| IOH9691 | 219053 | BC011400.1 | BC011400 |
| IOH10248 | 219055 | BC010562.1 | BC010562 |
| IOH10465 | 219056 | NM_138771.1 | NM_138771 |
| IOH10335 | 219057 | NM_144626.1 | NM_144626 |
| IOH5124 | 219058 | BC003178.1 | BC003178 |
| IOH22624 | 220876 | NM_033423.1 | NM_033423 |
| IOH10180 | 219042 | BC010498.1 | BC010498 |
| IOH4015 | 220902 | NM_014248.2 | NM014248 |
| IOH27210 | 220781 | BC031056.1 | BC031056 |
| IOH7180 | 217926 | NM_012383.2 | NM_012383 |
| IOH23176 | 220898 | NM_024164.2 | NM_024164 |
| IOH6746 | 217917 | NM_012200.2 | NM_012200 |
| IOH7199 | 217915 | NM_005792.1 | NM_005792 |
| IOH27392 | 220899 | BC033509.1 | BC033509 |
| IOH27448 | 220805 | BC038422.1 | BC038422 |
| IOH7460 | 217912 | BC008392.1 | BC008392 |
| IOH6706 | 217904 | NM_019613.2 | NM_019613 |
| IOH22386 | 220900 | NM_015488.1 | NM_015488 |
| IOH27534 | 220801 | BC032390.1 | BC032390 |
| IOH26830 | 220808 | BC034954.2 | BC034954 |
| IOH27198 | 220809 | NM_004566.1 | NM_004566 |
| IOH26798 | 220810 | BC035938.1 | BC035938 |
| IOH28390 | 220905 | NM_033519.1 | NM_033519 |
| IOH25776 | 220814 | BC034726.1 | BC034726 |
| IOH21725 | 220908 | NM_170699.1 | NM_170699 |
| IOH25788 | 220909 | NM_182665.1 | NM_182665 |
| IOH28389 | 220883 | NM_000910.1 | NM_000910 |
| IOH7474 | 217947 | BC007102.1 | BC007102 |
| IOH13194 | 220877 | NM_021170.2 | NM_021170 |
| IOH27690 | 220783 | NM_003692.1 | NM_003692 |
| IOH23122 | 220785 | NM_144684.1 | NM_144684 |
| IOH28328 | 220879 | NM_153445.1 | NM_153445 |
| IOH27154 | 220786 | NM_018189.1 | NM_018189 |
| IOH28529 | 220880 | XM_291436.1 | XM_291436 |
| IOH25820 | 220787 | NM_198081.1 | NM_198081 |
| IOH27185 | 220788 | BC039244.1 | BC039244 |
| IOH27505 | 220802 | BC045634.1 | BC045634 |
| IOH26861 | 220789 | NM_006100.1 | NM_006100 |
| IOH27669 | 220782 | BC031964.1 | BC031964 |
| IOH14368 | 220884 | NM_001436.2 | NM_001436 |
| IOH27270 | 220885 | BC039252.1 | BC039252 |
| IOH27729 | 220886 | NM_198181.1 | NM_198181 |
| IOH27746 | 220792 | NM_053006.1 | NM_053006 |
| IOH22581 | 220887 | NM_144770.1 | NM_144770 |
| IOH27237 | 220793 | BC036071.1 | BC036071 |
| IOH21856 | 220794 | NM_006869.1 | NM_006869 |
| IOH22385 | 220888 | BC024243.2 | BC024243 |
| IOH25740 | 224795 | NM_002734.1 | NM_002734 |
| IOH28221 | 220892 | AB065869.1 | AB065869 |
| IOH25832 | 220799 | NM_144595.1 | NM_144595 |
| IOH28158 | 220882 | AB065674.1 | AB065674 |
| IOH22420 | 218753 | BC022189.2 | BC022189 |
| IOH11454 | 218768 | BC027978.1 | BC027978 |
| IOH14802 | 218739 | BC015569.1 | BC015569 |
| IOH22400 | 218740 | BC028425.1 | BC028425 |
| IOH22436 | 218742 | BC021188.2 | BC021188 |
| IOH22462 | 218743 | NM_015605.4 | NM_015605 |
| IOH11793 | 218744 | NM_002287.2 | NM_002287 |
| IOH14435 | 218745 | BC009207.2 | BC009207 |
| IOH14162 | 218746 | NM_001353.3 | NM_001353 |
| IOH21422 | 218747 | BC009631.1 | BC009631 |
| IOH21447 | 218748 | BC020985.1 | BC020985 |
| IOH21486 | 218750 | NM_018370.1 | NM_018370 |
| IOH21471 | 218737 | BC016486.1 | BC016486 |
| IOH22403 | 218752 | NM_144588.2 | NM_144588 |
| IOH21444 | 218736 | BC020979.1 | BC020979 |
| IOH22437 | 218754 | BC021189.2 | BC021189 |
| IOH22464 | 218755 | BC036532.2 | BC036532 |
| IOH14523 | 218757 | BC013905.2 | BC013905 |
| IOH13629 | 218758 | BC018771.1_{—} | BC018771 |
| IOH21424 | 218759 | BC015219.1 | BC015219 |
| IOH21448 | 218760 | NM_000585.1 | NM_000585 |
| IOH21474 | 218761 | BC013112.2 | BC013112 |
| IOH21488 | 218762 | NM_006571.2 | NM_006571 |
| IOH14530 | 218763 | BC027729.1 | BC027729 |
| IOH22422 | 218765 | BC022083.2 | BC022083 |
| IOH10174 | 219030 | NM_138480.1 | NM_1389480 |
| IOH14605 | 218751 | BC014264.2 | BC014264 |
| IOH22434 | 218718 | NM_153224.2 | NM_153224 |
| IOH22407 | 218705 | NM_018710.1 | NM_018710 |
| IOH22428 | 218706 | BC032957.1 | BC032957 |
| IOH22455 | 218707 | NM_004170.2 | NM_004170 |
| IOH11762 | 218708 | BC025742.1 | BC025742 |
| IOH14150 | 218709 | NM_007108.1 | NM_007108 |
| IOH14433 | 218710 | NM_016319.1 | NM_016319 |
| IOH21411 | 218711 | BC034245.1 | BC034245 |
| IOH21430 | 218712 | BC021622.1 | BC021622 |
| IOH21462 | 218713 | NM_152715.1 | NM_152715 |
| IOH21481 | 218714 | NM_173344.1 | NM_173344 |
| IOH13580 | 218715 | BC019239.1 | BC019239 |
| IOH21483 | 218738 | NM_138461.1 | NM_138461 |
| IOH22412 | 218717 | BC022077.1 | BC022077 |
| IOH13570 | 218769 | NM_024674.1 | NM_024674 |
| IOH22457 | 218719 | BC036540.2 | BC036540 |
| IOH14481 | 218721 | BC013959.1 | BC013959 |
| IOH13947 | 218722 | BC017337.1 | BC017337 |
| IOH21413 | 218723 | NM_032459.1 | NM_032459 |
| IOH21442 | 218724 | NM_021945.1 | NM_021945 |
| IOH21470 | 218725 | BC024939.1 | BC024939 |
| IOH21482 | 218726 | NM_020239.2 | NM_020239 |
| IOH14665 | 218727 | BC017572.1 | BC017572 |
| IOH22398 | 218728 | BC024245.2 | BC024245 |
| IOH22414 | 218729 | BC030711.2 | BC030711 |
| IOHI3956 | 218734 | NM_024760.1 | NM_024760 |
| IOH22397 | 218716 | NM_030755.1 | NM_030755 |
| IOH10056 | 219017 | NM_002952.2 | NM_002952 |
| IOH22449 | 218766 | BC033035.1 | BC033035 |
| IOH13334 | 218998 | NM_138446.1 | NM_138446 |
| IOH3700 | 218314 | BC004144.1 | BC004144 |
| IOH5156 | 218300 | NM_024516.1 | NM_024516 |
| IOH4417 | 218295 | BC000121.1 | BC000121 |
| IOH10118 | 219006 | NM_138801.1 | NM_138801 |
| IOH4415 | 218283 | BC001741.1 | BC001741 |
| IOH10343 | 219008 | NM_152690.1 | NM_152690 |
| IOH10545 | 219009 | BC013613.1 | BC013613 |
| IOH3168 | 219277 | NM_006275.2 | NM_006275 |
| IOH4626 | 218275 | NM_006232.2 | NM_006232 |
| IOH10283 | 218996 | BC014776.1 | BC014776 |
| IOH4017 | 218269 | NM_016286.1 | NM_016286 |
| IOH3721 | 218315 | BC000215.1 | BC000215 |
| IOH3713 | 218267 | NM_146388.1 | NM_146388 |
| IOH4623 | 218263 | NM_000801.2 | NM_000801 |
| IOH4438 | 218260 | NM_000437.2 | NM_000437 |
| IOH4407 | 218259 | BC000120.1 | BC000120 |
| IOH13142 | 219022 | BC012131.1 | BC012131 |
| IOH5456 | 218258 | NM_173089.1 | NM_173089 |
| IOH4012 | 218257 | BC001433.1 | BC001433 |
| IOH7183 | 217949 | BC005312.1 | BC005312 |
| IOH3846 | 219027 | NM_020676.2 | NM_020676 |
| IOH22871 | 220911 | NM_153208.1 | NM_153208 |
| IOH4410 | 218271 | BC000190.1 | BC000190 |
| IOH21410 | 218793 | BC034275.1 | BC034275 |
| IOH21405 | 218770 | NM_024060.1 | NM_024060 |
| IOH21426 | 218771 | NM173541.1 | NM173541 |
| IOH21450 | 218772 | NM_021709.1 | NM_021709 |
| IOH21475 | 218773 | BC023152.1 | BC023152 |
| IOH21490 | 218774 | NM_152634.1 | NM_152634 |
| IOH14227 | 218775 | NM_005601.2 | NM_005601 |
| IOH14763 | 218781 | NM_025161.2 | NM_025161 |
| IOH21409 | 218782 | NM_173192.1 | NM_173192 |
| IOH21427 | 218783 | NM_153702.1 | NM_153702 |
| IOH21454 | 218784 | BC018404.1 | BC018404 |
| IOH21476 | 218785 | BC016640.1 | BC016640 |
| IOH10533 | 218997 | BC018206.1 | BC018206 |
| IOH14815 | 218792 | BC011680.1 | BC011680 |
| IOH7206 | 217939 | BC005339.1 | BC005339 |
| IOH21428 | 218794 | NM_174926.1 | NM_174926 |
| IOH21458 | 218795 | BC031469.1 | BC031469 |
| IOH14039 | 218797 | BC023982.1 | BC023982 |
| IOH13283 | 218986 | NM_032014.1 | NM_032014 |
| IOH3978 | 218327 | BC001394.1 | BC001394 |
| IOH3706 | 218325 | NM_002402.1 | NM_002402 |
| IOH5159 | 218323 | BC004906.1 | BC004906 |
| IOH4908 | 218992 | NM_002014.2 | NM_002014 |
| IOH5134 | 218322 | NM_001384.2 | NM_001384 |
| IOH4474 | 218319 | NM_030810.1 | NM_030810 |
| IOH22406 | 218787 | NM_005038.1 | NM_005038 |
| IOH4088 | 220099 | NM_032636.2 | NM_032636 |
| IOH6705 | 217893 | NM_005586.2 | NM_005586 |
| IOH14064 | 220075 | NM_004582.2 | NM_004582 |
| IOH7131 | 220077 | NM_018466.2 | NM_018466 |
| IOH5661 | 220079 | NM_004569.1 | NM_004569 |
| IOH10491 | 220081 | NM_001769.2 | NM_001769 |
| IOH9914 | 220082 | BC009712.1 | BC009712 |
| IOH12720 | 220085 | BC009956.1 | BC009956 |
| IOH3658 | 220087 | NM_004881.1 | NM_004881 |
| IOH9786 | 220090 | NM_005380.1 | NM_005380 |
| IOH12125 | 220091 | NM_019101.2 | NM_019101 |
| IOH10694 | 220094 | BC020517.1 | BC020517 |
| IOH11450 | 220072 | NM_019895.1 | NM_019895 |
| IOH4981 | 220097 | NM_032641.1 | NM_032641 |
| IOH7016 | 220069 | BC008054.1 | BC008054 |
| IOH7207 | 220101 | BC005187.1 | BC005187 |
| IOH3991 | 220103 | BC001430.1 | BC001430 |
| IOH11448 | 220106 | BC011968.1 | BC011968 |
| IOH10395 | 220107 | NM_024946.1 | NM_024946 |
| IOH4051 | 220108 | BC002568.1 | BC002568 |
| IOH10241 | 220109 | NM_004489.3 | NM_004489 |
| IOH4735 | 220110 | BC000108.1 | BC000108 |
| IOH9888 | 220112 | NM_003650.2 | NM_003650 |
| IOH7193 | 217903 | BC005258.1 | BC005258 |
| IOH7482 | 217901 | NM_003338.2 | NM_003338 |
| IOH11751 | 220034 | NM_006002.2 | NM_006002 |
| IOH14515 | 220096 | BC020746.1 | BC020746 |
| IOH3794 | 220053 | BC001105.1 | BC001105 |
| IOH26872 | 220816 | NM_002242.2 | NM_002242 |
| IOH13408 | 220038 | BC019107.1 | BC019107 |
| IOH3287 | 220040 | NM_002074.2 | NM_002074 |
| IOH12964 | 220041 | NM_144646.1 | NM_144646 |
| IOH10522 | 220042 | NM_024775.8 | NM_024775 |
| IOH13182 | 220046 | BC021295.2 | BC021295 |
| IOH12787 | 220047 | NM_148975.1 | NM_148975 |
| IOH14799 | 220048 | BC022344.1 | BC022344 |
| IOH6364 | 220049 | NM_000802.2 | NM_000802 |
| IOH13381 | 220050 | BC017296.2 | BC017296 |
| IOH5857 | 220074 | BC007320.2 | BC007320 |
| IOH4957 | 220052 | NM_007370.2 | NM_007370 |
| IOH6703 | 217892 | BC007835.1 | BC007835 |
| IOH12167 | 220054 | BC012575.1 | BC012575 |
| IOH3292 | 220058 | BC009010.1 | BC009010 |
| IOH5013 | 220059 | BC004440.1 | BC004440 |
| IOH5505 | 220060 | NM_013342.1 | NM_013342 |
| IOH13661 | 220061 | NM_016052.1 | NM_016052 |
| IOH14512 | 220062 | BC020744.1 | BC020744 |
| IOH5147 | 220063 | BC003132.1 | BC003132 |
| IOH13005 | 220064 | BC010943.1 | BC010943 |
| IOH13730 | 220065 | BC020754.1 | BC020754 |
| IOH12789 | 220066 | BC020651.1 | BC020651 |
| IOH12082 | 220067 | BC009327.2 | BC009327 |
| IOH10076 | 220051 | BC014897.1 | BC014897 |
| IOH5732 | 221003 | NM_012289.2 | NM_012289 |
| IOH7457 | 217900 | BC008478.1 | BC008478 |
| IOH6647 | 219623 | NM_003311.2 | NM_003311 |
| IOH5963 | 219628 | BC006456.1 | BC006456 |
| IOH22146 | 219629 | BC035314.1 | BC035314 |
| IOH3041 | 219633 | NM_018983.2 | NM_018983 |
| IOH10608 | 219634 | NM_032146.2 | NM_032146 |
| IOH13548 | 219636 | NM_005040.1 | NM_005040 |
| IOH23082 | 219640 | BC021250.1 | BC021250 |
| IOH3394 | 219641 | BC009046.1 | BC009046 |
| IOH6811 | 220999 | BC007213.1 | BC007213 |
| IOH3060 | 221000 | NM_020165.2 | NM_020165 |
| IOH21729 | 219618 | NM_018527.1 | NM_018527 |
| IOH3053 | 221002 | BC001258.1 | BC001258 |
| IOH22703 | 219613 | BC031592.1 | BC031592 |
| IOH5306 | 221004 | BC002702.1 | BC002702 |
| IOH4511 | 221005 | NM_016630.2 | NM_016630 |
| IOH3456 | 221006 | BC000306.1 | BC000306 |
| IOH4394 | 221007 | BC000238.1 | BC000238 |
| IOH4172 | 221008 | NM_005371.2 | NM_005371 |
| IOH4240 | 221009 | BC000645.1 | BC000645 |
| IOH3462 | 221010 | NM_002810.1 | NM_002810 |
| IOH6840 | 221011 | BC007557.1 | BC007557 |
| IOH3075 | 221012 | BC001247.1 | BC001247 |
| IOH4744 | 221013 | NM_005659.1 | NM_005659 |
| IOH22396 | 218704 | NM_145173.1 | NM_145173 |
| IOH4743 | 221001 | NM_016091.1 | NM_016091 |
| IOH10937 | 217737 | NM_022755.2 | NM_022755 |
| IOH5185 | 218999 | NM_031445.1 | NM_031445 |
| IOH7198 | 217881 | BC007003.1 | BC007003 |
| IOH7191 | 217879 | BC007009.1 | BC007009 |
| IOH7444 | 217876 | BC005893.1 | BC005893 |
| IOH7194 | 217869 | NM_001906.1 | NM_001906 |
| IOH5230 | 219011 | BC004234.1 | BC004234 |
| IOH7475 | 217865 | BC005914.1 | BC005914 |
| IOH12034 | 217760 | BC027617.1 | BC027617 |
| IOH4984 | 219014 | BC003597.1 | BC003597 |
| IOH14651 | 217751 | NM_002966.1 | NM_002966 |
| IOH11737 | 217749 | BC027607.1 | BC027607 |
| IOH22166 | 219621 | NM_024786.1 | NM_024786 |
| IOH11653 | 217738 | NM_173501.1 | NM_173501 |
| IOH11316 | 220033 | NM_012400.2 | NM_012400 |
| IOH13616 | 217729 | NM_001911.1 | NM_001911 |
| IOH11315 | 217724 | NM_002364.1 | NM_002364 |
| IOH7270 | 216485 | BC007023.1 | BC007023 |
| IOH14716 | 216477 | NM_018291.2 | NM_018291 |
| IOH10668 | 217713 | NM_145268.1 | NM_145268 |
| IOH11096 | 217712 | NM_033105.1 | NM_033105 |
| IOH6460 | 219598 | BC006393.1 | BC006393 |
| IOH7295 | 219599 | NM_002994.2 | NM_002994 |
| IOH22574 | 219607 | BC029520.1 | BC029520 |
| IOH21870 | 219608 | BC033819.1 | BC033819 |
| IOH12287 | 219609 | BC020868.1 | BC020868 |
| IOH27734 | 220945 | BC040606.1 | BC040606 |
| IOH10619 | 220954 | BC022231.1 | BC022231 |
| IOH5873 | 220935 | NM_004549.2 | NM_004549 |
| IOH27547 | 220841 | NM_152542.2 | NM_152542 |
| IOH27482 | 220842 | BC039306.1 | BC039306 |
| IOH13267 | 220937 | NM_022818.2 | NM_022818 |
| IOH25853 | 220843 | NM_182607.2 | NM_182607 |
| IOH28263 | 220938 | AB065734.1 | AB065734 |
| IOH28238 | 220939 | AB065812.1 | AB065812 |
| IOH25850 | 220845 | BC043193.2 | BC043193 |
| IOH27111 | 220846 | BC032861.1 | BC032861 |
| IOH27401 | 220849 | NM_012113.1 | NM_012113 |
| IOH25805 | 220934 | BC039152.1 | BC039152 |
| IOH27486 | 220850 | BC036193.1 | BC036193 |
| IOH27319 | 220946 | BC047056.1 | BC047056 |
| IOH27747 | 220852 | BC041366.2 | BC041366 |
| IOH22178 | 220853 | BC031999.1 | BC031999 |
| IOH5904 | 220947 | NM_017594.2 | NM_017594 |
| IOH13412 | 220948 | NM_138786.1 | NM_138786 |
| IOH27478 | 220854 | BC040527.1 | BC040527 |
| IOH28581 | 220949 | AB065663.1 | AB065663 |
| IOH27515 | 220855 | BC031231.1 | BC031231 |
| IOH25823 | 220858 | BC037906.1 | BC037906 |
| IOH12808 | 220036 | NM_015399.1 | NM_015399 |
| IOH26818 | 220832 | BC030640.1 | BC030640 |
| IOH5628 | 221015 | NM_012191.1 | NM_012191 |
| IOH14740 | 220912 | NM_001216.1 | NM_001216 |
| IOH27358 | 220818 | NM_152723.1 | NM_152723 |
| IOH5681 | 220913 | NM_000972.2 | NM_000972 |
| IOH25737 | 220819 | BC038354.1 | BC038354 |
| IOH28500 | 220914 | XM_060307.1 | XM_060307 |
| IOH25797 | 220821 | NM_153719.2 | NM_153719 |
| IOH25831 | 220922 | BC041339.1 | BC041339 |
| IOH25844 | 220829 | BC043175.1 | BC043175 |
| IOH27467 | 220830 | NM_032047.2 | NM_032047 |
| IOH27450 | 220840 | BC037253.1 | BC037253 |
| IOH28501 | 220926 | XM_060315.1 | XM_060315 |
| IOH20993 | 220955 | NM_021962.1 | NM_021962 |
| IOH28527 | 220927 | XM_062285.1 | XM_062285 |
| IOH27543 | 220833 | NM_000167.1 | NM_000167 |
| IOH27329 | 220834 | NM_173619.1 | NM_173619 |
| IOH28257 | 220929 | AB065758.1 | AB065758 |
| IOH27423 | 220835 | NM_024430.1 | NM_024430 |
| IOH27502 | 220836 | NM_178863.2 | NM_178863 |
| IOH28163 | 220930 | AF137396.2 | AF137396 |
| IOH27369 | 220837 | NM_153356.1 | NM_153356 |
| IOH27153 | 220838 | BC032852.2 | BC032852 |
| IOH20956 | 220932 | NM_006225.1 | NM_006225 |
| IOH27245 | 220933 | BC041793.1 | BC041793 |
| IOH11558 | 220925 | NM_182554.1 | NM_182554 |
| IOH13335 | 219736 | NM_138788.1 | NM_138788 |
| IOH27212 | 220859 | BC036015.1 | BC036015 |
| IOH12508 | 219703 | BC014577.1 | BC014577 |
| IOH21553 | 219705 | NM_001585.1 | NM_001585 |
| IOH22183 | 219707 | NM_000710.2 | NM_000710 |
| IOH12498 | 219708 | NM_144975.1 | NM_144975 |
| IOH9781 | 219710 | BC010691.1 | BC0I0691 |
| IOH10008 | 219717 | BC017168.1 | BC017168 |
| IOH14316 | 219719 | BC009775.1 | BC009775 |
| IOH12277 | 219721 | NM_016527.1 | NM_016527 |
| IOH12342 | 219694 | NM_030774.2 | NM_030774 |
| IOH21781 | 219732 | NM_152287.2 | NM_152287 |
| IOH4800 | 219693 | BC001873.1 | BC001873 |
| IOH6499 | 219737 | NM_018941.1 | NM_018941 |
| IOH7172 | 220021 | BC005245.1 | BC005245 |
| IOH11058 | 220022 | NM_016422.2 | NM_016422 |
| IOH12058 | 220023 | BC022379.1 | BC022379 |
| IOH12842 | 220024 | NM_144578.1 | NM_144578 |
| IOH13793 | 220025 | BC017865.1 | BC017865 |
| IOH12973 | 220026 | NM_152430.1 | NM_152430 |
| IOH13243 | 220027 | BC021092.1 | BC021092 |
| IOH3742 | 220029 | NM_016504.1 | NM_016504 |
| IOH9897 | 220030 | BC009621.1 | BC009621 |
| IOH6336 | 220031 | NM_32499.1 | NM_032499 |
| IOH3054 | 219661 | NM_003675.2 | NM_003675 |
| IOH27376 | 220956 | NM_052841.2 | NM_052841 |
| IOH27355 | 220957 | NM_182623.1 | NM_182623 |
| IOH26853 | 220864 | BC032838.2 | BC032838 |
| IOH22623 | 220958 | NM_002521.1 | NM_002821 |
| IOH27539 | 220865 | NM_003370.1 | NM_003370 |
| IOH10746 | 219646 | NM_152443.1 | NM_152443 |
| IOH5210 | 219647 | BC003653.1 | BC003653 |
| IOH7384 | 219648 | NM_006479.2 | NM_006479 |
| IOH21782 | 219649 | BC033665.1 | BC033665 |
| IOH21713 | 219652 | NM_182980.1 | NM_182980 |
| IOH7253 | 219655 | NM_006136.1 | NM_006136 |
| IOH5297 | 219702 | BC002653.1 | BC002653 |
| IOH12290 | 219660 | BC022316.1 | BC022316 |
| IOH27433 | 220817 | NM_000913.1 | NM_000913 |
| IOH3631 | 219666 | BC000412.1 | BC000412 |
| IOH21515 | 219672 | BC033591.1 | BC033591 |
| IOH12543 | 219673 | NM_022788.2 | NM_022788 |
| IOH12753 | 219677 | NM_032784.2 | NM_032784 |
| IOH5426 | 219682 | NM_002914.1 | NM_002914 |
| IOH10934 | 219683 | BC025726.1 | BC025726 |
| IOH22511 | 219685 | BC029483.1 | BC029483 |
| IOH4342 | 219687 | BC000683.1 | BC000683 |
| IOH11017 | 219690 | BC012924.1 | BC012924 |
| IOH5253 | 219692 | NM_006140.2 | NM_006140 |
| IOH22790 | 219658 | BC031653.1 | BC031653 |
| IOH4028 | 220342 | NM_018107.2 | NM_018107 |
| IOH14546 | 220324 | NM_004494.1 | NM_004494 |
| IOH5969 | 220325 | BC008364.1 | BC008364 |
| IOH22693 | 220326 | BC034389.1 | BC034389 |
| IOH12245 | 220332 | NM_145245.1 | NM_145245 |
| IOH10823 | 220333 | NM_004589.1 | NM_004589 |
| IOH6517 | 220335 | BC007742.1 | BC007742 |
| IOH21590 | 220337 | NM_152567.1 | NM_152567 |
| IOH22755 | 220338 | BC029220.1 | BC029220 |
| IOH12948 | 220339 | BC017810.1 | BC017810 |
| IOH22548 | 220317 | BC031068.1 | BC031068 |
| IOH22738 | 220343 | BC029158.1 | BC029158 |
| IOH6401 | 220344 | NM_139156.1 | NM_139156 |
| IOH9645 | 220345 | BC010451.1 | BC010451 |
| IOH11023 | 220346 | BC019247.1 | BC019247 |
| IOH2949 | 220347 | BC000158.2 | BC000158 |
| IOH12711 | 220348 | NM_015343.1 | NM_015343 |
| IOH21842 | 220349 | BC033864.1 | BC033864 |
| IOH21821 | 220374 | NM_014305.1 | NM_014305 |
| IOH12784 | 220375 | NM_032478.1 | NM_032478 |
| IOH5017 | 220376 | BC004424.1 | BC004424 |
| IOH10922 | 220377 | BC026184.2 | BC026184 |
| IOH11263 | 217181 | NM_013246.1 | NM_013246 |
| IOH3307 | 220340 | NM_000327.2 | NM_000327 |
| IOH22719 | 220302 | NM_005749.2 | NM_005749 |
| IOH26809 | 220684 | BC035936.1 | BC035936 |
| IOH12876 | 217183 | NM_016487.1 | NM_016487 |
| IOH12088 | 217184 | BC010907.1 | BC010907 |
| IOH12868 | 217185 | BC010929.1 | BC010929 |
| IOH12920 | 217186 | BC009423.1 | BC009423 |
| IOH12968 | 217187 | BC009485.1 | BC009485 |
| IOH12627 | 217189 | NM_138807.1 | NM_138807 |
| IOH13241 | 217192 | NM_153217.1 | NM_153217 |
| IOH12144 | 217193 | BC014538.1 | BC014538 |
| IOH13498 | 217194 | BC010901.1 | BC010901 |
| IOH12952 | 217195 | NM_052822.1 | NM_052822 |
| IOH13758 | 220322 | NM_002784.2 | NM_002784 |
| IOH10524 | 217199 | NM_138414.1 | NM_138414 |
| IOH13683 | 220303 | BC009797.1 | BC009797 |
| IOH12389 | 220304 | NM_030664.2 | NM_030664 |
| IOH21872 | 220305 | NM_052938.2 | NM_052938 |
| IOH4700 | 220306 | BC000014.1 | BC000014 |
| IOH9728 | 220307 | BC011379.1 | BC011379 |
| IOH3819 | 220309 | NM_003720.1 | NM_003720 |
| IOH11952 | 220312 | BC022081.2 | BC022081 |
| IOH7540 | 220313 | NM_032929.1 | NM_032929 |
| IOH21715 | 220314 | NM_145109.1 | NM_145109 |
| IOH13154 | 220315 | BC017880.1 | BC017880 |
| IOH13312 | 217198 | NM_022483.2 | NM_022483 |
| IOH4081 | 216778 | NM_017668.1 | NM_017668 |
| IOH13657 | 220380 | NM_005666.1 | NM_005666 |
| IOH3301 | 216761 | NM_138390.1 | NM_138390 |
| IOH3366 | 216762 | BC008253.1 | BC008253 |
| IOH14139 | 216764 | NM_018948.2 | NM_018948 |
| IOH3944 | 216765 | NM_001757.1 | NM_001757 |
| IOH4079 | 216766 | NM_005620.1 | NM_005620 |
| IOH4136 | 216767 | NM_000375.1 | NM_000375 |
| IOH4171 | 216768 | NM_024047.2 | NM_024047 |
| IOH2504 | 216770 | NM_005032.2 | NM_005032 |
| IOH3015 | 216771 | BC000993.2 | BC000993 |
| IOH3304 | 216773 | BC008145.1 | BC008145 |
| IOH4274 | 216758 | NM_024051.1 | NM_024051 |
| IOH3948 | 216777 | NM_001549.1 | NM_001549 |
| IOH4220 | 216757 | BC001023.1 | BC001023 |
| IOH4142 | 216779 | BC002622.1 | BC002622 |
| IOH4184 | 216780 | BC000586.1 | BC000586 |
| IOH4234 | 216781 | NM_138820.1 | NM_138820 |
| IOH2894 | 216782 | NM_024033.1 | NM_024033 |
| IOH3019 | 216783 | NM_006324.1 | NM_006324 |
| IOH3260 | 216784 | NM_024049.1 | NM_024049 |
| IOH3372 | 216786 | NM_080651.1 | NM_080651 |
| IOH3953 | 216789 | NM_015449.1 | NM_015449 |
| IOH4112 | 216790 | NM_004146.3 | NM_004146 |
| IOH4145 | 216791 | BC000535.1 | BC000535 |
| IOH4186 | 216792 | NM_000854.2 | NM_000854 |
| IOH4237 | 215793 | BC001017.1 | BC001017 |
| IOH14516 | 216775 | BC015684.2 | BC015684 |
| IOH11024 | 216739 | NM_174930.2 | NM_174930 |
| IOH2986 | 220384 | NM_006142.1 | NM_006142 |
| IOH14261 | 220387 | BC012547.1 | BC012547 |
| IOH10984 | 220388 | NM_178525.2 | NM_178525 |
| IOH5587 | 220391 | NM_005268.1 | NM_005268 |
| IOH4093 | 220392 | NM_004155.2 | NM_004155 |
| IOH13690 | 224395 | NM_014214.1 | NM_014214 |
| IOH10977 | 216727 | BC022454.2 | BC022454 |
| IOH967 | 216730 | BC002493.1 | BC002493 |
| IOH4127 | 216731 | NM_014221.1 | NM_014221 |
| IOH3237 | 216760 | BC000885.1 | BC000885 |
| IOH3330 | 216738 | BC008605.1 | BC008605 |
| IOH14670 | 216740 | BC021258.1 | BC021258 |
| IOH3933 | 216741 | NM_005697.3 | NM_005697 |
| IOH4069 | 216742 | NM_007008.1 | NM_007008 |
| IOH4130 | 216743 | NM_018124.2 | NM_018124 |
| IOH4219 | 216745 | NM_014077.1 | NM_014077 |
| IOH3086 | 236748 | NM_003244.1 | NM_003244 |
| IOH3354 | 216750 | NM_020445.1 | NM_020445 |
| IOH10757 | 216751 | BC022524.1 | BC022524 |
| IOH14570 | 216752 | BC021303.1 | BC021303 |
| IOH4076 | 216754 | NM_003662.1 | NM_003662 |
| IOH4170 | 216756 | NM_015492.2 | NM_015492 |
| IOH3291 | 216737 | NM_138474.1 | NM_138474 |
| IOH14182 | 220740 | BC010349.1 | BC010349 |
| IOH14782 | 220754 | BC017353.1 | BC017353 |
| IOH14254 | 220727 | BC015818.1 | BC015818 |
| IOH7291 | 220729 | NM_005651.1 | NM_005651 |
| IOH14451 | 220730 | BC018632.1 | BC018632 |
| IOH27724 | 220731 | BC038713.1 | BC038713 |
| IOH22322 | 220732 | BC028682.2 | BC028682 |
| IOH27335 | 220733 | NM_001608.1 | NM_001608 |
| IOH25799 | 220735 | NM_173830.3 | NM_173830 |
| IOH21965 | 220736 | NM_032868.1 | NM_032868 |
| IOH25906 | 220737 | BC035882.1 | BC035882 |
| IOH26825 | 220722 | NM_177966.3 | NM_177966 |
| IOH14848 | 220739 | BC021573.1 | BC021573 |
| IOH27535 | 220720 | NM_003211.1 | NM_003211 |
| IOH12001 | 220742 | NM_32858.1 | NM_032858 |
| IOH25842 | 220743 | NM_172159.2 | NM_172159 |
| IOH25885 | 220744 | NM_178553.2 | NM_178553 |
| IOH27322 | 220745 | BC031589.1 | BC031589 |
| IOH27372 | 220746 | BC033495.1 | BC033495 |
| IOH25811 | 220747 | BC023247.1 | BC023247 |
| IOH26807 | 220748 | BC040457.1 | BC040457 |
| IOH27106 | 220749 | BC037278.1 | BC037278 |
| IOH14142 | 220751 | NM_001375.1 | NM_001375 |
| IOH5524 | 220752 | NM_031439.1 | NM_031439 |
| IOH12159 | 217182 | BC012573.1 | BC012573 |
| IOH4956 | 220738 | NM_021146.2 | NM_021146 |
| IOH7568 | 220705 | BC008492.1 | BC008492 |
| IOH5858 | 216483 | BC005857.1 | BC005857 |
| IOH25900 | 220689 | BC041811.1 | BC041811 |
| IOH10880 | 220690 | BC027322.1 | BC027322 |
| IOH14312 | 220691 | BC008884.1 | BC008884 |
| IOH6569 | 220693 | NM_032342.1 | NM_032342 |
| IOH11575 | 220694 | NM_175609.1 | NM_175609 |
| IOH3266 | 220695 | NM_007076.1 | NM_007076 |
| IOH27749 | 220697 | BC037878.1 | BC037878 |
| IOH27405 | 220698 | BC035359.1 | BC035359 |
| IOH27206 | 220699 | BC036019.1 | BC036019 |
| IOH27741 | 220701 | BC037779.2 | BC037779 |
| IOH7352 | 220702 | NM_016371.1 | NM_016371 |
| IOH6246 | 220726 | NM_006877.1 | NM_006877 |
| IOH12181 | 220704 | BC012604.1 | BC012604 |
| IOH25867 | 220755 | NM_153716.1 | NM_153716 |
| IOH7527 | 220706 | BC005896.1 | BC005896 |
| IOH11355 | 220707 | NM_001308.1 | NM_001308 |
| IOH27679 | 220708 | BC035079.2 | BC035079 |
| IOH21615 | 220709 | BC031222.1 | BC031222 |
| IOH26808 | 220710 | BC038710.1 | BC038710 |
| IOH27524 | 220712 | BC036246.1 | BC036246 |
| IOH25815 | 220713 | BC028295.1 | BC028295 |
| IOH4945 | 220714 | BC003568.1 | BC003568 |
| IOH13936 | 220715 | NM_181703.1 | NM_181703 |
| IOH14365 | 220716 | BC017475.1 | BC017475 |
| IOH11838 | 220717 | NM_006217.2 | NM_006217 |
| IOH13760 | 220719 | BC014550.1 | BC014550 |
| IOH11211 | 220703 | NM_017436.2 | NM_017436 |
| IOH12271 | 217159 | NM_020466.3 | NM_020466 |
| IOH11398 | 220753 | NM_002898.1 | NM_002898 |
| IOH10239 | 217141 | NM_138333.1 | NM_138333 |
| IOH11084 | 217143 | BC015323.1 | BC015323 |
| IOH12222 | 217146 | BC010915.1 | BC010915 |
| IOH12798 | 217147 | BC014532.1 | BC014532 |
| IOH12838 | 217148 | NM_006299.2 | NM_006299 |
| IOH12145 | 217149 | BC014539.1 | BC014539 |
| IOH13421 | 217150 | BC017098.1 | BC017098 |
| IOH12306 | 217151 | NM_022104.1 | NW_022104 |
| IOH10498 | 217152 | BC011959.1 | BC011959 |
| IOH12334 | 217154 | NM_007083.2 | NM_007083 |
| IOH10730 | 217155 | NM_016289.2 | NM_016289 |
| IOH12103 | 217139 | NM_148904.2 | NM_148904 |
| IOH12345 | 217158 | NM_003986.1 | NM_003986 |
| IOH12811 | 217137 | NM_006834.2 | NM_006834 |
| IOH12855 | 217160 | NM_014596.3 | NM_014596 |
| IOH12897 | 217161 | BC011011.1 | BC011011 |
| IOH13048 | 217163 | NM_152302.1 | NM_152302 |
| IOH12821 | 217173 | NM_016940.1 | NM_016940 |
| IOH12586 | 217175 | BC010405.2 | BC010405 |
| IOH10516 | 217176 | BC018346.1 | BC018346 |
| IOH10874 | 217177 | NM_006788.2 | NM_006788 |
| IOH12192 | 217178 | NM_021255.1 | NM_021255 |
| IOH11180 | 217179 | NM_017612.1 | NM_017612 |
| IOH11264 | 217157 | NM_052817.1 | NM_052817 |
| IOH11149 | 217108 | BC016911.1 | BC016911 |
| IOH21967 | 220756 | NM_014079.1 | NM_014079 |
| IOH27668 | 220759 | BC034318.1 | BC034318 |
| IOH27738 | 220760 | BC041876.1 | BC041876 |
| IOH3277 | 220761 | BC008090.1 | BC008090 |
| IOH4907 | 220762 | BC001778.1 | BC001778 |
| IOH7335 | 220763 | NM_033213.1 | NM_033213 |
| IOH14157 | 220764 | NM_032924.2 | NM_032924 |
| IOH26905 | 220766 | BC051698.1 | BC051698 |
| IOH26848 | 220767 | NM_153353.2 | NM_153353 |
| IOH27730 | 220768 | BC039362.1 | BC039362 |
| IOH27128 | 220769 | NM_153343.2 | NM_153343 |
| IOH25790 | 220770 | BC021906.1 | BC021906 |
| IOH13488 | 217140 | BC026058.1 | BC026058 |
| IOH13135 | 217106 | NM_032213.2 | NM_032213 |
| IOH3311 | 216797 | BC009025.1 | BC009025 |
| IOH11042 | 217109 | BC026213.1 | BC026213 |
| IOH12956 | 217110 | NM_145055.1 | NM_145055 |
| IOH12069 | 217111 | BC010904.1 | BC010904 |
| IOH12723 | 217113 | NM_013338.2 | NM_013338 |
| IOH12717 | 217118 | NM_015878.2 | NM_015878 |
| IOH10995 | 217121 | BC016914.1 | BC016914 |
| IOH12297 | 217122 | BC019337.1 | BC019337 |
| IOH12346 | 217123 | BC012626.1 | BC012626 |
| IOH12616 | 217127 | BC017376.2 | BC017376 |
| IOH12128 | 217128 | BC014299.2 | BC014299 |
| IOH11229 | 217131 | NM_006685.2 | NM_006685 |
| IOH12916 | 217136 | NM_005368.1 | NM_005368 |
| IOH22979 | 220771 | NM_018083.1 | NM_018083 |
| IOH13470 | 220202 | BC017926.1 | BC017926 |
| IOH3931 | 220130 | BC002490.1 | BC002490 |
| IOH14646 | 220132 | NM_020378.2 | NM_020378 |
| IOH21862 | 220133 | NM_152499.1 | NM_152499 |
| IOH5353 | 220137 | NM_018137.1 | NM_018137 |
| IOH12436 | 220142 | BC011934.1 | BC011934 |
| IOH22864 | 220144 | BC031671.1 | BC031671 |
| IOH12083 | 220145 | BC014455.1 | BC014455 |
| IOH21792 | 220148 | BC033854.1 | BC033854 |
| IOH9690 | 220128 | NM_007021.1 | NM_007021 |
| IOH14283 | 220154 | NM_000948.1 | NM_000948 |
| IOH13538 | 220127 | NM_014488.2 | NM_014488 |
| IOH13203 | 220157 | NM_003975.1 | NM_003975 |
| IOH5241 | 220158 | NM_016608.1 | NM_016608 |
| IOH6588 | 220166 | BC006104.1 | BC006104 |
| IOH23124 | 220168 | BC029428.1 | BC029428 |
| IOH6878 | 220179 | NM_032753.2 | NM_032753 |
| IOH12214 | 220186 | NM_016364.2 | NM_016364 |
| IOH23140 | 220191 | BC029424.1 | BC029424 |
| IOH23143 | 220192 | BC029458.1 | BC029458 |
| IOH3025 | 216795 | BC000937.2 | BC000937 |
| IOH13252 | 219257 | NM_080590.1 | NM_080590 |
| IOH12052 | 219192 | NM_145051.1 | NM_145051 |
| IOH10942 | 219247 | NM_144594.1 | NM_144594 |
| IOH12556 | 220129 | NM_005725.2 | NM_005725 |
| IOH12086 | 220203 | BC020626.1 | BC020626 |
| IOH23121 | 219258 | BC018782.1 | BC018782 |
| IOH11169 | 220114 | NM_138450.1 | NM_138450 |
| IOH13180 | 220120 | BC017344.1 | BC017344 |
| IOH12453 | 220122 | BC011765.2 | BC011765 |
| IOH22705 | 220124 | NM_173586.1 | NM_173586 |
| IOH21589 | 220125 | NM_152465.1 | NM_152465 |
| IOH13354 | 220126 | BC009968.2 | BC009968 |
| IOH21779 | 219252 | NM_145280.1 | NM_145280 |
| IOH6636 | 217968 | BC006142.2 | BC006142 |
| IOH4759 | 217975 | BC000038.1 | BC000038 |
| IOH3992 | 217962 | NM_005720.1 | NM_005720 |
| IOH7236 | 218014 | NM_032330.1 | NM_032330 |
| IOH6818 | 218017 | NM_032926.1 | NM_032926 |
| IOH12304 | 220619 | NM_138432.1 | NM_138432 |
| IOH9712 | 220587 | BC011526.1 | BC011526 |
| IOH13898 | 220588 | NM_002109.3 | NM_002109 |
| IOH10969 | 220591 | NM_032138.2 | NM_032138 |
| IOH28294 | 220604 | AB065630.1 | AB065630 |
| IOH13441 | 219594 | BC022253.1 | BC022253 |
| IOH3871 | 220626 | NM_007189.1 | NM_007189 |
| IOH13218 | 220627 | BC021090.1 | BC021090 |
| IOH12715 | 220638 | NM_015671.2 | NM_015671 |
| IOH12872 | 220649 | BC022270.1 | BC022270 |
| IOH4802 | 220655 | BC001214.1 | BC001214 |
| IOH27507 | 220656 | NM_175738.2 | NM_175738 |
| IOH14552 | 220661 | NM_04286.2 | NM_004286 |
| IOH3563 | 220611 | NM_015698.2 | NM_015698 |
| IOH10201 | 217054 | BC009006.1 | BC009006 |
| IOH22862 | 219597 | BC029652.1 | BC029652 |
| IOH11318 | 217037 | BC016395.1 | BC016395 |
| IOH10845 | 217039 | BC016848.1 | BC016848 |
| IOH11302 | 217040 | BC018113.1 | BC018113 |
| IOH10199 | 217042 | NM_018279.2 | NM_018279 |
| IOH10298 | 217044 | NM_080678.1 | NM_080678 |
| IOH10317 | 217045 | BC017724.1 | BC017724 |
| IOH10346 | 217046 | NM_007260.2 | NM_007260 |
| IOH10391 | 217047 | NM_020424.2 | NM_020424 |
| IOH11268 | 217051 | BC015479.1 | BC015479 |
| IOH10345 | 217034 | BC016979.1 | BC016979 |
| IOH10314 | 217033 | NM_031297.1 | NM_031297 |
| IOH10268 | 217055 | NM_006054.1 | NM_006054 |
| IOH10300 | 217056 | NM_001636.1 | NM_001636 |
| IOH10392 | 217059 | NM_152637.1 | NM_152637 |
| IOH10793 | 217060 | NM_017853.1 | NM_017853 |
| IOH11052 | 217061 | NM_012419.3 | NM_012419 |
| IOH11246 | 217063 | NM_015423.2 | NM_015423 |
| IOH10925 | 217065 | NM_013401.2 | NM_013401 |
| IOH10269 | 217067 | NM_052877.1 | NM_052877 |
| IOH10302 | 217068 | NM_031910.2 | NM_031910 |
| IOH10325 | 217069 | NM_033046.1 | NM_033046 |
| IOH11235 | 217052 | NM_014372.1 | NM_014372 |
| IOH11243 | 217012 | NM_006579.1 | NM_006579 |
| IOH14480 | 220683 | NM_019894.1 | NM_019894 |
| IOH11681 | 216799 | BC001550.1 | BC001550 |
| IOH3912 | 216800 | NM_021159.2 | NM_021159 |
| IOH3959 | 216801 | NM_016049.1 | NM_016049 |
| IOH4188 | 216804 | BC000651.1 | BC000651 |
| IOH3059 | 216807 | NM_002870.1 | NM_002870 |
| IOH3272 | 216808 | BC001286.1 | BC001286 |
| IOH13806 | 216810 | NM_002469.1 | NM_002469 |
| IOH3920 | 216811 | BC001120.1 | BC001120 |
| IOH4117 | 216813 | BC002616.1 | BC002616 |
| IOH4208 | 216815 | NM_014060.1 | NM_014060 |
| IOH4250 | 216816 | BC000607.1 | BC000607 |
| IOH10961 | 217036 | NM_004331.1 | NM_004331 |
| IOH3070 | 216818 | BC000809.1 | BC000809 |
| IOH10789 | 217075 | BC015239.1 | BC015239 |
| IOH10805 | 217013 | NM_002491.1 | NM_002491 |
| IOH10842 | 217014 | NM_052935.1 | NM_052935 |
| IOH10242 | 217019 | NM_058169.1 | NM_058169 |
| IOH10309 | 217021 | BC016942.1 | BC016942 |
| IOH10384 | 217023 | NM_032044.1 | NM_032044 |
| IOH11028 | 217026 | NM_145206.1 | NM_145206 |
| IOH11236 | 217028 | BC015468.1 | BC015468 |
| IOH10198 | 217030 | BC010241.1 | BC010241 |
| IOH10297 | 217032 | BC010555.1 | BC010555 |
| IOH2958 | 216817 | BC001001.2 | BC001001 |
| IOH14654 | 219562 | BC015667.2 | BC015667 |
| IOH22174 | 219563 | NM_002963.2 | NM_002963 |
| IOH22742 | 219564 | BC031650.1 | BC031650 |
| IOH23108 | 219567 | NM_001671.2 | NM_001671 |
| IOH6921 | 219568 | BC007602.1 | BC007602 |
| IOH23099 | 219573 | NM_015666.2 | NM_015666 |
| IOH5167 | 219574 | NM_032326.1 | NM_032326 |
| IOH22771 | 219575 | NM_004291.1 | NM_004291 |
| IOH10368 | 217070 | NM_003492.1 | NM_003492 |
| IOH5740 | 213577 | BC002940.1 | BC002940 |
| IOH6650 | 219556 | BC006148.1 | BC006148 |
| IOH21859 | 219581 | NM_139242.1 | NM_139242 |
| IOH13169 | 219582 | BC010167.2 | BC010167 |
| IOH22696 | 219583 | BC029121.1 | BC029121 |
| IOH22756 | 219584 | NM_152614.1 | NM_152614 |
| IOH23072 | 219585 | BC015842.1 | BC015842 |
| IOH22794 | 219588 | NM_002608.1 | NM_002608 |
| IOH22119 | 219591 | BC029760.1 | BC029760 |
| IOH21708 | 219592 | NM_152776.1 | NM_152776 |
| IOH3263 | 216796 | BC009009.1 | BC009009 |
| IOH21765 | 219576 | BC032775.1 | BC032775 |
| IOH10824 | 217095 | NM_014061.3 | NM_014061 |
| IOH10129 | 219595 | NM_016614.1 | NM_016614 |
| IOH110446 | 217076 | NM_002927.3 | NM_002927 |
| IOH10948 | 217077 | BC015409.1 | BC015409 |
| IOH10272 | 217079 | NM_005724.3 | NM_005724 |
| IOH10304 | 217080 | NM_138800.1 | NM_138800 |
| IOH10328 | 217081 | BC015329.1 | BC015329 |
| IOH10372 | 217082 | BC020962.1 | BC020962 |
| IOH11057 | 217086 | BC015535.1 | BC015535 |
| IOH11259 | 217089 | NM_002362.2 | NM_002362 |
| IOH10281 | 217091 | NM_032809.2 | NM_032809 |
| IOH9663 | 219559 | BC010458.1 | BC010458 |
| IOH10375 | 217094 | BC016857.1 | BC016857 |
| IOH14835 | 219557 | NM_174923.1 | NM_174923 |
| IOH11027 | 217096 | NM_138808.1 | NM_138808 |
| IOH10971 | 217100 | BC015413.1 | BC015413 |
| IOH10229 | 217101 | NM_016176.2 | NM_016176 |
| IOH10289 | 217102 | NM_052837.1 | NM_052837 |
| IOH10308 | 217103 | BC016941.1 | BC016941 |
| IOH10340 | 217104 | BC016934.1 | BC016934 |
| IOH10379 | 217105 | BC020966.1 | BC020966 |
| IOH22849 | 219551 | BC027486.1 | BC027486 |
| IOH22562 | 219552 | BC029524.1 | BC029524 |
| IOH23080 | 219555 | BC015878.1 | BC015878 |
| IOH10852 | 217074 | NM_003792.1 | NM_003792 |
| IOH10306 | 217092 | NM_006978.1 | NM_006978 |
| IOH12788 | 219789 | NM_177552.1 | NM_177552 |
| IOH5541 | 219804 | NM_004578.2 | NM_004578 |
| IOH3269 | 219768 | NM_003825.2 | NM_003825 |
| IOH9701 | 219769 | BC010642.1 | BC010642 |
| IOH3256 | 219770 | BC001244.1 | BC001244 |
| IOH13784 | 219771 | BC015066.1 | BC015066 |
| IOH22826 | 219777 | NM_031491.1 | NM_031481 |
| IOH14352 | 219778 | NM_005614.2 | NM_005614 |
| IOH14450 | 219779 | NM_003278.1 | NM_003278 |
| IOH14289 | 219780 | NM_006007.1 | NM_006007 |
| IOH13742 | 219781 | BC010959.1 | BC010959 |
| IOH3965 | 219782 | NM_004357.2 | NM_004357 |
| IOH3081 | 219784 | NM_016098.1 | NM_016098 |
| IOH2916 | 219766 | NM_015646.1 | NM_015646 |
| IOH7254 | 219788 | BC005218.1 | BC005218 |
| IOH12177 | 219765 | BC014991.1 | BC014991 |
| IOH5958 | 219790 | BC008365.1 | BC008365 |
| IOH14099 | 219791 | BC011842.2 | BC011842 |
| IOH6329 | 219792 | BC006288.1 | BC006288 |
| IOH14184 | 219793 | BC011006.1 | BC011006 |
| IOH10868 | 219794 | NM_145006.1 | NM_145006 |
| IOH11073 | 219795 | BC012947.1 | BC012947 |
| IOH14044 | 219796 | BC021286.1 | BC021286 |
| IOH6278 | 219797 | BC007689.2 | BC007689 |
| IOH10802 | 219800 | NM_145286.1 | NM_145286 |
| IOH14443 | 219801 | NM_020980.2 | NM_020980 |
| IOH14506 | 219802 | NM_152267.2 | NM_152267 |
| IOH13864 | 216619 | NM_005558.2 | NM_005558 |
| IOH11390 | 219785 | BC015492.1 | BC015492 |
| IOH2929 | 219748 | BC003377.1 | BC003377 |
| IOH27228 | 220688 | NM_019109.1 | NM_019109 |
| IOH5421 | 216624 | NM_016103.1 | NM_016103 |
| IOH6672 | 216625 | NM_002867.2 | NM_002867 |
| IOH10734 | 216626 | BC020495.1 | BC020495 |
| IOH14575 | 216627 | NM_006270.2 | NM_006270 |
| IOH9688 | 216628 | NM_004422.1 | NM_004422 |
| IOH13239 | 216629 | NM_018969.2 | NM_018969 |
| IOH21132 | 216630 | NM_024046.1 | NM_024046 |
| IOH22568 | 219741 | NM_152587.2 | NM_152587 |
| IOH4077 | 219742 | BC002520.1 | BC002520 |
| IOH14113 | 219744 | BC009762.2 | BC009762 |
| IOH7448 | 219745 | BC008438.1 | BC008438 |
| IOH14238 | 219767 | BC021241.2 | BC021241 |
| IOH13789 | 219747 | BC010963.1 | BC010963 |
| IOH3028 | 219805 | NM_031227.1 | NM_031227 |
| IOH5164 | 219754 | BC004896.1 | BC004896 |
| IOH13706 | 219752 | NM_003106.2 | NM_003106 |
| IOH6738 | 219753 | BC007806.1 | BC007806 |
| IOH11628 | 219754 | NM_144593.1 | NM_144593 |
| IOH11804 | 219755 | BC028728.1 | BC028728 |
| IOH14448 | 219756 | BC017101.1 | BC017101 |
| IOH14519 | 219757 | BC014521.1 | BC014521 |
| IOH14186 | 219758 | NM_015975.3 | NM_015975 |
| IOH11799 | 219759 | NM_001008.2 | NM_001008 |
| IOH3847 | 219760 | NM_016468.2 | NM_016468 |
| IOH12799 | 219763 | NM_024713.1 | NM_024713 |
| IOH5099 | 219764 | NM_001154.2 | NM_001154 |
| IOH10850 | 219746 | NM_152667.1 | NM_152667 |
| IOH12227 | 219983 | BC009779.1 | BC009779 |
| IOH5640 | 219803 | NM_031472.1 | NM_031472 |
| IOH14089 | 219945 | BC014095.2 | BC014095 |
| IOH5465 | 219947 | BC004938.1 | BC004938 |
| IOH14627 | 219948 | BC021995.1 | BC021995 |
| IOH12733 | 219950 | NM_144654.1 | NM_144654 |
| IOH12301 | 219951 | NM_006643.2 | NM_006643 |
| IOH10186 | 219953 | BC010504.1 | BC010504 |
| IOH12212 | 219955 | BC012609.1 | BC012609 |
| IOH6217 | 219963 | NM_033177.2 | NM_033177 |
| IOH14248 | 219964 | BC014665.1 | BC014665 |
| IOH13812 | 219966 | NM_003666.1 | NM_003666 |
| IOH10741 | 219967 | NM_053285.1 | NM_053285 |
| IOH10347 | 219942 | NM_002194.2 | NM_002194 |
| IOH4736 | 219977 | BC000111.1 | BC000111 |
| IOH3316 | 219941 | NM_138379.1 | NM_138379 |
| IOH12689 | 219984 | BC012192.1 | BC012192 |
| IOH12915 | 219995 | NM_016305.1 | NM_016305 |
| IOH10208 | 219996 | BC013648.1 | BC013648 |
| IOH13007 | 220000 | NM_002243.2 | NM_002243 |
| IOH9923 | 220001 | NM_005103.3 | NM_005103 |
| IOH3184 | 220004 | BC006793.1 | BC006793 |
| IOH5273 | 220006 | BC002629.1 | BC002629 |
| IOH10197 | 220010 | BC008141.1 | BC008141 |
| IOH10264 | 220013 | BC016440.1 | BC016440 |
| IOH9764 | 220014 | BC018445.1 | BC018445 |
| IOH4911 | 220015 | BC001709.1 | BC001709 |
| IOH10296 | 220017 | BC012881.1 | BC012881 |
| IOH14388 | 219975 | NM_003943.1 | NM_003943 |
| IOH5875 | 219829 | NM_018129.1 | NM_018129 |
| IOH3275 | 219806 | NM_007241.2 | NM_007241 |
| IOH2956 | 219807 | NM_030920.1 | NM_030920 |
| IOH12991 | 219812 | NM_033416.1 | NM_033416 |
| IOH23147 | 219813 | BC029399.1 | BC029399 |
| IOH12754 | 219814 | BC010889.1 | BC010889 |
| IOH5954 | 219815 | NM_006241.2 | NM_006241 |
| IOH6926 | 219816 | BC007312.1 | BC007312 |
| IOH11176 | 219817 | BC012919.1 | BC012919 |
| IOH12664 | 219818 | NM_138412.1 | NM_138412 |
| IOH3923 | 219819 | NM_005333.1 | NM_005333 |
| IOH14467 | 219823 | NM_001760.2 | NM_001760 |
| IOH2920 | 219825 | BC000903.2 | BC000903 |
| IOH3201 | 219943 | BC001964.1 | BC001964 |
| IOH4156 | 219827 | NM_019606.3 | NM_019606 |
| IOH10344 | 216618 | BC016964.1 | BC016964 |
| IOH12105 | 219830 | BC015118.1 | BC015118 |
| IOH3283 | 219831 | BC008990.1 | BC008990 |
| IOH3251 | 219926 | NM_024058.1 | NM_024058 |
| IOH14527 | 219927 | NM_172341.1 | NM_172341 |
| IOH12891 | 219929 | BC013319.1 | BC013319 |
| IOH9750 | 219930 | BC016614.1 | BC016614 |
| IOH6391 | 219931 | NM_033661.1 | NM_033661 |
| IOH3325 | 219935 | BC008091.1 | BC008091 |
| IOH12592 | 219936 | BC010181.1 | BC010181 |
| IOH5376 | 219938 | NM_007233.1 | NM_007233 |
| IOH4363 | 219939 | NM_005272.2 | NM_005272 |
| IOH10698 | 219940 | NM_182488.1 | NM_182488 |
| IOH6081 | 219826 | BC005876.1 | BC005876 |
| IOH20996 | 216539 | NM_006504.2 | NM_006504 |
| IOH7013 | 216552 | BC007324.1 | BC007324 |
| IOH11251 | 216523 | BC025708.1 | BC025708 |
| IOH12770 | 216524 | NM_052946.1 | NM_052946 |
| IOH14193 | 216526 | NM_144624.1 | NM_144624 |
| IOH21152 | 216527 | NM_005248.1 | NM_005248 |
| IOH5340 | 216528 | BC002706.1 | BC002706 |
| IOH4753 | 216529 | BC000729.1 | BC000729 |
| IOH6313 | 216530 | NM_000858.2 | NM_000858 |
| IOH6708 | 216531 | NM_002045.1 | NM_002045 |
| IOH5978 | 216532 | NM_001827.1 | NM_001827 |
| IOH12559 | 216534 | BC013992.1 | BC013992 |
| IOH13992 | 216535 | NM_013410.1 | NM_013410 |
| IOH7357 | 216521 | BC005371.1 | BC005371 |
| IOH2412 | 216537 | NM_003583.2 | NM_003583 |
| IOH7134 | 216520 | BC008374.1 | BC008374 |
| IOH6325 | 216540 | NM_007240.1 | NM_007240 |
| IOH13715 | 216541 | NM_177554.1 | NM_177554 |
| IOH5691 | 216542 | BC004522.1 | BC004522 |
| IOH7574 | 216543 | NM_001664.1 | NM_001664 |
| IOH12834 | 216544 | BC018942.1 | BC018942 |
| IOH11309 | 216545 | BC024004.1 | BC024004 |
| IOH3294 | 216546 | NM_001736.1 | NM_001736 |
| IOH11033 | 216547 | NM_004720.3 | NM_004720 |
| IOH13042 | 216549 | NM_003130.1 | NM_003130 |
| IOH4141 | 216550 | NM_054033.1 | NM_054033 |
| IOH13214 | 216623 | NM_033256.1 | NM_033256 |
| IOH14360 | 216536 | NM_001625.1 | NM_001625 |
| IOH12669 | 216499 | BC014552.1 | BC014552 |
| IOH21154 | 216480 | NM_017490.1 | NM_017490 |
| IOH6979 | 216484 | NM_000269.1 | NM_000269 |
| IOH10122 | 216486 | NM_000431.1 | NM_000431 |
| IOH12980 | 216487 | BC015186.1 | BC015186 |
| IOH11014 | 216488 | NM_005565.2 | NM_005565 |
| IOH11645 | 216489 | NM_001721.2 | NM_001721 |
| IOH14591 | 216490 | BC021278.1 | BC021278 |
| IOH20967 | 216492 | NM_020439.1 | NM_020439 |
| IOH5163 | 216493 | NM_001800.2 | NM_001800 |
| IOH5481 | 216494 | NM_018110.2 | NM_018110 |
| IOH6258 | 216495 | NM_033019.1 | NM_033019 |
| IOH7002 | 216496 | NM_018571.4 | NM_018571 |
| IOH10488 | 216522 | BC018345.1 | BC018345 |
| IOH10145 | 216498 | NM_005391.1 | NM_005391 |
| IOH11625 | 216553 | BC028719.1 | BC028719 |
| IOH11097 | 216500 | NM_004417.2 | NM_004417 |
| IOH5211 | 216505 | NM_001823.2 | NM_001823 |
| IOH4633 | 216506 | NM_002044.1 | NM_002044 |
| IOH6284 | 216507 | BC006231.1 | BC006231 |
| IOH7132 | 216508 | NM_006748.1 | NM_006748 |
| IOH7287 | 216509 | BC007462.1 | BC007462 |
| IOH10919 | 216511 | NM_145025.1 | NM_145025 |
| IOH11402 | 216513 | NM_024779.2 | NM_024779 |
| IOH14775 | 216514 | BC024291.1 | BC024291 |
| IOH21038 | 216515 | NM_005233.2 | NM_005233 |
| IOH4674 | 216518 | NM_031361.1 | NM_031361 |
| IOH6288 | 216519 | BC006233.1 | BC006233 |
| IOH7271 | 216497 | BC005298.1 | BC005298 |
| IOH5158 | 216605 | BC005153.1 | BC005153 |
| IOH21299 | 216551 | NM_024025.1 | NM_024025 |
| IOH10104 | 216591 | NM_022337.1 | NM_022337 |
| IOH1753 | 216592 | NM_001667.1 | NM_001667 |
| IOH3460 | 216593 | NM_002436.2 | NM_002436 |
| IOH6697 | 216596 | NM_020299.2 | NM_020299 |
| IOH14446 | 216597 | BC022305.1 | BC022305 |
| IOH5443 | 216599 | NM_003712.1 | NM_003712 |
| IOH12943 | 216600 | BC009196.1 | BC009196 |
| IOH14614 | 216601 | BC021289.1 | BC021289 |
| IOH6072 | 216602 | NM_023940.1 | NM_023940 |
| IOH14587 | 216589 | NM_002710.1 | NM_002710 |
| IOH14475 | 216604 | NM_002884.1 | NM_002884 |
| IOH12805 | 216588 | NM_014241.2 | NM_014241 |
| IOH9624 | 216606 | NM_003382.2 | NM_003382 |
| IOH1987 | 216607 | NM_015727.1 | NM_015727 |
| IOH11395 | 216609 | BC028739.2 | BC028739 |
| IOH7464 | 216610 | NM_016301.2 | NM_016301 |
| IOH5608 | 216611 | NM_005605.2 | NM_005605 |
| IOH12269 | 216612 | BC020700.1 | BC020700 |
| IOH4164 | 216613 | BC000566.1 | BC000566 |
| IOH6101 | 216614 | NM_017595.2 | NM_017595 |
| IOH10511 | 216615 | NM_004283.2 | NM_004283 |
| IOH14604 | 216616 | NM_002070.1 | NM_002070 |
| IOH5175 | 216617 | BC005155.1 | BC005155 |
| IOH10139 | 216603 | NM_021252.2 | NM_021252 |
| IOH14797 | 216569 | NM_022777.1 | NM_022777 |
| IOH5472 | 216554 | BC004247.1 | BC004247 |
| IOH9848 | 216555 | NM_002068.1 | NM_002068 |
| IOH10825 | 216556 | NM_145313.1 | NM_145313 |
| IOH1937 | 216557 | NM_006822.1 | NM_006822 |
| IOH3305 | 216558 | BC008094.1 | BC008094 |
| IOH12614 | 216559 | BC009877.1 | BC009877 |
| IOH4559 | 216560 | NM_024076.1 | NM_024076 |
| IOH12967 | 216561 | BC009961.1 | BC009961 |
| IOH4659 | 216562 | BC000103.1 | BC000103 |
| IOH3815 | 216563 | NM_007236.2 | NM_007236 |
| IOH7224 | 216564 | NM_002721.3 | NM_002721 |
| IOH4847 | 216566 | BC003088.1 | BC003088 |
| IOH4954 | 216590 | NM_001663.2 | NM_001663 |
| IOH12833 | 216568 | NM_014310.3 | NM_014310 |
| IOH12030 | 218896 | NM_002704.1 | NM_002704 |
| IOH5698 | 216572 | NM_031436.1 | NM_031436 |
| IOH12198 | 216573 | NM_005832.2 | NM_005832 |
| IOH4436 | 216574 | NM_002903.1 | NM_002903 |
| IOH3548 | 216575 | NM_001467.2 | NM_001467 |
| IOH7558 | 216576 | BC008493.1 | BC008493 |
| IOH13822 | 216577 | NM_016361.2 | NM_016361 |
| IOH10011 | 216579 | NM_006861.2 | NM_006861 |
| IOH12810 | 216580 | NM_016530.1 | NM_016530 |
| IOH14673 | 216581 | NM_004251.2 | NM_004251 |
| IOH5739 | 216584 | NM_020677.1 | NM_020677 |
| IOH5913 | 216586 | NM_172016.1 | NM_172016 |
| IOH5237 | 216587 | NM_004090.1 | NM_004090 |
| IOH10004 | 216567 | NM_020673.1 | NM_020673. |
| IOH14287 | 219845 | NM_053045.1 | NM_053045 |
| IOH11993 | 219861 | BC020976.1 | BC020976 |
| IOH21099 | 219540 | NM_020185.2 | NM_020185 |
| IOH21339 | 219541 | NM_016508.2 | NM_016508 |
| IOH22332 | 219545 | NM_024745.1 | NM_024745 |
| IOH21538 | 219548 | BC032249.1 | BC032249 |
| IOH5031 | 219834 | NM_032308.1 | NM_032308 |
| IOH7456 | 219835 | NM_145792.1 | NM_145792 |
| IOH4806 | 219836 | BC001907.1 | BC001907 |
| IOH5889 | 219838 | BC008037.2 | BC008037 |
| IOH9807 | 219840 | BC009047.1 | BC009047 |
| IOH3994 | 219841 | NM_020467.2 | NM_020467 |
| IOH13242 | 219537 | BC015625.1 | BC015625 |
| IOH3136 | 219844 | NM_005340.1 | NM_005340 |
| IOH22318 | 219534 | BC030597.1 | BC030597 |
| IOH2912 | 219846 | BC003366.1 | BC003366 |
| IOH3243 | 219847 | NM_007362.2 | NM_007362 |
| IOH10494 | 219848 | NM_016058.1 | NM_016058 |
| IOH5367 | 219851 | BC002758.1 | BC002758 |
| IOH4100 | 219852 | NM_006468.3 | NM_006468 |
| IOH3240 | 219853 | BC001256.1 | BC001256 |
| IOH4556 | 219854 | NM_005274.1 | NM_005274 |
| IOH3382 | 219855 | BC008651.1 | BC008651 |
| IOH10623 | 219857 | BC015155.1 | BC015155 |
| IOH13168 | 218894 | NM_032574.1 | NM_032574 |
| IOH13650 | 219843 | BC018953.1 | BC018953 |
| IOH21787 | 219480 | BC033851.1 | BC033851 |
| IOH4703 | 219454 | BC000712.1 | BC000712 |
| IOH22829 | 219455 | BC027465.1 | BC027465 |
| IOH5310 | 219456 | BC002769.1 | BC002769 |
| IOH21007 | 219457 | BC031549.1 | BC031549 |
| IOH21418 | 219459 | BC034718.1 | BC034718 |
| IOH13910 | 219464 | NM_005510.2 | NM_005510 |
| IOH6373 | 219465 | NM_024901.2 | NM_024901 |
| IOH21512 | 219468 | BC030253.1 | BC030253 |
| IOH21026 | 219469 | NM_022048.1 | NM_022048 |
| IOH21419 | 219471 | BC011392.1 | BC011392 |
| IOH22249 | 219473 | BC036649.1 | BC036649 |
| IOH22290 | 219474 | BC030776.1 | BC030776 |
| IOH13175 | 219538 | NM_138790.1 | NM_138790 |
| IOH22410 | 219476 | BC030020.2 | BC030020 |
| IOH4057 | 219862 | BC001408.1 | BC001408 |
| IOH22297 | 219486 | BC034483.1 | BC034483 |
| IOH6500 | 219492 | NM_032694.1 | NM_032694 |
| IOH21472 | 219496 | BC019954.1 | BC019954 |
| IOH22299 | 219498 | NM_032491.2 | NM_032491 |
| IOH22369 | 219499 | NM_006202.1 | NM_006202 |
| IOH21592 | 219503 | NM_152394.2 | NM_152394 |
| IOH22389 | 219511 | BC030653.2 | BC030653 |
| IOH20954 | 219516 | NM_178152.1 | NM_178152 |
| IOH21323 | 219518 | NM_001277.1 | NM_001277 |
| IOH21336 | 219530 | NM_014326.2 | NM_014326 |
| IOH21451 | 219531 | BC034247.1 | BC034247 |
| IOH22282 | 219533 | BC034468.1 | BC034468 |
| IOH22340 | 219475 | NM_033103.1 | NM_033103 |
| IOH7163 | 219915 | NM_004102.2 | NM_004102 |
| IOH12123 | 219859 | NM_173362.2 | NM_173362 |
| IOH14013 | 219897 | NM_005147.1 | NM_005147 |
| IOH13637 | 219898 | BC015754.1 | BC015754 |
| IOH13536 | 219899 | NM_005842.2 | NM_005842 |
| IOH2980 | 219900 | BC000962.2 | BC000962 |
| IOH5105 | 219901 | BC004969.1 | BC004969 |
| IOH5325 | 219902 | NM_024312.1 | NM_024312 |
| IOH5254 | 219903 | BC002656.1 | BC002656 |
| IOH11669 | 219905 | NM_152773.2 | NM_152773 |
| IOH5830 | 219906 | BC007407.1 | BC007407 |
| IOH3804 | 219907 | BC004179.1 | BC004179 |
| IOH6880 | 219908 | BC007282.1 | BC007282 |
| IOH6966 | 219895 | NM_032920.1 | NM_032920 |
| IOH11511 | 219913 | BC028039.1 | BC028039 |
| IOH3328 | 219893 | BC008567.1 | BC008567 |
| IOH3511 | 219916 | NM_006022.1 | NM_006022 |
| IOH14253 | 219917 | BC010896.1 | BC010896 |
| IOH12025 | 219918 | BC027866.1 | BC027866 |
| IOH5656 | 219919 | NM_015610.1 | NM_015610 |
| IOH11880 | 219920 | NM_003447.1 | NM_003447 |
| IOH14723 | 219921 | BC011928.2 | BC011928 |
| IOH6345 | 219922 | BC008803.1 | BC008803 |
| IOH4359 | 219923 | NM_021992.1 | NM_021992 |
| IOH6980 | 219925 | NM_032886.1 | NM_032886 |
| IOH13940 | 220678 | NM_144620.1 | NM_144620 |
| IOH10654 | 220681 | NM_007249.3 | NM_007249 |
| IOH7170 | 220682 | BC006986.1 | BC006986 |
| IOH9842 | 219910 | BC009734.1 | BC009734 |
| IOH12626 | 219880 | NM_012396.1 | NM_012396 |
| IOH14667 | 219863 | BC020786.1 | BC020786 |
| IOH12518 | 219865 | BC010172.2 | BC010172 |
| IOH4263 | 219866 | NM_000999.2 | NM_000999 |
| IOH13535 | 219867 | BC016754.1 | BC016754 |
| IOH4447 | 219868 | BC001716.1 | BC001716 |
| IOH5650 | 219869 | BC004885.1 | BC004885 |
| IOH11279 | 219870 | BC017064.1 | BC017064 |
| IOH12898 | 219871 | BC10900.1 | BC010900 |
| IOH09869 | 219874 | NM_017837.2 | NM_017837 |
| IOH4273 | 219875 | BC002430.1 | BC002430 |
| IOH4189 | 219876 | NM_014366.1 | NM_014366 |
| IOH3865 | 219877 | BC001694.1 | BC001694 |
| IOH5510 | 219896 | NM_024061.1 | NM_024061 |
| IOH10463 | 219879 | BC013687.1 | BC013687 |
| IOH11381 | 219451 | NM_005641.2 | NM_005641 |
| IOH6968 | 219881 | BC007639.1 | BC007639 |
| IOH7274 | 219882 | NM_031427.1 | NM_031427 |
| IOH13646 | 219883 | BC015059.1 | BC015059 |
| IOH5952 | 219884 | NM_001660.2 | NM_001660 |
| IOH11106 | 219885 | NM_006838.1 | NM_006838 |
| IOH4913 | 219886 | BC002954.1 | BC002954 |
| IOH14170 | 219887 | BC022361.1 | BC022361 |
| IOH6338 | 219888 | BC006259.2 | BC006259 |
| IOH4850 | 219889 | NM_178191.1 | NM_178191 |
| IOH21487 | 219890 | NM_052861.1 | NM_052861 |
| IOH4965 | 219891 | BC001868.1 | BC001868 |
| IOH14751 | 219892 | BC015091.2 | BC015091 |
| IOH5727 | 219878 | BC002934.1 | BC002934 |
| IOH12223 | 218954 | NM_002555.2 | NM_002555 |
| IOH14755 | 219453 | BC018747.1 | BC018747 |
| IOH14111 | 218932 | NM_145271.1 | NM_145271 |
| IOH12986 | 218933 | NM_000200.1 | NM_000200 |
| IOH10884 | 218934 | NM_145254.1 | NM_145254 |
| IOH11035 | 218935 | BC018028.1 | BC018028 |
| IOH12529 | 218938 | BC010414.1 | BC010414 |
| IOH12944 | 218939 | BC009393.2 | BC009393 |
| IOH12382 | 218940 | NM_000608.1 | NM_000608 |
| IOH13353 | 218941 | NM_138794.1 | NM_138794 |
| IOH12649 | 218942 | NM_033281.2 | NM_033281 |
| IOH12242 | 218943 | NM_145300.1 | NM_145300 |
| IOH11127 | 218946 | NM_004202.1 | NM_004202 |
| IOH13435 | 218930 | BC017381.1 | BC017383 |
| IOH12548 | 218950 | BC009873.1 | BC009873 |
| IOH12601 | 218927 | BC009366.1 | BC009366 |
| IOH13307 | 218955 | NM_025065.4 | NM_025065 |
| IOH10921 | 218956 | BC016900.1 | BC016900 |
| IOH12487 | 218957 | BC010426.1 | BC010426 |
| IOH11137 | 218958 | BC020942.1 | BC020942 |
| IOH11067 | 218959 | NM_080739.1 | NM_080739 |
| IOH12519 | 218961 | NM_017503.2 | NM_017503 |
| IOH12579 | 218962 | BC012783.2 | BC012783 |
| IOH12074 | 218964 | BC014307.1 | BC014307 |
| IOH13306 | 218965 | BC017399.1 | BC017399 |
| IOH12816 | 218966 | NM_006216.2 | NM_006216 |
| IOH12539 | 218967 | NM_018215.1 | NM_018215 |
| IOH11147 | 218968 | BC012493.1 | BC012493 |
| IOH13317 | 218948 | NM_052950.2 | NM_052950 |
| IOH10849 | 218912 | NM_144717.1 | NM_144717 |
| IOH21059 | 216479 | NM_003656.3 | NM_003656 |
| IOH12727 | 218897 | NM_018413.2 | NM_018413 |
| IOH13016 | 218898 | BC012984.2 | BC012984 |
| IOH11006 | 218899 | NM_003766.2 | NM_003766 |
| IOH10955 | 218900 | BC027473.1 | BC027473 |
| IOH13426 | 218901 | BC014089.2 | BC014089 |
| IOH12121 | 218902 | NM_014035.1 | NM_014035 |
| IOH13230 | 218903 | NM_130777.1 | NM_130777 |
| IOH12337 | 218904 | NM_006476.2 | NM_006476 |
| IOH12458 | 218905 | BC013935.1 | BC013935 |
| IOH12647 | 218906 | NM_005726.2 | NM_005726 |
| IOH12275 | 218907 | NM_144982.1 | NM_144982 |
| IOH12225 | 218931 | NM_002621.1 | NM_002621 |
| IOH11093 | 218910 | NM_012473.2 | NM_012473 |
| IOH10783 | 218971 | NM_145013.1 | NM_145013 |
| IOH12533 | 218913 | NM_005376.1 | NM_005376 |
| IOH12454 | 218914 | NM_138482.1 | NM_138482 |
| IOH12084 | 218916 | BC021680.1 | BC021680 |
| IOH13071 | 218917 | NM_145303.1 | NM_145303 |
| IOH13075 | 218918 | NM_138573.1 | NM_138573 |
| IOH12288 | 218919 | NM_032570.1 | NM_032570 |
| IOH11647 | 218920 | NM_024561.1 | NM_024561 |
| IOH12120 | 218921 | BC012569.1 | BC012569 |
| IOH10420 | 218922 | NM_004089.1 | NM_004089 |
| IOH10822 | 218924 | BC025791.1 | BC025791 |
| IOH12648 | 218925 | NM_032125.1 | NM_032125 |
| IOH12476 | 218926 | NM_022054.2 | NM_022054 |
| IOH12165 | 218909 | BC011014.1 | BC011014 |
| IOH4541 | 219431 | BC001174.1 | BC001174 |
| IOH22628 | 219415 | BC029032.1 | BC029032 |
| IOH10380 | 219416 | NM_138792.1 | NM_138792 |
| IOH22889 | 219417 | NM_005550.2 | NM_005550 |
| IOH23047 | 219418 | NM_152576.1 | NM_152576 |
| IOH5894 | 219419 | NM_000404.1 | NM_000404 |
| IOH21749 | 219420 | NM_178523.2 | NM_178523 |
| IOH22763 | 219422 | BC031661.1 | BC031661 |
| IOH21756 | 219423 | BC033710.1 | BC033710 |
| IOH13504 | 219424 | NM_138436.1 | NM_138436 |
| IOH6468 | 219425 | NM_000281.1 | NM_000281 |
| IOH12235 | 219426 | BC017943.1 | BC017943 |
| IOH10509 | 219428 | BC013051.1 | BC013051 |
| IOH12557 | 218969 | NM_138397.1 | NM_139397 |
| IOH3444 | 219430 | NM_001819.1 | NM_001819 |
| IOH22190 | 219411 | BC031827.1 | BC031827 |
| IOH6765 | 219432 | NM_032908.1 | NM_032908 |
| IOH12282 | 219435 | BC020867.1 | BC020967 |
| IOH10009 | 219437 | NM_021218 | NM_021218 |
| IOH13414 | 219438 | NM_031210.1 | NM_031210 |
| IOH22940 | 219441 | BC030005.1 | BC030005 |
| IOH3500 | 219442 | NM_006831.1 | NM_006831 |
| IOH4587 | 219443 | BC000091.1 | BC000091 |
| IOH21581 | 219444 | BC029568.1 | BC029568 |
| IOH22117 | 219447 | BC013103.1 | BC013103 |
| IOH12990 | 219448 | BC010155.2 | BC010155 |
| IOH3154 | 219450 | NM_138386.1 | NM_138386 |
| IOH13085 | 218895 | NM_022142.3 | NM_022142 |
| IOH22939 | 219429 | BC030636.1 | BC030636 |
| IOH23129 | 219375 | NM_006519.1 | NM_006519 |
| IOH22963 | 219452 | NM_002095.1 | NM_002095 |
| IOH12071 | 218972 | NM_138463.1 | NM_138463 |
| IOH12646 | 218973 | BC011578.1 | BC011578 |
| IOH12127 | 218976 | BC021682.1 | BC021682 |
| IOH10917 | 218982 | NM_031950.1 | NM_031950 |
| IOH12659 | 218985 | BC009230.2 | BC009230 |
| IOH13888 | 219362 | BC017869.1 | BC017869 |
| IOH22577 | 219363 | NM_152914.1 | NM_152914 |
| IOH6467 | 219365 | BC006370.2 | BC006370 |
| IOH22461 | 219367 | NM_153350.2 | NM_153350 |
| IOH2960 | 219368 | NM_024059.2 | NM_024059 |
| IOH11667 | 219369 | BC017046.1 | BC017046 |
| IOH21844 | 219414 | NM_005423.1 | NM_005423 |
| IOH22727 | 219374 | BC029799.1 | BC029799 |
| IOH21569 | 219413 | BC028113.1 | BC028113 |
| IGH21513 | 219377 | NM_015973.1 | NM_015973 |
| IOH6669 | 219378 | BC007207.1 | BC007207 |
| IOH10913 | 219380 | NM_004567.2 | NM_004567 |
| IOH11817 | 219381 | NM_002197.1 | NM_002197 |
| IOH21704 | 219384 | BC032347.1 | BC032347 |
| IOH22492 | 219391 | NM_145028.1 | NM_145028 |
| IOH3770 | 219395 | BC001669.1 | BC001669 |
| IOH22121 | 219396 | BC013171.1 | BC013171 |
| IOH3092 | 219404 | NM_017512.1 | NM_017512 |
| IOH3744 | 219407 | BC004159.1 | BC004159 |
| IOH10277 | 219408 | NM_138491.1 | NM_138491 |
| IOH22760 | 219410 | BC031655.1 | BC031655 |
| IOH11199 | 218970 | BC022471.1 | BC022471 |
| IOH14733 | 219372 | BC009245.1 | BC009245 |

TABLE 6
AccNumber

NM_001893.3
NM_001894.2
NM_004196.2
NM_052987.1
NM_001826.1
NM_016507.1
NM_020547.1
NM_015850.2
NM_023030.1
NM_004635.2
NM_003137.2
NM_002576.2
NM_005030.2
NM_004071.1
NM_002748.2
NM_002732.2
NM_001786.2
NM_004431.1
NM_004442.3
NM_002253.1
NM_003010.1
NM_042066.8
NM_005922.1
NM_005923.3
NM_005965.2
NM_006254.1
NM_005400.1
NM_002731.1
NM_001654.1
NM_003688.1
NM_004938.1
NM_002314.2
NM_002742.1
NM_002738.2
NM_001619.2
NM_003691.1
NM_003942.1
NM_003188.2
NM_004834.2
NM_005990.1
NM_003674.1
NM_002613.1
NM_003384.1
NM_003600.1
NM_003607.1
NM_004586.1
NM_004217.1
NM_003242.2
NM_002741.1
NM_006281.1
NM_006852.1
NM_007064.1
NM_017572.1
NM_017593.2
NM_018401.1
NM_020397.1
NM_021133.1
NM_018650.1
NM_021643.1
NM_003952.1
NM_005884.2
NM_013233.1
NM_025195.1
NM_012395.1
NM_013257.2
NM_013392.1
NM_005465.2
NM_006035.2
NM_006282.1
NM_005813.2
NM_020168.3
NM_020328.1
NM_002752.3
NM_002754.3
NM_004383.1
NM_001259.2
NM_001892.2
NM_001106.2
NM_001896.1
NM_002756.2
NM_000061.1
NM_022972.1
NM_004445.1
NM_005235.1
NM_004443.2
NM_004560.2
NM_005157.2
NM_001616.2
NM_004441.2
NM_001982.1
NM_000459.1
NM_004444.2
NM_006343.1
NM_000075.2
NM_001258.1
NM_001261.2
NM_001799.2
NM_004935.1
BC000479.1
NM_016440.1
NM_016735.1
NM_001203.1
NM_005163.1
NM_005204.2
NM_005627.1
NM_002037.1
NM_002350.1
BC001280.1
NM_015978.1
NM_005012.1
NM_003576.2
NM_013254.2
NM_005417.2
NM_032409.1
NM_004103.2
NM_001396.2
NM_004226.1
NM_015112.1
NM_005228.1
NM_006213.1
NM_005246.1
NM_014920.1
NM_005906.2
NM_033115.1
NM_012424.2
NM_004759.2
NM_006622.1
NM_014002.1
NM_014496.1
NM_007194.1
NM_002745.2
NM_002447.1
NM_013355.1
NM_032844.1
NM_006258.1
NM_017719.2
NM_031414.2
NM_001626.2
NM_006256.1
NM_018423.1
NM_032237.1
NM_002750.2
NM_102578.1
BC001662.1
BC017715.1
BC001274.1
BC000442.1
BC006106.1
NM_003948.2
BC003614.1
NM_002744.2
BC005408.1
NM_033621.1
BC008302.1
BC000471.1
BC002541.1
BC002755.1
BC008716.1
BC001968.1
BC008838.1
BC000251.1
BC002637.1
BC016652.1
BC012761.1
BC008726.1
BC020972.1
BC011668.1
BC004207.1
BC003065.1
BC002695.1
BC018111.1
BC013879.1
NM_018492.2
NM_024776.1
NM_024800.1
BC014037.1

TABLE 7

| COLONY_NAME | COLONY_ID | ACCNO | trunCACC | CONCENTRATION |
|---|---|---|---|---|
| IOH10670 | 216928 | NM_001637.1 | NM_001637 | 65 |
| IOH13082 | 216944 | BC013393.2 | BC013393 | 2172 |
| IOH10699 | 216927 | BC024187.2 | BC024187 | 22 |
| IOH13295 | 216946 | BC012330.1 | BC012330 | 336 |
| IOH12655 | 216947 | BC012072.1 | BC012072 | 81 |
| IOH12800 | 216948 | BC014194.1 | BC014194 | 56 |
| IOH10808 | 216949 | NM_152613.1 | NM_152613 | 96 |
| IOH11247 | 216950 | NM_024411.1 | NM_024411 | 198 |
| IOH13403 | 216952 | BC011878.2 | BC011878 | 92 |
| IOH13383 | 216954 | NM_145042.1 | NM_145042 | 82 |
| IOH13411 | 216955 | BC009253.1 | BC009253 | 2232 |
| IOH12828 | 216956 | NM_145061.1 | NM_145061 | 432 |
| IOH12732 | 216957 | NM_052838.2 | NM_052838 | 2627 |
| IOH13260 | 216943 | NM_145043.1 | NM_145043 | 2789 |
| IOH13348 | 216903 | NM_144676.1 | NM_144676 | 52 |
| IOH12335 | 216890 | BC022319.1 | BC022319 | 431 |
| IOH12946 | 216891 | BC022300.1 | BC022300 | 122 |
| IOH10305 | 221173 | BC020555.1 | BC020555 | 91 |
| IOH12236 | 216895 | BC013902.1 | BC013902 | 31 |
| IOH27257 | 220804 | NM_000286.1 | NM_000286 | 64 |
| IOH5639 | 219024 | BC004505.1 | BC004505 | 843 |
| IOH4675 | 219025 | BC000742.1 | BC000742 | 998 |
| IOH4986 | 219026 | BC004965.1 | BC004965 | 736 |
| IOH4978 | 219028 | BC003604.1 | BC003604 | 228 |
| IOH9638 | 219029 | BC010464.1 | BC010464 | 186 |
| IOH10382 | 219032 | BC017085.1 | BC017085 | 597 |
| IOH26854 | 220773 | BC030578.1 | BC030578 | 111 |
| IOH10365 | 219020 | NM_152269.1 | NM_152269 | 113 |
| IOH21921 | 220806 | NM_000566.1 | NM_000566 | 46 |
| IOH5155 | 218987 | BC004219.1 | BC004219 | 1342 |
| IOH10191 | 219007 | BC009108.1 | BC009108 | 1667 |
| IOH4935 | 218990 | NM_006272.1 | NM_006272 | 5365 |
| IOH4375 | 218991 | NM_058199.1 | NM_058199 | 155 |
| IOH10070 | 218993 | BC016280.1 | BC016280 | 1082 |
| IOH10110 | 218994 | BC015904.1 | BC015904 | 116 |
| IOH10190 | 218995 | NM_152471.1 | NM_152471 | 5362 |
| IOH5559 | 219000 | NM_032676.1 | NM_032676 | 5366 |
| IOH5231 | 219023 | BC004233.1 | BC004233 | 5367 |
| IOH4958 | 219002 | NM_004781.2 | NM_004781 | 2834 |
| IOH5629 | 219012 | NM_032691.1 | NM_032691 | 4365 |
| IOH5397 | 219015 | NM_024319.1 | NM_024319 | 964 |
| IOH4971 | 219016 | NM_021974.2 | NM_021974 | 4777 |
| IOH10125 | 219018 | NM_020422.2 | NM_020422 | 281 |
| IOH10205 | 219019 | NM_138470.1 | NM_138470 | 165 |
| IOH5544 | 219001 | NM_031448.2 | NM_031448 | 5368 |
| IOH13364 | 216994 | BC012176.1 | BC012176 | 420 |
| IOH12495 | 216977 | NM_018959.1 | NM_018959 | 300 |
| IOH12981 | 216978 | NM_001084.2 | NM_001084 | 356 |
| IOH13450 | 216979 | NM_178858.3 | NM_178858 | 230 |
| IOH12049 | 216980 | BC009510.1 | BC009510 | 202 |
| IOH13360 | 216981 | NM_020375.1 | NM_020375 | 847 |
| IOH12590 | 216983 | NM_144492.1 | NM_144492 | 360 |
| IOH12410 | 216989 | NM_004838.2 | NM_004838 | 1039 |
| IOH13398 | 216995 | NM_005710.1 | NM_005710 | 1909 |
| IOH3084 | 219820 | NM_005000.2 | NM_005000 | 128 |
| IOH13361 | 217005 | BC014658.1 | BC014658 | 584 |
| IOH12774 | 217006 | BC014146.2 | BC014146 | 129 |
| IOH11070 | 216986 | BC025990.1 | BC025990 | 167 |
| IOH5547 | 219013 | NM_030572.1 | NM_030572 | 854 |
| IOH12531 | 218983 | BC011906.1 | BC011906 | 129 |
| IOH10550 | 219021 | BC012373.1 | BC012373 | 186 |
| IOH11753 | 217714 | BC028351.1 | BC028351 | 3230 |
| IOH12886 | 216852 | BC022272.1 | BC022272 | 161 |
| IOH13125 | 216851 | BC020749.1 | BC020749 | 158 |
| IOH1900 | 216848 | NM_000067.1 | NM_000067 | 875 |
| IOH13346 | 216859 | NM_005702.1 | NM_005702 | 47 |
| IOH13409 | 216846 | BC022043.1 | BC022043 | 641 |
| IOH13256 | 216850 | BC017347.1 | BC017347 | 254 |
| IOH12757 | 216867 | NM_032601.2 | NM_032601 | 545 |
| IOH13382 | 216880 | NM_173825.1 | NM_173825 | 77 |
| IOH12113 | 216877 | BC020630.1 | BC020630 | 201 |
| IOH12966 | 216876 | NM_152396.1 | NM_152396 | 67 |
| IOH12079 | 216875 | BC022258.1 | BC022258 | 1065 |
| IOH12061 | 216856 | BC022257.1 | BC022257 | 3926 |
| IOH12653 | 216871 | BC017249.1 | BC017249 | 152 |
| IOH12055 | 216853 | BC020843.1 | BC020843 | 160 |
| IOH12078 | 216864 | NM_005797.2 | NM_005797 | 308 |
| IOH12327 | 216863 | NM_138957.1 | NM_138957 | 448 |
| IOH1903 | 216860 | NM_004929.2 | NM_004929 | 1663 |
| IOH13380 | 216838 | NM_138818.1 | NM_138818 | 73 |
| IOH13388 | 216857 | BC020835.1 | BC020835 | 331 |
| IOH1913 | 216872 | NM_005138.1 | NM_005138 | 196 |
| IOH13476 | 216827 | BC026236.1 | BC026236 | 31 |
| IOH22638 | 221174 | NM_003006.2 | NM_003006 | 183 |
| IOH3506 | 221175 | BC000450.1 | BC000450 | 54 |
| IOH23036 | 221176 | BC022429.1 | BC022429 | 491 |
| IOH14340 | 221178 | NM_021158.1 | NM_021158 | 109 |
| IOH13630 | 221179 | NM_021104.1 | NM_021104 | 142 |
| IOH5674 | 221180 | NM_015510.2 | NM_015510 | 328 |
| IOH5508 | 221181 | BC004242.1 | BC004242 | 4577 |
| IOH5450 | 221182 | NM_020531.2 | NM_020531 | 39 |
| IOH9642 | 221183 | BC013609.1 | BC013609 | 35 |
| IOH3753 | 221186 | BC001064.1 | BC001064 | 4924 |
| IOH1875 | 216824 | NM_015971.2 | NM_015971 | 50 |
| IOH12140 | 216840 | BC017780.1 | BC017780 | 210 |
| IOH12138 | 216842 | NM_130782.1 | NM_130782 | 55 |
| IOH12143 | 216828 | BC017781.1 | BC017781 | 63 |
| IOH13022 | 216830 | BC020898.1 | BC020898 | 83 |
| IOH12831 | 216832 | BC020658.1 | BC020658 | 112 |
| IOH13254 | 216835 | NM_0173474.2 | NM_0173474 | 46 |
| IOH1877 | 216836 | NM_005086.3 | NM_005086 | 188 |
| IOH14765 | 217704 | BC015634.1 | BC015634 | 4651 |
| IOH10856 | 217700 | NM_145021.1 | NM_145021 | 64 |
| IOH2052 | 216837 | NM_006755.1 | NM_006755 | 25 |
| IOH1960 | 216896 | NM_018438.2 | NM_018438 | 23 |
| IOH12921 | 216839 | NM_000536.1 | NM_000536 | 19 |
| IOH12434 | 216887 | BC017873.1 | BC017873 | 270 |
| IOH12104 | 216841 | NM_080816.1 | NM_080816 | 54 |
| IOH2022 | 216825 | NM_002198.1 | NM_002198 | 54 |
| IOH12569 | 216945 | BC012124.1 | BC012124 | 163 |
| IOH13432 | 216894 | BC019080.2 | BC019080 | 29 |
| IOH12840 | 216930 | NM_022720.2 | NM_022720 | 1121 |
| IOH13462 | 216932 | NM_138453.1 | NM_138453 | 2379 |
| IOH13484 | 216934 | NM_138408.1 | NM_138408 | 463 |
| IOH12045 | 216935 | NM_005220.1 | NM_005220 | 20 |
| IOH12802 | 216936 | BC014218.2 | BC014218 | 2605 |
| IOH10695 | 216938 | NM_000442.2 | NM_000442 | 107 |
| IOH10975 | 216940 | NM_138722.1 | NM_138722 | 1349 |
| IOH12682 | 216941 | BC011924.1 | BC011924 | 83 |
| IOH12796 | 216942 | NM_030815.1 | NM_030815 | 986 |
| IOH12116 | 221169 | BC018928.1 | BC018928 | 360 |
| IOH2323 | 216897 | NM_000526.3 | NM_000526 | 23 |
| IOH13489 | 216898 | BC022377.1 | BC022377 | 1059 |
| IOH12322 | 216899 | BC017864.1 | BC017864 | 153 |
| IOH13453 | 216929 | BC011923.1 | BC011923 | 154 |
| IOH5756 | 216902 | BC008069.2 | BC008069 | 155 |
| IOH12194 | 216888 | BC017786.1 | BC017786 | 77 |
| IOH12152 | 216910 | BC020688.1 | BC020688 | 102 |
| IOH12442 | 216911 | NM_138701.1 | NM_138701 | 149 |
| IOH13027 | 216912 | BC022407.1 | BC022407 | 756 |
| IOH13026 | 216913 | NM_014485.1 | NM_014485 | 1522 |
| IOH12740 | 216914 | BC020596.1 | BC020596 | 387 |
| IOH12057 | 216915 | BC020620.1 | BC020620 | 821 |
| IOH12704 | 216920 | NM_052978.1 | NM_052978 | 195 |
| IOH13276 | 216922 | NM_022780.2 | NM_022780 | 114 |
| IOH13355 | 216923 | BC014409.1 | BC014409 | 1518 |
| IOH12778 | 216924 | BC014148.2 | BC014148 | 69 |
| IOH13019 | 216901 | BC022405.1 | BC022405 | 169 |
| IOH4364 | 221066 | BC000116.1 | BC000116 | 819 |
| IOH9626 | 221172 | BC011353.1 | BC011353 | 31 |
| IOH5552 | 221051 | NM_032303.1 | NM_032303 | 80 |
| IOH5433 | 221052 | BC002834.1 | BC002834 | 758 |
| IOH3146 | 221053 | BC006769.1 | BC006769 | 431 |
| IOH4355 | 221054 | BC004349.1 | BC004349 | 322 |
| IOH3554 | 221055 | NM_003908.1 | NM_003908 | 518 |
| IOH3644 | 221056 | NM_002861.1 | NM_002861 | 1387 |
| IOH6092 | 221060 | NM_001324.1 | NM_001324 | 1044 |
| IOH4946 | 221061 | NM_058179.1 | NM_058179 | 1424 |
| IOH5673 | 221062 | BC004889.1 | BC004889 | 822 |
| IOH5205 | 221063 | NM_032314.1 | NM_032314 | 66 |
| IOH4905 | 221049 | BC001600.1 | BC001600 | 1544 |
| IOH3221 | 221065 | BC001250.1 | BC001250 | 405 |
| IOH5918 | 221048 | NM_015926.2 | NM_015926 | 399 |
| IOH3569 | 221067 | NM_004632.2 | NM_004632 | 407 |
| IOH3655 | 221068 | NM_004990.2 | NM_004990 | 524 |
| IOH6219 | 221072 | NM_007065.2 | NM_007065 | 1685 |
| IOH3216 | 221073 | NM_018091.2 | NM_018091 | 1097 |
| IOH5713 | 221074 | NM_024322.1 | NM_024322 | 1678 |
| IOH3438 | 221077 | NM_006623.1 | NM_006623 | 5376 |
| IOH4383 | 221078 | NM_004698.1 | NM_004698 | 693 |
| IOH3592 | 221079 | BC000463.1 | BC000463 | 1663 |
| IOH3468 | 221084 | BC000440.1 | BC000440 | 217 |
| IOH4508 | 221087 | BC000277.1 | BC000277 | 4181 |
| IOH4388 | 221089 | NM_000026.1 | NM_000026 | 3065 |
| IOH5448 | 221064 | BC004258.1 | BC004258 | 924 |
| IOH6052 | 221033 | BC004359.1 | BC004359 | 88 |
| IOH3720 | 221018 | BC001946.1 | BC001946 | 47 |
| IOH4312 | 221019 | NM_0177727.2 | NM_017727 | 124 |
| IOH3627 | 221020 | BC000525.1 | BC000525 | 758 |
| IOH6947 | 221023 | BC008337.1 | BC008337 | 116 |
| IOH5867 | 221024 | BC005889.2 | BC005889 | 1016 |
| IOH4822 | 221025 | NM_006194.1 | NM_006194 | 39 |
| IOH5666 | 221026 | BC005134.1 | BC005134 | 1325 |
| IOH5475 | 221027 | BC004248.1 | BC004248 | 70 |
| IOH5395 | 221028 | NM_006303.2 | NM_006303 | 747 |
| IOH4609 | 221029 | BC000788.1 | BC000788 | 2972 |
| IOH3758 | 221030 | BC003595.1 | BC003595 | 502 |
| IOH5671 | 221050 | NM_013319.1 | NM_013319 | 216 |
| IOH3630 | 221032 | BC002361.1 | BC002361 | 98 |
| IOH22295 | 221095 | NM_014364.1 | NM_014364 | 28 |
| IOH3490 | 221034 | NM_003756.1 | NM_003756 | 433 |
| IOH5905 | 221036 | NM_002298.2 | NM_002298 | 2240 |
| IOH4855 | 221037 | BC001889.1 | BC001889 | 1229 |
| IOH5668 | 221038 | BC004888.2 | BC004888 | 260 |
| IOH5513 | 221039 | NM_032704.1 | NM_032704 | 166 |
| IOH5136 | 221041 | NM_000358.1 | NM_000358 | 56 |
| IOH4045 | 221042 | BC001449.1 | BC001449 | 925 |
| IOH3508 | 221043 | NM_002805.1 | NM_002805 | 55 |
| IOH3633 | 221044 | NM_000284.1 | NM_000284 | 188 |
| IOH6276 | 221045 | BC006191.1 | BC006191 | 838 |
| IOH6997 | 221047 | BC008023.1 | BC008023 | 512 |
| IOH4328 | 221031 | BC000698.1 | BC000698 | 471 |
| IOH3022 | 221154 | BC000953.2 | BC000953 | 181 |
| IOH9675 | 221137 | BC011460.1 | BC011460 | 26 |
| IOH10459 | 221139 | BC013119.1 | BC013119 | 87 |
| IOH21691 | 221140 | BC030525.1 | BC030525 | 476 |
| IOH23012 | 221141 | NM_080423.1 | NM_080423 | 4040 |
| IOH22682 | 221142 | NM_005060.2 | NM_005060 | 145 |
| IOH22374 | 221143 | BC029660.1 | BC029660 | 284 |
| IOH21440 | 221144 | BC022237.1 | BC022237 | 2398 |
| IOH12694 | 221146 | NM_032775.1 | NM_032775 | 35 |
| IOH3606 | 221147 | BC002360.1 | BC002360 | 131 |
| IOH4968 | 221148 | NM_018070.2 | NM_018070 | 3168 |
| IOH10105 | 221149 | BC015814.1 | BC015814 | 634 |
| IOH22892 | 221093 | BC012824.1 | BC012824 | 33 |
| IOH23015 | 221153 | BC021701.1 | BC021701 | 537 |
| IOH14075 | 221132 | NM_013446.2 | NM_013446 | 48 |
| IOH22379 | 221155 | BC028983.1 | BC028983 | 110 |
| IOH21478 | 221156 | BC013796.1 | BC013796 | 22 |
| IOH12752 | 221157 | NM_015938.2 | NM_015938 | 54 |
| IOH9977 | 221160 | BC015805.1 | BC015805 | 5364 |
| IOH22604 | 221162 | NM_021969.1 | NM_021969 | 51 |
| IOH23025 | 221163 | NM_139062.1 | NM_139062 | 456 |
| IOH21412 | 221164 | NM_014702.1 | NM_014702 | 87 |
| IOH10956 | 221166 | NM_006147.1 | NM_006147 | 151 |
| IOH14558 | 221168 | BC022329.1 | BC022329 | 630 |
| IOH12628 | 216967 | NM_018696.1 | NM_018696 | 2000 |
| IOH4593 | 221170 | BC000001.1 | BC000001 | 385 |
| IOH5520 | 221150 | BC004925.1 | BC004925 | 76 |
| IOH21571 | 221114 | BC030290.1 | BC030290 | 51 |
| IOH12584 | 216958 | NM_020384.1 | NM_020384 | 704 |
| IOH13621 | 221096 | BC016276.1 | BC016276 | 86 |
| IOH12547 | 221097 | BC021101.1 | BC021101 | 48 |
| IOH12702 | 221098 | BC012079.1 | BC012079 | 145 |
| IOH4842 | 221099 | NM_130788.1 | NM_130788 | 63 |
| IOH3832 | 221100 | BC000769.1 | BC000769 | 662 |
| IOH9647 | 221101 | BC011454.1 | BC011454 | 74 |
| IOH2968 | 221103 | NM_000282.1 | NM_000282 | 30 |
| IOH22910 | 221105 | BC004122.1 | BC004122 | 3953 |
| IOH22301 | 221107 | BC030773.2 | BC030773 | 140 |
| IOH13631 | 221108 | BC013005.2 | BC013005 | 43 |
| IOH4671 | 221136 | NM_004401.1 | NM_004401 | 2629 |
| IOH9673 | 221113 | BC018426.1 | BC018426 | 288 |
| IOH12481 | 221134 | BC009249.1 | BC009249 | 382 |
| IOH22973 | 221117 | BC011713.2 | BC011713 | 797 |
| IOH22341 | 221119 | BC030592.2 | BC030592 | 227 |
| IOH14429 | 221120 | BC010047.1 | BC010047 | 204 |
| IOH12488 | 221121 | BC024272.1 | BC024272 | 85 |
| IOH13023 | 221122 | NM_015193.1 | NM_015193 | 1238 |
| IOH9674 | 221125 | BC011519.1 | BC011519 | 60 |
| IOH21874 | 221126 | NM_015696.2 | NM_015696 | 218 |
| IOH6993 | 221128 | BC008359.1 | BC008359 | 496 |
| IOH22994 | 221129 | BC014237.1 | BC014237 | 94 |
| IOH22345 | 221131 | NM_006948.1 | NM_006948 | 1640 |
| IOH22631 | 221094 | BC029054.1 | BC029054 | 121 |
| IOH4976 | 221111 | NM_002708.1 | NM_002708 | 31 |
| IOH14131 | 217555 | BC021561.1 | BC021561 | 1347 |
| IOH12494 | 216965 | NM_004105.2 | NM_004105 | 452 |
| IOH14207 | 217538 | NM_033317.1 | NM_033317 | 170 |
| IOH14124 | 217539 | NM_017952.2 | NM_017952 | 55 |
| IOH13986 | 217541 | BC017262.1 | BC017262 | 46 |
| IOH14004 | 217543 | BC021559.1 | BC021559 | 194 |
| IOH14178 | 217544 | NM_144608.1 | NM_144608 | 189 |
| IOH14458 | 217548 | BC017237.1 | BC017237 | 804 |
| IOH14168 | 217549 | BC010176.1 | BC010176 | 750 |
| IOH14717 | 217550 | NM_138443.1 | NM_138443 | 111 |
| IOH14361 | 217552 | NM_152373.2 | NM_152373 | 83 |
| IOH14488 | 217536 | BC010137.1 | BC010137 | 199 |
| IOH14682 | 217554 | BC021551.1 | BC021551 | 449 |
| IOH14151 | 217531 | NM_033161.2 | NM_033161 | 70 |
| IOH13887 | 217556 | BC028840.1 | BC028840 | 193 |
| IOH14194 | 217557 | BC025345.1 | BC025345 | 2423 |
| IOH14694 | 217558 | NM_002539.1 | NM_002539 | 278 |
| IOH13839 | 217559 | NM_145063.1 | NM_145063 | 1483 |
| IOH13752 | 217560 | NM_007111.2 | NM_007111 | 210 |
| IOH13703 | 217565 | BC021930.1 | BC021930 | 446 |
| IOH14146 | 217566 | NM_006567.1 | NM_006567 | 227 |
| IOH14071 | 217567 | BC025281.1 | BC025281 | 224 |
| IOH14021 | 217569 | NM_016641.2 | NM_016641 | 412 |
| IOH14539 | 217570 | BC011779.2 | BC011779 | 225 |
| IOH13727 | 217571 | BC010081.2 | BC010081 | 1079 |
| IOH14674 | 217553 | NM_016093.2 | NM_016093 | 52 |
| IOH14513 | 217514 | BC011888.1 | BC011888 | 204 |
| IOH14554 | 217500 | NM_017660.2 | NM_017660 | 33 |
| IOH14463 | 217501 | BC011739.2 | BC011739 | 29 |
| IOH14811 | 217502 | NM_058163.1 | NM_058163 | 5375 |
| IOH14566 | 217503 | NM_003315.1 | NM_003315 | 187 |
| IOH14819 | 217504 | BC018667.1 | BC018667 | 205 |
| IOH14669 | 217505 | NM_138355.1 | NM_138355 | 5373 |
| IOH14855 | 217506 | NM_138387.2 | NM_138387 | 79 |
| IOH14059 | 217507 | NM_016207.2 | NM_016207 | 281 |
| IOH14693 | 217508 | BC026032.1 | BC026032 | 192 |
| IOH13934 | 217509 | BC024269.1 | BC024269 | 94 |
| IOH14625 | 217537 | NM_002622.3 | NM_002622 | 265 |
| IOH14650 | 217513 | BC011812.1 | BC011812 | 55 |
| IOH4058 | 218328 | BC002526.1 | BC002526 | 538 |
| IOH14526 | 217515 | NM_005435.2 | NM_005435 | 1772 |
| IOH14106 | 217518 | BC018736.1 | BC018736 | 36 |
| IOH14632 | 217519 | NM_004722.2 | NM_004722 | 207 |
| IOH14623 | 217521 | NM_032855.1 | NM_032855 | 467 |
| IOH14622 | 217524 | BC010064.2 | BC010064 | 33 |
| IOH13517 | 217525 | NM_052844.1 | NM_052844 | 580 |
| IOH14206 | 217526 | BC011885.1 | BC011885 | 262 |
| IOH13544 | 217527 | NM_052845.1 | NM_052845 | 2522 |
| IOH13653 | 217528 | BC016381.1 | BC016381 | 35 |
| IOH14642 | 217529 | BC021263.1 | BC021263 | 4027 |
| IOH14571 | 217512 | NM_145169.1 | NM_145169 | 383 |
| IOH5665 | 216458 | NM_033003.1 | NM_033003 | 5372 |
| IOH3593 | 218467 | BC002373.1 | BC002373 | 5279 |
| IOH23043 | 218476 | NM_014055.1 | NM_014055 | 2169 |
| IOH9811 | 218487 | BC009696.1 | BC009696 | 1911 |
| IOH9857 | 218499 | NM_138730.1 | NM_138730 | 1623 |
| IOH5745 | 218504 | BC006199.1 | BC006199 | 1685 |
| IOH3515 | 218513 | BC000503.1 | BC000503 | 1121 |
| IOH4929 | 216447 | NM_003405.2 | NM_003405 | 5359 |
| IOH6324 | 216448 | NM_031464.1 | NM_031464 | 4986 |
| IOH6735 | 216449 | NM_006374.2 | NM_006374 | 5376 |
| IOH10972 | 216451 | NM_007202.2 | NM_007202 | 240 |
| IOH14689 | 217572 | BC011811.1 | BC011811 | 100 |
| IOH14401 | 216454 | BC017236.1 | BC017236 | 3117 |
| IOH23069 | 218442 | NM_018439.1 | NM_018439 | 4668 |
| IOH5842 | 216459 | NM_016283.2 | NM_016283 | 4658 |
| IOH6368 | 216460 | NM_003821.2 | NM_003821 | 87 |
| IOH5022 | 216461 | NM_020990.2 | NM_020990 | 3129 |
| IOH10843 | 216463 | BC014794.1 | BC014794 | 102 |
| IOH13323 | 216464 | BC020225.1 | BC020225 | 88 |
| IOH5678 | 216470 | BC004518.1 | BC004518 | 410 |
| IOH6779 | 216472 | BC007872.1 | BC007872 | 5373 |
| IOH7258 | 216473 | NM_001239.2 | NM_001239 | 5371 |
| IOH9871 | 216474 | NM_002658.1 | NM_002658 | 5364 |
| IOH11046 | 216475 | NM_016282.2 | NM_016282 | 3789 |
| IOH13291 | 216476 | BC020221.1 | BC020221 | 3465 |
| IOH13877 | 216453 | NM_001744.2 | NM_001744 | 5377 |
| IOH4360 | 218352 | NM_016497.2 | NM_016497 | 4334 |
| IOH14020 | 217497 | NM_006521.3 | NM_006521 | 231 |
| IOH4285 | 218330 | BC002484.1 | BC002484 | 799 |
| IOH4338 | 218331 | NM_058217.1 | NM_058217 | 473 |
| IOH3166 | 218332 | BC006838.1 | BC006838 | 179 |
| IOH3230 | 218333 | BC000884.1 | BC000884 | 1927 |
| IOH3518 | 218334 | BC000452.1 | BC000452 | 4320 |
| IOH4354 | 218340 | NM_024043.1 | NM_024043 | 605 |
| IOH4341 | 218343 | BC000691.1 | BC000691 | 3126 |
| IOH3171 | 218344 | BC006839.1 | BC006839 | 150 |
| IOH3523 | 218346 | NM_024348.2 | NM_024348 | 277 |
| IOH4232 | 218347 | NM_003609.2 | NM_003609 | 4252 |
| IOH9793 | 218463 | BC016582.1 | BC016582 | 276 |
| IOH4083 | 218350 | BC001426.1 | BC001426 | 4641 |
| IOH6290 | 218447 | NM_032933.1 | NM_032933 | 142 |
| IOH4381 | 218353 | NM_004832.1 | NM_004832 | 5375 |
| IOH4301 | 218354 | NM_017706.2 | NM_017706 | 142 |
| IOH4343 | 218355 | NM_006651.2 | NM_006651 | 4098 |
| IOH3421 | 218357 | NM_004493.1 | NM_004493 | 1310 |
| IOH4362 | 218364 | BC000226.1 | BC000226 | 3669 |
| IOH3196 | 218380 | NM_003254.1 | NM_003254 | 226 |
| IOH3469 | 218381 | NM_006110.1 | NM_006110 | 1785 |
| IOH7008 | 218436 | BC008031.1 | BC008031 | 4731 |
| IOH7570 | 218437 | BC008461.1 | BC008461 | 268 |
| IOH9772 | 218439 | BC013158.1 | BC013158 | 146 |
| IOH13543 | 217573 | BC014001.1 | BC014001 | 258 |
| IOH3352 | 218348 | NM_080658.1 | NM_080658 | 752 |
| IOH7547 | 217298 | BC007110.1 | BC007110 | 144 |
| IOH11281 | 216999 | BC025700.1 | BC025700 | 1474 |
| IOH12571 | 217000 | NM_016310.2 | NM_016310 | 440 |
| IOH12379 | 217001 | BC026126.1 | BC026126 | 1339 |
| IOH12355 | 217002 | NM_016484.1 | NM_016484 | 2663 |
| IOH12380 | 217004 | BC012109.1 | BC012109 | 3887 |
| IOH10848 | 217008 | NM_024685.1 | NM_024685 | 126 |
| IOH10731 | 217009 | BC021172.2 | BC021172 | 1705 |
| IOH10645 | 217010 | NM_000023.1 | NM_000023 | 129 |
| IOH12850 | 217011 | BC011916.1 | BC011916 | 367 |
| IOH9833 | 217294 | NM_145244.1 | NM_145244 | 392 |
| IOH14129 | 217316 | BC018625.1 | BC018625 | 137 |
| IOH9972 | 217297 | BC013571.1 | BC013571 | 1419 |
| IOH13199 | 216992 | NM_145041.1 | NM_145041 | 5351 |
| IOH5749 | 217300 | NM_001168.1 | NM_001168 | 3023 |
| IOH5792 | 217301 | NM_004051.1 | NM_004051 | 528 |
| IOH6546 | 217303 | NM_014571.2 | NM_014571 | 50 |
| IOH9908 | 217307 | BC0132437.1 | BC0132437 | 446 |
| IOH9978 | 217309 | NM_006333.1 | NM_006333 | 2728 |
| IOH7548 | 217310 | BC005911.1 | BC005911 | 5314 |
| IOH7567 | 217311 | NM_080650.1 | NM_080650 | 5269 |
| IOH5751 | 217312 | NM_001673.2 | NM_001673 | 489 |
| IOH5797 | 217313 | NM_004309.2 | NM_004309 | 2551 |
| IOH5956 | 217314 | BC007658.1 | BC007658 | 965 |
| IOH9906 | 217295 | NM_145306.1 | NM_145306 | 1175 |
| IOH10642 | 217688 | NM_138812.1 | NM_138812 | 469 |
| IOH10722 | 216961 | BC018063.1 | BC018063 | 324 |
| IOH10800 | 216963 | NM_152314.1 | NM_152314 | 416 |
| IOH12777 | 216964 | BC011936.1 | BC011936 | 1584 |
| IOH12909 | 216966 | NM_016836.1 | NM_016836 | 42 |
| IOH4597 | 221014 | NM_003801.2 | NM_003801 | 40 |
| IOH12068 | 216968 | BC009506.1 | BC009506 | 270 |
| IOH13265 | 216969 | NM_053050.2 | NM_053050 | 1249 |
| IOH13248 | 216971 | BC011576.1 | BC011576 | 296 |
| IOH11158 | 216972 | BC026325.1 | BC026325 | 394 |
| IOH10837 | 216973 | NM_145047.1 | NM_145047 | 103 |
| IOH10911 | 216974 | NM_024695.1 | NM_024695 | 1350 |
| IOH10910 | 216998 | BC014607.2 | BC014607 | 1784 |
| IOH13320 | 216976 | NM_024610.2 | NM_024610 | 502 |
| IOH11253 | 216997 | NM_015417.2 | NM_015417 | 1268 |
| IOH13855 | 217679 | NM_138392.1 | NM_138392 | 1958 |
| IOH10664 | 217677 | NM_144647.1 | NM_144647 | 5374 |
| IOH10958 | 217676 | NM_016230.2 | NM_016230 | 2054 |
| IOH10809 | 216984 | NM_145314.1 | NM_145314 | 65 |
| IOH11034 | 216985 | BC022462.1 | BC022462 | 124 |
| IOH10931 | 216987 | BC025729.1 | BC025729 | 129 |
| IOH13153 | 216988 | NM_032122.2 | NM_032122 | 285 |
| IOH12635 | 216990 | BC024208.1 | BC024208 | 1123 |
| IOH13079 | 216991 | NM_021809.2 | NM_021809 | 959 |
| IOH13483 | 216993 | NM_138415.1 | NM_138415 | 164 |
| IOH9858 | 217318 | NM_019103.1 | NM_019103 | 117 |
| IOH11059 | 216975 | NM_021245.2 | NM_021245 | 120 |
| IOH14073 | 217485 | BC024281.1 | BC024281 | 3646 |
| IOH14750 | 217365 | NM_002028.2 | NM_002028 | 619 |
| IOH9894 | 217366 | BC009674.1 | BC009674 | 618 |
| IOH9968 | 217368 | BC013569.1 | BC013569 | 5369 |
| IOH7532 | 217369 | BC007104.1 | BC007104 | 5373 |
| IOH7438 | 217371 | BC008407.1 | BC008407 | 2600 |
| IOH5772 | 217372 | BC005823.1 | BC005823 | 793 |
| IOH5829 | 217373 | NM_017966.1 | NM_017966 | 228 |
| IOH6528 | 217374 | BC005055.1 | BC005055 | 4336 |
| IOH9947 | 217378 | NM_138787.1 | NM_138787 | 4035 |
| IOH14704 | 217387 | NM_002648.1 | NM_002648 | 1621 |
| IOH6566 | 217315 | NM_024493.1 | NM_024493 | 3012 |
| IOH14846 | 217484 | BC021120.1 | BC021120 | 321 |
| IOH5828 | 217361 | NM_007255.1 | NM_007255 | 128 |
| IOH13935 | 217486 | NM_022369.2 | NM_022369 | 46 |
| IOH14671 | 217487 | NM_003104.2 | NM_003104 | 2597 |
| IOH13726 | 217488 | BC011710.2 | BC011710 | 34 |
| IOH13845 | 217489 | NM_032476.1 | NM_032476 | 1771 |
| IOH14544 | 217490 | BC014057.1 | BC014057 | 205 |
| IOH13943 | 217491 | NM_001679.1 | NM_001679 | 198 |
| IOH14624 | 217493 | BC021253.2 | BC021253 | 1793 |
| IOH14788 | 217494 | BC018749.1 | BC018749 | 269 |
| IOH14790 | 217495 | BC022098.1 | BC022098 | 380 |
| IOH14762 | 217496 | NM_005347.2 | NM_005347 | 215 |
| IOH12587 | 216959 | NM_022154.2 | NM_022154 | 61 |
| IOH13954 | 217483 | NM_025108.1 | NM_025108 | 237 |
| IOH9864 | 217342 | NM_145252.1 | NM_145252 | 197 |
| IOH9933 | 217319 | NM_138793.1 | NM_138793 | 250 |
| IOH9993 | 217321 | NM_015987.2 | NM_015987 | 3019 |
| IOH7549 | 217322 | BC005930.1 | BC005930 | 205 |
| IOH7571 | 217323 | NM_006366.1 | NM_006366 | 1046 |
| IOH5753 | 217324 | NM_001561.3 | NM_001561 | 48 |
| IOH5964 | 217326 | NM_006460.1 | NM_006460 | 1635 |
| IOH9861 | 217330 | BC009738.1 | BC009738 | 4084 |
| IOH9936 | 217331 | BC015169.1 | BC015169 | 1242 |
| IOH7553 | 217334 | BC005902.1 | BC005902 | 698 |
| IOH5054 | 217335 | NM_004649.1 | NM_004649 | 5370 |
| IOH5754 | 217336 | NM_001983.1 | NM_001983 | 858 |
| IOH14081 | 217364 | BC021105.1 | BC021105 | 4015 |
| IOH14058 | 217341 | BC018732.1 | BC018732 | 951 |
| IOH14069 | 217363 | BC019102.1 | BC019102 | 445 |
| IOH9940 | 217343 | NM_004853.1 | NM_004853 | 5375 |
| IOH7554 | 217346 | NM_014267.2 | NM_014267 | 2519 |
| IOH5824 | 217349 | BC007414.2 | BC007414 | 67 |
| IOH6582 | 217351 | NM_032712.1 | NM_032712 | 39 |
| IOH14878 | 217353 | NM_003794.1 | NM_003794 | 175 |
| IOH9941 | 217355 | NM_022152.2 | NM_022152 | 62 |
| IOH9965 | 217356 | NM_000317.1 | NM_000317 | 5374 |
| IOH7556 | 217358 | BC008435.1 | BC008435 | 2295 |
| IOH7416 | 217359 | BC008440.1 | BC008440 | 1649 |
| IOH5762 | 217360 | NM_032359.1 | NM_032359 | 1601 |
| IOH13894 | 217498 | NM_021822.1 | NM_021822 | 99 |
| IOH13547 | 217340 | BC018766.1 | BC018766 | 368 |
| IOH21605 | 220775 | BC031265.1 | BC031265 | 398 |
| IOH4717 | 219063 | NM_014358.1 | NM_014358 | 188 |
| IOH10010 | 219064 | BC017117.1 | BC017117 | 297 |
| IOH9694 | 219065 | NM_001986.1 | NM_001986 | 3627 |
| IOH10184 | 219066 | BC010518.1 | BC010518 | 203 |
| IOH10251 | 219067 | BC013069.1 | BC013069 | 537 |
| IOH27248 | 220866 | NM_003358.1 | NM_003358 | 273 |
| IOH27133 | 220772 | BC035028.1 | BC035028 | 100 |
| IOH28287 | 220867 | AB065662.1 | AB065662 | 25 |
| IOH5012 | 217929 | NM_024668.1 | NM_024668 | 212 |
| IOH7202 | 217927 | BC005259.1 | BC005259 | 4739 |
| IOH5335 | 221016 | BC002751.1 | BC002751 | 424 |
| IOH23248 | 220774 | BC033196.1 | BC033196 | 1474 |
| IOH5409 | 219059 | NM_024314.1 | NM_024314 | 273 |
| IOH28296 | 220870 | AB065621.1 | AB065621 | 29 |
| IOH25778 | 220776 | NM_003878.1 | NM_003878 | 37 |
| IOH22820 | 220777 | NM_022141.1 | NM_022141 | 738 |
| IOH27453 | 220778 | NM_080745.1 | NM_080745 | 1262 |
| IOH3090 | 220872 | BC001284.1 | BC001284 | 41 |
| IOH22254 | 220779 | NM_139169.2 | NM_139169 | 1297 |
| IOH21330 | 220873 | NM_002739.1 | NM_002739 | 80 |
| IOH27325 | 220874 | NM_000486.2 | NM_000486 | 811 |
| IOH27700 | 220780 | BC037333.1 | BC037333 | 479 |
| IOH27414 | 220875 | NM_016511.1 | NM_016511 | 213 |
| IOH28297 | 220868 | AB065619.1 | AB065619 | 44 |
| IOH10418 | 219044 | BC020960.1 | BC020960 | 377 |
| IOH10216 | 219031 | BC016464.1 | BC016464 | 192 |
| IOH10556 | 219033 | NM_006681.1 | NM_006681 | 418 |
| IOH4589 | 219034 | NM_000262.1 | NM_000262 | 177 |
| IOH5233 | 219035 | NM_024114.1 | NM_024114 | 305 |
| IOH5499 | 219036 | BC004277.1 | BC004277 | 5369 |
| IOH4704 | 219037 | BC000772.1 | BC000772 | 2544 |
| IOH5492 | 219038 | NM_004887.2 | NM_004887 | 309 |
| IOH3851 | 219039 | BC001129.1 | BC001129 | 72 |
| IOH4814 | 219040 | BC005004.1 | BC005004 | 655 |
| IOH9639 | 219041 | BC008624.1 | BC008624 | 5361 |
| IOH4772 | 219061 | NM_004965.3 | NM_004965 | 5249 |
| IOH10240 | 219043 | NM_033414.1 | NM_033414 | 452 |
| IOH5507 | 219060 | NM_032301.1 | NM_032301 | 221 |
| IOH5121 | 219046 | NM_080702.1 | NM_080702 | 722 |
| IOH5351 | 219047 | BC002752.1 | BC002752 | 5358 |
| IOH9768 | 219049 | NM_080664.1 | NM_080664 | 2459 |
| IOH3853 | 219051 | BC001132.1 | BC001132 | 322 |
| IOH9964 | 219052 | NM_004545.1 | NM_004545 | 302 |
| IOH9691 | 219053 | BC011400.1 | BC011400 | 2948 |
| IOH10248 | 219055 | BC010562.1 | BC010562 | 280 |
| IOH10465 | 219056 | NM_138771.1 | NM_138771 | 2608 |
| IOH10335 | 219057 | NM_1144626.1 | NM_1144626 | 463 |
| IOH5124 | 219058 | BC003178.1 | BC003178 | 95 |
| IOH22624 | 220876 | NM_033423.1 | NM_033423 | 83 |
| IOH10180 | 219042 | BC010498.1 | BC010498 | 1370 |
| IOH4015 | 220902 | NM_014248.2 | NM_014248 | 1711 |
| IOH27210 | 220781 | BC031056.1 | BC031056 | 606 |
| IOH7180 | 217926 | NM_012383.2 | NM_012383 | 3853 |
| IOH23176 | 220898 | NM_024164.2 | NM_024164 | 51 |
| IOH6746 | 217917 | NM_012200.2 | NM_012200 | 132 |
| IOH7199 | 217915 | NM_005792.1 | NM_005792 | 5369 |
| IOH27392 | 220899 | BC033509.1 | BC033509 | 307 |
| IOH27448 | 220805 | BC038422.1 | BC038422 | 25 |
| IOH7460 | 217912 | BC008392.1 | BC008392 | 686 |
| IOH6706 | 217904 | NM_019613.2 | NM_019613 | 49 |
| IOH22386 | 220900 | NM_015488.1 | NM_015488 | 42 |
| IOH27534 | 220801 | BC032390.1 | BC032390 | 57 |
| IOH26830 | 220808 | BC034954.2 | BC034954 | 92 |
| IOH27198 | 220809 | NM_004566.1 | NM_004566 | 22 |
| IOH26798 | 220810 | BC035938.1 | BC035938 | 34 |
| IOH28390 | 220905 | NM_033519.1 | NM_033519 | 34 |
| IOH25776 | 220814 | BC034726.1 | BC034726 | 725 |
| IOH21725 | 220908 | NM_170699.1 | NM_170699 | 92 |
| IOH25788 | 220909 | NM_182665.1 | NM_182665 | 445 |
| IOH28389 | 220883 | NM_000910.1 | NM_000910 | 48 |
| IOH7474 | 217947 | BC007102.1 | BC007102 | 2876 |
| IOH13194 | 220877 | NM_021170.2 | NM_021170 | 114 |
| IOH27690 | 220783 | NM_003692.1 | NM_003692 | 26 |
| IOH23122 | 220785 | NM_144684.1 | NM_144684 | 27 |
| IOH28328 | 220879 | NM_153445.1 | NM_153445 | 25 |
| IOH27154 | 220786 | NM_018189.1 | NM_018189 | 132 |
| IOH28529 | 220880 | XM_291436.1 | XM_291436 | 138 |
| IOH25820 | 220787 | NM_198081.1 | NM_198081 | 119 |
| IOH27185 | 220788 | BC039244.1 | BC039244 | 132 |
| IOH27505 | 220802 | BC045634.1 | BC045634 | 226 |
| IOH26861 | 220789 | NM_006100.1 | NM_006100 | 210 |
| IOH27669 | 220782 | BC031964.1 | BC031964 | 80 |
| IOH14368 | 220884 | NM_001436.2 | NM_001436 | 25 |
| IOH27270 | 220885 | BC039252.1 | BC039252 | 22 |
| IOH27729 | 220886 | NM_198181.1 | NM_198181 | 465 |
| IOH27746 | 220792 | NM_053006.1 | NM_053006 | 69 |
| IOH22581 | 220887 | NM_144770.1 | NM_144770 | 63 |
| IOH27237 | 220793 | BC036071.1 | BC036071 | 34 |
| IOH21856 | 220794 | NM_006869.1 | NM_006869 | 157 |
| IOH22385 | 220888 | BC024243.2 | BC024243 | 63 |
| IOH25740 | 220795 | NM_002734.1 | NM_002734 | 146 |
| IOH28221 | 220892 | AB065869.1 | AB065869 | 26 |
| IOH25832 | 220799 | NM_144595.1 | NM_144595 | 72 |
| IOH28158 | 220882 | AB065674.1 | AB065674 | 147 |
| IOH22420 | 218753 | BC022189.2 | BC022189 | 83 |
| IOH11454 | 218768 | BC027978.1 | BC027978 | 268 |
| IOH14802 | 218739 | BC015569.1 | BC015569 | 925 |
| IOH22400 | 218740 | BC028425.1 | BC028425 | 100 |
| IOH22436 | 218742 | BC021188.2 | BC021188 | 729 |
| IOH22462 | 218743 | NM_015605.4 | NM_015605 | 3875 |
| IOH11793 | 218744 | NM_002287.2 | NM_002287 | 218 |
| IOH14435 | 218745 | BC009207.2 | BC009207 | 2011 |
| IOH14162 | 218746 | NM_001353.3 | NM_001353 | 1532 |
| IOH21422 | 218747 | BC009631.1 | BC009631 | 154 |
| IOH21447 | 218748 | BC020985.1 | BC020985 | 5375 |
| IOH21486 | 218750 | NM_018370.1 | NM_018370 | 1142 |
| IOH21471 | 218737 | BC016486.1 | BC016486 | 1609 |
| IOH22403 | 218752 | NM_144588.2 | NM_144588 | 148 |
| IOH21444 | 218736 | BC020979.1 | BC020979 | 1583 |
| IOH22437 | 218754 | BC021189.2 | BC021189 | 5365 |
| IOH22464 | 218755 | BC036532.2 | BC036532 | 838 |
| IOH14523 | 218757 | BC013905.2 | BC013905 | 5373 |
| IOH13629 | 218758 | BC018771.1 | BC018771 | 60 |
| IOH21424 | 218759 | BC015219.1 | BC015219 | 2989 |
| IOH21448 | 218760 | NM_000585.1 | NM_000585 | 743 |
| IOH21474 | 218761 | BC013112.2 | BC013112 | 850 |
| IOH21488 | 218762 | NM_006571.2 | NM_006571 | 2624 |
| IOH14530 | 218763 | BC027729.1 | BC027729 | 1894 |
| IOH22422 | 218765 | BC022083.2 | BC022083 | 544 |
| IOH10174 | 219030 | NM_138480.1 | NM_138480 | 1058 |
| IOH14605 | 218751 | BC014264.2 | BC014264 | 5349 |
| IOH22434 | 218718 | NM_153224.2 | NM_153224 | 186 |
| IOH22407 | 218705 | NM_018710.1 | NM_018710 | 134 |
| IOH22428 | 218706 | BC032957.1 | BC032957 | 40 |
| IOH22455 | 218707 | NM_004170.2 | NM_004170 | 102 |
| IOH11762 | 218708 | BC02742.1 | BC025742 | 28 |
| IOH14150 | 218709 | NM_007108.1 | NM_007108 | 1607 |
| IOH14433 | 218710 | NM_016319.1 | NM_016319 | 460 |
| IOH21411 | 218711 | BC034245.1 | BC034245 | 674 |
| IOH21430 | 218712 | BC021622.1 | BC021622 | 468 |
| IOH21462 | 218713 | NM_152715.1 | NM_152715 | 901 |
| IOH21481 | 218714 | NM_173344.1 | NM_173344 | 46 |
| IOH13580 | 218715 | BC019239.1 | BC019239 | 2075 |
| IOH21483 | 218738 | NM_138461.1 | NM_138461 | 108 |
| IOH22412 | 218717 | BC022077.1 | BC022077 | 34 |
| IOH13570 | 218769 | NM_024674.1 | NM_024674 | 5376 |
| IOH22457 | 218719 | BC036540.2 | BC036540 | 736 |
| IOH14481 | 218721 | BC013959.1 | BC013959 | 1191 |
| IOH13947 | 218722 | BC017337.1 | BC017337 | 43 |
| IOH21413 | 218723 | NM_032459.1 | NM_032459 | 4389 |
| IOH21442 | 218724 | NM_021945.1 | NM_021945 | 242 |
| IOH21470 | 218725 | BC024939.1 | BC024939 | 41 |
| IOH21482 | 218726 | NM_020239.2 | NM_020239 | 242 |
| IOH14665 | 218727 | BC017572.1 | BC017572 | 893 |
| IOH22398 | 218728 | BC024245.2 | BC024245 | 953 |
| IOH22414 | 218729 | BC030711.2 | BC030711 | 1589 |
| IOH13956 | 218734 | NM_024760.1 | NM_024760 | 86 |
| IOH22397 | 218716 | NM_030755.1 | NM_030755 | 522 |
| IOH10056 | 219017 | NM_002952.2 | NM_002952 | 3677 |
| IOH22449 | 218766 | BC033035.1 | BC033035 | 5367 |
| IOH13334 | 218998 | NM_138446.1 | NM_138446 | 2202 |
| IOH3700 | 218314 | BC004144.1 | BC004144 | 67 |
| IOH5156 | 218300 | NM_024516.1 | NM_024516 | 5365 |
| IOH4417 | 218295 | BC000121.1 | BC000121 | 3422 |
| IOH10118 | 219006 | NM_138801.1 | NM_138801 | 355 |
| IOH4415 | 218283 | BC001741.1 | BC001741 | 5376 |
| IOH10343 | 219008 | NM_152690.1 | NM_152690 | 266 |
| IOH10545 | 219009 | BC013613.1 | BC013613 | 133 |
| IOH3168 | 218277 | NM_006275.2 | NM_006275 | 4190 |
| IOH4626 | 218275 | NM_006232.2 | NM_006232 | 1712 |
| IOH10283 | 218996 | BC014776.1 | BC014776 | 5370 |
| IOH4017 | 218269 | NM_016286.1 | NM_016286 | 5376 |
| IOH3721 | 218315 | BC000215.1 | BC000215 | 1976 |
| IOH3713 | 218267 | NM_146388.1 | NM_146388 | 59 |
| IOH4623 | 218263 | NM_000801.2 | NM_000801 | 5362 |
| IOH4438 | 218260 | NM_000437.2 | NM_000437 | 83 |
| IOH4407 | 218259 | BC000120.1 | BC000120 | 553 |
| IOH13142 | 219022 | BC012131.1 | BC012131 | 3242 |
| IOH5456 | 218258 | NM_173089.1 | NM_173089 | 2586 |
| IOH4012 | 218257 | BC001433.1 | BC001433 | 175 |
| IOH7183 | 217949 | BC005312.1 | BC005312 | 38 |
| IOH3846 | 219027 | NM_020676.2 | NM_020676 | 142 |
| IOH22871 | 220911 | NM_153208.1 | NM_153208 | 154 |
| IOH4410 | 218271 | BC000190.1 | BC000190 | 369 |
| IOH21410 | 218793 | BC034275.1 | BC034275 | 1098 |
| IOH21405 | 218770 | NM_024060.1 | NM_024060 | 5145 |
| IOH21426 | 218771 | NM_173541.1 | NM_173541 | 1271 |
| IOH21450 | 218772 | NM_021709.1 | NM_021709 | 4055 |
| IOH21475 | 218773 | BC023152.1 | BC023152 | 4414 |
| IOH21490 | 218774 | NM_152634.1 | NM_152634 | 649 |
| IOH14227 | 218775 | NM_005601.2 | NM_005601 | 897 |
| IOH14763 | 218781 | NM_025161.2 | NM_025161 | 222 |
| IOH21409 | 218782 | NM_173192.1 | NM_173192 | 3853 |
| IOH21427 | 218783 | NM_153702.1 | NM_153702 | 346 |
| IOH21454 | 218784 | BC018404.1 | BC018404 | 1646 |
| IOH21476 | 218785 | BC016640.1 | BC016640 | 152 |
| IOH10533 | 218997 | BC018206.1 | BC018206 | 5368 |
| IOH14815 | 218792 | BC011680.1 | BC011680 | 136 |
| IOH7206 | 217939 | BC005339.1 | BC005339 | 1842 |
| IOH21428 | 218794 | NM_174926.1 | NM_174926 | 240 |
| IOH21458 | 218795 | BC031469.1 | BC031469 | 1060 |
| IOH14039 | 218797 | BC023982.1 | BC023982 | 1661 |
| IOH13283 | 218986 | NM_032014.1 | NM_032014 | 156 |
| IOH3978 | 218327 | BC001394.1 | BC001394 | 4298 |
| IOH3706 | 218325 | NM_002402.1 | NM_002402 | 149 |
| IOH5159 | 218323 | BC004906.1 | BC004906 | 29 |
| IOH4908 | 218992 | NM_002014.2 | NM_002014 | 3035 |
| IOH5134 | 218322 | NM_001384.2 | NM_001384 | 22 |
| IOH4474 | 218319 | NM_030810.1 | NM_030810 | 2422 |
| IOH22406 | 218787 | NM_005038.1 | NM_005038 | 5375 |
| IOH4088 | 220099 | NM_032636.2 | NM_032636 | 284 |
| IOH6705 | 217893 | NM_005586.2 | NM_005586 | 128 |
| IOH14064 | 220075 | NM_004582.2 | NM_004582 | 323 |
| IOH7131 | 220077 | NM_018466.2 | NM_018466 | 136 |
| IOH5661 | 220079 | NM_004569.1 | NM_004569 | 2095 |
| IOH10491 | 220081 | NM_001769.2 | NM_001769 | 1583 |
| IOH9914 | 220082 | BC009712.1 | BC009712 | 393 |
| IOH12720 | 220085 | BC009956.1 | BC009956 | 64 |
| IOH3658 | 220087 | NM_004881.1 | NM_004881 | 1764 |
| IOH9786 | 220090 | NM_005380.1 | NM_005380 | 113 |
| IOH12125 | 220091 | NM_019101.2 | NM_019101 | 2402 |
| IOH10694 | 220094 | BC020517.1 | BC020517 | 98 |
| IOH11450 | 220072 | NM_019895.1 | NM_019895 | 2140 |
| IOH4981 | 220097 | NM_032641.1 | NM_032641 | 136 |
| IOH7016 | 220069 | BC008054.1 | BC008054 | 156 |
| IOH7207 | 220101 | BC005187.1 | BC005187 | 1204 |
| IOH3991 | 220103 | BC001430.1 | BC001430 | 92 |
| IOH11448 | 220106 | BC011968.1 | BC011968 | 464 |
| IOH10395 | 220107 | NM_024946.1 | NM_024946 | 100 |
| IOH4051 | 220108 | BC002568.1 | BC002568 | 30 |
| IOH10241 | 220109 | NM_004489.3 | NM_004489 | 156 |
| IOH4735 | 220110 | BC000108.1 | BC000108 | 1552 |
| IOH9888 | 220112 | NM_003650.2 | NM_003650 | 762 |
| IOH7193 | 217903 | BC005258.1 | BC005258 | 83 |
| IOH7482 | 217901 | NM_003338.2 | NM_003338 | 565 |
| IOH11751 | 220034 | NM_006002.2 | NM_006002 | 94 |
| IOH14515 | 220096 | BC020746.1 | BC020746 | 715 |
| IOH3794 | 220053 | BC001105.1 | BC001105 | 43 |
| IOH26872 | 220816 | NM_002242.2 | NM_002242 | 739 |
| IOH13408 | 220038 | BC019107.1 | BC019107 | 498 |
| IOH3287 | 220040 | NM_002074.2 | NM_002074 | 758 |
| IOH12964 | 220041 | NM_144646.1 | NM_144646 | 174 |
| IOH10522 | 220042 | NM_024775.8 | NM_024775 | 1152 |
| IOH13182 | 220046 | BC021295.2 | BC021295 | 859 |
| IOH12787 | 220047 | NM_148975.1 | NM_148975 | 356 |
| IOH14799 | 220048 | BC022344.1 | BC022344 | 1807 |
| IOH6364 | 220049 | NM_000802.2 | NM_000802 | 423 |
| IOH13381 | 220050 | BC017296.2 | BC017296 | 50 |
| IOH5857 | 220074 | BC007320.2 | BC007320 | 384 |
| IOH4957 | 220052 | NM_007370.2 | NM_007370 | 36 |
| IOH6703 | 217892 | BC007835.1 | BC007835 | 146 |
| IOH12167 | 220054 | BC012575.1 | BC012575 | 1015 |
| IOH3292 | 220058 | BC009010.1 | BC009010 | 1177 |
| IOH5013 | 220059 | BC004440.1 | BC004440 | 1339 |
| IOH5505 | 220060 | NM_013342.1 | NM_013342 | 1121 |
| IOH13661 | 220061 | NM_016052.1 | NM_016052 | 1918 |
| IOH14512 | 220062 | BC020744.1 | BC020744 | 42 |
| IOH5147 | 220063 | BC003132.1 | BC003132 | 367 |
| IOH13005 | 220064 | BC010943.1 | BC010943 | 1223 |
| IOH13730 | 220065 | BC020754.1 | BC020754 | 126 |
| IOH12789 | 220066 | BC020651.1 | BC020651 | 129 |
| IOH12082 | 220067 | BC009327.2 | BC009327 | 4550 |
| IOH10076 | 220051 | BC014897.1 | BC014897 | 974 |
| IOH5732 | 221003 | NM_012289.2 | NM_012289 | 2781 |
| IOH7457 | 217900 | BC008478.1 | BC008478 | 364 |
| IOH6647 | 219623 | NM_003311.2 | NM_003311 | 127 |
| IOH5963 | 219628 | BC006456.1 | BC006456 | 53 |
| IOH22146 | 219629 | BC035314.1 | BC035314 | 228 |
| IOH3041 | 219633 | NM_018983.2 | NM_018983 | 141 |
| IOH10608 | 219634 | NM_032146.2 | NM_032146 | 143 |
| IOH13548 | 219636 | NM_005040.1 | NM_005040 | 140 |
| IOH23082 | 219640 | BC021250.1 | BC021250 | 64 |
| IOH3394 | 219641 | BC009046.1 | BC009046 | 199 |
| IOH6811 | 220999 | BC007213.1 | BC007213 | 52 |
| IOH3060 | 221000 | NM_020165.2 | NM_020165 | 108 |
| IOH21729 | 219618 | NM_018527.1 | NM_018527 | 45 |
| IOH3053 | 221002 | BC001258.1 | BC001258 | 1592 |
| IOH22703 | 219613 | BC031592.1 | BC031592 | 126 |
| IOH5306 | 221004 | BC002702.1 | BC002702 | 64 |
| IOH4511 | 221005 | NM_016630.2 | NM_016630 | 1313 |
| IOH3456 | 221006 | BC000306.1 | BC000306 | 441 |
| IOH4394 | 221007 | BC000238.1 | BC000238 | 605 |
| IOH4172 | 221008 | NM_005371.2 | NM_005371 | 3863 |
| IOH4240 | 221009 | BC000645.1 | BC000645 | 51 |
| IOH3462 | 221010 | NM_002810.1 | NM_002810 | 947 |
| IOH6840 | 221011 | BC007557.1 | BC007557 | 139 |
| IOH3075 | 221012 | BC001247.1 | BC001247 | 1063 |
| IOH4744 | 221013 | NM_005659.1 | NM_005659 | 4931 |
| IOH22396 | 218704 | NM_145173.1 | NM_145173 | 1447 |
| IOH4743 | 221001 | NM_016091.1 | NM_016091 | 45 |
| IOH10937 | 217737 | NM_022755.2 | NM_022755 | 3517 |
| IOH5185 | 218999 | NM_031445.1 | NM_031445 | 586 |
| IOH7198 | 217881 | BC007003.1 | BC007003 | 151 |
| IOH7191 | 217879 | BC007009.1 | BC007009 | 5362 |
| IOH7444 | 217876 | BC005893.1 | BC005893 | 2531 |
| IOH7194 | 217869 | NM_001906.1 | NM_001906 | 460 |
| IOH5230 | 219011 | BC004234.1 | BC004234 | 286 |
| IOH7475 | 217865 | BC005914.1 | BC005914 | 681 |
| IOH12034 | 217760 | BC027617.1 | BC027617 | 5372 |
| IOH4984 | 219014 | BC003597.1 | BC003597 | 229 |
| IOH14651 | 217751 | NM_002966.1 | NM_002966 | 121 |
| IOH11737 | 217749 | BC027607.1 | BC027607 | 725 |
| IOH22166 | 219621 | NM_024786.1 | NM_024786 | 39 |
| IOH11653 | 217738 | NM_173501.1 | NM_173501 | 1510 |
| IOH11316 | 220033 | NM_012400.2 | NM_012400 | 1129 |
| IOH13616 | 217729 | NM_001911.1 | NM_001911 | 276 |
| IOH11315 | 217724 | NM_002364.1 | NM_002364 | 5371 |
| IOH7270 | 216485 | BC007023.1 | BC007023 | 838 |
| IOH14716 | 216477 | NM_018291.2 | NM_018291 | 164 |
| IOH10668 | 217713 | NM_145268.1 | NM_145268 | 2573 |
| IOH11096 | 217712 | NM_033105.1 | NM_033105 | 1495 |
| IOH6460 | 219598 | BC006393.1 | BC006393 | 30 |
| IOH7295 | 219599 | NM_002994.2 | NM_002994 | 508 |
| IOH22574 | 219607 | BC029520.1 | BC029520 | 122 |
| IOH21870 | 219608 | BC033819.1 | BC033819 | 49 |
| IOH12287 | 219609 | BC020868.1 | BC020868 | 131 |
| IOH27734 | 220945 | BC040606.1 | BC040606 | 64 |
| IOH10619 | 220954 | BC022231.1 | BC022231 | 188 |
| IOH5873 | 220935 | NM_004549.2 | NM_004549 | 716 |
| IOH27547 | 220841 | NM_152542.2 | NM_152542 | 220 |
| IOH27482 | 220842 | BC039306.1 | BC039306 | 1110 |
| IOH13267 | 220937 | NM_022818.2 | NM_022818 | 463 |
| IOH25853 | 220843 | NM_182607.2 | NM_182607 | 310 |
| IOH28263 | 220938 | AB065734.1 | AB065734 | 133 |
| IOH28238 | 220939 | AB065812.1 | AB065812 | 41 |
| IOH25850 | 220845 | BC043193.2 | BC043193 | 60 |
| IOH27111 | 220846 | BC032861.1 | BC032861 | 22 |
| IOH27401 | 220849 | NM_012113.1 | NM_012113 | 94 |
| IOH25805 | 220934 | BC039152.1 | BC039152 | 65 |
| IOH27486 | 220850 | BC036193.1 | BC036193 | 125 |
| IOH27319 | 220946 | BC047056.1 | BC047056 | 1055 |
| IOH27747 | 220852 | BC041366.2 | BC041366 | 2576 |
| IOH22178 | 220853 | BC031999.1 | BC031999 | 3395 |
| IOH5904 | 220947 | NM_017594.2 | NM_017594 | 1167 |
| IOH13412 | 220948 | NM_138786.1 | NM_138786 | 1218 |
| IOH27478 | 220854 | BC040527.1 | BC040527 | 454 |
| IOH28581 | 220949 | AB065663.1 | AB065663 | 55 |
| IOH27515 | 220855 | BC031231.1 | BC031231 | 2285 |
| IOH25823 | 220858 | BC037906.1 | BC037906 | 2771 |
| IOH12808 | 220036 | NM_015399.1 | NM_015399 | 196 |
| IOH26818 | 220832 | BC030640.1 | BC030640 | 136 |
| IOH5628 | 221015 | NM_012191.1 | NM_012191 | 2886 |
| IOH14740 | 220912 | NM_001216.1 | NM_001216 | 109 |
| IOH27358 | 220818 | NM_152723.1 | NM_152723 | 5378 |
| IOH5681 | 220913 | NM_000972.2 | NM_000972 | 27 |
| IOH25737 | 220819 | BC038354.1 | BC038354 | 28 |
| IOH28500 | 220914 | XM_060307.1 | XM_060307 | 32 |
| IOH25797 | 220821 | NM_153719.2 | NM_153719 | 3694 |
| IOH25831 | 220922 | BC041339.1 | BC041339 | 142 |
| IOH25844 | 220829 | BC043175.1 | BC043175 | 44 |
| IOH27467 | 220830 | NM_032047.2 | NM_032047 | 51 |
| IOH27450 | 220840 | BC037253.1 | BC037253 | 76 |
| IOH28501 | 220926 | XM_060315.1 | XM_060315 | 54 |
| IOH20993 | 220955 | NM_021962.1 | NM_021962 | 5380 |
| IOH28527 | 220927 | XM_062285.1 | XM_062285 | 1690 |
| IOH27543 | 220833 | NM_000167.1 | NM_000167 | 109 |
| IOH27329 | 220834 | NM_173619.1 | NM_173619 | 23 |
| IOH28257 | 220929 | AB065758.1 | AB065758 | 52 |
| IOH27423 | 220835 | NM_024430.1 | NM_024430 | 34 |
| IOH27502 | 220836 | NM_178863.2 | NM_178863 | 43 |
| IOH28163 | 220930 | AF137396.2 | AF137396 | 21 |
| IOH27369 | 220837 | NM_153356.1 | NM_153356 | 5374 |
| IOH27153 | 220838 | BC032852.2 | BC032852 | 4664 |
| IOH20956 | 220932 | NM_006225.1 | NM_006225 | 283 |
| IOH27245 | 220933 | BC041793.1 | BC041793 | 85 |
| IOH11558 | 220925 | NM_182554.1 | NM_182554 | 340 |
| IOH13335 | 219736 | NM_138788.1 | NM_138788 | 33 |
| IOH27212 | 220859 | BC036015.1 | BC036015 | 56 |
| IOH12508 | 219703 | BC014577.1 | BC041577 | 42 |
| IOH21553 | 219705 | NM_001585.1 | NM_001585 | 70 |
| IOH22183 | 219707 | NM_000710.2 | NM_000710 | 2320 |
| IOH12498 | 219708 | NM_144975.1 | NM_144975 | 295 |
| IOH9781 | 219710 | BC010691.1 | BC010691 | 37 |
| IOH10008 | 219717 | BC017168.1 | BC017168 | 105 |
| IOH14316 | 219719 | BC009775.1 | BC009775 | 72 |
| IOH12277 | 219721 | NM_016527.1 | NM_016527 | 2442 |
| IOH12342 | 219694 | NM_030774.2 | NM_030774 | 250 |
| IOH21781 | 219732 | NM_152287.2 | NM_152287 | 486 |
| IOH4800 | 219693 | BC001873.1 | BC001873 | 96 |
| IOH6499 | 219737 | NM_018941.1 | NM_018941 | 27 |
| IOH7172 | 220021 | BC005245.1 | BC005245 | 372 |
| IOH11058 | 220022 | NM_016422.2 | NM_016422 | 91 |
| IOH12058 | 220023 | BC022379.1 | BC022379 | 204 |
| IOH12842 | 220024 | NM_144578.1 | NM_144578 | 1944 |
| IOH13793 | 220025 | BC017865.1 | BC017865 | 72 |
| IOH12973 | 220026 | NM_152430.1 | NM_152430 | 1887 |
| IOH13243 | 220027 | BC021092.1 | BC021092 | 2156 |
| IOH3742 | 220029 | NM_016504.1 | NM_016504 | 389 |
| IOH9897 | 220030 | BC009621.1 | BC009621 | 662 |
| IOH6336 | 220031 | NM_032499.1 | NM_032499 | 883 |
| IOH3054 | 219661 | NM_003675.2 | NM_003675 | 33 |
| IOH27376 | 220956 | NM_052841.2 | NM_052841 | 5376 |
| IOH27355 | 220957 | NM_182623.1 | NM_182623 | 610 |
| IOH26853 | 220864 | BC032838.2 | BC032838 | 146 |
| IOH22623 | 220958 | NM_002521.1 | NM_002521 | 117 |
| IOH27539 | 220865 | NM_003370.1 | NM_003370 | 140 |
| IOH10746 | 219646 | NM_152443.1 | NM_152443 | 34 |
| IOH5210 | 219647 | BC003653.1 | BC003653 | 25 |
| IOH7384 | 219648 | NM_006479.2 | NM_006479 | 92 |
| IOH21782 | 219649 | BC033665.1 | BC033665 | 31 |
| IOH21713 | 219652 | NM_182980.1 | NM_182980 | 19 |
| IOH7253 | 219655 | NM_006136.1 | NM_006136 | 20 |
| IOH5297 | 219702 | BC002653.1 | BC002653 | 54 |
| IOH12290 | 219660 | BC022316.1 | BC022316 | 42 |
| IOH27433 | 220817 | NM_000913.1 | NM_000913 | 37 |
| IOH3631 | 219666 | BC000412.1 | BC000412 | 211 |
| IOH21515 | 219672 | BC033591.1 | BC033591 | 71 |
| IOH12543 | 219673 | NM_022788.2 | NM_022788 | 170 |
| IOH12753 | 219677 | NM_032784.2 | NM_032784 | 28 |
| IOH5426 | 219682 | NM_002914.1 | NM_002914 | 194 |
| IOH10934 | 219683 | BC025726.1 | BC025726 | 1150 |
| IOH22511 | 219685 | BC029483.1 | BC029483 | 44 |
| IOH4342 | 219687 | BC000683.1 | BC000683 | 42 |
| IOH11017 | 219690 | BC012924.1 | BC012924 | 70 |
| IOH5253 | 219692 | NM_006140.2 | NM_006140 | 111 |
| IOH22790 | 219658 | BC031653.1 | BC031653 | 80 |
| IOH4028 | 220342 | NM_018107.2 | NM_018107 | 85 |
| IOH14546 | 220324 | NM_004494.1 | NM_004494 | 589 |
| IOH5969 | 220325 | BC008364.1 | BC008364 | 2258 |
| IOH22693 | 220326 | BC034389.1 | BC034389 | 3632 |
| IOH12245 | 220332 | NM_145245.1 | NM_145245 | 297 |
| IOH10823 | 220333 | NM_004589.1 | NM_004589 | 82 |
| IOH6517 | 220335 | BC007742.1 | BC007742 | 446 |
| IOH21590 | 220337 | NM_152567.1 | NM_152567 | 40 |
| IOH22755 | 220338 | BC029220.1 | BC029220 | 530 |
| IOH12948 | 220339 | BC017810.1 | BC017810 | 835 |
| IOH22548 | 220317 | BC031068.1 | BC031068 | 123 |
| IOH22738 | 220343 | BC029158.1 | BC029158 | 30 |
| IOH6401 | 220344 | NM_139156.1 | NM_139156 | 53 |
| IOH9645 | 220345 | BC010451.1 | BC010451 | 219 |
| IOH11023 | 220346 | BC019247.1 | BC019247 | 23 |
| IOH2949 | 220347 | BC000158.2 | BC000158 | 30 |
| IOH12711 | 220348 | NM_015343.1 | NM_015343 | 51 |
| IOH21842 | 220349 | BC033864.1 | BC033864 | 214 |
| IOH21821 | 220374 | NM_014305.1 | NM_014305 | 204 |
| IOH12784 | 220375 | NM_032478.1 | NM_032478 | 200 |
| IOH5017 | 220376 | BC004424.1 | BC004424 | 51 |
| IOH10922 | 220377 | BC026184.2 | BC026184 | 20 |
| IOH11263 | 217181 | NM_013246.1 | NM_013246 | 63 |
| IOH3307 | 220340 | NM_000327.2 | NM_000327 | 76 |
| IOH22719 | 220302 | NM_005749.2 | NM_005749 | 39 |
| IOH26809 | 220684 | BC035936.1 | BC035936 | 202 |
| IOH12876 | 217183 | NM_016487.1 | NM_016487 | 133 |
| IOH12088 | 217184 | BC010907.1 | BC010907 | 54 |
| IOH12868 | 217185 | BC010929.1 | BC010929 | 37 |
| IOH12920 | 217186 | BC009423.1 | BC009423 | 61 |
| IOH12968 | 217187 | BC009485.1 | BC009485 | 759 |
| IOH12627 | 217189 | NM_138807.1 | NM_138807 | 25 |
| IOH13241 | 217192 | NM_153217.1 | NM_153217 | 27 |
| IOH12144 | 217193 | BC014538.1 | BC014538 | 46 |
| IOH13498 | 217194 | BC010901.1 | BC010901 | 654 |
| IOH12952 | 217195 | NM_052822.1 | NM_052822 | 76 |
| IOH13758 | 220322 | NM_002784.2 | NM_002784 | 22 |
| IOH10524 | 217199 | NM_138414.1 | NM_138414 | 4866 |
| IOH13683 | 220303 | BC009797.1 | BC009797 | 282 |
| IOH12389 | 220304 | NM_030664.2 | NM_030664 | 32 |
| IOH21872 | 220305 | NM_052938.2 | NM_052938 | 31 |
| IOH4700 | 220306 | BC000014.1 | BC000014 | 23 |
| IOH9728 | 220307 | BC011379.1 | BC011379 | 159 |
| IOH3819 | 220309 | NM_003720.1 | NM_003720 | 278 |
| IOH11952 | 220312 | BC022081.2 | BC022081 | 48 |
| IOH7540 | 220313 | NM_032929.1 | NM_032929 | 417 |
| IOH21715 | 220314 | NM_145109.1 | NM_145109 | 3106 |
| IOH13154 | 220315 | BC017880.1 | BC017880 | 21 |
| IOH13312 | 217198 | NM_022483.2 | NM_022483 | 33 |
| IOH4081 | 216778 | NM_017668.1 | NM_017668 | 1026 |
| IOH13657 | 220380 | NM_005666.1 | NM_005666 | 45 |
| IOH3301 | 216761 | NM_138390.1 | NM_138390 | 114 |
| IOH3366 | 216762 | BC008253.1 | BC008253 | 890 |
| IOH14139 | 216764 | NM_018948.2 | NM_018948 | 49 |
| IOH3944 | 216765 | NM_001757.1 | NM_001757 | 23 |
| IOH4079 | 216766 | NM_005620.1 | NM_005620 | 961 |
| IOH4136 | 216767 | NM_000375.1 | NM_000375 | 959 |
| IOH4171 | 216768 | NM_024047.2 | NM_024047 | 166 |
| IOH2504 | 216770 | NM_005032.2 | NM_005032 | 537 |
| IOH3015 | 216771 | BC000993.2 | BC000993 | 26 |
| IOH3304 | 216773 | BC008145.1 | BC008145 | 1777 |
| IOH4274 | 216758 | NM_024051.1 | NM_024051 | 840 |
| IOH3948 | 216777 | NM_001549.1 | NM_001549 | 478 |
| IOH4220 | 216757 | BC001023.1 | BC001023 | 20 |
| IOH4142 | 216779 | BC002622.1 | BC002622 | 36 |
| IOH4184 | 216780 | BC000586.1 | BC000586 | 113 |
| IOH4234 | 216781 | NM_138820.1 | NM_138820 | 502 |
| IOH2894 | 216782 | NM_024033.1 | NM_024033 | 743 |
| IOH3019 | 216783 | NM_006324.1 | NM_006324 | 897 |
| IOH3260 | 216784 | NM_024049.1 | NM_024049 | 987 |
| IOH3372 | 216786 | NM_080651.1 | NM_080651 | 74 |
| IOH3953 | 216789 | NM_015449.1 | NM_015449 | 21 |
| IOH4112 | 216790 | NM_004146.3 | NM_004146 | 158 |
| IOH4145 | 216791 | BC000535.1 | BC000535 | 43 |
| IOH4186 | 216792 | NM_000854.2 | NM_000854 | 265 |
| IOH4237 | 216793 | BC001017.1 | BC001017 | 528 |
| IOH14516 | 216775 | BC015684.2 | BC015684 | 88 |
| IOH11024 | 216739 | NM_174930.2 | NM_174930 | 294 |
| IOH2986 | 220384 | NM_006142.1 | NM_006142 | 1560 |
| IOH14261 | 220387 | BC012547.1 | BC012547 | 686 |
| IOH10984 | 220388 | NM_178525.2 | NM_178525 | 25 |
| IOH5587 | 220391 | NM_005268.1 | NM_005268 | 19 |
| IOH4093 | 220392 | NM_004155.2 | NM_004155 | 1979 |
| IOH13690 | 220395 | NM_014214.1 | NM_014214 | 783 |
| IOH10977 | 216727 | BC022454.2 | BC022454 | 23 |
| IOH3967 | 216730 | BC002493.1 | BC002493 | 491 |
| IOH4127 | 216731 | NM_014221.1 | NM_014221 | 1004 |
| IOH3237 | 216760 | BC000885.1 | BC000885 | 265 |
| IOH3330 | 216738 | BC008605.1 | BC008605 | 594 |
| IOH14670 | 216740 | BC021258.1 | BC021258 | 43 |
| IOH3933 | 216741 | NM_005697.3 | NM_005697 | 96 |
| IOH4069 | 216742 | NM_007008.1 | NM_007008 | 814 |
| IOH4130 | 216743 | NM_018124.2 | NM_018124 | 27 |
| IOH4219 | 216745 | NM_014077.1 | NM_014077 | 70 |
| IOH3086 | 216748 | NM_003244.1 | NM_003244 | 20 |
| IOH3354 | 216750 | NM_020445.1 | NM_020445 | 53 |
| IOH10757 | 216751 | BC022524.1 | BC022524 | 2026 |
| IOH14570 | 216752 | BC021303.1 | BC021303 | 171 |
| IOH4076 | 216754 | NM_003662.1 | NM_003662 | 1290 |
| IOH4170 | 216756 | NM_015492.2 | NM_015492 | 531 |
| IOH3291 | 216737 | NM_138474.1 | NM_138474 | 494 |
| IOH14182 | 220740 | BC010349.1 | BC010349 | 80 |
| IOH14782 | 220745 | BC017353.1 | BC017353 | 80 |
| IOH14254 | 220727 | BC015818.1 | BC015818 | 73 |
| IOH7291 | 220729 | NM_005651.1 | NM_005651 | 196 |
| IOH14451 | 220730 | BC018632.1 | BC018632 | 394 |
| IOH27724 | 220731 | BC038713.1 | BC038713 | 30 |
| IOH22322 | 220732 | BC028682.2 | BC028682 | 40 |
| IOH27335 | 220733 | NM_001608.1 | NM_001608 | 2776 |
| IOH25799 | 220735 | NM_173830.3 | NM_173830 | 5240 |
| IOH21965 | 220736 | NM_032868.1 | NM_032868 | 600 |
| IOH25906 | 220737 | BC035882.1 | BC035882 | 833 |
| IOH26825 | 220722 | NM_177966.3 | NM_177966 | 257 |
| IOH14848 | 220739 | BC021573.1 | BC021573 | 37 |
| IOH27535 | 220720 | NM_003211.1 | NM_003211 | 239 |
| IOH12001 | 220742 | NM_032858.1 | NM_032858 | 36 |
| IOH25842 | 220743 | NM_172159.2 | NM_172159 | 40 |
| IOH25885 | 220744 | NM_178553.2 | NM_178553 | 29 |
| IOH27322 | 220745 | BC031589.1 | BC031589 | 93 |
| IOH27372 | 220746 | BC033495.1 | BC033495 | 54 |
| IOH25811 | 220747 | BC023247.1 | BC023247 | 1575 |
| IOH26807 | 220748 | BC040457.1 | BC040457 | 279 |
| IOH27106 | 220749 | BC037278.1 | BC037278 | 2405 |
| IOH14142 | 220751 | NM_001375.1 | NM_001375 | 51 |
| IOH5524 | 220752 | NM_031439.1 | NM_031439 | 26 |
| IOH12159 | 217182 | BC012573.1 | BC012573 | 61 |
| IOH4956 | 220738 | NM_021146.2 | NM_021146 | 265 |
| IOH7568 | 220705 | BC008492.1 | BC008492 | 3280 |
| IOH5858 | 216483 | BC005857.1 | BC005857 | 1303 |
| IOH25900 | 220689 | BC041811.1 | BC041811 | 1892 |
| IOH10880 | 220690 | BC027322.1 | BC027322 | 78 |
| IOH14312 | 220691 | BC008884.1 | BC008884 | 83 |
| IOH6569 | 220693 | NM_032342.1 | NM_032342 | 132 |
| IOH11575 | 220694 | NM_175609.1 | NM_175609 | 105 |
| IOH3266 | 220695 | NM_007076.1 | NM_007076 | 400 |
| IOH27749 | 220697 | BC037878.1 | BC037878 | 5371 |
| IOH27405 | 220698 | BC035359.1 | BC035359 | 62 |
| IOH27206 | 220699 | BC036019.1 | BC036019 | 390 |
| IOH27741 | 220701 | BC037779.2 | BC037779 | 1374 |
| IOH7352 | 220702 | NM_016371.1 | NM_016371 | 46 |
| IOH6246 | 220726 | NM_006877.1 | NM_006877 | 2003 |
| IOH12181 | 220704 | BC012604.1 | BC012604 | 201 |
| IOH25867 | 220755 | NM_153716.1 | NM_153716 | 877 |
| IOH7527 | 220706 | BC005896.1 | BC005896 | 1039 |
| IOH11355 | 220707 | NM_001308.1 | NM_001308 | 2015 |
| IOH27679 | 220708 | BC035079.2 | BC035079 | 62 |
| IOH21615 | 220709 | BC031222.1 | BC031222 | 136 |
| IOH26808 | 220710 | BC038710.1 | BC038710 | 177 |
| IOH27524 | 220712 | BC036246.1 | BC036246 | 1091 |
| IOH25815 | 220713 | BC028295.1 | BC028295 | 110 |
| IOH4945 | 220714 | BC003568.0 | BC003568 | 1190 |
| IOH13936 | 220715 | NM_181703.1 | NM_181703 | 1355 |
| IOH14365 | 220716 | BC017475.1 | BC017475 | 945 |
| IOH11838 | 220717 | NM_006217.2 | NM_006217 | 611 |
| IOH13760 | 220719 | BC014550.1 | BC014550 | 197 |
| IOH11211 | 220703 | NM_017436.2 | NM_017436 | 240 |
| IOH12271 | 217159 | NM_020466.3 | NM_020466 | 52 |
| IOH11398 | 220753 | NM_002898.1 | NM_002898 | 1009 |
| IOH10239 | 217141 | NM_138333.1 | NM_138333 | 3413 |
| IOH11084 | 217143 | BC015323.1 | BC015323 | 80 |
| IOH12222 | 217146 | BC010915.1 | BC010915 | 736 |
| IOH12798 | 217147 | BC014532.1 | BC014532 | 1705 |
| IOH12838 | 217148 | NM_006299.2 | NM_006299 | 891 |
| IOH12145 | 217149 | BC014539.1 | BC014539 | 87 |
| IOH13421 | 217150 | BC017098.1 | BC017098 | 36 |
| IOH12306 | 217151 | NM_022104.1 | NM_022104 | 3045 |
| IOH10498 | 217152 | BC011959.1 | BC011959 | 2666 |
| IOH12334 | 217154 | NM_007083.2 | NM_007083 | 178 |
| IOH10730 | 217155 | NM_016289.2 | NM_016289 | 1452 |
| IOH12103 | 217139 | NM_148904.2 | NM_148904 | 142 |
| IOH12345 | 217158 | NM_003986.1 | NM_003986 | 372 |
| IOH12811 | 217137 | NM_006834.2 | NM_006834 | 1271 |
| IOH12855 | 217160 | NM_014596.3 | NM_014596 | 1389 |
| IOH12897 | 217161 | BC011011.1 | BC011011 | 32 |
| IOH13048 | 217163 | NM_152302.1 | NM_152302 | 1224 |
| IOH12821 | 217173 | NM_016940.1 | NM_016940 | 1246 |
| IOH12586 | 217175 | BC010405.2 | BC010405 | 271 |
| IOH10516 | 217176 | BC018346.1 | BC018346 | 2471 |
| IOH10874 | 217177 | NM_006788.2 | NM_006788 | 966 |
| IOH12192 | 217178 | NM_021255.1 | NM_021255 | 2198 |
| IOH11180 | 217179 | NM_017612.1 | NM_017612 | 464 |
| IOH11264 | 217157 | NM_052817.1 | NM_052817 | 75 |
| IOH11149 | 217108 | BC016911.1 | BC016911 | 30 |
| IOH21967 | 220756 | NM_014079.1 | NM_014079 | 55 |
| IOH27668 | 220759 | BC034318.1 | BC034318 | 275 |
| IOH27738 | 220760 | BC041876.1 | BC041876 | 49 |
| IOH3277 | 220761 | BC008090.1 | BC008090 | 1130 |
| IOH4907 | 220762 | BC001778.1 | BC001778 | 35 |
| IOH7335 | 220763 | NM_033213.1 | NM_033213 | 120 |
| IOH14157 | 220764 | NM_032924.2 | NM_032924 | 81 |
| IOH26805 | 220766 | BC051698.1 | BC051698 | 513 |
| IOH26848 | 220767 | NM_153353.2 | NM_153353 | 3707 |
| IOH27730 | 220768 | BC039362.1 | BC039362 | 143 |
| IOH27128 | 220769 | NM_153343.2 | NM_153343 | 2048 |
| IOH25790 | 220770 | BC021906.1 | BC021906 | 19 |
| IOH13488 | 217140 | BC026058.1 | BC026058 | 23 |
| IOH13135 | 217106 | NM_032213.2 | NM_032213 | 112 |
| IOH3311 | 216797 | BC009025.1 | BC009025 | 43 |
| IOH11042 | 217109 | BC026213.1 | BC026213 | 2691 |
| IOH12956 | 217110 | NM_145055.1 | NM_145055 | 604 |
| IOH12069 | 217111 | BC010904.1 | BC010904 | 44 |
| IOH12723 | 217113 | NM_013338.2 | NM_013338 | 174 |
| IOH12717 | 217118 | NM_015878.2 | NM_015878 | 34 |
| IOH10995 | 217121 | BC016914.1 | BC016914 | 106 |
| IOH12297 | 217122 | BC019337.1 | BC019337 | 68 |
| IOH12346 | 217123 | BC012626.1 | BC012626 | 678 |
| IOH12616 | 217127 | BC017376.2 | BC017376 | 1599 |
| IOH12128 | 217128 | BC014299.2 | BC014299 | 266 |
| IOH11229 | 217131 | NM_006685.2 | NM_006685 | 179 |
| IOH12916 | 217136 | NM_005368.1 | NM_005368 | 4411 |
| IOH22979 | 220771 | NM_018083.1 | NM_018083 | 3168 |
| IOH13470 | 220202 | BC017926.1 | BC017926 | 112 |
| IOH3931 | 220130 | BC002490.1 | BC002490 | 789 |
| IOH14646 | 220132 | NM_020378.2 | NM_020378 | 58 |
| IOH21862 | 220133 | NM_152499.1 | NM_152499 | 149 |
| IOH5353 | 220137 | NM_018137.1 | NM_018137 | 155 |
| IOH12436 | 220142 | BC011934.1 | BC011934 | 457 |
| IOH22864 | 220144 | BC031671.1 | BC031671 | 32 |
| IOH12083 | 220145 | BC014455.1 | BC014455 | 25 |
| IOH21792 | 220148 | BC033854.1 | BC033854 | 40 |
| IOH9690 | 220128 | NM_007021.1 | NM_007021 | 44 |
| IOH14283 | 220154 | NM_000948.1 | NM_000948 | 77 |
| IOH13538 | 220127 | NM_014488.2 | NM_014488 | 156 |
| IOH13203 | 220157 | NM_003975.1 | NM_003975 | 29 |
| IOH5241 | 220158 | NM_016608.1 | NM_016608 | 25 |
| IOH6588 | 220166 | BC006104.1 | BC006104 | 96 |
| IOH23124 | 220168 | BC029428.1 | BC029428 | 305 |
| IOH6878 | 220179 | NM_032753.2 | NM_032753 | 48 |
| IOH12214 | 220186 | NM_016364.2 | NM_016364 | 38 |
| IOH23140 | 220191 | BC029424.1 | BC029424 | 52 |
| IOH23143 | 220192 | BC029458.1 | BC029458 | 19 |
| IOH3025 | 216795 | BC000937.2 | BC000937 | 333 |
| IOH13252 | 219257 | NM_080590.1 | NM_080590 | 24 |
| IOH12052 | 219192 | NM_145051.1 | NM_145051 | 73 |
| IOH10942 | 219247 | NM_144594.1 | NM_144594 | 26 |
| IOH12556 | 220129 | NM_005725.2 | NM_005725 | 43 |
| IOH12086 | 220203 | BC020626.1 | BC020626 | 349 |
| IOH23121 | 219258 | BC018782.1 | BC018782 | 20 |
| IOH11169 | 220114 | NM_138450.1 | NM_138450 | 522 |
| IOH13180 | 220120 | BC017344.1 | BC017344 | 41 |
| IOH12453 | 220122 | BC011765.2 | BC011765 | 149 |
| IOH22705 | 220124 | NM_173586.1 | NM_173586 | 21 |
| IOH21589 | 220125 | NM_152465.1 | NM_152465 | 56 |
| IOH13354 | 220126 | BC009968.2 | BC009968 | 166 |
| IOH21779 | 219252 | NM_145280.1 | NM_145280 | 43 |
| IOH6636 | 217968 | BC006142.2 | BC006142 | 28 |
| IOH4759 | 217975 | BC000038.1 | BC000038 | 98 |
| IOH3992 | 217962 | NM_005720.1 | NM_005720 | 223 |
| IOH7236 | 218014 | NM_032330.1 | NM_032330 | 53 |
| IOH6818 | 218017 | NM_032926.1 | NM_032926 | 19 |
| IOH12304 | 220619 | NM_138432.1 | NM_138432 | 82 |
| IOH9712 | 220587 | BC011526.1 | BC011526 | 32 |
| IOH13898 | 220588 | NM_002109.3 | NM_002109 | 26 |
| IOH10969 | 220591 | NM_032138.2 | NM_032138 | 71 |
| IOH28294 | 220604 | AB065630.1 | AB065630 | 33 |
| IOH13441 | 219594 | BC022253.1 | BC022253 | 167 |
| IOH3871 | 220626 | NM_007189.1 | NM_007189 | 93 |
| IOH13218 | 220627 | BC021090.1 | BC021090 | 121 |
| IOH12715 | 220638 | NM_015671.2 | NM_015671 | 39 |
| IOH12872 | 220649 | BC022270.1 | BC022270 | 118 |
| IOH4802 | 220655 | BC001214.1 | BC001214 | 122 |
| IOH27507 | 220656 | NM_175738.2 | NM_175738 | 280 |
| IOH14552 | 220661 | NM_004286.2 | NM_004286 | 95 |
| IOH3563 | 220611 | NM_015698.2 | NM_015698 | 161 |
| IOH10201 | 217054 | BC009006.1 | BC009006 | 25 |
| IOH22862 | 219597 | BC029652.1 | BC029652 | 38 |
| IOH11318 | 217037 | BC016395.1 | BC016395 | 1191 |
| IOH10845 | 217039 | BC016848.1 | BC016848 | 69 |
| IOH11302 | 217040 | BC018113.1 | BC018113 | 160 |
| IOH10199 | 217042 | NM_018279.2 | NM_018279 | 61 |
| IOH10298 | 217044 | NM_080678.1 | NM_080678 | 1454 |
| IOH10317 | 217045 | BC017724.1 | BC017724 | 577 |
| IOH10346 | 217046 | NM_007260.2 | NM_007260 | 2223 |
| IOH10391 | 217047 | NM_020424.2 | NM_020424 | 92 |
| IOH11268 | 217051 | BC015479.1 | BC015479 | 25 |
| IOH10345 | 217034 | BC016979.1 | BC016979 | 353 |
| IOH10314 | 217033 | NM_031297.1 | NM_031297 | 170 |
| IOH10268 | 217055 | NM_006054.1 | NM_006054 | 492 |
| IOH10300 | 217056 | NM_001636.1 | NM_001636 | 343 |
| IOH10392 | 217059 | NM_152637.1 | NM_152637 | 28 |
| IOH10793 | 217060 | NM_017853.1 | NM_017853 | 1088 |
| IOH11052 | 217061 | NM_012419.3 | NM_012419 | 2048 |
| IOH11246 | 217063 | NM_015423.2 | NM_015423 | 779 |
| IOH10925 | 217065 | NM_013401.2 | NM_013401 | 1483 |
| IOH10269 | 217067 | NM_052877.1 | NM_052877 | 114 |
| IOH10302 | 217068 | NM_031910.2 | NM_031910 | 124 |
| IOH10325 | 217069 | NM_033046.1 | NM_033046 | 340 |
| IOH11235 | 217052 | NM_014372.1 | NM_014372 | 823 |
| IOH11243 | 217012 | NM_006579.1 | NM_006579 | 245 |
| IOH14480 | 220683 | NM_019894.1 | NM_019894 | 81 |
| IOH11681 | 216799 | BC001550.1 | BC001550 | 2772 |
| IOH3912 | 216800 | NM_021159.2 | NM_021159 | 840 |
| IOH3959 | 216801 | NM_016049.1 | NM_016049 | 1022 |
| IOH4188 | 216804 | BC000651.1 | BC000651 | 211 |
| IOH3059 | 216807 | NM_002870.1 | NM_002870 | 93 |
| IOH3272 | 216808 | BC001286.1 | BC001286 | 844 |
| IOH13806 | 216810 | NM_002469.1 | NM_002469 | 674 |
| IOH3920 | 216811 | BC001120.1 | BC001120 | 1728 |
| IOH4117 | 216813 | BC002616.1 | BC002616 | 576 |
| IOH4208 | 216815 | NM_014060.1 | NM_014060 | 684 |
| IOH4250 | 216816 | BC000607.1 | BC000607 | 183 |
| IOH10961 | 217036 | NM_004331.1 | NM_004331 | 877 |
| IOH3070 | 216818 | BC000809.1 | BC000809 | 204 |
| IOH10789 | 217075 | BC015239.1 | BC015239 | 221 |
| IOH10805 | 217013 | NM_002491.1 | NM_002491 | 326 |
| IOH10842 | 217014 | NM_052935.1 | NM_052935 | 35 |
| IOH10242 | 217019 | NM_058169.1 | NM_058169 | 390 |
| IOH10309 | 217021 | BC016942.1 | BC016942 | 640 |
| IOH10384 | 217023 | NM_032044.1 | NM_032044 | 30 |
| IOH11028 | 217026 | NM_145206.1 | NM_145206 | 1605 |
| IOH11236 | 217028 | BC015468.1 | BC015468 | 43 |
| IOH10198 | 217030 | BC010241.1 | BC010241 | 45 |
| IOH10297 | 217032 | BC010555.1 | BC010555 | 437 |
| IOH2958 | 216817 | BC001001.2 | BC001001 | 594 |
| IOH14654 | 219562 | BC015667.2 | BC015667 | 46 |
| IOH22174 | 219563 | NM_002963.2 | NM_002963 | 1037 |
| IOH22742 | 219564 | BC031650.1 | BC031650 | 102 |
| IOH23108 | 219567 | NM_001671.2 | NM_001671 | 86 |
| IOH6921 | 219568 | BC007602.1 | BC007602 | 100 |
| IOH23099 | 219573 | NM_015666.2 | NM_015666 | 54 |
| IOH5167 | 219574 | NM_032326.1 | NM_032326 | 43 |
| IOH22771 | 219575 | NM_004291.1 | NM_004291 | 77 |
| IOH10368 | 217070 | NM_003492.1 | NM_003492 | 49 |
| IOH5740 | 219577 | BC002940.1 | BC002940 | 691 |
| IOH6650 | 219556 | BC006148.1 | BC006148 | 41 |
| IOH21859 | 219581 | NM_139242.1 | NM_139242 | 38 |
| IOH13169 | 219582 | BC010167.2 | BC010167 | 115 |
| IOH22696 | 219583 | BC029121.1 | BC029121 | 26 |
| IOH22756 | 219584 | NM_152614.1 | NM_152614 | 24 |
| IOH23072 | 219585 | BC015842.1 | BC015842 | 1415 |
| IOH22794 | 219588 | NM_002608.1 | NM_002608 | 66 |
| IOH22119 | 219591 | BC029760.1 | BC029760 | 1267 |
| IOH21708 | 219592 | NM_152776.1 | NM_152776 | 30 |
| IOH3263 | 216796 | BC009009.1 | BC009009 | 32 |
| IOH21765 | 219576 | BC032775.1 | BC032775 | 178 |
| IOH10824 | 217095 | NM_014061.3 | NM_014061 | 43 |
| IOH10129 | 219595 | NM_016614.1 | NM_016614 | 728 |
| IOH11040 | 217076 | NM_002927.3 | NM_002927 | 263 |
| IOH10948 | 217077 | BC015409.1 | BC015409 | 114 |
| IOH10272 | 217079 | NM_005724.3 | NM_005724 | 75 |
| IOH10304 | 217080 | NM_138800.1 | NM_138800 | 22 |
| IOH10328 | 217081 | BC015329.1 | BC015329 | 2126 |
| IOH10372 | 217082 | BC020962.1 | BC020962 | 74 |
| IOH11057 | 217086 | BC015535.1 | BC015535 | 62 |
| IOH11259 | 217089 | NM_002362.2 | NM_002362 | 1042 |
| IOH10281 | 217091 | NM_032809.2 | NM_032809 | 77 |
| IOH9663 | 219559 | BC010458.1 | BC010458 | 112 |
| IOH10375 | 217094 | BC016857.1 | BC016857 | 590 |
| IOH14835 | 219557 | NM_174923.1 | NM_174923 | 220 |
| IOH11027 | 217096 | NM_138808.1 | NM_138808 | 20 |
| IOH10971 | 217100 | BC015413.1 | BC015413 | 27 |
| IOH10229 | 217101 | NM_016176.2 | NM_016176 | 159 |
| IOH10289 | 217102 | NM_052837.1 | NM_052837 | 70 |
| IOH10308 | 217103 | BC016941.1 | BC016941 | 27 |
| IOH10340 | 217104 | BC016934.1 | BC016934 | 23 |
| IOH10379 | 217105 | BC020966.1 | BC020966 | 43 |
| IOH22849 | 219551 | BC027486.1 | BC027486 | 447 |
| IOH22562 | 219552 | BC029524.1 | BC029524 | 418 |
| IOH23080 | 219555 | BC015878.1 | BC015878 | 242 |
| IOH10852 | 217074 | NM_003792.1 | NM_003792 | 380 |
| IOH10306 | 217092 | NM_006978.1 | NM_006978 | 1042 |
| IOH12788 | 219789 | NM_177552.1 | NM_177552 | 514 |
| IOH5541 | 219804 | NM_004578.2 | NM_004578 | 260 |
| IOH3269 | 219768 | NM_003825.2 | NM_003825 | 5370 |
| IOH9701 | 219769 | BC010642.1 | BC010642 | 368 |
| IOH3256 | 219770 | BC001244.1 | BC001244 | 878 |
| IOH13784 | 219771 | BC015066.1 | BC015066 | 153 |
| IOH22826 | 219777 | NM_031481.1 | NM_031481 | 27 |
| IOH14352 | 219778 | NM_005614.2 | NM_005614 | 39 |
| IOH14450 | 219779 | NM_003278.1 | NM_003278 | 49 |
| IOH14289 | 219780 | NM_006007.1 | NM_006007 | 592 |
| IOH13742 | 219781 | BC010959.1 | BC010959 | 202 |
| IOH3965 | 219782 | NM_004357.2 | NM_004357 | 4860 |
| IOH3081 | 219784 | NM_016098.1 | NM_016098 | 105 |
| IOH2916 | 219766 | NM_015646.1 | NM_015646 | 787 |
| IOH7254 | 219788 | BC005218.1 | BC005218 | 53 |
| IOH12177 | 219765 | BC014991.1 | BC014991 | 141 |
| IOH5958 | 219790 | BC008365.1 | BC008365 | 801 |
| IOH14099 | 219791 | BC011842.2 | BC011842 | 1646 |
| IOH6329 | 219792 | BC006288.1 | BC006288 | 179 |
| IOH14184 | 219793 | BC011006.1 | BC011006 | 1611 |
| IOH10868 | 219794 | NM_145006.1 | NM_145006 | 254 |
| IOH11073 | 219795 | BC012947.1 | BC012947 | 2230 |
| IOH14044 | 219796 | BC021286.1 | BC021286 | 2654 |
| IOH6278 | 219797 | BC007689.2 | BC007689 | 1529 |
| IOH10802 | 219800 | NM_145286.1 | NM_145286 | 1015 |
| IOH14443 | 219801 | NM_020980.2 | NM_020980 | 625 |
| IOH14506 | 219802 | NM_152267.2 | NM_152267 | 23 |
| IOH13864 | 216619 | NM_005558.2 | NM_005558 | 310 |
| IOH11390 | 219785 | BC015492.1 | BC015492 | 1120 |
| IOH2929 | 219748 | BC003377.1 | BC003377 | 77 |
| IOH27228 | 220688 | NM_019109.1 | NM_019109 | 55 |
| IOH5421 | 216624 | NM_016103.1 | NM_016103 | 358 |
| IOH6672 | 216625 | NM_002867.2 | NM_002867 | 3330 |
| IOH10734 | 216626 | BC020495.1 | BC020495 | 75 |
| IOH14575 | 216627 | NM_006270.2 | NM_006270 | 2277 |
| IOH9688 | 216628 | NM_004422.1 | NM_004422 | 102 |
| IOH13239 | 216629 | NM_018969.2 | NM_018969 | 54 |
| IOH21132 | 216630 | NM_024046.1 | NM_024046 | 455 |
| IOH22568 | 219741 | NM_152587.2 | NM_152587 | 2606 |
| IOH4077 | 219742 | BC002520.1 | BC002520 | 287 |
| IOH14113 | 219744 | BC009762.2 | BC009762 | 266 |
| IOH7448 | 219745 | BC008438.1 | BC008438 | 823 |
| IOH14238 | 219767 | BC021241.2 | BC021241 | 1484 |
| IOH13789 | 219747 | BC010963.1 | BC010963 | 549 |
| IOH3028 | 219805 | NM_031227.1 | NM_031227 | 2193 |
| IOH5164 | 219750 | BC004896.1 | BC004896 | 67 |
| IOH13706 | 219752 | NM_003106.2 | NM_003106 | 410 |
| IOH6738 | 219753 | BC007806.1 | BC007806 | 71 |
| IOH11628 | 219754 | NM_144593.1 | NM_144593 | 100 |
| IOH11804 | 219755 | BC028728.1 | BC028728 | 250 |
| IOH14448 | 219756 | BC017101.1 | BC017101 | 1363 |
| IOH14519 | 219757 | BC014521.1 | BC014521 | 592 |
| IOH14186 | 219758 | NM_015975.3 | NM_015975 | 5374 |
| IOH11799 | 219759 | NM_001008.2 | NM_001008 | 29 |
| IOH3847 | 219760 | NM_016468.2 | NM_016468 | 253 |
| IOH12799 | 219763 | NM_024713.1 | NM_024713 | 67 |
| IOH5099 | 219764 | NM_001154.2 | NM_001154 | 1051 |
| IOH10850 | 219746 | NM_152667.1 | NM_152667 | 52 |
| IOH12227 | 219983 | BC009779.1 | BC009779 | 1886 |
| IOH5640 | 219803 | NM_031472.1 | NM_031472 | 4271 |
| IOH14089 | 219945 | BC014095.2 | BC014095 | 5370 |
| IOH5465 | 219947 | BC004938.1 | BC004938 | 1918 |
| IOH14627 | 219948 | BC021995.1 | BC021995 | 837 |
| IOH12733 | 219950 | NM_144654.1 | NM_144654 | 223 |
| IOH12301 | 219951 | NM_006643.2 | NM_006643 | 3577 |
| IOH10186 | 219953 | BC010504.1 | BC010504 | 362 |
| IOH12212 | 219955 | BC012609.1 | BC012609 | 1583 |
| IOH6217 | 219963 | NM_033177.2 | NM_033177 | 78 |
| IOH14248 | 219964 | BC014665.1 | BC014665 | 4273 |
| IOH13812 | 219966 | NM_003666.1 | NM_003666 | 459 |
| IOH10741 | 219967 | NM_053285.1 | NM_053285 | 69 |
| IOH10347 | 219942 | NM_002194.2 | NM_002194 | 3196 |
| IOH4736 | 219977 | BC000111.1 | BC000111 | 118 |
| IOH3316 | 219941 | NM_138379.1 | NM_138379 | 21 |
| IOH12689 | 219984 | BC012192.1 | BC012192 | 36 |
| IOH12915 | 219995 | NM_016305.1 | NM_016305 | 3078 |
| IOH10208 | 219996 | BC013648.1 | BC013648 | 596 |
| IOH13007 | 220000 | NM_002243.2 | NM_002243 | 301 |
| IOH9923 | 220001 | NM_005103.3 | NM_005103 | 1011 |
| IOH3184 | 220004 | BC006793.1 | BC006793 | 112 |
| IOH5273 | 220006 | BC002629.1 | BC002629 | 506 |
| IOH10197 | 220010 | BC008141.1 | BC008141 | 1000 |
| IOH10264 | 220013 | BC016440.1 | BC016440 | 134 |
| IOH9764 | 220014 | BC018445.1 | BC018445 | 2112 |
| IOH4911 | 220015 | BC001709.1 | BC001709 | 5195 |
| IOH10296 | 220017 | BC012881.1 | BC012881 | 64 |
| IOH14388 | 219975 | NM_003943.1 | NM_003943 | 32 |
| IOH5875 | 219829 | NM_018129.1 | NM_018129 | 102 |
| IOH3275 | 219806 | NM_007241.2 | NM_007241 | 775 |
| IOH2956 | 219807 | NM_030920.1 | NM_030920 | 5374 |
| IOH12991 | 219812 | NM_033416.1 | NM_033416 | 52 |
| IOH23147 | 219813 | BC029399.1 | BC029399 | 352 |
| IOH12754 | 219814 | BC010889.1 | BC010889 | 4646 |
| IOH5954 | 219815 | NM_006241.2 | NM_006241 | 498 |
| IOH6926 | 219816 | BC007312.1 | BC007312 | 31 |
| IOH11176 | 219817 | BC012919.1 | BC012919 | 1634 |
| IOH12664 | 219818 | NM_138412.1 | NM_138412 | 2303 |
| IOH3923 | 219819 | NM_005333.1 | NM_005333 | 57 |
| IOH14467 | 219823 | NM_001760.2 | NM_001760 | 56 |
| IOH2920 | 219825 | BC000903.2 | BC000903 | 5364 |
| IOH3201 | 219943 | BC001964.1 | BC001964 | 24 |
| IOH4156 | 219827 | NM_019606.3 | NM_019606 | 514 |
| IOH10344 | 216618 | BC016964.1 | BC016964 | 118 |
| IOH12105 | 219830 | BC015118.1 | BC015118 | 242 |
| IOH3283 | 219831 | BC008990.1 | BC008990 | 5343 |
| IOH3251 | 219926 | NM_024058.1 | NM_024058 | 68 |
| IOH14527 | 219927 | NM_172341.1 | NM_172341 | 1089 |
| IOH12891 | 219929 | BC013319.1 | BC013319 | 25 |
| IOH9750 | 219930 | BC016614.1 | BC016614 | 68 |
| IOH6391 | 219931 | NM_033661.1 | NM_033661 | 5106 |
| IOH3325 | 219935 | BC008091.1 | BC008091 | 2308 |
| IOH12592 | 219936 | BC010181.1 | BC010181 | 4041 |
| IOH5376 | 219938 | NM_007233.1 | NM_007233 | 588 |
| IOH4363 | 219939 | NM_005272.2 | NM_005272 | 820 |
| IOH10698 | 219940 | NM_182488.1 | NM_182488 | 479 |
| IOH6081 | 219826 | BC005876.1 | BC005876 | 752 |
| IOH20996 | 216539 | NM_006504.2 | NM_006504 | 163 |
| IOH7013 | 216552 | BC007324.1 | BC007324 | 82 |
| IOH11251 | 216523 | BC025708.1 | BC025708 | 654 |
| IOH12770 | 216524 | NM_052946.1 | NM_052946 | 86 |
| IOH14193 | 216526 | NM_144624.1 | NM_144624 | 1027 |
| IOH21152 | 216527 | NM_005248.1 | NM_005248 | 1648 |
| IOH5340 | 216528 | BC002706.1 | BC002706 | 107 |
| IOH4753 | 216529 | BC000729.1 | BC000729 | 27 |
| IOH6313 | 216530 | NM_000858.2 | NM_000858 | 3858 |
| IOH6708 | 216531 | NM_002045.1 | NM_002045 | 4105 |
| IOH5978 | 216532 | NM_001827.1 | NM_001827 | 5370 |
| IOH12559 | 216534 | BC013992.1 | BC013992 | 5374 |
| IOH13992 | 216535 | NM_013410.1 | NM_013410 | 5196 |
| IOH7357 | 216521 | BC005371.1 | BC005371 | 5369 |
| IOH2412 | 216537 | NM_003583.2 | NM_003583 | 282 |
| IOH7134 | 216520 | BC008374.1 | BC008374 | 3701 |
| IOH6325 | 216540 | NM_007240.1 | NM_007240 | 3283 |
| IOH13715 | 216541 | NM_177554.1 | NM_177554 | 290 |
| IOH5691 | 216542 | BC004522.1 | BC004522 | 1565 |
| IOH7574 | 216543 | NM_001664.1 | NM_001664 | 5363 |
| IOH12834 | 216544 | BC018942.1 | BC018942 | 136 |
| IOH11309 | 216545 | BC024004.1 | BC024004 | 132 |
| IOH3294 | 216546 | NM_001736.1 | NM_001736 | 39 |
| IOH11033 | 216547 | NM_004720.3 | NM_004720 | 56 |
| IOH13042 | 216549 | NM_003130.1 | NM_003130 | 1115 |
| IOH4141 | 216550 | NM_054033.1 | NM_054033 | 1540 |
| IOH13214 | 216623 | NM_033256.1 | NM_033256 | 931 |
| IOH14360 | 216536 | NM_001625.1 | NM_001625 | 5370 |
| IOH12669 | 216499 | BC014552.1 | BC014552 | 1104 |
| IOH21154 | 216480 | NM_017490.1 | NM_017490 | 204 |
| IOH6979 | 216484 | NM_000269.1 | NM_000269 | 5376 |
| IOH10122 | 216486 | NM_000431.1 | NM_000431 | 5360 |
| IOH12980 | 216487 | BC015186.1 | BC015186 | 2121 |
| IOH11014 | 216488 | NM_005565.2 | NM_005565 | 5364 |
| IOH11645 | 216489 | NM_001721.2 | NM_001721 | 806 |
| IOH14591 | 216490 | BC021278.1 | BC021278 | 315 |
| IOH20967 | 216492 | NM_020439.1 | NM_020439 | 4211 |
| IOH5163 | 216493 | NM_001800.2 | NM_001800 | 5360 |
| IOH5481 | 216494 | NM_018110.2 | NM_018110 | 1807 |
| IOH6258 | 216495 | NM_033019.1 | NM_033019 | 5372 |
| IOH7002 | 216496 | NM_018571.4 | NM_018571 | 129 |
| IOH10488 | 216522 | BC018345.1 | BC018345 | 2413 |
| IOH10145 | 216498 | NM_005391.1 | NM_005391 | 483 |
| IOH11625 | 216553 | BC028719.1 | BC028719 | 198 |
| IOH11097 | 216500 | NM_004417.2 | NM_004417 | 916 |
| IOH5211 | 216505 | NM_001823.2 | NM_001823 | 4305 |
| IOH4633 | 216506 | NM_002044.1 | NM_002044 | 5214 |
| IOH6284 | 216507 | BC006231.1 | BC006231 | 244 |
| IOH7132 | 216508 | NM_006748.1 | NM_006748 | 139 |
| IOH7287 | 216509 | BC007462.1 | BC007462 | 5367 |
| IOH10918 | 216511 | NM_145025.1 | NM_145025 | 636 |
| IOH11402 | 216513 | NM_024779.2 | NM_024779 | 5374 |
| IOH14775 | 216514 | BC024291.1 | BC024291 | 5366 |
| IOH21038 | 216515 | NM_005233.2 | NM_005233 | 518 |
| IOH4674 | 216518 | NM_031361.1 | NM_031361 | 2288 |
| IOH6288 | 216519 | BC006233.1 | BC006233 | 4230 |
| IOH7271 | 216497 | BC005298.1 | BC005298 | 3295 |
| IOH5158 | 216605 | BC005153.1 | BC005153 | 724 |
| IOH21299 | 216551 | NM_024025.1 | NM_024025 | 89 |
| IOH10104 | 216591 | NM_022337.1 | NM_022337 | 4645 |
| IOH1753 | 216592 | NM_001667.1 | NM_001667 | 3990 |
| IOH3460 | 216593 | NM_002436.2 | NM_002436 | 741 |
| IOH6697 | 216596 | NM_020299.2 | NM_020299 | 1469 |
| IOH14446 | 216597 | BC022305.1 | BC022305 | 1523 |
| IOH5443 | 216599 | NM_003712.1 | NM_003712 | 71 |
| IOH12943 | 216600 | BC009196.1 | BC009196 | 109 |
| IOH14614 | 216601 | BC021289.1 | BC021289 | 22 |
| IOH6072 | 216602 | NM_023940.1 | NM_023940 | 2635 |
| IOH14587 | 216589 | NM_002710.1 | NM_002710 | 37 |
| IOH14475 | 216604 | NM_002884.1 | NM_002884 | 105 |
| IOH12805 | 216588 | NM_014241.2 | NM_014241 | 216 |
| IOH9624 | 216606 | NM_003382.2 | NM_003382 | 31 |
| IOH1987 | 216607 | NM_015727.1 | NM_015727 | 39 |
| IOH11395 | 216609 | BC028739.2 | BC028739 | 36 |
| IOH7464 | 216610 | NM_016301.2 | NM_016301 | 133 |
| IOH5608 | 216611 | NM_005605.2 | NM_005605 | 91 |
| IOH12269 | 216612 | BC020700.1 | BC020700 | 130 |
| IOH4164 | 216613 | BC000566.1 | BC000566 | 147 |
| IOH6101 | 216614 | NM_017595.2 | NM_017595 | 3826 |
| IOH10511 | 216615 | NM_004283.2 | NM_004283 | 756 |
| IOH14604 | 216616 | NM_002070.1 | NM_002070 | 4171 |
| IOH5175 | 216617 | BC005155.1 | BC005155 | 34 |
| IOH10139 | 216603 | NM_021252.2 | NM_021252 | 4950 |
| IOH14797 | 216569 | NM_022777.1 | NM_022777 | 913 |
| IOH5472 | 216554 | BC004247.1 | BC004247 | 2510 |
| IOH9848 | 216555 | NM_002068.1 | NM_002068 | 245 |
| IOH10825 | 216556 | NM_145313.1 | NM_145313 | 24 |
| IOH1937 | 216557 | NM_006822.1 | NM_006822 | 68 |
| IOH3305 | 216558 | BC008094.1 | BC008094 | 54 |
| IOH12614 | 216559 | BC009877.1 | BC009877 | 133 |
| IOH4559 | 216560 | NM_024076.1 | NM_024076 | 1391 |
| IOH12967 | 216561 | BC009961.1 | BC009961 | 1332 |
| IOH4659 | 216562 | BC000103.1 | BC000103 | 928 |
| IOH3815 | 216563 | NM_007236.2 | NM_007236 | 107 |
| IOH7224 | 216564 | NM_002721.3 | NM_002721 | 59 |
| IOH4847 | 216566 | BC003088.1 | BC003088 | 74 |
| IOH4954 | 216590 | NM_001663.2 | NM_001663 | 1643 |
| IOH12833 | 216568 | NM_014310.3 | NM_014310 | 808 |
| IOH12030 | 218896 | NM_002704.1 | NM_002704 | 469 |
| IOH5698 | 216572 | NM_031436.1 | NM_031436 | 541 |
| IOH12198 | 216573 | NM_005832.2 | NM_005832 | 57 |
| IOH4436 | 216574 | NM_002903.1 | NM_002903 | 1516 |
| IOH3548 | 216575 | NM_001467.2 | NM_001467 | 110 |
| IOH7558 | 216576 | BC008493.1 | BC008493 | 95 |
| IOH13822 | 216577 | NM_016361.2 | NM_016361 | 269 |
| IOH10011 | 216579 | NM_006861.2 | NM_006861 | 2763 |
| IOH12810 | 216580 | NM_016530.1 | NM_016530 | 165 |
| IOH14673 | 216581 | NM_004251.2 | NM_004251 | 3858 |
| IOH5739 | 216584 | NM_020677.1 | NM_020677 | 1953 |
| IOH5913 | 216586 | NM_172016.1 | NM_172016 | 110 |
| IOH5237 | 216587 | NM_004090.1 | NM_004090 | 3830 |
| IOH10004 | 216567 | NM_020673.1 | NM_020673 | 3098 |
| IOH14287 | 219845 | NM_053045.1 | NM_053045 | 201 |
| IOH11993 | 219861 | BC020976.1 | BC020976 | 919 |
| IOH21099 | 219540 | NM_020185.2 | NM_020185 | 257 |
| IOH21339 | 219541 | NM_016508.2 | NM_016508 | 414 |
| IOH22332 | 219545 | NM_024745.1 | NM_024745 | 788 |
| IOH21538 | 219548 | BC032249.1 | BC032249 | 52 |
| IOH5031 | 219834 | NM_032308.1 | NM_032308 | 4871 |
| IOH7456 | 219835 | NM_145792.1 | NM_145792 | 81 |
| IOH4806 | 219836 | BC001907.1 | BC001907 | 3556 |
| IOH5889 | 219838 | BC008037.2 | BC008037 | 3082 |
| IOH9807 | 219840 | BC009047.1 | BC009087 | 3119 |
| IOH3994 | 219841 | NM_020467.2 | NM_020467 | 3104 |
| IOH13242 | 219537 | BC015625.1 | BC015625 | 49 |
| IOH3136 | 219844 | NM_005340.1 | NM_005340 | 3260 |
| IOH22318 | 219534 | BC030597.1 | BC030597 | 230 |
| IOH2912 | 219846 | BC003366.1 | BC003366 | 180 |
| IOH3243 | 219847 | NM_007362.2 | NM_007362 | 5374 |
| IOH10494 | 219848 | NM_016058.1 | NM_016058 | 5365 |
| IOH5367 | 219851 | BC002758.1 | BC002758 | 470 |
| IOH4100 | 219852 | NM_006468.3 | NM_006468 | 2762 |
| IOH3240 | 219853 | BC001256.1 | BC001256 | 402 |
| IOH4556 | 219854 | NM_005274.1 | NM_005274 | 1804 |
| IOH3382 | 219855 | BC008651.1 | BC008651 | 74 |
| IOH10623 | 219857 | BC015155.1 | BC015155 | 126 |
| IOH13168 | 218894 | NM_032574.1 | NM_032574 | 468 |
| IOH13650 | 219843 | BC018953.1 | BC018953 | 254 |
| IOH21787 | 219480 | BC033851.1 | BC033851 | 1291 |
| IOH4703 | 219454 | BC000712.1 | BC000712 | 2368 |
| IOH22829 | 219455 | BC027465.1 | BC027465 | 644 |
| IOH5310 | 219456 | BC002769.1 | BC002769 | 1069 |
| IOH21007 | 219457 | BC031549.1 | BC031549 | 2037 |
| IOH21418 | 219459 | BC034718.1 | BC034718 | 480 |
| IOH13910 | 219464 | NM_005510.2 | NM_005510 | 2246 |
| IOH6373 | 219465 | NM_024901.2 | NM_024901 | 1432 |
| IOH21512 | 219468 | BC030253.1 | BC030253 | 1958 |
| IOH21026 | 219469 | NM_022048.1 | NM_022048 | 1205 |
| IOH21419 | 219471 | BC011392.1 | BC011392 | 2728 |
| IOH22249 | 219473 | BC036649.1 | BC036649 | 60 |
| IOH22290 | 219474 | BC030776.1 | BC030776 | 73 |
| IOH13175 | 219538 | NM_138790.1 | NM_138790 | 39 |
| IOH22410 | 219476 | BC030020.2 | BC030020 | 389 |
| IOH4057 | 219862 | BC001408.1 | BC001408 | 53 |
| IOH22297 | 219486 | BC034483.1 | BC034483 | 790 |
| IOH6500 | 219492 | NM_032694.1 | NM_032694 | 4234 |
| IOH21472 | 219496 | BC019954.1 | BC019954 | 287 |
| IOH22299 | 219498 | NM_032491.2 | NM_032491 | 736 |
| IOH22369 | 219499 | NM_006202.1 | NM_006202 | 186 |
| IOH21592 | 219503 | NM_152394.2 | NM_152394 | 33 |
| IOH22389 | 219511 | BC030653.2 | BC030653 | 2384 |
| IOH20954 | 219516 | NM_178152.1 | NM_178152 | 2342 |
| IOH21323 | 219518 | NM_001277.1 | NM_001277 | 2584 |
| IOH21336 | 219530 | NM_014326.2 | NM_014326 | 1053 |
| IOH21451 | 219531 | BC034247.1 | BC034247 | 417 |
| IOH22282 | 219533 | BC034468.1 | BC034468 | 71 |
| IOH22340 | 219475 | NM_033103.1 | NM_033103 | 207 |
| IOH7163 | 219915 | NM_004102.2 | NM_004102 | 5372 |
| IOH12123 | 219859 | NM_173362.2 | NM_173362 | 4749 |
| IOH14013 | 219897 | NM_005147.1 | NM_005147 | 46 |
| IOH13637 | 219898 | BC015754.1 | BC015754 | 774 |
| IOH13536 | 219899 | NM_005842.2 | NM_005842 | 346 |
| IOH2980 | 219900 | BC000962.2 | BC000962 | 2365 |
| IOH5105 | 219901 | BC004969.1 | BC004969 | 5363 |
| IOH5325 | 219902 | NM_024312.1 | NM_024312 | 1279 |
| IOH5254 | 219903 | BC002656.1 | BC002656 | 1267 |
| IOH11669 | 219905 | NM_152773.2 | NM_152773 | 1546 |
| IOH5830 | 219906 | BC007407.1 | BC007407 | 944 |
| IOH3804 | 219907 | BC004179.1 | BC004179 | 137 |
| IOH6880 | 219908 | BC007282.1 | BC007282 | 232 |
| IOH6966 | 219895 | NM_032920.1 | NM_032920 | 156 |
| IOH11511 | 219913 | BC028039.1 | BC028039 | 5368 |
| IOH3328 | 219893 | BC008567.1 | BC008567 | 5219 |
| IOH3511 | 219916 | NM_006022.1 | NM_006022 | 418 |
| IOH14253 | 219917 | BC010896.1 | BC010896 | 178 |
| IOH12025 | 219918 | BC027866.1 | BC027866 | 52 |
| IOH5656 | 219919 | NM_015610.1 | NM_015610 | 313 |
| IOH11880 | 219920 | NM_003447.1 | NM_003447 | 109 |
| IOH14723 | 219921 | BC011928.2 | BC011928 | 651 |
| IOH6345 | 219922 | BC008803.1 | BC008803 | 186 |
| IOH4359 | 219923 | NM_021992.1 | NM_021992 | 5371 |
| IOH6980 | 219925 | NM_032886.1 | NM_032886 | 56 |
| IOH13940 | 220678 | NM_144620.1 | NM_144620 | 1577 |
| IOH10654 | 220681 | NM_007249.3 | NM_007249 | 73 |
| IOH7170 | 220682 | BC006986.1 | BC006986 | 82 |
| IOH9842 | 219910 | BC009734.1 | BC009734 | 353 |
| IOH12626 | 219880 | NM_012396.1 | NM_012396 | 852 |
| IOH14667 | 219863 | BC020786.1 | BC020786 | 92 |
| IOH12518 | 219865 | BC010172.2 | BC010172 | 373 |
| IOH4263 | 219866 | NM_000999.2 | NM_000999 | 505 |
| IOH13535 | 219867 | BC016754.1 | BC016754 | 405 |
| IOH4447 | 219868 | BC001716.1 | BC001716 | 2543 |
| IOH5650 | 219869 | BC004885.1 | BC004885 | 524 |
| IOH11279 | 219870 | BC017064.1 | BC017064 | 188 |
| IOH12898 | 219871 | BC010900.1 | BC010900 | 157 |
| IOH9869 | 219874 | NM_017837.2 | NM_017837 | 44 |
| IOH4273 | 219875 | BC002430.1 | BC002430 | 103 |
| IOH4189 | 219876 | NM_014366.1 | NM_014366 | 243 |
| IOH3865 | 219877 | BC001694.1 | BC001694 | 5358 |
| IOH5510 | 219896 | NM_024061.1 | NM_024061 | 304 |
| IOH10463 | 219879 | BC013687.1 | BC013687 | 499 |
| IOH11381 | 219451 | NM_005641.2 | NM_005641 | 617 |
| IOH6968 | 219881 | BC007639.1 | BC007639 | 116 |
| IOH7274 | 219882 | NM_031427.1 | NM_031427 | 390 |
| IOH13646 | 219883 | BC015059.1 | BC015059 | 2985 |
| IOH5952 | 219884 | NM_001660.2 | NM_001660 | 5376 |
| IOH11106 | 219885 | NM_006838.1 | NM_006838 | 2134 |
| IOH4913 | 219886 | BC002954.1 | BC002954 | 425 |
| IOH14170 | 219887 | BC022361.1 | BC022361 | 525 |
| IOH6338 | 219888 | BC006259.2 | BC006259 | 120 |
| IOH4850 | 219889 | NM_178191.1 | NM_178191 | 723 |
| IOH21487 | 219890 | NM_052861.1 | NM_052861 | 129 |
| IOH4965 | 219891 | BC001868.1 | BC001868 | 244 |
| IOH14751 | 219892 | BC015091.2 | BC015091 | 535 |
| IOH5727 | 219878 | BC002934.1 | BC002934 | 567 |
| IOH12223 | 218954 | NM_002555.2 | NM_002555 | 469 |
| IOH14755 | 219453 | BC018747.1 | BC018747 | 258 |
| IOH14111 | 218932 | NM_145271.1 | NM_145271 | 224 |
| IOH12986 | 218933 | NM_000200.1 | NM_000200 | 2711 |
| IOH10884 | 218934 | NM_145254.1 | NM_145254 | 141 |
| IOH11035 | 218935 | BC018028.1 | BC018028 | 2152 |
| IOH12529 | 218938 | BC010414.1 | BC010414 | 2868 |
| IOH12944 | 218939 | BC009393.2 | BC009393 | 897 |
| IOH12382 | 218940 | NM_000608.1 | NM_000608 | 565 |
| IOH13353 | 218941 | NM_138794.1 | NM_138794 | 213 |
| IOH12649 | 218942 | NM_033281.2 | NM_033281 | 36 |
| IOH12242 | 218943 | NM_145300.1 | NM_145300 | 2004 |
| IOH11127 | 218946 | NM_004202.1 | NM_004202 | 43 |
| IOH13435 | 218930 | BC017381.1 | BC017381 | 2555 |
| IOH12548 | 218950 | BC009873.1 | BC009873 | 1244 |
| IOH12601 | 218927 | BC009366.1 | BC009366 | 159 |
| IOH13307 | 218955 | NM_025065.4 | NM_025065 | 3365 |
| IOH10921 | 218956 | BC016900.1 | BC016900 | 114 |
| IOH12487 | 218957 | BC010426.1 | BC010426 | 4709 |
| IOH11137 | 218958 | BC020942.1 | BC020942 | 277 |
| IOH11067 | 218959 | NM_080739.1 | NM_080739 | 32 |
| IOH12519 | 218961 | NM_017503.2 | NM_017503 | 249 |
| IOH12579 | 218962 | BC012783.2 | BC012783 | 1315 |
| IOH12074 | 218964 | BC014307.1 | BC014307 | 43 |
| IOH13306 | 218965 | BC017399.1 | BC017399 | 124 |
| IOH12816 | 218966 | NM_006216.2 | NM_006216 | 158 |
| IOH12539 | 218967 | NM_018215.1 | NM_018215 | 52 |
| IOH11147 | 218968 | BC012493.1 | BC012493 | 208 |
| IOH13317 | 218948 | NM_052950.2 | NM_052950 | 35 |
| IOH10849 | 218912 | NM_144717.1 | NM_144717 | 1052 |
| IOH21059 | 216479 | NM_003656.3 | NM_003656 | 5371 |
| IOH12727 | 218897 | NM_018413.2 | NM_018413 | 2005 |
| IOH13016 | 218898 | BC012984.2 | BC012984 | 906 |
| IOH11006 | 218899 | NM_003766.2 | NM_003766 | 1070 |
| IOH10955 | 218900 | BC027473.1 | BC027473 | 839 |
| IOH13426 | 218901 | BC014089.2 | BC014089 | 367 |
| IOH12121 | 218902 | NM_014035.1 | NM_014035 | 243 |
| IOH13230 | 218903 | NM_130777.1 | NM_130777 | 1085 |
| IOH12337 | 218904 | NM_006476.2 | NM_006476 | 253 |
| IOH12458 | 218905 | BC013935.1 | BC013935 | 34 |
| IOH12647 | 218906 | NM_005726.2 | NM_005726 | 136 |
| IOH12275 | 218907 | NM_144982.1 | NM_144982 | 65 |
| IOH12225 | 218931 | NM_002621.1 | NM_002621 | 616 |
| IOH11093 | 218910 | NM_012473.2 | NM_012473 | 167 |
| IOH10783 | 218971 | NM_145013.1 | NM_145013 | 35 |
| IOH12533 | 218913 | NM_005376.1 | NM_005376 | 414 |
| IOH12454 | 218914 | NM_138482.1 | NM_138482 | 2153 |
| IOH12084 | 218916 | BC021680.1 | BC021680 | 106 |
| IOH13071 | 218917 | NM_145303.1 | NM_145303 | 111 |
| IOH13075 | 218918 | NM_138573.1 | NM_138573 | 622 |
| IOH12288 | 218919 | NM_032570.1 | NM_032570 | 99 |
| IOH11647 | 218920 | NM_024561.1 | NM_024561 | 154 |
| IOH12120 | 218921 | BC012569.1 | BC012569 | 1926 |
| IOH10420 | 218922 | NM_004089.1 | NM_004089 | 1738 |
| IOH10822 | 218924 | BC025791.1 | BC025791 | 27 |
| IOH12648 | 218925 | NM_032125.1 | NM_032125 | 321 |
| IOH12476 | 218926 | NM_022054.2 | NM_022054 | 1467 |
| IOH12165 | 218909 | BC011014.1 | BC011014 | 548 |
| IOH4541 | 219431 | BC001174.1 | BC001174 | 20 |
| IOH22628 | 219415 | BC029032.1 | BC029032 | 254 |
| IOH10380 | 219416 | NM_138792.1 | NM_138792 | 43 |
| IOH22889 | 219417 | NM_005550.2 | NM_005550 | 873 |
| IOH23047 | 219418 | NM_152576.1 | NM_152576 | 4552 |
| IOH5894 | 219419 | NM_000404.1 | NM_000404 | 40 |
| IOH21749 | 219420 | NM_178523.2 | NM_178523 | 4365 |
| IOH22763 | 219422 | BC031661.1 | BC031661 | 297 |
| IOH21756 | 219423 | BC033710.1 | BC033710 | 799 |
| IOH13504 | 219424 | NM_138436.1 | NM_138436 | 1866 |
| IOH6468 | 219425 | NM_000281.1 | NM_000281 | 5369 |
| IOH12235 | 219426 | BC017943.1 | BC017943 | 5366 |
| IOH10509 | 219428 | BC013051.1 | BC013051 | 173 |
| IOH12557 | 218969 | NM_138397.1 | NM_138397 | 354 |
| IOH3444 | 219430 | NM_001819.1 | NM_001819 | 3686 |
| IOH22190 | 219411 | BC031827.1 | BC031827 | 2848 |
| IOH6765 | 219432 | NM_032908.1 | NM_032908 | 5366 |
| IOH12282 | 219435 | BC020867.1 | BC020867 | 238 |
| IOH10009 | 219437 | NM_021218.1 | NM_021218 | 5356 |
| IOH13414 | 219438 | NM_031210.1 | NM_031210 | 833 |
| IOH22940 | 219441 | BC030005.1 | BC030005 | 1281 |
| IOH3500 | 219442 | NM_006831.1 | NM_006831 | 1768 |
| IOH4587 | 219443 | BC000091.1 | BC000091 | 666 |
| IOH21581 | 219444 | BC029568.1 | BC029568 | 5366 |
| IOH22117 | 219447 | BC013103.1 | BC013103 | 187 |
| IOH12990 | 219448 | BC010155.2 | BC010155 | 4457 |
| IOH3154 | 219450 | NM_138386.1 | NM_138386 | 1904 |
| IOH13085 | 218895 | NM_022142.3 | NM_022142 | 1388 |
| IOH22939 | 219429 | BC030636.1 | BC030636 | 196 |
| IOH23129 | 219375 | NM_006519.1 | NM_006519 | 563 |
| IOH22963 | 219452 | NM_002095.1 | NM_002095 | 269 |
| IOH12071 | 218972 | NM_138463.1 | NM_138463 | 316 |
| IOH12646 | 218973 | BC011578.1 | BC011578 | 32 |
| IOH12127 | 218976 | BC021682.1 | BC021682 | 1282 |
| IOH10917 | 218982 | NM_031950.1 | NM_031950 | 82 |
| IOH12659 | 218985 | BC009230.2 | BC009230 | 2579 |
| IOH13888 | 219362 | BC017869.1 | BC017869 | 233 |
| IOH22577 | 219363 | NM_152914.1 | NM_152914 | 5370 |
| IOH6467 | 219365 | BC006370.2 | BC006370 | 2963 |
| IOH22461 | 219367 | NM_153350.2 | NM_153350 | 77 |
| IOH2960 | 219368 | NM_024059.2 | NM_024059 | 271 |
| IOH11667 | 219369 | BC017046.1 | BC017046 | 4183 |
| IOH21844 | 219414 | NM_005423.1 | NM_005423 | 3880 |
| IOH22727 | 219374 | BC029799.1 | BC029799 | 3265 |
| IOH21569 | 219413 | BC028113.1 | BC028113 | 5100 |
| IOH21513 | 219377 | NM_015973.1 | NM_015973 | 808 |
| IOH6669 | 219378 | BC007207.1 | BC007207 | 1242 |
| IOH10913 | 219380 | NM_004567.2 | NM_004567 | 5363 |
| IOH11817 | 219381 | NM_002197.1 | NM_002197 | 907 |
| IOH21704 | 219384 | BC032347.1 | BC032347 | 2255 |
| IOH22492 | 219391 | NM_145028.1 | NM_145028 | 100 |
| IOH3770 | 219395 | BC001669.1 | BC001669 | 35 |
| IOH22121 | 219396 | BC013171.1 | BC013171 | 5359 |
| IOH3092 | 219404 | NM_017512.1 | NM_017512 | 538 |
| IOH3744 | 219407 | BC004159.1 | BC004159 | 76 |
| IOH10277 | 219408 | NM_138491.1 | NM_138491 | 5368 |
| IOH22760 | 219410 | BC031655.1 | BC031655 | 166 |
| IOH11199 | 218970 | BC022471.1 | BC022471 | 576 |
| IOH14733 | 219372 | BC009245.1 | BC009245 | 4144 |

TABLE 8

| AccNumber | Concentration(nM) |
|---|---|
| NM_001893.3 | 163 |
| NM_001894.2 | 396 |
| NM_004196.2 | 88 |
| NM_052987.1 | 29 |
| NM_001826.1 | 3837 |
| NM_016507.1 | 242 |
| NM_020547.1 | 257 |
| NM_015850.2 | 468 |
| NM_023030.1 | 2591 |
| NM_004635.2 | 1338 |
| NM_003137.2 | 41 |
| NM_002576.2 | 68 |
| NM_005030.2 | 140 |
| NM_004071.1 | 253 |
| NM_002748.2 | 4610 |
| NM_002732.2 | 55 |
| NM_001786.2 | 2287 |
| NM_004431.1 | 318 |
| NM_004442.3 | 864 |
| NM_002253.1 | 34 |
| NM_003010.1 | 260 |
| XM_042066.8 | 34 |
| NM_005922.1 | 1851 |
| NM_005923.3 | 125 |
| NM_005965.2 | 129 |
| NM_006254.1 | 82 |
| NM_005400.1 | 121 |
| NM_002731.1 | 52 |
| NM_001654.1 | 22 |
| NM_003688.1 | 1028 |
| NM_004938.1 | 70 |
| NM_002314.2 | 40 |
| NM_002742.1 | 26 |
| NM_002738.2 | 95 |
| NM_001619.2 | 28 |
| NM_003691.1 | 2035 |
| NM_003942.1 | 270 |
| NM_003188.2 | 41 |
| NM_004834.2 | 29 |
| NM_005990.1 | 79 |
| NM_003674.1 | 122 |
| NM_002613.1 | 115 |
| NM_003384.1 | 26 |
| NM_003600.1 | 313 |
| NM_003607.1 | 1096 |
| NM_004586.1 | 32 |
| NM_004217.1 | 72 |
| NM_003242.2 | 1385 |
| NM_002741.1 | 51 |
| NM_006281.1 | 66 |
| NM_006852.1 | 1576 |
| NM_007064.1 | 83 |
| NM_017572.1 | 1485 |
| NM_017593.2 | 491 |
| NM_018401.1 | 61 |
| NM_020397.1 | 3327 |
| NM_021133.1 | 110 |
| NM_018650.1 | 169 |
| NM_021643.1 | 106 |
| NM_003952.1 | 46 |
| NM_005884.2 | 712 |
| NM_013233.1 | 1605 |
| NM_025195.1 | 648 |
| NM_012395.1 | 61 |
| NM_013257.2 | 23 |
| NM_013392.1 | 1064 |
| NM_005465.2 | 75 |
| NM_006035.2 | 80 |
| NM_006282.1 | 145 |
| NM_005813.2 | 41 |
| NM_020168.3 | 42 |
| NM_020328.1 | 64 |
| NM_002752.3 | 46 |
| NM_002754.3 | 200 |
| NM_004383.1 | 149 |
| NM_001259.2 | 138 |
| NM_001892.2 | 113 |
| NM_001106.2 | 126 |
| NM_001896.1 | 81 |
| NM_002756.2 | 274 |
| NM_000061.1 | 113 |
| NM_022972.1 | 92 |
| NM_004445.1 | 19 |
| NM_005235.1 | 334 |
| NM_004443.2 | 138 |
| NM_004560.2 | 211 |
| NM_005157.2 | 182 |
| NM_001616.2 | 135 |
| NM_004441.2 | 65 |
| NM_001982.1 | 43 |
| NM_000459.1 | 31 |
| NM_004444.2 | 85 |
| NM_006343.1 | 846 |
| NM_000075.2 | 512 |
| NM_001258.1 | 614 |
| NM_001261.2 | 49 |
| NM_001799.2 | 122 |
| NM_004935.1 | 1653 |
| BC000479.1 | 738 |
| NM . . . 016440.1 | 834 |
| NM_016735.1 | 118 |
| NM_001203.1 | 4306 |
| NM_{——}005163.1 | 109 |
| NM_005204.2 | 71 |
| NM_005627.1 | 35 |
| NM_002037.1 | 1699 |
| NM_002350.1 | 269 |
| BC001280.1 | 1017 |
| NM_015978.1 | 768 |
| NM_005012.1 | 1192 |
| NM_003576.2 | 830 |
| NM_013254.2 | 324 |
| NM_005417.2 | 24 |
| NM_032409.1 | 732 |
| NM_004103.2 | 22 |
| NM_001396.2 | 165 |
| NM_004226.1 | 1331 |
| NM_015112.1 | 128 |
| NM_005228.1 | 73 |
| NM_006213.1 | 380 |
| NM_005246.1 | 100 |
| NM_014920.1 | 1369 |
| NM_005906.2 | 768 |
| NM_033115.1 | 595 |
| NM_012424.2 | 38 |
| NM_004759.2 | 148 |
| NM_006622.1 | 361 |
| NM_014002.1 | 341 |
| NM_014496.1 | 190 |
| NM_007194.1 | 740 |
| NM_002745.2 | 30 |
| NM_002447.1 | 146 |
| NM_013355.1 | 400 |
| NM_032844.1 | 753 |
| NM_006258.1 | 32 |
| NM_017719.2 | 45 |
| NM_031414.2 | 3208 |
| NM_001626.2 | 26 |
| NM_006256.1 | 2434 |
| NM_018423.1 | 59 |
| NM_032237.1 | 701 |
| NM_002750.2 | 61 |
| NM_002578.1 | 42 |
| BC001662.1 | 35 |
| BC017715.1 | 259 |
| BC001274.1 | 1282 |
| BC000442.1 | 42 |
| BC006106.1 | 25 |
| NM_003948.2 | 113 |
| BC003614.1 | 69 |
| NM_002744.2 | 23 |
| BC005408.1 | 587 |
| NM_033621.1 | 232 |
| BC008302.1 | 179 |
| BC000471.1 | 22 |
| BC002541.1 | 31 |
| BC002755.1 | 265 |
| BC008716.1 | 20 |
| BC001968.1 | 63 |
| BC008838.1 | 961 |
| BC000251.1 | 23 |
| BC002637.1 | 2652 |
| BC016652.1 | 39 |
| BC012761.1 | 36 |
| BC008726.1 | 852 |
| BC020972.1 | 27 |
| BC011668.1 | 41 |
| BC004207.1 | 24 |
| BC003065.1 | 175 |
| BC002695.1 | 39 |
| BC018111_1 | 30 |
| BC013879.1 | 641 |
| NM_018492.2 | 62 |
| NM_024776.1 | 2328 |
| NM . . . 024800.1 | 189 |
| BC014037.1 | 40 |

TABLE 15

| Accno | Description |
|---|---|
| NM_000023.1 | >gi\|4506910\|ref\|NM_000023.1\| Homo sapiens sarcoglycan, alpha |
| | (50 kDa dystrophin-associated glycoprotein) (SGCA), mRNA |
| NM_000024.3 | >gi\|15718673\|ref\|NM_000024.3\| Homo sapiens adrenergic, beta-2-, |
| | receptor, surface (ADRB2), mRNA |
| NM_000033.2 | >gi\|7262392\|ref\|NM_000033.2\| Homo sapiens ATP-binding cassette, |
| | sub-family D (ALD), member 1 (ABCD1), mRNA |
| NM_000061.1 | >gi\|4557376\|ref\|NM_000061.1\| Homo sapiens Bruton |
| | agammaglobulinemia tyrosine kinase (BTK), mRNA |
| NM_000160.1 | >gi\|4503946\|ref\|NM_000160.1\| Homo sapiens glucagon receptor |
| | (GCGR), mRNA |
| NM_000166.2 | >gi\|31542846\|ref\|NM_000166.2\| Homo sapiens gap junction protein, |
| | beta 1, 32 kDa (connexin 32, Charcot-Marie-Tooth neuropathy, X- |
| | linked) (GJB1), mRNA |
| NM_000198.2 | >gi\|50726973\|ref\|NM_000198.2\| Homo sapiens hydroxy-delta-5- |
| | steroid dehydrogenase, 3 beta- and steroid delta-isomerase 2 |
| | (HSD3B2), mRNA |
| NM_000206.1 | >gi\|4557881\|ref\|NM_000206.1\| Homo sapiens interleukin 2 receptor, |
| | gamma (severe combined immunodeficiency) (IL2RG), mRNA |
| NM_000208.1 | >gi\|4557883\|ref\|NM_000208.1\| Homo sapiens insulin receptor |
| | (INSR), mRNA |
| NM_000231.1 | >gi\|4557846\|ref\|NM_000231.1\| Homo sapiens sarcoglycan, gamma |
| | (35 kDa dystrophin-associated glycoprotein) (SGCG), mRNA |
| NM_000286.1 | >gi\|4505720\|ref\|NM_000286.1\| Homo sapiens peroxisomal |
| | biogenesis factor 12 (PEX12), mRNA |
| NM_000327.2 | >gi\|19743809\|ref\|NM_000327.2\| Homo sapiens retinal outer segment |
| | membrane protein 1 (ROM1), mRNA |
| NM_000341.2 | >gi\|23308570\|ref\|NM_000341.2\| Homo sapiens solute carrier family |
| | 3 (cystine, dibasic and neutral amino acid transporters, activator of |
| | cystine, dibasic and neutral amino acid transport), member 1 |
| | (SLC3A1), mRNA |
| NM_000359.1 | >gi\|4507474\|ref\|NM_000359.1\| Homo sapiens transglutaminase 1 (K |
| | polypeptide epidermal type I, protein-glutamine-gamma- |
| | glutamyltransferase) (TGM1), mRNA |
| NM_000387.2 | >gi\|6006040\|ref\|NM_000387.2\| Homo sapiens solute carrier family |
| | 25 (carnitine/acylcarnitine translocase), member 20 (SLC25A20), |
| | nuclear gene encoding mitochondrial protein, mRNA |
| NM_000398.3 | >gi\|6552326\|ref\|NM_000398.3\| Homo sapiens diaphorase (NADH) |
| | (cytochrome b-5 reductase) (DIA1), transcript variant M, mRNA |
| NM_000430.2 | >gi\|6031206\|ref\|NM_000430.2\| Homo sapiens platelet-activating |
| | factor acetylhydrolase, isoform Ib, alpha subunit 45 kDa |
| | (PAFAH1B1), mRNA |
| NM_000442.2 | >gi\|21314616\|ref\|NM_000442.2\| Homo sapiens platelet/endothelial |
| | cell adhesion molecule (CD31 antigen) (PECAM1), mRNA |
| NM_000459.1 | >gi\|4557868\|ref\|NM_000459.1\| Homo sapiens TEK tyrosine kinase, |
| | endothelial (venous malformations, multiple cutaneous and mucosal) |
| | (TEK), mRNA |
| NM_000486.2 | >gi\|4755122\|ref\|NM_000486.2\| Homo sapiens aquaporin 2 (collecting |
| | duct) (AQP2), mRNA |
| NM_000499.2 | >gi\|13325053\|ref\|NM_000499.2\| Homo sapiens cytochrome P450, |
| | family 1, subfamily A, polypeptide 1 (CYP1A1), mRNA |
| NM_000594.2 | >gi\|25952110\|ref\|NM_000594.2\| Homo sapiens tumor necrosis factor |
| | (TNF superfamily, member 2) (TNF), mRNA |
| NM_000626.1 | >gi\|11038673\|ref\|NM_000626.1\| Homo sapiens CD79B antigen |
| | (immunoglobulin-associated beta) (CD79B), transcript variant 1, |
| | mRNA |
| NM_000675.3 | >gi\|17136146\|ref\|NM_000675.3\| Homo sapiens adenosine A2a |
| | receptor (ADORA2A), mRNA |
| NM_000714.3 | >gi\|21536444\|ref\|NM_000714.3\| Homo sapiens benzodiazapine |
| | receptor (peripheral) (BZRP), transcript variant PBR, mRNA |
| NM_000732.3 | >gi\|55775475\|ref\|NM_000732.3\| Homo sapiens CD3D antigen, delta |
| | polypeptide (TiT3 complex) (CD3D), mRNA |
| NM_000754.2 | >gi\|6466451\|ref\|NM_000754.2\| Homo sapiens catechol-O- |
| | methyltransferase (COMT), transcript variant MB-COMT, mRNA |
| NM_000764.2 | >gi\|15147329\|ref\|NM_000764.2\| Homo sapiens cytochrome P450, |
| | family 2, subfamily A, polypeptide 7 (CYP2A7), transcript variant 1, |
| | mRNA |
| NM_000810.2 | >gi\|6031207\|ref\|NM_000810.2\| Homo sapiens gamma-aminobutyric |
| | acid (GABA) A receptor, alpha 5 (GABRA5), mRNA |
| NM_000866.1 | >gi\|10835196\|ref\|NM_000866.1\| Homo sapiens 5-hydroxytryptamine |
| | (serotonin) receptor 1F (HTR1F), mRNA |
| NM_000910.1 | >gi\|4505446\|ref\|NM_000910.1\| Homo sapiens neuropeptide Y |
| | receptor Y2 (NPY2R), mRNA |
| NM_000953.2 | >gi\|38505191\|ref\|NM_000953.2\| Homo sapiens prostaglandin D2 |
| | receptor (DP) (PTGDR), mRNA |
| NM_000963.1 | >gi\|4506264\|ref\|NM_000963.1\| Homo sapiens prostaglandin- |
| | endoperoxide synthase 2 (prostaglandin G/H synthase and |
| | cyclooxygenase) (PTGS2), mRNA |
| NM_001013.2 | >gi\|14141192\|ref\|NM_001013.2\| Homo sapiens ribosomal protein S9 |
| | (RPS9), mRNA |
| NM_001014.2 | >gi\|13904867\|ref\|NM_001014.2\| Homo sapiens ribosomal protein |
| | S10 (RPS10), mRNA |
| NM_001015.2 | >gi\|14277698\|ref\|NM_001015.2\| Homo sapiens ribosomal protein |
| | S11 (RPS11), mRNA |
| NM_001016.2 | >gi\|14277699\|ref\|NM_001016.2\| Homo sapiens ribosomal protein |
| | S12 (RPS12), mRNA |
| NM_001018.2 | >gi\|14591911\|ref\|NM_001018.2\| Homo sapiens ribosomal protein |
| | S15 (RPS15), mRNA |
| NM_001020.2 | >gi\|14591912\|ref\|NM_001020.2\| Homo sapiens ribosomal protein |
| | S16 (RPS16), mRNA |
| NM_001021.2 | >gi\|14591913\|ref\|NM_001021.2\| Homo sapiens ribosomal protein |
| | S17 (RPS17), mRNA |
| NM_001022.3 | >gi\|48255921\|ref\|NM_001022.3\| Homo sapiens ribosomal protein |
| | S19 (RPS19), mRNA |
| NM_001023.2 | >gi\|14591915\|ref\|NM_001023.2\| Homo sapiens ribosomal protein |
| | S20 (RPS20), mRNA |
| NM_001042.1 | >gi\|4507010\|ref\|NM_001042.1\| Homo sapiens solute carrier family 2 |
| | (facilitated glucose transporter), member 4 (SLC2A4), mRNA |
| NM_001047.1 | >gi\|4507200\|ref\|NM_001047.1\| Homo sapiens steroid-5-alpha- |
| | reductase, alpha polypeptide 1 (3-oxo-5 alpha-steroid delta 4- |
| | dehydrogenase alpha 1) (SRD5A1), mRNA |
| NM_001084.2 | >gi\|21361165\|ref\|NM_001084.2\| Homo sapiens procollagen-lysine, 2- |
| | oxoglutarate 5-dioxygenase 3 (PLOD3), mRNA |
| NM_001086.1 | >gi\|4557226\|ref\|NM_001086.1\| Homo sapiens arylacetamide |
| | deacetylase (esterase) (AADAC), mRNA |
| NM_001094.4 | >gi\|34452696\|ref\|NM_001094.4\| Homo sapiens amiloride-sensitive |
| | cation channel 1, neuronal (degenerin) (ACCN1), transcript variant 2, |
| | mRNA |
| NM_001106.2 | >gi\|10862697\|ref\|NM_001106.2\| Homo sapiens activin A receptor, |
| | type IIB (ACVR2B), mRNA |
| NM_001150.1 | >gi\|4502094\|ref\|NM_001150.1\| Homo sapiens alanyl (membrane) |
| | aminopeptidase (aminopeptidase N, aminopeptidase M, microsomal |
| | aminopeptidase, CD13, p150) (ANPEP), mRNA |
| NM_001183.3 | >gi\|37577154\|ref\|NM_001183.3\| Homo sapiens ATPase, H+ |
| | transporting, lysosomal accessory protein 1 (ATP6AP1), mRNA |
| NM_001203.1 | >gi\|4502430\|ref\|NM_001203.1\| Homo sapiens bone morphogenetic |
| | protein receptor, type IB (BMPR1B), mRNA |
| NM_001216.1 | >gi\|9955947\|ref\|NM_001216.1\| Homo sapiens carbonic anhydrase IX |
| | (CA9), mRNA |
| NM_001296.3 | >gi\|37577160\|ref\|NM_001296.3\| Homo sapiens chemokine binding |
| | protein 2 (CCBP2), mRNA |
| NM_001306.2 | >gi\|21536298\|ref\|NM_001306.2\| Homo sapiens claudin 3 (CLDN3), |
| | mRNA |
| NM_001307.1 | >gi\|10835007\|ref\|NM_001307.1\| Homo sapiens claudin 7 (CLDN7), |
| | mRNA |
| NM_001361.1 | >gi\|16753222\|ref\|NM_001361.1\| Homo sapiens dihydroorotate |
| | dehydrogenase (DHODH), nuclear gene encoding mitochondrial |
| | protein, mRNA |
| NM_001425.1 | >gi\|4503562\|ref\|NM_001425.1\| Homo sapiens epithelial membrane |
| | protein 3 (EMP3), mRNA |
| NM_001448.2 | >gi\|21614524\|ref\|NM_001448.2\| Homo sapiens glypican 4 (GPC4), |
| | mRNA |
| NM_001544.2 | >gi\|12545400\|ref\|NM_001544.2\| Homo sapiens intercellular adhesion |
| | molecule 4, Landsteiner-Wiener blood group (ICAM4), transcript |
| | variant 1, mRNA |
| NM_001561.3 | >gi\|20070135\|ref\|NM_001561.3\| Homo sapiens tumor necrosis factor |
| | receptor superfamily, member 9 (TNFRSF9), mRNA |
| NM_001611.2 | >gi\|6138970\|ref\|NM_001611.2\| Homo sapiens acid phosphatase 5, |
| | tartrate resistant (ACP5), mRNA |
| NM_001616.2 | >gi\|10862696\|ref\|NM_001616.2\| Homo sapiens activin A receptor, |
| | type II (ACVR2), mRNA |
| NM_001629.2 | >gi\|15718674\|ref\|NM_001629.2\| Homo sapiens arachidonate 5- |
| | lipoxygenase-activating protein (ALOX5AP), mRNA |
| NM_001633.1 | >gi\|4502066\|ref\|NM_001633.1\| Homo sapiens alpha-1- |
| | microglobulin/bikunin precursor (AMBP), mRNA |
| NM_001636.1 | >gi\|27764862\|ref\|NM_001636.1\| Homo sapiens solute carrier family |
| | 25 (mitochondrial carrier; adenine nucleotide translocator), member 6 |
| | (SLC25A6), mRNA |
| NM_001637.1 | >gi\|4502114\|ref\|NM_001637.1\| Homo sapiens acyloxyacyl hydrolase |
| | (neutrophil) (AOAH), mRNA |
| NM_001664.1 | >gi\|10835048\|ref\|NM_001664.1\| Homo sapiens ras homolog gene |
| | family, member A (ARHA), mRNA |
| NM_001671.2 | >gi\|18426870\|ref\|NM_001671.2\| Homo sapiens asialoglycoprotein |
| | receptor 1 (ASGR1), mRNA |
| NM_001679.1 | >gi\|4502280\|ref\|NM_001679.1\| Homo sapiens ATPase, Na+/K+ |
| | transporting, beta 3 polypeptide (ATP1B3), mRNA |
| NM_001680.2 | >gi\|11125765\|ref\|NM_001680.2\| Homo sapiens FXYD domain |
| | containing ion transport regulator 2 (FXYD2), transcript variant a, |
| | mRNA |
| NM_001686.3 | >gi\|50345985\|ref\|NM_001686.3\| Homo sapiens ATP synthase, H+ |
| | transporting, mitochondrial F1 complex, beta polypeptide (ATP5B), |
| | nuclear gene encoding mitochondrial protein, mRNA |
| NM_001688.2 | >gi\|21361564\|ref\|NM_001688.2\| Homo sapiens ATP synthase, H+ |
| | transporting, mitochondrial F0 complex, subunit b, isoform 1 |
| | (ATP5F1), mRNA |
| NM_001696.2 | >gi\|19913433\|ref\|NM_001696.2\| Homo sapiens ATPase, H+ |
| | transporting, lysosomal 31 kDa, V1 subunit E isoform 1 (ATP6V1E1), |
| | mRNA |
| NM_001697.1 | >gi\|4502302\|ref\|NM_001697.1\| Homo sapiens ATP synthase, H+ |
| | transporting, mitochondrial F1 complex, O subunit (oligomycin |
| | sensitivity conferring protein) (ATP5O), mRNA |
| NM_001736.1 | >gi\|4502508\|ref\|NM_001736.1\| Homo sapiens complement |
| | component 5 receptor 1 (C5a ligand) (C5R1), mRNA |
| NM_001769.2 | >gi\|21237762\|ref\|NM_001769.2\| Homo sapiens CD9 antigen (p24) |
| | (CD9), mRNA |
| NM_001803.1 | >gi\|4502760\|ref\|NM_001803.1\| Homo sapiens CDW52 antigen |
| | (CAMPATH-1 antigen) (CDW52), mRNA |
| NM_001841.1 | >gi\|4502928\|ref\|NM_001841.1\| Homo sapiens cannabinoid receptor 2 |
| | (macrophage) (CNR2), mRNA |
| NM_001860.1 | >gi\|4507016\|ref\|NM_001860.1\| Homo sapiens solute carrier family |
| | 31 (copper transporters), member 2 (SLC31A2), mRNA |
| NM_001935.2 | >gi\|18765693\|ref\|NM_001935.2\| Homo sapiens dipeptidylpeptidase 4 |
| | (CD26, adenosine deaminase complexing protein 2) (DPP4), mRNA |
| NM_001993.2 | >gi\|10518499\|ref\|NM_001993.2\| Homo sapiens coagulation factor III |
| | (thromboplastin, tissue factor) (F3), mRNA |
| NM_002045.1 | >gi\|4503910\|ref\|NM_002045.1\| Homo sapiens growth associated |
| | protein 43 (GAP43), mRNA |
| NM_002068.1 | >gi\|4504038\|ref\|NM_002068.1\| Homo sapiens guanine nucleotide |
| | binding protein (G protein), alpha 15 (Gq class) (GNA15), mRNA |
| NM_002072.1 | >gi\|4504044\|ref\|NM_002072.1\| Homo sapiens guanine nucleotide |
| | binding protein (G protein), q polypeptide (GNAQ), mRNA |
| NM_002233.1 | >gi\|4504816\|ref\|NM_002233.1\| Homo sapiens potassium voltage- |
| | gated channel, shaker-related subfamily, member 4 (KCNA4), |
| | mRNA |
| NM_002242.2 | >gi\|34222089\|ref\|NM_002242.2\| Homo sapiens potassium inwardly- |
| | rectifying channel, subfamily J, member 13 (KCNJ13), mRNA |
| NM_002262.2 | >gi\|7669497\|ref\|NM_002262.2\| Homo sapiens killer cell lectin-like |
| | receptor subfamily D, member 1 (KLRD1), transcript variant 1, |
| | mRNA |
| NM_002413.3 | >gi\|22035639\|ref\|NM_002413.3\| Homo sapiens microsomal |
| | glutathione S-transferase 2 (MGST2), mRNA |
| NM_002436.2 | >gi\|6006024\|ref\|NM_002436.2\| Homo sapiens membrane protein, |
| | palmitoylated 1, 55 kDa (MPP1), mRNA |
| NM_002447.1 | >gi\|4505264\|ref\|NM_002447.1\| Homo sapiens macrophage |
| | stimulating 1 receptor (c-met-related tyrosine kinase) (MST1R), |
| | mRNA |
| NM_002558.2 | >gi\|27894283\|ref\|NM_002558.2\| Homo sapiens purinergic receptor |
| | P2X, ligand-gated ion channel, 1 (P2RX1), mRNA |
| NM_002608.1 | >gi\|4505680\|ref\|NM_002608.1\| Homo sapiens platelet-derived |
| | growth factor beta polypeptide (simian sarcoma viral (v-sis) |
| | oncogene homolog) (PDGFB), transcript variant 1, mRNA |
| NM_002617.3 | >gi\|24797087\|ref\|NM_002617.3\| Homo sapiens peroxisome |
| | biogenesis factor 10 (PEX10), transcript variant 2, mRNA |
| NM_002618.2 | >gi\|46047483\|ref\|NM_002618.2\| Homo sapiens peroxisome |
| | biogenesis factor 13 (PEX13), mRNA |
| NM_002688.2 | >gi\|9945438\|ref\|NM_002688.2\| Homo sapiens peanut-like 1 |
| | (Drosophila) (PNUTL1), mRNA |
| NM_002870.1 | >gi\|4506362\|ref\|NM_002870.1\| Homo sapiens RAB13, member RAS |
| | oncogene family (RAB13), mRNA |
| NM_002880.1 | >gi\|4506400\|ref\|NM_002880.1\| Homo sapiens v-raf-1 murine |
| | leukemia viral oncogene homolog 1 (RAF1), mRNA |
| NM_002922.2 | >gi\|21361446\|ref\|NM_002922.2\| Homo sapiens regulator of G- |
| | protein signalling 1 (RGS1), mRNA |
| NM_002944.2 | >gi\|19924164\|ref\|NM_002944.2\| Homo sapiens v-ros UR2 sarcoma |
| | virus oncogene homolog 1 (avian) (ROS1), mRNA |
| NM_002982.1 | >gi\|4506840\|ref\|NM_002982.1\| Homo sapiens small inducible |
| | cytokine A2 (monocyte chemotactic protein 1) (SCYA2), mRNA |
| NM_003002.1 | >gi\|4506864\|ref\|NM_003002.1\| Homo sapiens succinate |
| | dehydrogenase complex, subunit D, integral membrane protein |
| | (SDHD), nuclear gene encoding mitochondrial protein, mRNA |
| NM_003004.1 | >gi\|4506868\|ref\|NM_003004.1\| Homo sapiens secreted and |
| | transmembrane 1 (SECTM1), mRNA |
| NM_003006.2 | >gi\|6031197\|ref\|NM_003006.2\| Homo sapiens selectin P ligand |
| | (SELPLG), mRNA |
| NM_003045.3 | >gi\|51173150\|ref\|NM_003045.3\| Homo sapiens solute carrier family |
| | 7 (cationic amino acid transporter, y+ system), member 1 (SLC7A1), |
| | mRNA |
| NM_003049.1 | >gi\|4506970\|ref\|NM_003049.1\| Homo sapiens solute carrier family |
| | 10 (sodium/bile acid cotransporter family), member 1 (SLC10A1), |
| | mRNA |
| NM_003123.1 | >gi\|4507180\|ref\|NM_003123.1\| Homo sapiens sialophorin (gpL115, |
| | leukosialin, CD43) (SPN), mRNA |
| NM_003192.1 | >gi\|4507372\|ref\|NM_003192.1\| Homo sapiens tubulin-specific |
| | chaperone c (TBCC), mRNA |
| NM_003193.2 | >gi\|6006029\|ref\|NM_003193.2\| Homo sapiens tubulin-specific |
| | chaperone e (TBCE), mRNA |
| NM_003277.1 | >gi\|4502878\|ref\|NM_003277.1\| Homo sapiens claudin 5 |
| | (transmembrane protein deleted in velocardiofacial syndrome) |
| | (CLDN5), mRNA |
| NM_003358.1 | >gi\|4507810\|ref\|NM_003358.1\| Homo sapiens UDP-glucose |
| | ceramide glucosyltransferase (UGCG), mRNA |
| NM_003374.1 | >gi\|4507878\|ref\|NM_003374.1\| Homo sapiens voltage-dependent |
| | anion channel 1 (VDAC1), mRNA |
| NM_003382.2 | >gi\|21361556\|ref\|NM_003382.2\| Homo sapiens vasoactive intestinal |
| | peptide receptor 2 (VIPR2), mRNA |
| NM_003498.2 | >gi\|19923171\|ref\|NM_003498.2\| Homo sapiens stannin (SNN), |
| | mRNA |
| NM_003562.2 | >gi\|21361113\|ref\|NM_003562.2\| Homo sapiens solute carrier family |
| | 25 (mitochondrial carrier; oxoglutarate carrier), member 11 |
| | (SLC25A11), mRNA |
| NM_003630.1 | >gi\|4505726\|ref\|NM_003630.1\| Homo sapiens peroxisomal |
| | biogenesis factor 3 (PEX3), mRNA |
| NM_003712.1 | >gi\|4505976\|ref\|NM_003712.1\| Homo sapiens phosphatidic acid |
| | phosphatase type 2C (PPAP2C), mRNA |
| NM_003720.1 | >gi\|4505022\|ref\|NM_003720.1\| Homo sapiens Down syndrome |
| | critical region gene 2 (DSCR2), mRNA |
| NM_003764.2 | >gi\|33667037\|ref\|NM_003764.2\| Homo sapiens syntaxin 11 (STX11), |
| | mRNA |
| NM_003801.2 | >gi\|6031166\|ref\|NM_003801.2\| Homo sapiens GPAA1P anchor |
| | attachment protein 1 homolog (yeast) (GPAA1), mRNA |
| NM_003818.2 | >gi\|22035625\|ref\|NM_003818.2\| Homo sapiens CDP-diacylglycerol |
| | synthase (phosphatidate cytidylyltransferase) 2 (CDS2), mRNA |
| NM_003847.1 | >gi\|4505716\|ref\|NM_003847.1\| Homo sapiens peroxisomal |
| | biogenesis factor 11A (PEX11A), mRNA |
| NM_003853.2 | >gi\|27477087\|ref\|NM_003853.2\| Homo sapiens interleukin 18 |
| | receptor accessory protein (IL18RAP), mRNA |
| NM_003874.1 | >gi\|4502686\|ref\|NM_003874.1\| Homo sapiens CD84 antigen |
| | (leukocyte antigen) (CD84), mRNA |
| NM_003897.2 | >gi\|16554595\|ref\|NM_003897.2\| Homo sapiens immediate early |
| | response 3 (IER3), transcript variant short, mRNA |
| NM_004001.3 | >gi\|50511929\|ref\|NM_004001.3\| Homo sapiens Fc fragment of IgG, |
| | low affinity IIb, receptor (CD32) (FCGR2B), transcript variant 1, |
| | mRNA |
| NM_004070.2 | >gi\|31542310\|ref\|NM_004070.2\| Homo sapiens chloride channel Ka |
| | (CLCNKA), mRNA |
| NM_004148.2 | >gi\|31543289\|ref\|NM_004148.2\| Homo sapiens ninjurin 1 (NINJ1), |
| | mRNA |
| NM_004177.3 | >gi\|34147491\|ref\|NM_004177.3\| Homo sapiens syntaxin 3A |
| | (STX3A), mRNA |
| NM_004271.1 | >gi\|4758707\|ref\|NM_004271.1\| Homo sapiens lymphocyte antigen 86 |
| | (LY86), mRNA |
| NM_004304.3 | >gi\|29029631\|ref\|NM_004304.3\| Homo sapiens anaplastic lymphoma |
| | kinase (Ki-1) (ALK), mRNA |
| NM_004331.1 | >gi\|4757859\|ref\|NM_004331.1\| Homo sapiens BCL2/adenovirus E1B |
| | 19 kDa interacting protein 3-like (BNIP3L), mRNA |
| NM_004362.1 | >gi\|4758003\|ref\|NM_004362.1\| Homo sapiens calmegin (CLGN), |
| | mRNA |
| NM_004373.2 | >gi\|17999527\|ref\|NM_004373.2\| Homo sapiens cytochrome c oxidase |
| | subunit VIa polypeptide 1 (COX6A1), nuclear gene encoding |
| | mitochondrial protein, mRNA |
| NM_004382.2 | >gi\|19923244\|ref\|NM_004382.2\| Homo sapiens corticotropin |
| | releasing hormone receptor 1 (CRHR1), mRNA |
| NM_004383.1 | >gi\|4758077\|ref\|NM_004383.1\| Homo sapiens c-src tyrosine kinase |
| | (CSK), mRNA |
| NM_004431.1 | >gi\|4758277\|ref\|NM_004431.1\| Homo sapiens EphA2 (EPHA2), |
| | mRNA |
| NM_004438.1 | >gi\|4758279\|ref\|NM_004438.1\| Homo sapiens EphA4 (EPHA4), |
| | mRNA |
| NM_004439.4 | >gi\|56119208\|ref\|NM_004439.4\| Homo sapiens EPH receptor A5 |
| | (EPHA5), transcript variant 1, mRNA |
| NM_004440.1 | >gi\|4758281\|ref\|NM_004440.1\| Homo sapiens EphA7 (EPHA7), |
| | mRNA |
| NM_004441.2 | >gi\|21396502\|ref\|NM_004441.2\| Homo sapiens EphB1 (EPHB1), |
| | mRNA |
| NM_004441.3 | >gi\|55770893\|ref\|NM_004441.3\| Homo sapiens EPH receptor B1 |
| | (EPHB1), mRNA |
| NM_004442.3 | >gi\|21396503\|ref\|NM_004442.3\| Homo sapiens EphB2 (EPHB2), |
| | transcript variant 1, mRNA |
| NM_004443.2 | >gi\|17975767\|ref\|NM_004443.2\| Homo sapiens EphB3 (EPHB3), |
| | mRNA |
| NM_004444.2 | >gi\|17975769\|ref\|NM_004444.2\| Homo sapiens EphB4 (EPHB4), |
| | mRNA |
| NM_004445.1 | >gi\|4758291\|ref\|NM_004445.1\| Homo sapiens EphB6 (EPHB6), |
| | mRNA |
| NM_004469.2 | >gi\|19924297\|ref\|NM_004469.2\| Homo sapiens c-fos induced growth |
| | factor (vascular endothelial growth factor D) (FIGF), mRNA |
| NM_004475.1 | >gi\|4758393\|ref\|NM_004475.1\| Homo sapiens flotillin 2 (FLOT2), |
| | mRNA |
| NM_004485.2 | >gi\|21314630\|ref\|NM_004485.2\| Homo sapiens guanine nucleotide |
| | binding protein (G protein), gamma 4 (GNG4), mRNA |
| NM_004512.3 | >gi\|22212920\|ref\|NM_004512.3\| Homo sapiens interleukin 11 |
| | receptor, alpha (IL11RA), transcript variant 1, mRNA |
| NM_004528.2 | >gi\|22035640\|ref\|NM_004528.2\| Homo sapiens microsomal |
| | glutathione S-transferase 3 (MGST3), mRNA |
| NM_004542.1 | >gi\|4758771\|ref\|NM_004542.1\| Homo sapiens NADH dehydrogenase |
| | (ubiquinone) 1 alpha subcomplex, 3, 9 kDa (NDUFA3), mRNA |
| NM_004549.2 | >gi\|19923255\|ref\|NM_004549.2\| Homo sapiens NADH |
| | dehydrogenase (ubiquinone) 1, subcomplex unknown, 2, 14.5 kDa |
| | (NDUFC2), mRNA |
| NM_004588.3 | >gi\|56699490\|ref\|NM_004588.3\| Homo sapiens sodium channel, |
| | voltage-gated, type II, beta (SCN2B), mRNA |
| NM_004617.2 | >gi\|21265109\|ref\|NM_004617.2\| Homo sapiens transmembrane 4 |
| | superfamily member 4 (TM4SF4), mRNA |
| NM_004695.2 | >gi\|20127461\|ref\|NM_004695.2\| Homo sapiens solute carrier family |
| | 16 (monocarboxylic acid transporters), member 5 (SLC16A5), |
| | mRNA |
| NM_004710.2 | >gi\|22091455\|ref\|NM_004710.2\| Homo sapiens synaptogyrin 2 |
| | (SYNGR2), mRNA |
| NM_004720.3 | >gi\|11038657\|ref\|NM_004720.3\| Homo sapiens endothelial |
| | differentiation, lysophosphatidic acid G-protein-coupled receptor, 4 |
| | (EDG4), mRNA |
| NM_004722.2 | >gi\|14917110\|ref\|NM_004722.2\| Homo sapiens adaptor-related |
| | protein complex 4, mu 1 subunit (AP4M1), mRNA |
| NM_004732.1 | >gi\|4758619\|ref\|NM_004732.1\| Homo sapiens potassium voltage- |
| | gated channel, shaker-related subfamily, beta member 3 (KCNAB3), |
| | mRNA |
| NM_004738.1 | >gi\|4759301\|ref\|NM_004738.1\| Homo sapiens VAMP (vesicle- |
| | associated membrane protein)-associated protein B and C (VAPB), |
| | mRNA |
| NM_004766.1 | >gi\|4758031\|ref\|NM_004766.1\| Homo sapiens coatomer protein |
| | complex, subunit beta 2 (beta prime) (COPB2), mRNA |
| NM_004781.2 | >gi\|9257252\|ref\|NM_004781.2\| Homo sapiens vesicle-associated |
| | membrane protein 3 (cellubrevin) (VAMP3), mRNA |
| NM_004809.3 | >gi\|31543664\|ref\|NM_004809.3\| Homo sapiens stomatin (EPB72)- |
| | like 1 (STOML1), mRNA |
| NM_004813.1 | >gi\|4758897\|ref\|NM_004813.1\| Homo sapiens peroxisomal |
| | biogenesis factor 16 (PEX16), transcript variant 1, mRNA |
| NM_004853.1 | >gi\|4759187\|ref\|NM_004853.1\| Homo sapiens syntaxin 8 (STX8), |
| | mRNA |
| NM_004867.2 | >gi\|33589836\|ref\|NM_004867.2\| Homo sapiens integral membrane |
| | protein 2A (ITM2A), mRNA |
| NM_004869.2 | >gi\|17865801\|ref\|NM_004869.2\| Homo sapiens vacuolar protein |
| | sorting 4B (yeast) (VPS4B), mRNA |
| NM_004872.2 | >gi\|20070190\|ref\|NM_004872.2\| Homo sapiens chromosome 1 open |
| | reading frame 8 (C1orf8), mRNA |
| NM_004892.2 | >gi\|14591922\|ref\|NM_004892.2\| Homo sapiens SEC22 vesicle |
| | trafficking protein-like 1 (S. cerevisiae) (SEC22L1), mRNA |
| NM_004952.3 | >gi\|33359683\|ref\|NM_004952.3\| Homo sapiens ephrin-A3 (EFNA3), |
| | mRNA |
| NM_004955.1 | >gi\|4826715\|ref\|NM_004955.1\| Homo sapiens solute carrier family |
| | 29 (nucleoside transporters), member 1 (SLC29A1), mRNA |
| NM_005086.3 | >gi\|16933560\|ref\|NM_005086.3\| Homo sapiens sarcospan (Kras |
| | oncogene-associated gene) (SSPN), mRNA |
| NM_005092.2 | >gi\|40354198\|ref\|NM_005092.2\| Homo sapiens tumor necrosis factor |
| | (ligand) superfamily, member 18 (TNFSF18), mRNA |
| NM_005201.2 | >gi\|13929430\|ref\|NM_005201.2\| Homo sapiens chemokine (C—C |
| | motif) receptor 8 (CCR8), mRNA |
| NM_005205.2 | >gi\|17999529\|ref\|NM_005205.2\| Homo sapiens cytochrome c oxidase |
| | subunit VIa polypeptide 2 (COX6A2), nuclear gene encoding |
| | mitochondrial protein, mRNA |
| NM_005226.2 | >gi\|38788192\|ref\|NM_005226.2\| Homo sapiens endothelial |
| | differentiation, sphingolipid G-protein-coupled receptor, 3 (EDG3), |
| | mRNA |
| NM_005232.1 | >gi\|4885208\|ref\|NM_005232.11\| Homo sapiens EphA1 (EPHA1), |
| | mRNA |
| NM_005233.2 | >gi\|21361240\|ref\|NM_005233.2\| Homo sapiens EphA3 (EPHA3), |
| | mRNA |
| NM_005268.1 | >gi\|10835078\|ref\|NM_005268.1\| Homo sapiens gap junction protein, |
| | beta 5 (connexin 31.1) (GJB5), mRNA |
| NM_005272.2 | >gi\|22027523\|ref\|NM_005272.2\| Homo sapiens guanine nucleotide |
| | binding protein (G protein), alpha transducing activity polypeptide 2 |
| | (GNAT2), mRNA |
| NM_005274.1 | >gi\|4885286\|ref\|NM_005274.1\| Homo sapiens guanine nucleotide |
| | binding protein (G protein), gamma 5 (GNG5), mRNA |
| NM_005283.1 | >gi\|4885338\|ref\|NM_005283.1\| Homo sapiens chemokine (C motif) |
| | receptor 1 (XCR1), mRNA |
| NM_005290.1 | >gi\|4885298\|ref\|NM_005290.1\| Homo sapiens G protein-coupled |
| | receptor 15 (GPR15), mRNA |
| NM_005294.1 | >gi\|4885306\|ref\|NM_005294.1\| Homo sapiens G protein-coupled |
| | receptor 21 (GPR21), mRNA |
| NM_005299.1 | >gi\|4885316\|ref\|NM_005299.1\| Homo sapiens G protein-coupled |
| | receptor 31 (GPR31), mRNA |
| NM_005333.1 | >gi\|4885400\|ref\|NM_005333.1\| Homo sapiens holocytochrome c |
| | synthase (cytochrome c heme-lyase) (HCCS), mRNA |
| NM_005441.2 | >gi\|45827788\|ref\|NM_005441.2\| Homo sapiens chromatin assembly |
| | factor 1, subunit B (p60) (CHAF1B), mRNA |
| NM_005506.1 | >gi\|5031630\|ref\|NM_005506.1\| Homo sapiens scavenger receptor |
| | class B, member 2 (SCARB2), mRNA |
| NM_005567.2 | >gi\|6006016\|ref\|NM_005567.2\| Homo sapiens lectin, galactoside- |
| | binding, soluble, 3 binding protein (LGALS3BP), mRNA |
| NM_005592.1 | >gi\|5031926\|ref\|NM_005592.1\| Homo sapiens muscle, skeletal, |
| | receptor tyrosine kinase (MUSK), mRNA |
| NM_005697.3 | >gi\|16445417\|ref\|NM_005697.3\| Homo sapiens secretory carrier |
| | membrane protein 2 (SCAMP2), mRNA |
| NM_005698.2 | >gi\|16445418\|ref\|NM_005698.2\| Homo sapiens secretory carrier |
| | membrane protein 3 (SCAMP3), transcript variant 1, mRNA |
| NM_005723.2 | >gi\|21264582\|ref\|NM_005723.2\| Homo sapiens transmembrane 4 |
| | superfamily member 9 (TM4SF9), mRNA |
| NM_005725.2 | >gi\|21264579\|ref\|NM_005725.2\| Homo sapiens tetraspan 2 (TSPAN- |
| | 2), mRNA |
| NM_005727.2 | >gi\|21264577\|ref\|NM_005727.2\| Homo sapiens tetraspan 1 (TSPAN- |
| | 1), mRNA |
| NM_005803.2 | >gi\|6552331\|ref\|NM_005803.2\| Homo sapiens flotillin 1 (FLOT1), |
| | mRNA |
| NM_005842.2 | >gi\|22209007\|ref\|NM_005842.2\| Homo sapiens sprouty homolog 2 |
| | (Drosophila) (SPRY2), mRNA |
| NM_005855.1 | >gi\|5032018\|ref\|NM_005855.1\| Homo sapiens receptor (calcitonin) |
| | activity modifying protein 1 (RAMP1), mRNA |
| NM_005981.2 | >gi\|21264346\|ref\|NM_005981.2\| Homo sapiens sarcoma amplified |
| | sequence (SAS), mRNA |
| NM_006054.1 | >gi\|5174654\|ref\|NM_006054.1\| Homo sapiens reticulon 3 (RTN3), |
| | mRNA |
| NM_006100.1 | >gi\|5174696\|ref\|NM_006100.1\| Homo sapiens alpha2,3- |
| | sialyltransferase (ST3GALVI), mRNA |
| NM_006118.2 | >gi\|13435355\|ref\|NM_006118.2\| Homo sapiens HS1 binding protein |
| | (HAX1), mRNA |
| NM_006120.2 | >gi\|18765714\|ref\|NM_006120.2\| Homo sapiens major |
| | histocompatibility complex, class II, DM alpha (HLA-DMA), mRNA |
| NM_006137.4 | >gi\|14589878\|ref\|NM_006137.4\| Homo sapiens CD7 antigen (p41) |
| | (CD7), mRNA |
| NM_006280.1 | >gi\|5454089\|ref\|NM_006280.1\| Homo sapiens signal sequence |
| | receptor, delta (translocon-associated protein delta) (SSR4), mRNA |
| NM_006293.2 | >gi\|27597077\|ref\|NM_006293.2\| Homo sapiens TYRO3 protein |
| | tyrosine kinase (TYRO3), mRNA |
| NM_006307.2 | >gi\|21314639\|ref\|NM_006307.2\| Homo sapiens sushi-repeat- |
| | containing protein, X-linked (SRPX), mRNA |
| NM_006320.1 | >gi\|5453915\|ref\|NM_006320.1\| Homo sapiens progesterone receptor |
| | membrane component 2 (PGRMC2), mRNA |
| NM_006325.2 | >gi\|6042206\|ref\|NM_006325.2\| Homo sapiens RAN, member RAS |
| | oncogene family (RAN), mRNA |
| NM_006327.2 | >gi\|45433538\|ref\|NM_006327.2\| Homo sapiens translocase of inner |
| | mitochondrial membrane 23 homolog (yeast) (TIMM23), mRNA |
| NM_006351.1 | >gi\|5454123\|ref\|NM_006351.1\| Homo sapiens translocase of inner |
| | mitochondrial membrane 44 homolog (yeast) (TIMM44), mRNA |
| NM_006356.1 | >gi\|5453558\|ref\|NM_006356.1\| Homo sapiens ATP synthase, H+ |
| | transporting, mitochondrial F0 complex, subunit d (ATP5H), mRNA |
| NM_006366.1 | >gi\|5453592\|ref\|NM_006366.1\| Homo sapiens CAP, adenylate |
| | cyclase-associated protein, 2 (yeast) (CAP2), mRNA |
| NM_006476.2 | >gi\|21359881\|ref\|NM_006476.2\| Homo sapiens ATP synthase, H+ |
| | transporting, mitochondrial F0 complex, subunit g (ATP5L), mRNA |
| NM_006504.2 | >gi\|18860860\|ref\|NM_006504.2\| Homo sapiens protein tyrosine |
| | phosphatase, receptor type, E (PTPRE), transcript variant 1, mRNA |
| NM_006555.2 | >gi\|21071061\|ref\|NM_006555.2\| Homo sapiens SNARE protein Ykt6 |
| | (YKT6), mRNA |
| NM_006573.2 | >gi\|21314645\|ref\|NM_006573.2\| Homo sapiens tumor necrosis factor |
| | (ligand) superfamily, member 13b (TNFSF13B), mRNA |
| NM_006578.2 | >gi\|20336268\|ref\|NM_006578.2\| Homo sapiens guanine nucleotide |
| | binding protein (G protein), beta 5 (GNB5), transcript variant 1, |
| | mRNA |
| NM_006579.1 | >gi\|5729809\|ref\|NM_006579.1\| Homo sapiens emopamil binding |
| | protein (sterol isomerase) (EBP), mRNA |
| NM_006580.2 | >gi\|21536296\|ref\|NM_006580.2\| Homo sapiens claudin 16 |
| | (CLDN16), mRNA |
| NM_006611.1 | >gi\|5729898\|ref\|NM_006611.1\| Homo sapiens killer cell lectin-like |
| | receptor subfamily A, member 1 (KLRA1), mRNA |
| NM_006694.1 | >gi\|5729888\|ref\|NM_006694.1\| Homo sapiens jumping translocation |
| | breakpoint (JTB), mRNA |
| NM_006754.2 | >gi\|33239441\|ref\|NM_006754.2\| Homo sapiens synaptophysin-like |
| | protein (SYPL), transcript variant 1, mRNA |
| NM_006779.2 | >gi\|30089963\|ref\|NM_006779.2\| Homo sapiens CDC42 effector |
| | protein (Rho GTPase binding) 2 (CDC42EP2), mRNA |
| NM_006788.2 | >gi\|21361362\|ref\|NM_006788.2\| Homo sapiens ralA binding protein |
| | 1 (RALBP1), mRNA |
| NM_006804.2 | >gi\|31543656\|ref\|NM_006804.2\| Homo sapiens START domain |
| | containing 3 (STARD3), mRNA |
| NM_006815.2 | >gi\|21314646\|ref\|NM_006815.2\| Homo sapiens coated vesicle |
| | membrane protein (RNP24), mRNA |
| NM_006827.2 | >gi\|21359875\|ref\|NM_006827.2\| Homo sapiens transmembrane |
| | trafficking protein (TMP21), mRNA |
| NM_006854.2 | >gi\|8051609\|ref\|NM_006854.2\| Homo sapiens KDEL (Lys-Asp-Glu- |
| | Leu) endoplasmic reticulum protein retention receptor 2 (KDELR2), |
| | mRNA |
| NM_006858.2 | >gi\|27894322\|ref\|NM_006858.2\| Homo sapiens transmembrane |
| | emp24 domain containing 1 (TMED1), mRNA |
| NM_006869.1 | >gi\|6806912\|ref\|NM_006869.1\| Homo sapiens centaurin, alpha 1 |
| | (CENTA1), mRNA |
| NM_006931.1 | >gi\|5902089\|ref\|NM_006931.1\| Homo sapiens solute carrier family 2 |
| | (facilitated glucose transporter), member 3 (SLC2A3), mRNA |
| NM_006984.1 | >gi\|5921464\|ref\|NM_006984.1\| Homo sapiens claudin 10 (CLDN10), |
| | mRNA |
| NM_007008.1 | >gi\|5902015\|ref\|NM_007008.1\| Homo sapiens reticulon 4 (RTN4), |
| | mRNA |
| NM_007022.1 | >gi\|5901883\|ref\|NM_007022.1\| Homo sapiens putative tumor |
| | suppressor (101F6), mRNA |
| NM_007107.2 | >gi\|28416942\|ref\|NM_007107.2\| Homo sapiens signal sequence |
| | receptor, gamma (translocon-associated protein gamma) (SSR3), |
| | mRNA |
| NM_007172.2 | >gi\|24497450\|ref\|NM_007172.2\| Homo sapiens nucleoporin 50 kDa |
| | (NUP50), transcript variant 2, mRNA |
| NM_007176.1 | >gi\|6005718\|ref\|NM_007176.1\| Homo sapiens chromosome 14 open |
| | reading frame 1 (C14orf1), mRNA |
| NM_007189.1 | >gi\|27881505\|ref\|NM_007189.1\| Homo sapiens ATP-binding |
| | cassette, sub-family F (GCN20), member 2 (ABCF2), nuclear gene |
| | encoding mitochondrial protein, transcript variant 1, mRNA |
| NM_007223.1 | >gi\|6005771\|ref\|NM_007223.1\| Homo sapiens putative G protein |
| | coupled receptor (GPR), mRNA |
| NM_007242.3 | >gi\|34328905\|ref\|NM_007242.3\| Homo sapiens DEAD (Asp-Glu- |
| | Ala-As) box polypeptide 19 (DDX19), mRNA |
| NM_007255.1 | >gi\|6005951\|ref\|NM_007255.1\| Homo sapiens xylosylprotein beta |
| | 1,4-galactosyltransferase, polypeptide 7 (galactosyltransferase I) |
| | (B4GALT7), mRNA |
| NM_007285.5 | >gi\|20911042\|ref\|NM_007285.5\| Homo sapiens GABA(A) receptor- |
| | associated protein-like 2 (GABARAPL2), mRNA |
| NM_012096.1 | >gi\|6912241\|ref\|NM_012096.1\| Homo sapiens adaptor protein |
| | containing pH domain, PTB domain and leucine zipper motif |
| | (APPL), mRNA |
| NM_012113.1 | >gi\|6912283\|ref\|NM_012113.1\| Homo sapiens carbonic anhydrase |
| | XIV (CA14), mRNA |
| NM_012117.1 | >gi\|6912291\|ref\|NM_012117.1\| Homo sapiens chromobox homolog 5 |
| | (HP1 alpha homolog, Drosophila) (CBX5), mRNA |
| NM_012129.1 | >gi\|6912311\|ref\|NM_012129.1\| Homo sapiens claudin 12 (CLDN12), |
| | mRNA |
| NM_012232.2 | >gi\|42734429\|ref\|NM_012232.2\| Homo sapiens polymerase I and |
| | transcript release factor (PTRF), mRNA |
| NM_012261.1 | >gi\|7110632\|ref\|NM_012261.1\| Homo sapiens chromosome 20 open |
| | reading frame 103 (C20orf103), mRNA |
| NM_012339.3 | >gi\|54792144\|ref\|NM_012339.31\| Homo sapiens tetraspanin 15 |
| | (TSPAN15), mRNA |
| NM_012368.1 | >gi\|6912555\|ref\|NM_012368.1\| Homo sapiens olfactory receptor, |
| | family 2, subfamily C, member 1 (OR2C1), mRNA |
| NM_012369.1 | >gi\|6912557\|ref\|NM_012369.1\| Homo sapiens olfactory receptor, |
| | family 2, subfamily F, member 1 (OR2F1), mRNA |
| NM_012407.1 | >gi\|7110696\|ref\|NM_012407.1\| Homo sapiens protein kinase C, alpha |
| | binding protein (PRKCABP), mRNA |
| NM_012419.3 | >gi\|21361404\|ref\|NM_012419.3\| Homo sapiens regulator of G- |
| | protein signalling 17 (RGS17), mRNA |
| NM_012430.2 | >gi\|14591918\|ref\|NM_012430.2\| Homo sapiens SEC22 vesicle |
| | trafficking protein-like 2 (S. cerevisiae) (SEC22L2), mRNA |
| NM_013319.1 | >gi\|7019550\|ref\|NM_013319.1\| Homo sapiens transitional epithelia |
| | response protein (TERE1), mRNA |
| NM_013332.1 | >gi\|7019408\|ref\|NM_013332.1\| Homo sapiens hypoxia-inducible |
| | protein 2 (HIG2), mRNA |
| NM_013338.2 | >gi\|9665250\|ref\|NM_013338.2\| Homo sapiens Alg5, S. cerevisiae, |
| | homolog of (ALG5), mRNA |
| NM_013443.2 | >gi\|21361443\|ref\|NM_013443.2\| Homo sapiens CMP-NeuAC: (beta)- |
| | N-acetylgalactosaminide (alpha)2,6-sialyltransferase member VI |
| | (ST6GALNAC6), mRNA |
| NM_013943.1 | >gi\|7330334\|ref\|NM_013943.1\| Homo sapiens chloride intracellular |
| | channel 4 (CLIC4), mRNA |
| NM_014051.1 | >gi\|7662638\|ref\|NM_014051.1\| Homo sapiens transmembrane protein |
| | 14A (TMEM14A), mRNA |
| NM_014182.2 | >gi\|21314658\|ref\|NM_014182.2\| Homo sapiens ORM1-like 2 (S. cerevisiae) |
| | (ORMDL2), mRNA |
| NM_014184.1 | >gi\|7661823\|ref\|NM_014184.1\| Homo sapiens HSPC163 protein |
| | (HSPC163), mRNA |
| NM_014215.1 | >gi\|31657139\|ref\|NM_014215.1\| Homo sapiens insulin receptor- |
| | related receptor (INSRR), mRNA |
| NM_014251.1 | >gi\|7657580\|ref\|NM_014251.1\| Homo sapiens solute carrier family |
| | 25, member 13 (citrin) (SLC25A13), mRNA |
| NM_014270.3 | >gi\|54633308\|ref\|NM_014270.3\| Homo sapiens solute carrier family |
| | 7 (cationic amino acid transporter, y+ system), member 9 (SLC7A9), |
| | mRNA |
| NM_014310.3 | >gi\|22027485\|ref\|NM_014310.3\| Homo sapiens RASD family, |
| | member 2 (RASD2), mRNA |
| NM_014392.1 | >gi\|7657002\|ref\|NM_014392.1\| Homo sapiens DNA segment on |
| | chromosome 4 (unique) 234 expressed sequence (D4S234E), mRNA |
| NM_014437.2 | >gi\|21361422\|ref\|NM_014437.2\| Homo sapiens solute carrier family |
| | 39 (zinc transporter), member 1 (SLC39A1), mRNA |
| NM_014685.1 | >gi\|7661869\|ref\|NM_014685.1\| Homo sapiens homocysteine- |
| | inducible, endoplasmic reticulum stress-inducible, ubiquitin-like |
| | domain member 1 (HERPUD1), mRNA |
| NM_014713.2 | >gi\|13518239\|ref\|NM_014713.2\| Homo sapiens lysosomal-associated |
| | protein transmembrane 4 alpha (LAPTM4A), mRNA |
| NM_014752.1 | >gi\|7661907\|ref\|NM_014752.1\| Homo sapiens KIAA0102 gene |
| | product (KIAA0102), mRNA |
| NM_014765.1 | >gi\|7657256\|ref\|NM_014765.1\| Homo sapiens translocase of outer |
| | mitochondrial membrane 20 homolog (yeast) (TOMM20), mRNA |
| NM_015112.1 | >gi\|14149670\|ref\|NM_015112.1\| Homo sapiens microtubule |
| | associated serine/threonine kinase 2 (MAST2), mRNA |
| NM_015161.1 | >gi\|24308006\|ref\|NM_015161.1\| Homo sapiens ADP-ribosylation |
| | factor-like 6 interacting protein (ARL6IP), mRNA |
| NM_015727.1 | >gi\|7669545\|ref\|NM_015727.1\| Homo sapiens tachykinin receptor 1 |
| | (TACR1), transcript variant short, mRNA |
| NM_015994.2 | >gi\|19913437\|ref\|NM_015994.2\| Homo sapiens ATPase, H+ |
| | transporting, lysosomal 34 kDa, V1 subunit D (ATP6V1D), mRNA |
| NM_016013.2 | >gi\|49574509\|ref\|NM_016013.2\| Homo sapiens NADH |
| | dehydrogenase (ubiquinone) 1 alpha subcomplex, assembly factor 1 |
| | (NDUFAF1), mRNA |
| NM_016016.1 | >gi\|7706305\|ref\|NM_016016.1\| Homo sapiens CGI-69 protein (CGI- |
| | 69), mRNA |
| NM_016041.1 | >gi\|7705603\|ref\|NM_016041.1\| Homo sapiens CGI-101 protein (F- |
| | LAN-1), mRNA |
| NM_016057.1 | >gi\|7706336\|ref\|NM_016057.1\| Homo sapiens coatomer protein |
| | complex, subunit zeta 1 (COPZ1), mRNA |
| NM_016072.2 | >gi\|19923443\|ref\|NM_016072.2\| Homo sapiens CGI-141 protein |
| | (CGI-141), mRNA |
| NM_016103.1 | >gi\|7705826\|ref\|NM_016103.1\| Homo sapiens GTP-binding protein |
| | Sara (LOC51128), mRNA |
| NM_016145.1 | >gi\|7706664\|ref\|NM_016145.1\| Homo sapiens PTD008 protein |
| | (PTD008), mRNA |
| NM_016286.1 | >gi\|7705924\|ref\|NM_016286.1\| Homo sapiens dicarbonyl/L-xylulose |
| | reductase (DCXR), mRNA |
| NM_016321.1 | >gi\|7706682\|ref\|NM_016321.1\| Homo sapiens Rhesus blood group, C |
| | glycoprotein (RHCG), mRNA |
| NM_016322.2 | >gi\|19923482\|ref\|NM_016322.2\| Homo sapiens RAB14, member |
| | RAS oncogene family (RAB14), mRNA |
| NM_016338.3 | >gi\|39725949\|ref\|NM_016338.3\| Homo sapiens importin 11 (IPO11), |
| | mRNA |
| NM_016371.1 | >gi\|7705420\|ref\|NM_016371.1\| Homo sapiens hydroxysteroid (17- |
| | beta) dehydrogenase 7 (HSD17B7), mRNA |
| NM_016388.2 | >gi\|54607136\|ref\|NM_016388.2\| Homo sapiens T cell receptor |
| | interacting molecule (TCRIM), mRNA |
| NM_016462.1 | >gi\|7705500\|ref\|NM_016462.1\| Homo sapiens hypothetical protein |
| | HSPC194 (HSPC194), mRNA |
| NM_016467.1 | >gi\|7706008\|ref\|NM_016467.1\| Homo sapiens hypothetical protein |
| | LOC51240 (LOC51240), mRNA |
| NM_016511.1 | >gi\|7706062\|ref\|NM_016511.1\| Homo sapiens C-type lectin-like |
| | receptor-1 (CLEC1), mRNA |
| NM_016541.1 | >gi\|7706566\|ref\|NM_016541.1\| Homo sapiens guanine nucleotide |
| | binding protein (G protein), gamma 13 (GNG13), mRNA |
| NM_016561.1 | >gi\|7706090\|ref\|NM_016561.1\| Homo sapiens bifunctional apoptosis |
| | regulator (BFAR), mRNA |
| NM_016564.1 | >gi\|7706096\|ref\|NM_016564.1\| Homo sapiens BM88 antigen |
| | (BM88), mRNA |
| NM_016568.1 | >gi\|7706102\|ref\|NM_016568.1\| Homo sapiens G-protein coupled |
| | receptor SALPR (SALPR), mRNA |
| NM_016641.2 | >gi\|19923804\|ref\|NM_016641.2\| Homo sapiens membrane interacting |
| | protein of RGS16 (MIR16), mRNA |
| NM_016930.1 | >gi\|8394375\|ref\|NM_016930.1\| Homo sapiens syntaxin 18 (STX18), |
| | mRNA |
| NM_017506.1 | >gi\|9506798\|ref\|NM_017506.1\| Homo sapiens olfactory receptor, |
| | family 7, subfamily A, member 5 (OR7A5), mRNA |
| NM_017526.1 | >gi\|8923784\|ref\|NM_017526.1\| Homo sapiens leptin receptor gene- |
| | related protein (OBRGRP), mRNA |
| NM_017882.1 | >gi\|8923531\|ref\|NM_017882.1\| Homo sapiens ceroid-lipofuscinosis, |
| | neuronal 6, late infantile, variant (CLN6), mRNA |
| NM_018091.2 | >gi\|21361688\|ref\|NM_018091.2\| Homo sapiens likely ortholog of |
| | mouse elongation protein 3 homolog (S. cerevisiae) (ELP3), mRNA |
| NM_018153.2 | >gi\|16933552\|ref\|NM_018153.2\| Homo sapiens anthrax toxin |
| | receptor 1 (ANTXR1), transcript variant 3, mRNA |
| NM_018279.2 | >gi\|21361719\|ref\|NM_018279.2\| Homo sapiens hypothetical protein |
| | FLJ10936 (FLJ10936), mRNA |
| NM_018375.1 | >gi\|8922967\|ref\|NM_018375.1\| Homo sapiens solute carrier family |
| | 39 (zinc transporter), member 9 (SLC39A9), mRNA |
| NM_018668.2 | >gi\|18105057\|ref\|NM_018668.2\| Homo sapiens vacuolar protein |
| | sorting 33B (yeast) (VPS33B), mRNA |
| NM_018711.1 | >gi\|24308166\|ref\|NM_018711.1\| Homo sapiens hypothetical protein |
| | DKFZp761H039 (DKFZp761H039), mRNA |
| NM_018969.2 | >gi\|20070286\|ref\|NM_018969.2\| Homo sapiens super conserved |
| | receptor expressed in brain 3 (SREB3), mRNA |
| NM_019069.3 | >gi\|42544246\|ref\|NM_019069.3\| Homo sapiens WD repeat domain |
| | 5B (WDR5B), mRNA |
| NM_019101.2 | >gi\|22091451\|ref\|NM_019101.2\| Homo sapiens apolipoprotein M |
| | (APOM), mRNA |
| NM_019891.1 | >gi\|9845248\|ref\|NM_019891.1\| Homo sapiens ERO1-like beta (S. cerevisiae) |
| | (ERO1LB), mRNA |
| NM_020133.1 | >gi\|9910391\|ref\|NM_020133.1\| Homo sapiens 1-acylglycerol-3- |
| | phosphate O-acyltransferase 4 (lysophosphatidic acid acyltransferase, |
| | delta) (AGPAT4), mRNA |
| NM_020167.2 | >gi\|19923822\|ref\|NM_020167.2\| Homo sapiens neuromedin U |
| | receptor 2 (NMU2R), mRNA |
| NM_020239.2 | >gi\|12965169\|ref\|NM_020239.2\| Homo sapiens small protein effector |
| | 1 of Cdc42 (SPEC1), mRNA |
| NM_020384.1 | >gi\|9966780\|ref\|NM_020384.1\| Homo sapiens claudin 2 (CLDN2), |
| | mRNA |
| NM_020424.2 | >gi\|19923825\|ref\|NM_020424.2\| Homo sapiens hypothetical protein |
| | A-211C6.1 (LOC57149), mRNA |
| NM_020466.3 | >gi\|20070310\|ref\|NM_020466.3\| Homo sapiens hypothetical protein |
| | dJ122O8.2 (DJ122O8.2), mRNA |
| NM_020526.2 | >gi\|18201903\|ref\|NM_020526.2\| Homo sapiens EphA8 (EPHA8), |
| | mRNA |
| NM_020531.2 | >gi\|41327713\|ref\|NM_020531.2\| Homo sapiens chromosome 20 open |
| | reading frame 3 (C20orf3), mRNA |
| NM_020665.2 | >gi\|21361864\|ref\|NM_020665.2\| Homo sapiens kidney-specific |
| | membrane protein (NX17), mRNA |
| NM_020673.1 | >gi\|10190713\|ref\|NM_020673.1\| Homo sapiens RAB22A, member |
| | RAS oncogene family (RAB22A), mRNA |
| NM_020697.2 | >gi\|45433545\|ref\|NM_020697.2\| Homo sapiens potassium voltage- |
| | gated channel, delayed-rectifier, subfamily S, member 2 (KCNS2), |
| | mRNA |
| NM_020836.2 | >gi\|34147339\|ref\|NM_020836.2\| Homo sapiens brain-enriched |
| | guanylate kinase-associated protein (KIAA1446), mRNA |
| NM_020944.2 | >gi\|27413907\|ref\|NM_020944.2\| Homo sapiens glucosidase, beta |
| | (bile acid) 2 (GBA2), mRNA |
| NM_020975.2 | >gi\|21536315\|ref\|NM_020975.2\| Homo sapiens ret proto-oncogene |
| | (multiple endocrine neoplasia and medullary thyroid carcinoma 1, |
| | Hirschsprung disease) (RET), transcript variant 2, mRNA |
| NM_020980.2 | >gi\|11038652\|ref\|NM_020980.2\| Homo sapiens aquaporin 9 (AQP9), |
| | mRNA |
| NM_021105.1 | >gi\|10863876\|ref\|NM_021105.1\| Homo sapiens phospholipid |
| | scramblase 1 (PLSCR1), mRNA |
| NM_021137.2 | >gi\|22024386\|ref\|NM_021137.2\| Homo sapiens tumor necrosis factor, |
| | alpha-induced protein 1 (endothelial) (TNFAIP1), mRNA |
| NM_021203.2 | >gi\|14917112\|ref\|NM_021203.2\| Homo sapiens signal recognition |
| | particle receptor, B subunit (SRPRB), mRNA |
| NM_021627.2 | >gi\|54607090\|ref\|NM_021627.2\| Homo sapiens |
| | SUMO1/sentrin/SMT3 specific protease 2 (SENP2), mRNA |
| NM_021955.1 | >gi\|11386178\|ref\|NM_021955.1\| Homo sapiens guanine nucleotide |
| | binding protein (G protein), gamma transducing activity polypeptide |
| | 1 (GNGT1), mRNA |
| NM_021999.2 | >gi\|31542987\|ref\|NM_021999.2\| Homo sapiens integral membrane |
| | protein 2B (ITM2B), mRNA |
| NM_022006.1 | >gi\|11612658\|ref\|NM_022006.1\| Homo sapiens FXYD domain |
| | containing ion transport regulator 7 (FXYD7), mRNA |
| NM_022059.1 | >gi\|11545764\|ref\|NM_022059.1\| Homo sapiens chemokine (C—X—C |
| | motif) ligand 16 (CXCL16), mRNA |
| NM_022121.2 | >gi\|31377721\|ref\|NM_022121.2\| Homo sapiens PERP, TP53 |
| | apoptosis effector (PERP), mRNA |
| NM_022154.2 | >gi\|15147222\|ref\|NM_022154.2\| Homo sapiens up-regulated by |
| | BCG-CWS (LOC64116), mRNA |
| NM_022157.2 | >gi\|31542866\|ref\|NM_022157.2\| Homo sapiens Ras-related GTP |
| | binding C (RRAGC), mRNA |
| NM_022823.1 | >gi\|12383063\|ref\|NM_022823.1\| Homo sapiens fibronectin type III |
| | domain containing 4 (FNDC4), mRNA |
| NM_022916.2 | >gi\|18105055\|ref\|NM_022916.2\| Homo sapiens vacuolar protein |
| | sorting 33A (yeast) (VPS33A), mRNA |
| NM_023039.2 | >gi\|21362082\|ref\|NM_023039.2\| Homo sapiens ankyrin repeat, |
| | family A (RFXANK-like), 2 (ANKRA2), mRNA |
| NM_023942.1 | >gi\|13027615\|ref\|NM_023942.1\| Homo sapiens hypothetical protein |
| | MGC3036 (MGC3036), mRNA |
| NM_024059.2 | >gi\|15011885\|ref\|NM_024059.2\| Homo sapiens hypothetical protein |
| | MGC5356 (MGC5356), mRNA |
| NM_024076.1 | >gi\|13129063\|ref\|NM_024076.1\| Homo sapiens potassium channel |
| | tetramerisation domain containing 15 (KCTD15), mRNA |
| NM_024081.4 | >gi\|40255027\|ref\|NM_024081.4\| Homo sapiens proline rich Gla (G- |
| | carboxyglutamic acid) 4 (transmembrane) (PRRG4), mRNA |
| NM_024312.1 | >gi\|13236550\|ref\|NM_024312.1\| Homo sapiens MGC4170 protein |
| | (MGC4170), mRNA |
| NM_024411.1 | >gi\|13270472\|ref\|NM_024411.1\| Homo sapiens prodynorphin |
| | (PDYN), mRNA |
| NM_024712.2 | >gi\|19718770\|ref\|NM_024712.2\| Homo sapiens engulfment and cell |
| | motility 3 (ced-12 homolog, C. elegans) (ELMO3), mRNA |
| NM_024786.1 | >gi\|13376149\|ref\|NM_024786.1\| Homo sapiens zinc finger, DHHC |
| | domain containing 11 (ZDHHC11), mRNA |
| NM_024893.1 | >gi\|13376346\|ref\|NM_024893.1\| Homo sapiens chromosome 20 open |
| | reading frame 39 (C20orf39), mRNA |
| NM_025193.2 | >gi\|19923620\|ref\|NM_025193.2\| Homo sapiens hydroxy-delta-5- |
| | steroid dehydrogenase, 3 beta- and steroid delta-isomerase 7 |
| | (HSD3B7), mRNA |
| NM_030570.2 | >gi\|33149302\|ref\|NM_030570.2\| Homo sapiens uroplakin 3B |
| | (UPK3B), transcript variant 1, mRNA |
| NM_030579.1 | >gi\|13385593\|ref\|NM_030579.1\| Homo sapiens cytochrome b5 outer |
| | mitochondrial membrane precursor (CYB5-M), mRNA |
| NM_030755.1 | >gi\|13559515\|ref\|NM_030755.1\| Homo sapiens thioredoxin domain- |
| | containing (TXNDC), mRNA |
| NM_030772.1 | >gi\|13540536\|ref\|NM_030772.1\| Homo sapiens gap junction protein, |
| | alpha 10, 59 kDa (GJA10), mRNA |
| NM_030903.2 | >gi\|50726880\|ref\|NM_030903.2\| Homo sapiens olfactory receptor, |
| | family 2, subfamily W, member 1 (OR2W1), mRNA |
| NM_030908.1 | >gi\|13929211\|ref\|NM_030908.1\| Homo sapiens olfactory receptor, |
| | family 2, subfamily A, member 4 (OR2A4), mRNA |
| NM_030938.2 | >gi\|20070348\|ref\|NM_030938.2\| Homo sapiens likely ortholog of rat |
| | vacuole membrane protein 1 (VMP1), mRNA |
| NM_030969.2 | >gi\|18373333\|ref\|NM_030969.2\| Homo sapiens transmembrane |
| | protein 14B (TMEM14B), mRNA |
| NM_030971.3 | >gi\|494728361\|ref\|NM_030971.3\| Homo sapiens sideroflexin 3 |
| | (SFXN3), mRNA |
| NM_031412.1 | >gi\|13899218\|ref\|NM_031412.1\| Homo sapiens GABA(A) receptor- |
| | associated protein like 1 (GABARAPL1), mRNA |
| NM_031434.2 | >gi\|31543199\|ref\|NM_031434.2\| Homo sapiens chromosome 7 open |
| | reading frame 21 (C7orf21), mRNA |
| NM_031496.1 | >gi\|14165406\|ref\|NM_031496.1\| Homo sapiens protocadherin alpha 2 |
| | (PCDHA2), transcript variant 3, mRNA |
| NM_031896.3 | >gi\|22027498\|ref\|NM_031896.3\| Homo sapiens calcium channel, |
| | voltage-dependent, gamma subunit 7 (CACNG7), mRNA |
| NM_031936.2 | >gi\|19923637\|ref\|NM_031936.2\| Homo sapiens G protein-coupled |
| | receptor 61 (GPR61), mRNA |
| NM_031954.2 | >gi\|31377664\|ref\|NM_031954.2\| Homo sapiens potassium channel |
| | tetramerisation domain containing 10 (KCTD10), mRNA |
| NM_032036.2 | >gi\|50511937\|ref\|NM_032036.2\| Homo sapiens family with sequence |
| | similarity 14, member A (FAM14A), mRNA |
| NM_032047.2 | >gi\|15451895\|ref\|NM_032047.2\| Homo sapiens UDP- |
| | GlcNAc: betaGal beta-1,3-N-acetylglucosaminyltransferase 5 |
| | (B3GNT5), mRNA |
| NM_032122.2 | >gi\|19549326\|ref\|NM_032122.2\| Homo sapiens dystrobrevin binding |
| | protein 1 (DTNBP1), mRNA |
| NM_032318.1 | >gi\|14150087\|ref\|NM_032318.1\| Homo sapiens hippocampus |
| | abundant gene transcript-like 2 (HIATL2), mRNA |
| NM_032439.1 | >gi\|33469020\|ref\|NM_032439.1\| Homo sapiens phytanoyl-CoA |
| | hydroxylase interacting protein-like (PHYHIPL), mRNA |
| NM_033102.1 | >gi\|14916436\|ref\|NM_033102.1\| Homo sapiens prostein protein |
| | (Prostein), mRNA |
| NM_033105.1 | >gi\|14916442\|ref\|NM_033105.1\| Homo sapiens beta cysteine string |
| | protein (LOC85479), mRNA |
| NM_033540.2 | >gi\|45269136\|ref\|NM_033540.2\| Homo sapiens mitofusin 1 (MFN1), |
| | nuclear gene encoding mitochondrial protein, transcript variant 1, |
| | mRNA |
| NM_033542.2 | >gi\|56676380\|ref\|NM_033542.2\| Homo sapiens chromosome 20 open |
| | reading frame 35 (C20orf35), mRNA |
| NM_052822.1 | >gi\|16445413\|ref\|NM_052822.1\| Homo sapiens secretory carrier |
| | membrane protein 1 (SCAMP1), transcript variant 2, mRNA |
| NM_052837.1 | >gi\|16445420\|ref\|NM_052837.1\| Homo sapiens secretory carrier |
| | membrane protein 3 (SCAMP3), transcript variant 2, mRNA |
| NM_053278.1 | >gi\|16751916\|ref\|NM_053278.1\| Homo sapiens G protein-coupled |
| | receptor 102 (GPR102), mRNA |
| NM_054020.2 | >gi\|26051221\|ref\|NM_054020.2\| Homo sapiens cation channel, sperm |
| | associated 2 (CATSPER2), transcript variant 1, mRNA |
| NM_054030.1 | >gi\|16876450\|ref\|NM_054030.1\| Homo sapiens G protein-coupled |
| | receptor MRGX2 (MRGX2), mRNA |
| NM_079834.1 | >gi\|17738286\|ref\|NM_079834.1\| Homo sapiens secretory carrier |
| | membrane protein 4 (SCAMP4), mRNA |
| NM_080387.1 | >gi\|17933769\|ref\|NM_080387.1\| Homo sapiens C-type lectin-like |
| | receptor (CLEC-6), mRNA |
| NM_080548.1 | >gi\|18104990\|ref\|NM_080548.1\| Homo sapiens protein tyrosine |
| | phosphatase, non-receptor type 6 (PTPN6), transcript variant 2, |
| | mRNA |
| NM_080653.1 | >gi\|18087814\|ref\|NM_080653.1\| Homo sapiens ATPase, H+ |
| | transporting, lysosomal 31 kD, V1 subunit E-like 2 (ATP6V1EL2), |
| | mRNA |
| NM_080739.1 | >gi\|18152770\|ref\|NM_080739.1\| Homo sapiens chromosome 20 open |
| | reading frame 141 (C20orf141), mRNA |
| NM_080817.1 | >gi\|18201869\|ref\|NM_080817.1\| Homo sapiens G protein-coupled |
| | receptor 82 (GPR82), mRNA |
| NM_133496.3 | >gi\|41529827\|ref\|NM_133496.3\| Homo sapiens solute carrier family |
| | 30 (zinc transporter), member 7 (SLC30A7), mRNA |
| NM_138461.1 | >gi\|19923994\|ref\|NM_138461.1\| Homo sapiens hypothetical protein |
| | BC013113 (LOC116211), mRNA |
| NM_138463.1 | >gi\|19923998\|ref\|NM_138463.1\| Homo sapiens hypothetical protein |
| | BC014072 (LOC116238), mRNA |
| NM_138501.3 | >gi\|34222216\|ref\|NM_138501.3\| Homo sapiens glycoprotein, |
| | synaptic 2 (GPSN2), mRNA |
| NM_138573.1 | >gi\|20070379\|ref\|NM_138573.1\| Homo sapiens neuregulin 4 |
| | (LOC145957), mRNA |
| NM_138786.1 | >gi\|20270326\|ref\|NM_138786.1\| Homo sapiens hypothetical protein |
| | BC014339 (LOC116441), mRNA |
| NM_138959.1 | >gi\|20373170\|ref\|NM_138959.1\| Homo sapiens vang-like 1 (vangogh, |
| | Drosophila) (VANGL1), mRNA |
| NM_139313.1 | >gi\|21327686\|ref\|NM_139313.1\| Homo sapiens YME1-like 1 (S. cerevisiae) |
| | (YME1L1), nuclear gene encoding mitochondrial protein, |
| | transcript variant 2, mRNA |
| NM_144628.1 | >gi\|21389446\|ref\|NM_144628.1\| Homo sapiens chromosome 20 open |
| | reading frame 140 (C20orf140), mRNA |
| NM_144638.1 | >gi\|21389472\|ref\|NM_144638.1\| Homo sapiens hypothetical protein |
| | MGC29956 (MGC29956), mRNA |
| NM_144673.1 | >gi\|21389566\|ref\|NM_144673.1\| Homo sapiens chemokine-like |
| | factor super family 2 (CKLFSF2), mRNA |
| NM_144676.1 | >gi\|21389572\|ref\|NM_144676.1\| Homo sapiens hypothetical protein |
| | MGC23911 (MGC23911), mRNA |
| NM_145206.1 | >gi\|21624647\|ref\|NM_145206.1\| Homo sapiens vesicle transport |
| | through interaction with t-SNAREs homolog 1A (yeast) (VTI1A), |
| | mRNA |
| NM_145286.1 | >gi\|21686994\|ref\|NM_145286.1\| Homo sapiens stomatin (EPB72)- |
| | like 3 (STOML3), mRNA |
| NM_145793.1 | >gi\|22035691\|ref\|NM_145793.1\| Homo sapiens GDNF family |
| | receptor alpha 1 (GFRA1), transcript variant 2, mRNA |
| NM_148957.2 | >gi\|31652245\|ref\|NM_148957.2\| Homo sapiens tumor necrosis factor |
| | receptor superfamily, member 19 (TNFRSF19), transcript variant 2, |
| | mRNA |
| NM_148975.1 | >gi\|23110994\|ref\|NM_148975.1\| Homo sapiens membrane-spanning |
| | 4-domains, subfamily A, member 4 (MS4A4A), transcript variant 2, |
| | mRNA |
| NM_152353.1 | >gi\|22748764\|ref\|NM_152353.1\| Homo sapiens hypothetical protein |
| | MGC33839 (MGC33839), mRNA |
| NM_152430.1 | >gi\|22748910\|ref\|NM_152430.1\| Homo sapiens hypothetical protein |
| | MGC24137 (MGC24137), mRNA |
| NM_152690.1 | >gi\|24497590\|ref\|NM_152690.1\| Homo sapiens dolichyl-phosphate |
| | mannosyltransferase polypeptide 2, regulatory subunit (DPM2), |
| | transcript variant 2, mRNA |
| NM_152864.2 | >gi\|42476063\|ref\|NM_152864.2\| Homo sapiens chromosome 20 open |
| | reading frame 58 (C20orf58), mRNA |
| NM_153611.3 | >gi\|48976062\|ref\|NM_153611.3\| Homo sapiens hypothetical protein |
| | MGC20446 (MGC20446), mRNA |
| NM_172341.1 | >gi\|28144919\|ref\|NM_172341.1\| Homo sapiens presenilin enhancer 2 |
| | (PEN2), mRNA |
| NM_173470.1 | >gi\|27735036\|ref\|NM_173470.1\| Homo sapiens hypothetical protein |
| | LOC93380 (LOC93380), mRNA |
| NM_173605.1 | >gi\|27734696\|ref\|NM_173605.1\| Homo sapiens potassium channel |
| | regulator (KCNRG), mRNA |
| NM_174889.2 | >gi\|31341340\|ref\|NM_174889.2\| Homo sapiens hypothetical protein |
| | LOC91942 (LOC91942), mRNA |
| NM_174926.1 | >gi\|28376667\|ref\|NM_174926.1\| Homo sapiens hypothetical protein |
| | MGC17839 (MGC17839), mRNA |
| NM_175614.2 | >gi\|46370098\|ref\|NM_175614.2\| Homo sapiens NADH |
| | dehydrogenase (ubiquinone) 1 alpha subcomplex, 11, 14.7 kDa |
| | (NDUFA11), mRNA |
| NM_175733.2 | >gi\|31342257\|ref\|NM_175733.2\| Homo sapiens synaptotagmin IX |
| | (SYT9), mRNA |
| NM_177424.1 | >gi\|28933464\|ref\|NM_177424.1\| Homo sapiens syntaxin 12 (STX12), |
| | mRNA |
| NM_177996.1 | >gi\|30061490\|ref\|NM_177996.1\| Homo sapiens erythrocyte |
| | membrane protein band 4.1-like 1 (EPB41L1), transcript variant 2, |
| | mRNA |
| NM_178129.3 | >gi\|38373667\|ref\|NM_178129.3\| Homo sapiens purinergic receptor |
| | P2Y, G-protein coupled, 8 (P2RY8), mRNA |
| NM_178450.2 | >gi\|31341961\|ref\|NM_178450.2\| Homo sapiens hypothetical protein |
| | MGC48332 (MGC48332), mRNA |
| NM_178498.2 | >gi\|31341916\|ref\|NM_178498.2\| Homo sapiens hypothetical protein |
| | MGC52019 (MGC52019), mRNA |
| NM_178543.3 | >gi\|45545420\|ref\|NM_178543.3\| Homo sapiens ectonucleotide |
| | prophosphatase/phosphodiesterase 7 (ENPP7), mRNA |
| NM_178833.3 | >gi\|47271478\|ref\|NM_178833.3\| Homo sapiens hypothetical protein |
| | BC009732 (LOC133308), mRNA |
| NM_178858.3 | >gi\|34222270\|ref\|NM_178858.3\| Homo sapiens sideroflexin 2 |
| | (SFXN2), mRNA |
| NM_178863.2 | >gi\|31341469\|ref\|NM_178863.2\| Homo sapiens potassium channel |
| | tetramerisation domain containing 13 (KCTD13), mRNA |
| NM_182553.1 | >gi\|32698937\|ref\|NM_182553.1\| Homo sapiens hypothetical protein |
| | MGC50896 (MGC50896), mRNA |
| NM_182607.2 | >gi\|34222376\|ref\|NM_182607.2\| Homo sapiens hypothetical protein |
| | MGC44287 (MGC44287), mRNA |
| NM_198276.1 | >gi\|38093646\|ref\|NM_198276.1\| Homo sapiens transmembrane |
| | protein 17 (TMEM17), mRNA |
| NM_199328.1 | >gi\|40788010\|ref\|NM_199328.1\| Homo sapiens claudin 8 (CLDN8), |
| | mRNA |
| NM_207337.1 | >gi\|46559738\|ref\|NM_207337.1\| Homo sapiens hypothetical protein |
| | LOC196394 (LOC196394), mRNA |

TABLE 16
Transmembrane proteins: GO: 0004888

| | |
|---|---|
| NM_000024.3 | >gi\|15718673\|ref\|NM_000024.3\| Homo sapiens adrenergic, beta-2-, |
| | receptor, surface (ADRB2), mRNA |
| NM_000160.1 | >gi\|4503946\|ref\|NM_000160.1\| Homo sapiens glucagon receptor |
| | (GCGR), mRNA |
| NM_000206.1 | >gi\|4557881\|ref\|NM_000206.1\| Homo sapiens interleukin 2 receptor, |
| | gamma (severe combined immunodeficiency) (IL2RG), mRNA |
| NM_000208.1 | >gi\|4557883\|ref\|NM_000208.1\| Homo sapiens insulin receptor |
| | (INSR), mRNA |
| NM_000459.1 | >gi\|4557868\|ref\|NM_000459.1\| Homo sapiens TEK tyrosine kinase, |
| | endothelial (venous malformations, multiple cutaneous and mucosal) |
| | (TEK), mRNA |
| NM_000626.1 | >gi\|11038673\|ref\|NM_000626.1\| Homo sapiens CD79B antigen |
| | (immunoglobulin-associated beta) (CD79B), transcript variant 1, |
| | mRNA |
| NM_000675.3 | >gi\|17136146\|ref\|NM_000675.3\| Homo sapiens adenosine A2a |
| | receptor (ADORA2A), mRNA |
| NM_000732.3 | >gi\|55775475\|ref\|NM_000732.3\| Homo sapiens CD3D antigen, delta |
| | polypeptide (TiT3 complex) (CD3D), mRNA |
| NM_000810.2 | >gi\|6031207\|ref\|NM_000810.2\| Homo sapiens gamma-aminobutyric |
| | acid (GABA) A receptor, alpha 5 (GABRA5), mRNA |
| NM_000866.1 | >gi\|10835196\|ref\|NM_000866.1\| Homo sapiens 5-hydroxytryptamine |
| | (serotonin) receptor 1F (HTR1F), mRNA |
| NM_000910.1 | >gi\|4505446\|ref\|NM_000910.1\| Homo sapiens neuropeptide Y |
| | receptor Y2 (NPY2R), mRNA |
| NM_000953.2 | >gi\|38505191\|ref\|NM_000953.2\| Homo sapiens prostaglandin D2 |
| | receptor (DP) (PTGDR), mRNA |
| NM_001013.2 | >gi\|14141192\|ref\|NM_001013.2\| Homo sapiens ribosomal protein S9 |
| | (RPS9), mRNA |
| NM_001014.2 | >gi\|13904867\|ref\|NM_001014.2\| Homo sapiens ribosomal protein |
| | S10 (RPS10), mRNA |
| NM_001015.2 | >gi\|14277698\|ref\|NM_001015.2\| Homo sapiens ribosomal protein |
| | S11 (RPS11), mRNA |
| NM_001016.2 | >gi\|14277699\|ref\|NM_001016.2\| Homo sapiens ribosomal protein |
| | S12 (RPS 12), mRNA |
| NM_001018.2 | >gi\|14591911\|ref\|NM_001018.2\| Homo sapiens ribosomal protein |
| | S15 (RPS15), mRNA |
| NM_001021.2 | >gi\|14591913\|ref\|NM_001021.2\| Homo sapiens ribosomal protein |
| | S17 (RPS17), mRNA |
| NM_001022.3 | >gi\|48255921\|ref\|NM_001022.3\| Homo sapiens ribosomal protein |
| | S19 (RPS 19), mRNA |
| NM_001023.2 | >gi\|14591915\|ref\|NM_001023.2\| Homo sapiens ribosomal protein |
| | S20 (RPS20), mRNA |
| NM_001106.2 | >gi\|10862697\|ref\|NM_001106.2\| Homo sapiens activin A receptor, |
| | type IIB (ACVR2B), mRNA |
| NM_001203.1 | >gi\|4502430\|ref\|NM_001203.1\| Homo sapiens bone morphogenetic |
| | protein receptor, type IB (BMPR1B), mRNA |
| NM_001296.3 | >gi\|37577160\|ref\|NM_001296.3\| Homo sapiens chemokine binding |
| | protein 2 (CCBP2), mRNA |
| NM_001306.2 | >gi\|21536298\|ref\|NM_001306.2\| Homo sapiens claudin 3 (CLDN3), |
| | mRNA |
| NM_001616.2 | >gi\|10862696\|ref\|NM_001616.2\| Homo sapiens activin A receptor, |
| | type II (ACVR2), mRNA |
| NM_001671.2 | >gi\|18426870\|ref\|NM_001671.2\| Homo sapiens asialoglycoprotein |
| | receptor 1 (ASGR1), mRNA |
| NM_001736.1 | >gi\|4502508\|ref\|NM_001736.1\| Homo sapiens complement |
| | component 5 receptor 1 (C5a ligand) (C5R1), mRNA |
| NM_001841.1 | >gi\|4502928\|ref\|NM_001841.1\| Homo sapiens cannabinoid receptor 2 |
| | (macrophage) (CNR2), mRNA |
| NM_001993.2 | >gi\|10518499\|ref\|NM_001993.2\| Homo sapiens coagulation factor III |
| | (thromboplastin, tissue factor) (F3), mRNA |
| NM_002262.2 | >gi\|7669497\|ref\|NM_002262.2\| Homo sapiens killer cell lectin-like |
| | receptor subfamily D, member 1 (KLRD 1), transcript variant 1, |
| | mRNA |
| NM_002447.1 | >gi\|4505264\|ref\|NM_002447.1\| Homo sapiens macrophage |
| | stimulating 1 receptor (c-met-related tyrosine kinase) (MST1R), |
| | mRNA |
| NM_002944.2 | >gi\|19924164\|ref\|NM_002944.2\| Homo sapiens v-ros UR2 sarcoma |
| | virus oncogene homolog 1 (avian) (ROS1), mRNA |
| NM_003123.1 | >gi\|4507180\|ref\|NM_003123.1\| Homo sapiens sialophorin (gpL115, |
| | leukosialin, CD43) (SPN), mRNA |
| NM_003382.2 | >gi\|21361556\|ref\|NM_003382.2\| Homo sapiens vasoactive intestinal |
| | peptide receptor 2 (VIPR2), mRNA |
| NM_003853.2 | >gi\|27477087\|ref\|NM_003853.2\| Homo sapiens interleukin 18 |
| | receptor accessory protein (IL18RAP), mRNA |
| NM_004304.3 | >gi\|29029631\|ref\|NM_004304.3\| Homo sapiens anaplastic lymphoma |
| | kinase (Ki-1) (ALK), mRNA |
| NM_004382.2 | >gi\|19923244\|ref\|NM_004382.2\| Homo sapiens corticotropin |
| | releasing hormone receptor 1 (CRHR1), mRNA |
| NM_004431.1 | >gi\|4758277\|ref\|NM_004431.1\| Homo sapiens EphA2 (EPHA2), |
| | mRNA |
| NM_004438.1 | >gi\|4758279\|ref\|NM_004438.1\| Homo sapiens EphA4 (EPHA4), |
| | mRNA |
| NM_004439.4 | >gi\|56119208\|ref\|NM_004439.4\| Homo sapiens EPH receptor A5 |
| | (EPHA5), transcript variant 1, mRNA |
| NM_004440.1 | >gi\|4758281\|ref\|NM_004440.1\| Homo sapiens EphA7 (EPHA7), |
| | mRNA |
| NM_004441.2 | >gi\|21396502\|ref\|NM_004441.2\| Homo sapiens EphB1 (EPHB1), |
| | mRNA |
| NM_004441.3 | >gi\|55770893\|ref\|NM_004441.3\| Homo sapiens EPH receptor B1 |
| | (EPHB1), mRNA |
| NM_004442.3 | >gi\|21396503\|ref\|NM_004442.3\| Homo sapiens EphB2 (EPHB2), |
| | transcript variant 1, mRNA |
| NM_004443.2 | >gi\|17975767\|ref\|NM_004443.2\| Homo sapiens EphB3 (EPHB3), |
| | mRNA |
| NM_004444.2 | >gi\|17975769\|ref\|NM_004444.2\| Homo sapiens EphB4 (EPHB4), |
| | mRNA |
| NM_004445.1 | >gi\|4758291\|ref\|NM_004445.1\| Homo sapiens EphB6 (EPHB6), |
| | mRNA |
| NM_004512.3 | >gi\|22212920\|ref\|NM_004512.3\| Homo sapiens interleukin 11 |
| | receptor, alpha (IL11RA), transcript variant 1, mRNA |
| NM_004720.3 | >gi\|11038657\|ref\|NM_004720.3\| Homo sapiens endothelial |
| | differentiation, lysophosphatidic acid G-protein-coupled receptor, 4 |
| | (EDG4), mRNA |
| NM_004952.3 | >gi\|33359683\|ref\|NM_004952.3\| Homo sapiens ephrin-A3 (EFNA3), |
| | mRNA |
| NM_005201.2 | >gi\|13929430\|ref\|NM_005201.2\| Homo sapiens chemokine (C—C |
| | motif) receptor 8 (CCR8), mRNA |
| NM_005226.2 | >gi\|38788192\|ref\|NM_005226.2\| Homo sapiens endothelial |
| | differentiation, sphingolipid G-protein-coupled receptor, 3 (EDG3), |
| | mRNA |
| NM_005232.1 | >gi\|4885208\|ref\|NM_005232.1\| Homo sapiens EphA1 (EPHA1), |
| | mRNA |
| NM_005233.2 | >gi\|21361240\|ref\|NM_005233.2\| Homo sapiens EphA3 (EPHA3), |
| | mRNA |
| NM_005272.2 | >gi\|22027523\|ref\|NM_005272.2\| Homo sapiens guanine nucleotide |
| | binding protein (G protein), alpha transducing activity polypeptide 2 |
| | (GNAT2), mRNA |
| NM_005283.1 | >gi\|4885338\|ref\|NM_005283.1\| Homo sapiens chemokine (C motif) |
| | receptor 1 (XCR1), mRNA |
| NM_005290.1 | >gi\|4885298\|ref\|NM_005290.1\| Homo sapiens G protein-coupled |
| | receptor 15 (GPR15), mRNA |
| NM_005294.1 | >gi\|4885306\|ref\|NM_005294.1\| Homo sapiens G protein-coupled |
| | receptor 21 (GPR21), mRNA |
| NM_005299.1 | >gi\|4885316\|ref\|NM_005299.1\| Homo sapiens G protein-coupled |
| | receptor 31 (GPR31), mRNA |
| NM_005567.2 | >gi\|6006016\|ref\|NM_005567.2\| Homo sapiens lectin, galactoside- |
| | binding, soluble, 3 binding protein (LGALS3BP), mRNA |
| NM_005592.1 | >gi\|5031926\|ref\|NM_005592.1\| Homo sapiens muscle, skeletal, |
| | receptor tyrosine kinase (MUSK), mRNA |
| NM_006293.2 | >gi\|27597077\|ref\|NM_006293.2\| Homo sapiens TYRO3 protein |
| | tyrosine kinase (TYRO3), mRNA |
| NM_006504.2 | >gi\|18860860\|ref\|NM_006504.2\| Homo sapiens protein tyrosine |
| | phosphatase, receptor type, E (PTPRE), transcript variant 1, mRNA |
| NM_006579.1 | >gi\|5729809\|ref\|NM_006579.1\| Homo sapiens emopamil binding |
| | protein (sterol isomerase) (EBP), mRNA |
| NM_006611.1 | >gi\|5729898\|ref\|NM_006611.1\| Homo sapiens killer cell lectin-like |
| | receptor subfamily A, member 1 (KLRA1), mRNA |
| NM_007223.1 | >gi\|6005771\|ref\|NM_007223.1\| Homo sapiens putative G protein |
| | coupled receptor (GPR), mRNA |
| NM_012368.1 | >gi\|6912555\|ref\|NM_012368.1\| Homo sapiens olfactory receptor, |
| | family 2, subfamily C, member 1 (OR2C1), mRNA |
| NM_012369.1 | >gi\|6912557\|ref\|NM_012369.1\| Homo sapiens olfactory receptor, |
| | family 2, subfamily F, member 1 (OR2F1), mRNA |
| NM_014215.1 | >gi\|31657139\|ref\|NM_014215.1\| Homo sapiens insulin receptor- |
| | related receptor (INSRR), mRNA |
| NM_015727.1 | >gi\|7669545\|ref\|NM_015727.1\| Homo sapiens tachykinin receptor 1 |
| | (TACR1), transcript variant short, mRNA |
| NM_016511.1 | >gi\|7706062\|ref\|NM_016511.1\| Homo sapiens C-type lectin-like |
| | receptor-1 (CLEC1), mRNA |
| NM_016568.1 | >gi\|7706102\|ref\|NM_016568.1\| Homo sapiens G-protein coupled |
| | receptor SALPR (SALPR), mRNA |
| NM_017506.1 | >gi\|9506798\|ref\|NM_017506.1\| Homo sapiens olfactory receptor, |
| | family 7, subfamily A, member 5 (OR7A5), mRNA |
| NM_018969.2 | >gi\|20070286\|ref\|NM_018969.2\| Homo sapiens super conserved |
| | receptor expressed in brain 3 (SREB3), mRNA |
| NM_020167.2 | >gi\|19923822\|ref\|NM_020167.2\| Homo sapiens neuromedin U |
| | receptor 2 (NMU2R), mRNA |
| NM_020526.2 | >gi\|18201903\|ref\|NM_020526.2\| Homo sapiens EphA8 (EPHA8), |
| | mRNA |
| NM_022059.1 | >gi\|11545764\|ref\|NM_022059.1\| Homo sapiens chemokine (C—X —C |
| | motif) ligand 16 (CXCL16), mRNA |
| NM_030903.2 | >gi\|50726880\|ref\|NM_030903.2\| Homo sapiens olfactory receptor, |
| | family 2, subfamily W, member 1 (OR2W1), mRNA |
| NM_030908.1 | >gi\|13929211\|ref\|NM_030908.1\| Homo sapiens olfactory receptor, |
| | family 2, subfamily A, member 4 (OR2A4), mRNA |
| NM_031936.2 | >gi\|19923637\|ref\|NM_031936.2\| Homo sapiens G protein-coupled |
| | receptor 61 (GPR61), mRNA |
| NM_053278.1 | >gi\|16751916\|ref\|NM_053278.1\| Homo sapiens G protein-coupled |
| | receptor 102 (GPR102), mRNA |
| NM_054030.1 | >gi\|16876450\|ref\|NM_054030.1\| Homo sapiens G protein-coupled |
| | receptor MRGX2 (MRGX2), mRNA |
| NM_080817.1 | >gi\|18201869\|ref\|NM_080817.1\| Homo sapiens G protein-coupled |
| | receptor 82 (GPR82), mRNA |
| NM_145793.1 | >gi\|22035691\|ref\|NM_145793.1\| Homo sapiens GDNF family |
| | receptor alpha 1 (GFRA1), transcript variant 2, mRNA |
| NM_148957.2 | >gi\|31652245\|ref\|NM_148957.2\| Homo sapiens tumor necrosis factor |
| | receptor superfamily, member 19 (TNFRSF19), transcript variant 2, |
| | mRNA |
| NM_152430.1 | >gi\|22748910\|ref\|NM_152430.1\| Homo sapiens hypothetical protein |
| | MGC24137 (MGC24137), mRNA |
| NM_177435.1 | >gi\|29171749\|ref\|NM_177435.1\| Homo sapiens peroxisome |
| | proliferative activated receptor, delta (PPARD), transcript variant 2, |
| | mRNA |
| NM_178129.3 | >gi\|38373667\|ref\|NM_178129.3\| Homo sapiens purinergic receptor |
| | P2Y, G-protein coupled, 8 (P2RY8), mRNA |

TABLE 17
GPCRs: GO: 0004930

| | |
|---|---|
| NM_000024.3 | >gi\|15718673\|ref\|NM_000024.3\| Homo sapiens adrenergic, beta-2-, |
| | receptor, surface (ADRB2), mRNA |
| NM_000160.1 | >gi\|4503946\|ref\|NM_000160.1\| Homo sapiens glucagon receptor |
| | (GCGR), mRNA |
| NM_000675.3 | >gi\|17136146\|ref\|NM_000675.3\| Homo sapiens adenosine A2a |
| | receptor (ADORA2A), mRNA |
| NM_000866.1 | >gi\|10835196\|ref\|NM_000866.1\| Homo sapiens 5-hydroxytryptamine |
| | (serotonin) receptor 1F (HTR1F), mRNA |
| NM_000910.1 | >gi\|4505446\|ref\|NM_000910.1\| Homo sapiens neuropeptide Y |
| | receptor Y2 (NPY2R), mRNA |
| NM_000953.2 | >gi\|38505191\|ref\|NM_000953.2\| Homo sapiens prostaglandin D2 |
| | receptor (DP) (PTGDR), mRNA |
| NM_001013.2 | >gi\|14141192\|ref\|NM_001013.2\| Homo sapiens ribosomal protein S9 |
| | (RPS9), mRNA |
| NM_001014.2 | >gi\|13904867\|ref\|NM_001014.2\| Homo sapiens ribosomal protein |
| | S10 (RPS10), mRNA |
| NM_001016.2 | >gi\|14277699\|ref\|NM_001016.2\| Homo sapiens ribosomal protein |
| | S12 (RPS12), mRNA |
| NM_001018.2 | >gi\|14591911\|ref\|NM_001018.2\| Homo sapiens ribosomal protein |
| | S15 (RPS15), mRNA |
| NM_001021.2 | >gi\|14591913\|ref\|NM_001021.2\| Homo sapiens ribosomal protein |
| | S17 (RPS17), mRNA |
| NM_001022.3 | >gi\|48255921\|ref\|NM_001022.3\| Homo sapiens ribosomal protein |
| | S19 (RPS19), mRNA |
| NM_001296.3 | >gi\|37577160\|ref\|NM_001296.3\| Homo sapiens chemokine binding |
| | protein 2 (CCBP2), mRNA |
| NM_001736.1 | >gi\|4502508\|ref\|NM_001736.1\| Homo sapiens complement |
| | component 5 receptor 1 (C5a ligand) (C5R1), mRNA |
| NM_001841.1 | >gi\|4502928\|ref\|NM_001841.1\| Homo sapiens cannabinoid receptor 2 |
| | (macrophage) (CNR2), mRNA |
| NM_003382.2 | >gi\|21361556\|ref\|NM_003382.2\| Homo sapiens vasoactive intestinal |
| | peptide receptor 2 (VIPR2), mRNA |
| NM_004382.2 | >gi\|19923244\|ref\|NM_004382.2\| Homo sapiens corticotropin |
| | releasing hormone receptor 1 (CRHR1), mRNA |
| NM_004720.3 | >gi\|11038657\|ref\|NM_004720.3\| Homo sapiens endothelial |
| | differentiation, lysophosphatidic acid G-protein-coupled receptor, 4 |
| | (EDG4), mRNA |
| NM_005201.2 | >gi\|13929430\|ref\|NM_005201.2\| Homo sapiens chemokine (C—C |
| | motif) receptor 8 (CCR8), mRNA |
| NM_005226.2 | >gi\|38788192\|ref\|NM_005226.2\| Homo sapiens endothelial |
| | differentiation, sphingolipid G-protein-coupled receptor, 3 (EDG3), |
| | mRNA |
| NM_005272.2 | >gi\|22027523\|ref\|NM_005272.2\| Homo sapiens guanine nucleotide |
| | binding protein (G protein), alpha transducing activity polypeptide 2 |
| | (GNAT2), mRNA |
| NM_005283.1 | >gi\|4885338\|ref\|NM_005283.1\| Homo sapiens chemokine (C motif) |
| | receptor 1 (XCR1), mRNA |
| NM_005290.1 | >gi\|4885298\|ref\|NM_005290.1\| Homo sapiens G protein-coupled |
| | receptor 15 (GPR15), mRNA |
| NM_005294.1 | >gi\|4885306\|ref\|NM_005294.1\| Homo sapiens G protein-coupled |
| | receptor 21 (GPR21), mRNA |
| NM_005299.1 | >gi\|4885316\|ref\|NM_005299.1\| Homo sapiens G protein-coupled |
| | receptor 31 (GPR31), mRNA |
| NM_007223.1 | >gi\|6005771\|ref\|NM_007223.1\| Homo sapiens putative G protein |
| | coupled receptor (GPR), mRNA |
| NM_012368.1 | >gi\|6912555\|ref\|NM_012368.1\| Homo sapiens olfactory receptor, |
| | family 2, subfamily C, member 1 (OR2C1), mRNA |
| NM_012369.1 | >gi\|6912557\|ref\|NM_012369.1\| Homo sapiens olfactory receptor, |
| | family 2, subfamily F, member 1 (OR2F1), mRNA |
| NM_015727.1 | >gi\|7669545\|ref\|NM_015727.1\| Homo sapiens tachykinin receptor 1 |
| | (TACR1), transcript variant short, mRNA |
| NM_016568.1 | >gi\|7706102\|ref\|NM_016568.1\| Homo sapiens G-protein coupled |
| | receptor SALPR (SALPR), mRNA |
| NM_017506.1 | >gi\|9506798\|ref\|NM_017506.1\| Homo sapiens olfactory receptor, |
| | family 7, subfamily A, member 5 (OR7A5), mRNA |
| NM_018969.2 | >gi\|20070286\|ref\|NM_018969.2\| Homo sapiens super conserved |
| | receptor expressed in brain 3 (SREB3), mRNA |
| NM_020167.2 | >gi\|19923822\|ref\|NM_020167.2\| Homo sapiens neuromedin U |
| | receptor 2 (NMU2R), mRNA |
| NM_030903.2 | >gi\|50726880\|ref\|NM_030903.2\| Homo sapiens olfactory receptor, |
| | family 2, subfamily W, member 1 (OR2W1), mRNA |
| NM_030908.1 | >gi\|13929211\|ref\|NM_030908.1\| Homo sapiens olfactory receptor, |
| | family 2, subfamily A, member 4 (OR2A4), mRNA |
| NM_031936.2 | >gi\|19923637\|ref\|NM_031936.2\| Homo sapiens G protein-coupled |
| | receptor 61 (GPR61), mRNA |
| NM_053278.1 | >gi\|16751916\|ref\|NM_053278.1\| Homo sapiens G protein-coupled |
| | receptor 102 (GPR102), mRNA |
| NM_054030.1 | >gi\|16876450\|ref\|NM_054030.1\| Homo sapiens G protein-coupled |
| | receptor MRGX2 (MRGX2), mRNA |
| NM_080817.1 | >gi\|18201869\|ref\|NM_080817.1\| Homo sapiens G protein-coupled |
| | receptor 82 (GPR82), mRNA |
| NM_152430.1 | >gi\|22748910\|ref\|NM_152430.1\| Homo sapiens hypothetical protein |
| | MGC24137 (MGC24137), mRNA |
| NM_177435.1 | >gi\|29171749\|ref\|NM_177435.1\| Homo sapiens peroxisome |
| | proliferative activated receptor, delta (PPARD), transcript variant 2, |
| | mRNA |
| NM_178129.3 | >gi\|38373667\|ref\|NM_178129.3\| Homo sapiens purinergic receptor |
| | P2Y, G-protein coupled, 8 (P2RY8), mRNA |

### REFERENCES CITED

Many modifications and variations of this invention can be made without departing from its spirit and scope, as will be apparent to those skilled in the art. The specific embodiments described herein are offered by way of example only, and the invention is to be limited only by the terms of the appended claims, along with the full scope of equivalents to which such claims are entitled. Such modifications are intended to fall within the scope of the appended claims.

All references, patent and non-patent, cited herein are incorporated herein by reference in their entireties and for all purposes to the same extent as if each individual publication or patent or patent application was specifically and individually indicated to be incorporated by reference in its entirety for all purposes.

1. A positionally addressable array comprising 100 human proteins from the proteins listed in Table 9, Table 11, and Table 13, immobilized on a substrate.

2. The positionally addressable array of claim 1, wherein the array comprises 500 human proteins from the proteins listed in Table 9, Table 11, and Table 13.

3. The positionally addressable array of claim 1, wherein the array comprises 1000 human proteins from the proteins listed in Table 9, Table 11, and Table 13.

4. The positionally addressable array of claim 1, wherein the array comprises 2500 human proteins from the proteins listed in Table 9, Table 11, and Table 13.

5. The positionally addressable array of claim 1, wherein the array comprises 5000 human proteins from the proteins listed in Table 9, Table 11, and Table 13.

6. The positionally addressable array of claim 1, wherein the array comprises 100 of the membrane proteins of Table 15.

7. A positionally addressable array of claim 1, wherein the array comprises 250 of the membrane proteins of Table 15.

8. The positionally addressable array of claim 7, wherein the array comprises 50 of the transmembrane proteins of Table 16.

9. The positionally addressable array of claim 7, wherein the array comprises all of the transmembrane proteins of Table 16.

10. The positionally addressable array of claim 7, wherein the array comprises at least 25 of the G protein coupled receptors (GPCRs) of Table 17.

11. The positionally addressable array of claim 10, wherein the array comprises all of the GPCRs of Table 17.

12. The positionally addressable array of claim 1, wherein proteins are present on the array at a density of between 500 proteins/cm² and 10,000 proteins/cm².

13. The positionally addressable array of claim 1, wherein the proteins are non-denatured proteins.

14. The positionally addressable array of claim 1, wherein the proteins are full-length proteins.

15. The positionally addressable array of claim 1, wherein the proteins are non-denatured, full-length, recombinant fusion proteins comprising a tag.

16. The positionally addressable array of claim 1, wherein the substrate is a functionalized glass slide.

17. The positionally addressable array of claim 16, wherein the functionalized glass slide comprises a polymer comprising an acrylate group, wherein the polymer overlays a glass surface.

18. The positionally addressable array of claim 17, wherein the substrate is a Protein slides II functionalized glass protein microarray substrate available from Full Moon Biosystems

19-22. (canceled)

23. A method for identifying a substrate of an enzyme, comprising contacting the enzyme with a positionally addressable array comprising at least 100 proteins immobilized on a functionalized glass slide, and identifying a protein on the positionally addressable array that is modified by the enzyme, wherein a modifying of the protein by the enzyme indicates that the protein is a substrate for the enzyme.

24. The method of claim 23, wherein the functionalized glass slide comprises a three-dimensional porous surface comprising a polymer overlaying a glass surface.

25. The method of claim 24, wherein the three-dimensional porous surface comprises a polymer comprising acrylate, overlaying a glass surface.

26. The method of claim 25, wherein the functionalized glass substrate comprises multiple functional protein-specific binding sites.

27. The method of claim 26, wherein the substrate is a Protein slides II protein microarray substrate available from Full Moon Biosystems, Inc.

28. The method of claim 23, wherein the enzyme activity is a chemical group transferring enzymatic activity.

29. The method of claim 23, wherein the enzyme activity is kinase activity, protease activity, phosphatase activity, glycosidase, or acetylase activity.

30. The method of claim 23, wherein the enzyme activity is kinase activity.

31-43. (canceled)

44. A method for making an array of proteins, comprising:
cloning each open reading frame from a population of open reading frames into a baculovirus vector to generate a recombinant baculovirus vector comprising a promoter that directs expression of a fusion protein comprising the open reading frame linked to a tag;
expressing the fusion proteins generated for each of the population of open reading frames using insect cells;
isolating the fusion proteins using affinity chromatography directed to the tag; and
spotting the isolated proteins on a substrate.

45. The method of claim 44, wherein the cells are sf9 cells.

46. The method of claim 44, wherein the array of proteins comprises 1000 full length mammalian proteins.

47. The method of claim 46, wherein the proteins are human proteins.

48. The method of claim 47, wherein the proteins comprise at least 250 membrane proteins of Table 15.

48. The method of claim 48, wherein the proteins comprise at least 50 transmembrane proteins of Table 16.

50. The method of claim 49, wherein the proteins comprise at least 25 G-protein coupled receptor proteins of Table 17.

51. The method of claim 44, wherein the tag is a GST tag.

52. The method of claim 48, wherein the proteins are expressed, isolated, and spotted in a high-thoughput manner, and under non-denaturing conditions.

53-61. (canceled)
